SEQUENCE LISTING (818 sequences)

SEQ ID NO: 1 - protein, 23 aa, synthetic construct
MDFQVQIFSFLLISASVIMSRMA

SEQ ID NO: 2 - protein, 21 aa, synthetic construct
MALPVTALLLPLALLLHAARP

SEQ ID NO: 3 - protein, 6 aa, synthetic construct
TFTTYT

SEQ ID NO: 4 - protein, 9 aa, synthetic construct
INPSSGCSD

SEQ ID NO: 5 - protein, 9 aa, synthetic construct
INPSSGYSD

SEQ ID NO: 6 - protein, 14 aa, synthetic construct
RADYGNYEYTWFAY

SEQ ID NO: 7 - protein, 10 aa, synthetic construct
ASQNVGTNVA

SEQ ID NO: 8 - protein, 7 aa, synthetic construct
SASYRYS

SEQ ID NO: 9 - protein, 7 aa, synthetic construct
QQYHTYP

SEQ ID NO: 10 - protein, 5 aa, synthetic construct
AYYWS

SEQ ID NO: 11 - protein, 16 aa, synthetic construct
DINHGGGTNYNPSLKS

SEQ ID NO: 12 - protein, 4 aa, synthetic construct
LTAY

SEQ ID NO: 13 - protein, 11 aa, synthetic construct
RASQGISSWLT

SEQ ID NO: 14 - protein, 7 aa, synthetic construct
AASSLQS

SEQ ID NO: 15 - protein, 9 aa, synthetic construct
QQYDSYPIT

SEQ ID NO: 16 - protein, 4 aa, synthetic construct
KPDK

SEQ ID NO: 17-19 - skipped (3 entries)

SEQ ID NO: 20 - protein, 246 aa, synthetic construct
QVQLQQSGAELARPGASVKMSCKASGYTFTTYTIHWVRRRPGHDLEWIGYINPSSGCSDYNQNFKGKTTLTADKSSNTAYMQLNSLTSEDSAVYYCARRADYGNYEYTWFAYWGQGTTVTVSSSGGGSGGGGSGGGGSVIELTQSPKFMSTSVGDRVNVTYKASQNVGTNVAWFQQKPGQSPKVLIYSASYRYSGVPDRFTGSGSGTDFTLTISNVQSEDLAEYFCQQYHTYPLTFGGGTKLEIKR

SEQ ID NO: 21 - protein, 246 aa, synthetic construct
QVQLQQSGAELARPGASVKMSCKASGYTFTTYTIHWVRQRPGHDLEWIGYINPSSGYSDYNQNFKGKTTLTADKSSNTAYMQLNSLTSEDSAVYYCARRADYGNYEYTWFAYWGQGTTVTVSSGGGGSGGGGSGGGGSDIELTQSPKFMSTSVGDRVNVTYKASQNVGTNVAWFQQKPGQSPKVLIYSASYRYSGVPDRFTGSGSGTDFTLTISNVQSEDLAEYFCQQYHTYPLTFGGGTKLEIKR

SEQ ID NO: 22 - protein, 246 aa, synthetic construct
QVQLQQSGAELARPGASVKMSCKASGYTFTTYTIHWVRRRPGHDLEWIGYINPSSGYSDYNQNFKGKTTLTADKSSNTAYMQLNSLTSEDSAVYYCARRADYGNYEYTWFAYWGQGTTVTVSSSGGGSGGGGSGGGGSVIELTQSPKFMSTSVGDRVNVTYKASQNVGTNVAWFQQKPGQSPKVLIYSASYRYSGVPDRFTGSGSGTDFTLTISNVQSEDLAEYFCQQYHTYPLTFGGGTKLEIKR

SEQ ID NO: 23 - protein, 237 aa, synthetic construct
DIQMTQSPTSLSASVGDRVTITCRASQGISSWLTWYQQKPEKAPKSLIYAASSLQSGVPSRFSGSGSGTDFTLTISSLQPEDFATYYCQQYDSYPITFGQGTRLEIKGSTSGSGKPGSGEGSTKGQVQLQQWGAGLLKPSETLSLTCAVYGGSFSAYYWSWIRQPPGKGLEWIGDINHGGGTNYNPSLKSRVTISVDTSKNQFSLKLNSVTAADTAVYYCASLTAYWGQGSLVTVSS

SEQ ID NO: 24 - protein, 123 aa, synthetic construct
QVQLQQSGAELARPGASVKMSCKASGYTFTTYTIHWVRRRPGHDLEWIGYINPSSGCSDYNQNFKGKTTLTADKSSNTAYMQLNSLTSEDSAVYYCARRADYGNYEYTWFAYWGQGTTVTVSS

SEQ ID NO: 25 - protein, 111 aa, synthetic construct
VIELTQSPKFMSTSVGDRVNVTYKASQNVGTNVAWFQQKPGQSPKVLIYSASYRYSGVPDRFTGSGSGTDFTLTISNVQSEDLAEYFCQQYHTYPLTFGGGTKLEIKRSDP

SEQ ID NO: 26 - protein, 123 aa, synthetic construct
QVQLQQSGAELARPGASVKMSCKASGYTFTTYTIHWVRQRPGHDLEWIGYINPSSGYSDYNQNFKGKTTLTADKSSNTAYMQLNSLTSEDSAVYYCARRADYGNYEYTWFAYWGQGTTVTVSS

SEQ ID NO: 27 - protein, 111 aa, synthetic construct
DIELTQSPKFMSTSVGDRVNVTYKASQNVGTNVAWFQQKPGQSPKVLIYSASYRYSGVPDRFTGSGSGTDFTLTISNVQSEDLAEYFCQQYHTYPLTFGGGTKLEIKRSDP

SEQ ID NO: 28 - protein, 123 aa, synthetic construct
QVQLQQSGAELARPGASVKMSCKASGYTFTTYTIHWVRRRPGHDLEWIGYINPSSGYSDYNQNFKGKTTLTADKSSNTAYMQLNSLTSEDSAVYYCARRADYGNYEYTWFAYWGQGTTVTVSS

SEQ ID NO: 29 - protein, 123 aa, synthetic construct
EVKLQQSGAELARPGASVKMSCKASGYTFTTYTIHWVRQRPGHDLEWIGYINPSSGYSDYNQNFKGKTTLTADKSSNTAYMQLNSLTSEDSAVYYCARRADYGNYEYTWFAYWGQGTTVTVSS

SEQ ID NO: 30 - protein, 111 aa, synthetic construct
DIELTQSPKFMSTSVGDRVNVTCKASQNVGTNVAWFQQKPGQSPKVLIYSASYRYSGVPDRFTGSGSGTDFTLTISNVQSEDLAEYFCQQYHTYPLTFGGGTKLEIKRSDP

SEQ ID NO: 31 - protein, 112 aa, synthetic construct
QVQLQQWGAGLLKPSETLSLTCAVYGGSFSAYYWSWIRQPPGKGLEWIGDINHGGGTNYNPSLKSRVTISVDTSKNQFSLKLNSVTAADTAVYYCASLTAYWGQGSLVTVSS

SEQ ID NO: 32 - protein, 107 aa, synthetic construct
DIQMTQSPTSLSASVGDRVTITCRASQGISSWLTWYQQKPEKAPKSLIYAASSLQSGVPSRFSGSGSGTDFTLTISSLQPEDFATYYCQQYDSYPITFGQGTRLEIK

SEQ ID NO: 33-39 - skipped (7 entries)

SEQ ID NO: 40 - protein, 5 aa, synthetic construct
DGGGS

SEQ ID NO: 41 - protein, 5 aa, synthetic construct
TGEKP

SEQ ID NO: 42 - protein, 4 aa, synthetic construct
GGRR

SEQ ID NO: 43 - protein, 25 aa, synthetic construct
  VARIANT 6..25: note = GGGGS repeats may be deleted
GGGGSGGGGSGGGGSGGGGSGGGGS

SEQ ID NO: 44 - protein, 18 aa, synthetic construct
KESGSVSSEQLAQFRSLD

SEQ ID NO: 45 - protein, 8 aa, synthetic construct
GGRRGGGS

SEQ ID NO: 46 - protein, 9 aa, synthetic construct
LRQRDGERP

SEQ ID NO: 47 - protein, 12 aa, synthetic construct
LRQKDGGGSERP

SEQ ID NO: 48 - protein, 16 aa, synthetic construct
LRQKDGGGSGGGSERP

SEQ ID NO: 49 - protein, 18 aa, synthetic construct
GSTSGSGKPGSGEGSTKG

SEQ ID NO: 50 - protein, 45 aa, synthetic construct
TTTPAPRPPTPAPTIASQPLSLRPEACRPAAGGAVHTRGLDFACD

SEQ ID NO: 51 - protein, 39 aa, synthetic construct
IEVMYPPPYLDNEKSNGTIIHVKGKHLCPSPLFPGPSKP

SEQ ID NO: 52 - protein, 219 aa, synthetic construct
PCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 107 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 27 aa, synthetic construct
FWVLVVVGGVLACYSLLVTVAFIIFWV

SEQ ID NO: 55 - protein, 21 aa, synthetic construct
IYIWAPLAGTCGVLLLSLVIT

SEQ ID NO: 56 - skipped

SEQ ID NO: 57 - protein, 41 aa, synthetic construct
RSKRSRLLHSDYMNMTPRRPGPTRKHYQPYAPPRDFAAYRS

SEQ ID NO: 58 - protein, 41 aa, synthetic construct
RSKRSRLLHSDYMNMTPRRPGPTRKHYQAYAAARDFAAYRS

SEQ ID NO: 59 - protein, 42 aa, synthetic construct
KRGRKKLLYIFKQPFMRPVQTTQEEDGCSCRFPEEEEGGCEL

SEQ ID NO: 60 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 113 aa, synthetic construct
LRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - protein, 666 aa, synthetic construct
QVQLQQSGAELARPGASVKMSCKASGYTFTTYTIHWVRRRPGHDLEWIGYINPSSGCSDYNQNFKGKTTLTADKSSNTAYMQLNSLTSEDSAVYYCARRADYGNYEYTWFAYWGQGTTVTVSSSGGGSGGGGSGGGGSVIELTQSPKFMSTSVGDRVNVTYKASQNVGTNVAWFQQKPGQSPKVLIYSASYRYSGVPDRFTGSGSGTDFTLTISNVQSEDLAEYFCQQYHTYPLTFGGGTKLEIKRSDPAEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQAYAAARDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - protein, 554 aa, synthetic construct
QVQLQQSGAELARPGASVKMSCKASGYTFTTYTIHWVRRRPGHDLEWIGYINPSSGCSDYNQNFKGKTTLTADKSSNTAYMQLNSLTSEDSAVYYCARRADYGNYEYTWFAYWGQGTTVTVSSSGGGSGGGGSGGGGSVIELTQSPKFMSTSVGDRVNVTYKASQNVGTNVAWFQQKPGQSPKVLIYSASYRYSGVPDRFTGSGSGTDFTLTISNVQSEDLAEYFCQQYHTYPLTFGGGTKLEIKRSDPAEPKSPDKTHTCPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQAYAAARDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - protein, 666 aa, synthetic construct
QVQLQQSGAELARPGASVKMSCKASGYTFTTYTIHWVRRRPGHDLEWIGYINPSSGCSDYNQNFKGKTTLTADKSSNTAYMQLNSLTSEDSAVYYCARRADYGNYEYTWFAYWGQGTTVTVSSSGGGSGGGGSGGGGSVIELTQSPKFMSTSVGDRVNVTYKASQNVGTNVAWFQQKPGQSPKVLIYSASYRYSGVPDRFTGSGSGTDFTLTISNVQSEDLAEYFCQQYHTYPLTFGGGTKLEIKRSDPAEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 66 - protein, 480 aa, synthetic construct
QVQLQQSGAELARPGASVKMSCKASGYTFTTYTIHWVRRRPGHDLEWIGYINPSSGCSDYNQNFKGKTTLTADKSSNTAYMQLNSLTSEDSAVYYCARRADYGNYEYTWFAYWGQGTTVTVSSSGGGSGGGGSGGGGSVIELTQSPKFMSTSVGDRVNVTYKASQNVGTNVAWFQQKPGQSPKVLIYSASYRYSGVPDRFTGSGSGTDFTLTISNVQSEDLAEYFCQQYHTYPLTFGGGTKLEIKRSDPATTTPAPRPPTPAPTIASQPLSLRPEACRPAAGGAVHTRGLDFACDKDPKFWVLVVVGGVLACYSLLVTVAFIIFWVRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - protein, 476 aa, synthetic construct
QVQLQQSGAELARPGASVKMSCKASGYTFTTYTIHWVRRRPGHDLEWIGYINPSSGCSDYNQNFKGKTTLTADKSSNTAYMQLNSLTSEDSAVYYCARRADYGNYEYTWFAYWGQGTTVTVSSSGGGSGGGGSGGGGSVIELTQSPKFMSTSVGDRVNVTYKASQNVGTNVAWFQQKPGQSPKVLIYSASYRYSGVPDRFTGSGSGTDFTLTISNVQSEDLAEYFCQQYHTYPLTFGGGTKLEIKRSDPATTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - protein, 480 aa, synthetic construct
QVQLQQSGAELARPGASVKMSCKASGYTFTTYTIHWVRQRPGHDLEWIGYINPSSGYSDYNQNFKGKTTLTADKSSNTAYMQLNSLTSEDSAVYYCARRADYGNYEYTWFAYWGQGTTVTVSSGGGGSGGGGSGGGGSDIELTQSPKFMSTSVGDRVNVTYKASQNVGTNVAWFQQKPGQSPKVLIYSASYRYSGVPDRFTGSGSGTDFTLTISNVQSEDLAEYFCQQYHTYPLTFGGGTKLEIKRSDPATTTPAPRPPTPAPTIASQPLSLRPEACRPAAGGAVHTRGLDFACDKDPKFWVLVVVGGVLACYSLLVTVAFIIFWVRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 69 - protein, 480 aa, synthetic construct
QVQLQQSGAELARPGASVKMSCKASGYTFTTYTIHWVRRRPGHDLEWIGYINPSSGYSDYNQNFKGKTTLTADKSSNTAYMQLNSLTSEDSAVYYCARRADYGNYEYTWFAYWGQGTTVTVSSSGGGSGGGGSGGGGSVIELTQSPKFMSTSVGDRVNVTYKASQNVGTNVAWFQQKPGQSPKVLIYSASYRYSGVPDRFTGSGSGTDFTLTISNVQSEDLAEYFCQQYHTYPLTFGGGTKLEIKRSDPATTTPAPRPPTPAPTIASQPLSLRPEACRPAAGGAVHTRGLDFACDKDPKFWVLVVVGGVLACYSLLVTVAFIIFWVRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 70 - protein, 456 aa, synthetic construct
DIQMTQSPTSLSASVGDRVTITCRASQGISSWLTWYQQKPEKAPKSLIYAASSLQSGVPSRFSGSGSGTDFTLTISSLQPEDFATYYCQQYDSYPITFGQGTRLEIKGSTSGSGKPGSGEGSTKGQVQLQQWGAGLLKPSETLSLTCAVYGGSFSAYYWSWIRQPPGKGLEWIGDINHGGGTNYNPSLKSRVTISVDTSKNQFSLKLNSVTAADTAVYYCASLTAYWGQGSLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71 - protein, 456 aa, synthetic construct
DIQMTQSPTSLSASVGDRVTITCRASQGISSWLTWYQQKPEKAPKSLIYAASSLQSGVPSRFSGSGSGTDFTLTISSLQPEDFATYYCQQYDSYPITFGQGTRLEIKGSTSGSGKPGSGEGSTKGQVQLQQWGAGLLKPSETLSLTCAVYGGSFSAYYWSWIRQPPGKGLEWIGDINHGGGTNYNPSLKSRVTISVDTSKNQFSLKLNSVTAADTAVYYCASLTAYWGQGSLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQAYAAARDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 72 - protein, 462 aa, synthetic construct
DIQMTQSPTSLSASVGDRVTITCRASQGISSWLTWYQQKPEKAPKSLIYAASSLQSGVPSRFSGSGSGTDFTLTISSLQPEDFATYYCQQYDSYPITFGQGTRLEIKGSTSGSGKPGSGEGSTKGQVQLQQWGAGLLKPSETLSLTCAVYGGSFSAYYWSWIRQPPGKGLEWIGDINHGGGTNYNPSLKSRVTISVDTSKNQFSLKLNSVTAADTAVYYCASLTAYWGQGSLVTVSSTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 73 - protein, 481 aa, synthetic construct
MALPVTALLLPLALLLHAARPDIQMTQSPTSLSASVGDRVTITCRASQGISSWLTWYQQKPEKAPKSLIYAASSLQSGVPSRFSGSGSGTDFTLTISSLQPEDFATYYCQQYDSYPITFGQGTRLEIKGSTSGSGKPGSGEGSTKGQVQLQQWGAGLLKPSETLSLTCAVYGGSFSAYYWSWIRQPPGKGLEWIGDINHGGGTNYNPSLKSRVTISVDTSKNQFSLKLNSVTAADTAVYYCASLTAYWGQGSLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - protein, 483 aa, synthetic construct
MALPVTALLLPLALLLHAARPDIQMTQSPTSLSASVGDRVTITCRASQGISSWLTWYQQKPEKAPKSLIYAASSLQSGVPSRFSGSGSGTDFTLTISSLQPEDFATYYCQQYDSYPITFGQGTRLEIKGSTSGSGKPGSGEGSTKGQVQLQQWGAGLLKPSETLSLTCAVYGGSFSAYYWSWIRQPPGKGLEWIGDINHGGGTNYNPSLKSRVTISVDTSKNQFSLKLNSVTAADTAVYYCASLTAYWGQGSLVTVSSTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75-230 - skipped (156 entries)

SEQ ID NO: 231 - RNA, 100 nt, synthetic construct
  source 1..100: mol_type = other RNA
  misc_feature 1..4: note = Phosphorothioate linkage
  misc_feature 97..100: note = Phosphorothioate linkage
  modified_base 1..3: mod_base = OTHER; note = 2'-O-methyl nucleotide
  modified_base 29..40: mod_base = OTHER; note = 2'-O-methyl nucleotide
  modified_base 69..100: mod_base = OTHER; note = 2'-O-methyl nucleotide
ctctcagctggtacacggcagttttagagctagaaatagcaagttaaaataaggctagtccgttatcaacttgaaaaagtggcaccgagtcggtgctttt

SEQ ID NO: 232 - skipped

SEQ ID NO: 233 - RNA, 20 nt, synthetic construct
  source 1..20: mol_type = other RNA
ctctcagctggtacacggca

SEQ ID NO: 234 - skipped

SEQ ID NO: 235 - DNA, 2067 bp, synthetic construct
  source 1..2067: mol_type = other DNA
atggacttccaggtgcagatcttcagcttcctgctgatcagcgccagcgtgatcatgagccggatggcccaggtgcagctgcagcagagcggcgccgagctggcccggcccggcgccagcgtgaagatgagctgcaaggccagcggctacaccttcaccacctacaccatccactgggtgcggcggcggcccggccacgacctggagtggatcggctacatcaaccccagcagcggctgcagcgactacaaccagaacttcaagggcaagaccaccctgaccgccgacaagagcagcaacaccgcctacatgcagctgaacagcctgaccagcgaggacagcgccgtgtactactgcgcccggcgggccgactacggcaactacgagtacacctggttcgcctactggggccagggcaccaccgtgaccgtgagcagcagcggcggcggcagcggcggcggcggcagcggcggcggcggcagcgtgatcgagctgacccagagccccaagttcatgagcaccagcgtgggcgaccgggtgaacgtgacctacaaggccagccagaacgtgggcaccaacgtggcctggttccagcagaagcccggccagagccccaaggtgctgatctacagcgccagctaccggtacagcggcgtgcccgaccggttcaccggcagcggcagcggcaccgacttcaccctgaccatcagcaacgtgcagagcgaggacctggccgagtacttctgccagcagtaccacacctaccccctgaccttcggcggcggcaccaagctggagatcaagcggagcgaccccgccgagcccaagagccccgacaagacccacacctgccccccctgccccgccccccccgtggccggccccagcgtgttcctgttcccccccaagcccaaggacaccctgatgatcgcccggacccccgaggtgacctgcgtggtggtggacgtgagccacgaggaccccgaggtgaagttcaactggtacgtggacggcgtggaggtgcacaacgccaagaccaagccccgggaggagcagtacaacagcacctaccgggtggtgagcgtgctgaccgtgctgcaccaggactggctgaacggcaaggagtacaagtgcaaggtgagcaacaaggccctgcccgcccccatcgagaagaccatcagcaaggccaagggccagccccgggagccccaggtgtacaccctgccccccagccgggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccggtggcagcagggcaacgtgttcagctgcagcgtgatgcacgaggccctgcacaaccactacacccagaagagcctgagcctgagccccggcaagaaggaccccaagttctgggtgctggtggtggtgggcggcgtgctggcctgctacagcctgctggtgaccgtggccttcatcatcttctgggtgcggagcaagcggagccggctgctgcacagcgactacatgaacatgaccccccggcggcccggccccacccggaagcactaccaggcctacgccgccgcccgggacttcgccgcctaccggagcctgcgggtgaagttcagccggagcgccgacgcccccgcctaccagcagggccagaaccagctgtacaacgagctgaacctgggccggcgggaggagtacgacgtgctggacaagcggcggggccgggaccccgagatgggcggcaagccccggcggaagaacccccaggagggcctgtacaacgagctgcagaaggacaagatggccgaggcctacagcgagatcggcatgaagggcgagcggcggcggggcaagggccacgacggcctgtaccagggcctgagcaccgccaccaaggacacctacgacgccctgcacatgcaggccctgcccccccgg

SEQ ID NO: 236 - skipped

SEQ ID NO: 237 - DNA, 1731 bp, synthetic construct
  source 1..1731: mol_type = other DNA
atggacttccaggtgcagatcttcagcttcctgctgatcagcgccagcgtgatcatgagccggatggcccaggtgcagctgcagcagagcggcgccgagctggcccggcccggcgccagcgtgaagatgagctgcaaggccagcggctacaccttcaccacctacaccatccactgggtgcggcggcggcccggccacgacctggagtggatcggctacatcaaccccagcagcggctgcagcgactacaaccagaacttcaagggcaagaccaccctgaccgccgacaagagcagcaacaccgcctacatgcagctgaacagcctgaccagcgaggacagcgccgtgtactactgcgcccggcgggccgactacggcaactacgagtacacctggttcgcctactggggccagggcaccaccgtgaccgtgagcagcagcggcggcggcagcggcggcggcggcagcggcggcggcggcagcgtgatcgagctgacccagagccccaagttcatgagcaccagcgtgggcgaccgggtgaacgtgacctacaaggccagccagaacgtgggcaccaacgtggcctggttccagcagaagcccggccagagccccaaggtgctgatctacagcgccagctaccggtacagcggcgtgcccgaccggttcaccggcagcggcagcggcaccgacttcaccctgaccatcagcaacgtgcagagcgaggacctggccgagtacttctgccagcagtaccacacctaccccctgaccttcggcggcggcaccaagctggagatcaagcggagcgaccccgccgagcccaagagccccgacaagacccacacctgccccggccagccccgggagccccaggtgtacaccctgccccccagccgggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccggtggcagcagggcaacgtgttcagctgcagcgtgatgcacgaggccctgcacaaccactacacccagaagagcctgagcctgagccccggcaagaaggaccccaagttctgggtgctggtggtggtgggcggcgtgctggcctgctacagcctgctggtgaccgtggccttcatcatcttctgggtgcggagcaagcggagccggctgctgcacagcgactacatgaacatgaccccccggcggcccggccccacccggaagcactaccaggcctacgccgccgcccgggacttcgccgcctaccggagcctgcgggtgaagttcagccggagcgccgacgcccccgcctaccagcagggccagaaccagctgtacaacgagctgaacctgggccggcgggaggagtacgacgtgctggacaagcggcggggccgggaccccgagatgggcggcaagccccggcggaagaacccccaggagggcctgtacaacgagctgcagaaggacaagatggccgaggcctacagcgagatcggcatgaagggcgagcggcggcggggcaagggccacgacggcctgtaccagggcctgagcaccgccaccaaggacacctacgacgccctgcacatgcaggccctgcccccccgg

SEQ ID NO: 238 - skipped

SEQ ID NO: 239 - DNA, 2070 bp, synthetic construct
  source 1..2070: mol_type = other DNA
atggacttccaggtgcagatcttcagcttcctgctgatcagcgccagcgtgatcatgagccggatggcccaggtgcagctgcagcagagcggcgccgagctggcccggcccggcgccagcgtgaagatgagctgcaaggccagcggctacaccttcaccacctacaccatccactgggtgcggcggcggcccggccacgacctggagtggatcggctacatcaaccccagcagcggctgcagcgactacaaccagaacttcaagggcaagaccaccctgaccgccgacaagagcagcaacaccgcctacatgcagctgaacagcctgaccagcgaggacagcgccgtgtactactgcgcccggcgggccgactacggcaactacgagtacacctggttcgcctactggggccagggcaccaccgtgaccgtgagcagcagcggcggcggcagcggcggcggcggcagcggcggcggcggcagcgtgatcgagctgacccagagccccaagttcatgagcaccagcgtgggcgaccgggtgaacgtgacctacaaggccagccagaacgtgggcaccaacgtggcctggttccagcagaagcccggccagagccccaaggtgctgatctacagcgccagctaccggtacagcggcgtgcccgaccggttcaccggcagcggcagcggcaccgacttcaccctgaccatcagcaacgtgcagagcgaggacctggccgagtacttctgccagcagtaccacacctaccccctgaccttcggcggcggcaccaagctggagatcaagcggagcgaccccgccgagcccaagagccccgacaagacccacacctgccccccctgccccgccccccccgtggccggccccagcgtgttcctgttcccccccaagcccaaggacaccctgatgatcgcccggacccccgaggtgacctgcgtggtggtggacgtgagccacgaggaccccgaggtgaagttcaactggtacgtggacggcgtggaggtgcacaacgccaagaccaagccccgggaggagcagtacaacagcacctaccgggtggtgagcgtgctgaccgtgctgcaccaggactggctgaacggcaaggagtacaagtgcaaggtgagcaacaaggccctgcccgcccccatcgagaagaccatcagcaaggccaagggccagccccgggagccccaggtgtacaccctgccccccagccgggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccggtggcagcagggcaacgtgttcagctgcagcgtgatgcacgaggccctgcacaaccactacacccagaagagcctgagcctgagccccggcaagaaggaccccaagttctgggtgctggtggtggtgggcggcgtgctggcctgctacagcctgctggtgaccgtggccttcatcatcttctgggtgcggagcaagcggagccggctgctgcacagcgactacatgaacatgaccccccggcggcccggccccacccggaagcactaccagccctacgcccccccccgggacttcgccgcctaccggagcctgcgggtgaagttcagccggagcgccgacgcccccgcctaccagcagggccagaaccagctgtacaacgagctgaacctgggccggcgggaggagtacgacgtgctggacaagcggcggggccgggaccccgagatgggcggcaagccccggcggaagaacccccaggagggcctgtacaacgagctgcagaaggacaagatggccgaggcctacagcgagatcggcatgaagggcgagcggcggcggggcaagggccacgacggcctgtaccagggcctgagcaccgccaccaaggacacctacgacgccctgcacatgcaggccctgcccccccggtaa

SEQ ID NO: 240 - skipped

SEQ ID NO: 241 - DNA, 1509 bp, synthetic construct
  source 1..1509: mol_type = other DNA
atggacttccaggtgcagatcttcagcttcctgctgatcagcgccagcgtgatcatgagccggatggcccaggtgcagctgcagcagagcggcgccgagctggcccggcccggcgccagcgtgaagatgagctgcaaggccagcggctacaccttcaccacctacaccatccactgggtgcggcggcggcccggccacgacctggagtggatcggctacatcaaccccagcagcggctgcagcgactacaaccagaacttcaagggcaagaccaccctgaccgccgacaagagcagcaacaccgcctacatgcagctgaacagcctgaccagcgaggacagcgccgtgtactactgcgcccggcgggccgactacggcaactacgagtacacctggttcgcctactggggccagggcaccaccgtgaccgtgagcagcagcggcggcggcagcggcggcggcggcagcggcggcggcggcagcgtgatcgagctgacccagagccccaagttcatgagcaccagcgtgggcgaccgggtgaacgtgacctacaaggccagccagaacgtgggcaccaacgtggcctggttccagcagaagcccggccagagccccaaggtgctgatctacagcgccagctaccggtacagcggcgtgcccgaccggttcaccggcagcggcagcggcaccgacttcaccctgaccatcagcaacgtgcagagcgaggacctggccgagtacttctgccagcagtaccacacctaccccctgaccttcggcggcggcaccaagctggagatcaagcggagcgaccccgccaccaccacccccgccccccggccccccacccccgcccccaccatcgccagccagcccctgagcctgcggcccgaggcctgccggcccgccgccggcggcgccgtgcacacccggggcctggacttcgcctgcgacaaggaccccaagttctgggtgctggtggtggtgggcggcgtgctggcctgctacagcctgctggtgaccgtggccttcatcatcttctgggtgcggagcaagcggagccggctgctgcacagcgactacatgaacatgaccccccggcggcccggccccacccggaagcactaccagccctacgcccccccccgggacttcgccgcctaccggagcctgcgggtgaagttcagccggagcgccgacgcccccgcctaccagcagggccagaaccagctgtacaacgagctgaacctgggccggcgggaggagtacgacgtgctggacaagcggcggggccgggaccccgagatgggcggcaagccccggcggaagaacccccaggagggcctgtacaacgagctgcagaaggacaagatggccgaggcctacagcgagatcggcatgaagggcgagcggcggcggggcaagggccacgacggcctgtaccagggcctgagcaccgccaccaaggacacctacgacgccctgcacatgcaggccctgcccccccgg

SEQ ID NO: 242 - skipped

SEQ ID NO: 243 - DNA, 1431 bp, synthetic construct
  source 1..1431: mol_type = other DNA
atggccctgcccgtgaccgccctgctgctgcccctggccctgctgctgcacgccgcccggcccgacatccagatgacccagagccccaccagcctgagcgccagcgtgggcgaccgggtgaccatcacctgccgggccagccagggcatcagcagctggctgacctggtaccagcagaagcccgagaaggcccccaagagcctgatctacgccgccagcagcctgcagagcggcgtgcccagccggttcagcggcagcggcagcggcaccgacttcaccctgaccatcagcagcctgcagcccgaggacttcgccacctactactgccagcagtacgacagctaccccatcaccttcggccagggcacccggctggagatcaagggcagcaccagcggcagcggcaagcccggcagcggcgagggcagcaccaagggccaggtgcagctgcagcagtggggcgccggcctgctgaagcccagcgagaccctgagcctgacctgcgccgtgtacggcggcagcttcagcgcctactactggagctggatccggcagccccccggcaagggcctggagtggatcggcgacatcaaccacggcggcggcaccaactacaaccccagcctgaagagccgggtgaccatcagcgtggacaccagcaagaaccagttcagcctgaagctgaacagcgtgaccgccgccgacaccgccgtgtactactgcgccagcctgaccgcctactggggccagggcagcctggtgaccgtgagcagcatcgaggtgatgtacccccccccctacctggacaacgagaagagcaacggcaccatcatccacgtgaagggcaagcacctgtgccccagccccctgttccccggccccagcaagcccttctgggtgctggtggtggtgggcggcgtgctggcctgctacagcctgctggtgaccgtggccttcatcatcttctgggtgcggagcaagcggagccggctgctgcacagcgactacatgaacatgaccccccggcggcccggccccacccggaagcactaccagccctacgcccccccccgggacttcgccgcctaccggagccgggtgaagttcagccggagcgccgacgcccccgcctaccagcagggccagaaccagctgtacaacgagctgaacctgggccggcgggaggagtacgacgtgctggacaagcggcggggccgggaccccgagatgggcggcaagccccggcggaagaacccccaggagggcctgtacaacgagctgcagaaggacaagatggccgaggcctacagcgagatcggcatgaagggcgagcggcggcggggcaagggccacgacggcctgtaccagggcctgagcaccgccaccaaggacacctacgacgccctgcacatgcaggccctgcccccccgg

SEQ ID NO: 244 - skipped

SEQ ID NO: 245 - DNA, 1431 bp, synthetic construct
  source 1..1431: mol_type = other DNA
atggccctgcccgtgaccgccctgctgctgcccctggccctgctgctgcacgccgcccggcccgacatccagatgacccagagccccaccagcctgagcgccagcgtgggcgaccgggtgaccatcacctgccgggccagccagggcatcagcagctggctgacctggtaccagcagaagcccgagaaggcccccaagagcctgatctacgccgccagcagcctgcagagcggcgtgcccagccggttcagcggcagcggcagcggcaccgacttcaccctgaccatcagcagcctgcagcccgaggacttcgccacctactactgccagcagtacgacagctaccccatcaccttcggccagggcacccggctggagatcaagggcagcaccagcggcagcggcaagcccggcagcggcgagggcagcaccaagggccaggtgcagctgcagcagtggggcgccggcctgctgaagcccagcgagaccctgagcctgacctgcgccgtgtacggcggcagcttcagcgcctactactggagctggatccggcagccccccggcaagggcctggagtggatcggcgacatcaaccacggcggcggcaccaactacaaccccagcctgaagagccgggtgaccatcagcgtggacaccagcaagaaccagttcagcctgaagctgaacagcgtgaccgccgccgacaccgccgtgtactactgcgccagcctgaccgcctactggggccagggcagcctggtgaccgtgagcagcatcgaggtgatgtacccccccccctacctggacaacgagaagagcaacggcaccatcatccacgtgaagggcaagcacctgtgccccagccccctgttccccggccccagcaagcccttctgggtgctggtggtggtgggcggcgtgctggcctgctacagcctgctggtgaccgtggccttcatcatcttctgggtgcggagcaagcggagccggctgctgcacagcgactacatgaacatgaccccccggcggcccggccccacccggaagcactaccaggcctacgccgccgcccgggacttcgccgcctaccggagccgggtgaagttcagccggagcgccgacgcccccgcctaccagcagggccagaaccagctgtacaacgagctgaacctgggccggcgggaggagtacgacgtgctggacaagcggcggggccgggaccccgagatgggcggcaagccccggcggaagaacccccaggagggcctgtacaacgagctgcagaaggacaagatggccgaggcctacagcgagatcggcatgaagggcgagcggcggcggggcaagggccacgacggcctgtaccagggcctgagcaccgccaccaaggacacctacgacgccctgcacatgcaggccctgcccccccgg

SEQ ID NO: 246 - skipped

SEQ ID NO: 247 - DNA, 1452 bp, synthetic construct
  source 1..1452: mol_type = other DNA
atggccctgcccgtgaccgccctgctgctgcccctggccctgctgctgcacgccgcccggcccgacatccagatgacccagagccccaccagcctgagcgccagcgtgggcgaccgggtgaccatcacctgccgggccagccagggcatcagcagctggctgacctggtaccagcagaagcccgagaaggcccccaagagcctgatctacgccgccagcagcctgcagagcggcgtgcccagccggttcagcggcagcggcagcggcaccgacttcaccctgaccatcagcagcctgcagcccgaggacttcgccacctactactgccagcagtacgacagctaccccatcaccttcggccagggcacccggctggagatcaagggcagcaccagcggcagcggcaagcccggcagcggcgagggcagcaccaagggccaggtgcagctgcagcagtggggcgccggcctgctgaagcccagcgagaccctgagcctgacctgcgccgtgtacggcggcagcttcagcgcctactactggagctggatccggcagccccccggcaagggcctggagtggatcggcgacatcaaccacggcggcggcaccaactacaaccccagcctgaagagccgggtgaccatcagcgtggacaccagcaagaaccagttcagcctgaagctgaacagcgtgaccgccgccgacaccgccgtgtactactgcgccagcctgaccgcctactggggccagggcagcctggtgaccgtgagcagcaccaccacccccgccccccggccccccacccccgcccccaccatcgccagccagcccctgagcctgcggcccgaggcctgccggcccgccgccggcggcgccgtgcacacccggggcctggacttcgcctgcgacttctgggtgctggtggtggtgggcggcgtgctggcctgctacagcctgctggtgaccgtggccttcatcatcttctgggtgcggagcaagcggagccggctgctgcacagcgactacatgaacatgaccccccggcggcccggccccacccggaagcactaccagccctacgcccccccccgggacttcgccgcctaccggagccgggtgaagttcagccggagcgccgacgcccccgcctaccagcagggccagaaccagctgtacaacgagctgaacctgggccggcgggaggagtacgacgtgctggacaagcggcggggccgggaccccgagatgggcggcaagccccggcggaagaacccccaggagggcctgtacaacgagctgcagaaggacaagatggccgaggcctacagcgagatcggcatgaagggcgagcggcggcggggcaagggccacgacggcctgtaccagggcctgagcaccgccaccaaggacacctacgacgccctgcacatgcaggccctgcccccccggtaa

SEQ ID NO: 248 - skipped

SEQ ID NO: 249 - DNA, 1443 bp, synthetic construct
  source 1..1443: mol_type = other DNA
atggccttaccagtgaccgccttgctcctgccgctggccttgctgctccacgccgccaggccggatattcagatgactcaatctccaacctctctctcagcaagtgttggcgacagagttaccatcacttgcagggcaagtcagggtatcagcagctggctgacatggtatcaacagaagccagagaaagctcctaagagtctcatatacgctgcaagctcactccagtcaggagtgccctccaggttctccgggtccggatcagggactgactttaccctcactattagcagcctccagccagaggacttcgctacatactactgtcagcagtatgacagttaccctatcacctttggccagggaactcggctggagatcaagggttctacttctggtagcgggaaacccggtagcggtgaaggcagcactaagggacaggttcagctccaacagtggggcgctgggctgctgaaacccagcgagactctgagtctcacttgcgcagtgtacggtggttcattcagcgcctactattggagttggataaggcagcctccagggaaaggcctggaatggatcggcgacatcaatcatggcggtggaaccaactataacccatctctgaagagtcgcgtgacaatttctgttgataccagcaagaatcagtttagtctgaaactgaactccgtgactgccgcagatactgccgtttactactgtgccagcctgaccgcatactggggccagggtagcctcgtcaccgtgtctagtaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 250 - skipped

SEQ ID NO: 251 - DNA, 1449 bp, synthetic construct
  source 1..1449: mol_type = other DNA
atggccttaccagtgaccgccttgctcctgccgctggccttgctgctccacgccgccaggccggatattcagatgactcaatctccaacctctctctcagcaagtgttggcgacagagttaccatcacttgcagggcaagtcagggtatcagcagctggctgacatggtatcaacagaagccagagaaagctcctaagagtctcatatacgctgcaagctcactccagtcaggagtgccctccaggttctccgggtccggatcagggactgactttaccctcactattagcagcctccagccagaggacttcgctacatactactgtcagcagtatgacagttaccctatcacctttggccagggaactcggctggagatcaagggttctacttctggtagcgggaaacccggtagcggtgaaggcagcactaagggacaggttcagctccaacagtggggcgctgggctgctgaaacccagcgagactctgagtctcacttgcgcagtgtacggtggttcattcagcgcctactattggagttggataaggcagcctccagggaaaggcctggaatggatcggcgacatcaatcatggcggtggaaccaactataacccatctctgaagagtcgcgtgacaatttctgttgataccagcaagaatcagtttagtctgaaactgaactccgtgactgccgcagatactgccgtttactactgtgccagcctgaccgcatactggggccagggtagcctcgtcaccgtgtctagtaccactaccccagcaccgaggccacccaccccggctcctaccatcgcctcccagcctctgtccctgcgtccggaggcatgtagacccgcagctggtggggccgtgcatacccggggtcttgacttcgcctgcgatttctgggtgctggtcgttgtgggcggcgtgctggcctgctacagcctgctggtgacagtggccttcatcatcttttgggtgaggagcaagcggagcagactgctgcacagcgactacatgaacatgaccccccggaggcctggccccacccggaagcactaccagccctacgcccctcccagggatttcgccgcctaccggagccgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 252 - skipped

SEQ ID NO: 253 - DNA, 1497 bp, synthetic construct
  source 1..1497: mol_type = other DNA
atggacttccaggtgcagatcttcagcttcctgctgatcagcgccagcgtgatcatgagccggatggcccaggtgcagctgcagcagagcggcgccgagctggcccggcccggcgccagcgtgaagatgagctgcaaggccagcggctacaccttcaccacctacaccatccactgggtgcggcggcggcccggccacgacctggagtggatcggctacatcaaccccagcagcggctgcagcgactacaaccagaacttcaagggcaagaccaccctgaccgccgacaagagcagcaacaccgcctacatgcagctgaacagcctgaccagcgaggacagcgccgtgtactactgcgcccggcgggccgactacggcaactacgagtacacctggttcgcctactggggccagggcaccaccgtgaccgtgagcagcagcggcggcggcagcggcggcggcggcagcggcggcggcggcagcgtgatcgagctgacccagagccccaagttcatgagcaccagcgtgggcgaccgggtgaacgtgacctacaaggccagccagaacgtgggcaccaacgtggcctggttccagcagaagcccggccagagccccaaggtgctgatctacagcgccagctaccggtacagcggcgtgcccgaccggttcaccggcagcggcagcggcaccgacttcaccctgaccatcagcaacgtgcagagcgaggacctggccgagtacttctgccagcagtaccacacctaccccctgaccttcggcggcggcaccaagctggagatcaagcggagcgaccccgccaccaccacccccgccccccggccccccacccccgcccccaccatcgccagccagcccctgagcctgcggcccgaggcctgccggcccgccgccggcggcgccgtgcacacccggggcctggacttcgcctgcgacttctgggtgctggtggtggtgggcggcgtgctggcctgctacagcctgctggtgaccgtggccttcatcatcttctgggtgcggagcaagcggagccggctgctgcacagcgactacatgaacatgaccccccggcggcccggccccacccggaagcactaccagccctacgcccccccccgggacttcgccgcctaccggagcctgcgggtgaagttcagccggagcgccgacgcccccgcctaccagcagggccagaaccagctgtacaacgagctgaacctgggccggcgggaggagtacgacgtgctggacaagcggcggggccgggaccccgagatgggcggcaagccccggcggaagaacccccaggagggcctgtacaacgagctgcagaaggacaagatggccgaggcctacagcgagatcggcatgaagggcgagcggcggcggggcaagggccacgacggcctgtaccagggcctgagcaccgccaccaaggacacctacgacgccctgcacatgcaggccctgcccccccgg

SEQ ID NO: 254 - skipped

SEQ ID NO: 255 - DNA, 1509 bp, synthetic construct
  source 1..1509: mol_type = other DNA
atggacttccaggtgcagatcttcagcttcctgctgatcagcgccagcgtgatcatgagccggatggcccaggtgcagctgcagcagagcggcgccgagctggcccggcccggcgccagcgtgaagatgagctgcaaggccagcggctacaccttcaccacctacaccatccactgggtgcggcagcggcccggccacgacctggagtggatcggctacatcaaccccagcagcggctacagcgactacaaccagaacttcaagggcaagaccaccctgaccgccgacaagagcagcaacaccgcctacatgcagctgaacagcctgaccagcgaggacagcgccgtgtactactgcgcccggcgggccgactacggcaactacgagtacacctggttcgcctactggggccagggcaccaccgtgaccgtgagcagcggcggcggcggcagcggcggcggcggcagcggcggcggcggcagcgacatcgagctgacccagagccccaagttcatgagcaccagcgtgggcgaccgggtgaacgtgacctacaaggccagccagaacgtgggcaccaacgtggcctggttccagcagaagcccggccagagccccaaggtgctgatctacagcgccagctaccggtacagcggcgtgcccgaccggttcaccggcagcggcagcggcaccgacttcaccctgaccatcagcaacgtgcagagcgaggacctggccgagtacttctgccagcagtaccacacctaccccctgaccttcggcggcggcaccaagctggagatcaagcggagcgaccccgccaccaccacccccgccccccggccccccacccccgcccccaccatcgccagccagcccctgagcctgcggcccgaggcctgccggcccgccgccggcggcgccgtgcacacccggggcctggacttcgcctgcgacaaggaccccaagttctgggtgctggtggtggtgggcggcgtgctggcctgctacagcctgctggtgaccgtggccttcatcatcttctgggtgcggagcaagcggagccggctgctgcacagcgactacatgaacatgaccccccggcggcccggccccacccggaagcactaccagccctacgcccccccccgggacttcgccgcctaccggagcctgcgggtgaagttcagccggagcgccgacgcccccgcctaccagcagggccagaaccagctgtacaacgagctgaacctgggccggcgggaggagtacgacgtgctggacaagcggcggggccgggaccccgagatgggcggcaagccccggcggaagaacccccaggagggcctgtacaacgagctgcagaaggacaagatggccgaggcctacagcgagatcggcatgaagggcgagcggcggcggggcaagggccacgacggcctgtaccagggcctgagcaccgccaccaaggacacctacgacgccctgcacatgcaggccctgcccccccgg

SEQ ID NO: 256 - skipped

SEQ ID NO: 257 - DNA, 1509 bp, synthetic construct
  source 1..1509: mol_type = other DNA
atggacttccaggtgcagatcttcagcttcctgctgatcagcgccagcgtgatcatgagccggatggcccaggtgcagctgcagcagagcggcgccgagctggcccggcccggcgccagcgtgaagatgagctgcaaggccagcggctacaccttcaccacctacaccatccactgggtgcggcggcggcccggccacgacctggagtggatcggctacatcaaccccagcagcggctacagcgactacaaccagaacttcaagggcaagaccaccctgaccgccgacaagagcagcaacaccgcctacatgcagctgaacagcctgaccagcgaggacagcgccgtgtactactgcgcccggcgggccgactacggcaactacgagtacacctggttcgcctactggggccagggcaccaccgtgaccgtgagcagcagcggcggcggcagcggcggcggcggcagcggcggcggcggcagcgtgatcgagctgacccagagccccaagttcatgagcaccagcgtgggcgaccgggtgaacgtgacctacaaggccagccagaacgtgggcaccaacgtggcctggttccagcagaagcccggccagagccccaaggtgctgatctacagcgccagctaccggtacagcggcgtgcccgaccggttcaccggcagcggcagcggcaccgacttcaccctgaccatcagcaacgtgcagagcgaggacctggccgagtacttctgccagcagtaccacacctaccccctgaccttcggcggcggcaccaagctggagatcaagcggagcgaccccgccaccaccacccccgccccccggccccccacccccgcccccaccatcgccagccagcccctgagcctgcggcccgaggcctgccggcccgccgccggcggcgccgtgcacacccggggcctggacttcgcctgcgacaaggaccccaagttctgggtgctggtggtggtgggcggcgtgctggcctgctacagcctgctggtgaccgtggccttcatcatcttctgggtgcggagcaagcggagccggctgctgcacagcgactacatgaacatgaccccccggcggcccggccccacccggaagcactaccagccctacgcccccccccgggacttcgccgcctaccggagcctgcgggtgaagttcagccggagcgccgacgcccccgcctaccagcagggccagaaccagctgtacaacgagctgaacctgggccggcgggaggagtacgacgtgctggacaagcggcggggccgggaccccgagatgggcggcaagccccggcggaagaacccccaggagggcctgtacaacgagctgcagaaggacaagatggccgaggcctacagcgagatcggcatgaagggcgagcggcggcggggcaagggccacgacggcctgtaccagggcctgagcaccgccaccaaggacacctacgacgccctgcacatgcaggccctgcccccccgg

SEQ ID NO: 258 - skipped

SEQ ID NO: 259 - DNA, 4289 bp, synthetic construct
  source 1..4289: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatcttgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgcggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatgtgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatgcggccgccaccatggacttccaggtgcagatcttcagcttcctgctgatcagcgccagcgtgatcatgagccggatggcccaggtgcagctgcagcagagcggcgccgagctggcccggcccggcgccagcgtgaagatgagctgcaaggccagcggctacaccttcaccacctacaccatccactgggtgcggcggcggcccggccacgacctggagtggatcggctacatcaaccccagcagcggctgcagcgactacaaccagaacttcaagggcaagaccaccctgaccgccgacaagagcagcaacaccgcctacatgcagctgaacagcctgaccagcgaggacagcgccgtgtactactgcgcccggcgggccgactacggcaactacgagtacacctggttcgcctactggggccagggcaccaccgtgaccgtgagcagcagcggcggcggcagcggcggcggcggcagcggcggcggcggcagcgtgatcgagctgacccagagccccaagttcatgagcaccagcgtgggcgaccgggtgaacgtgacctacaaggccagccagaacgtgggcaccaacgtggcctggttccagcagaagcccggccagagccccaaggtgctgatctacagcgccagctaccggtacagcggcgtgcccgaccggttcaccggcagcggcagcggcaccgacttcaccctgaccatcagcaacgtgcagagcgaggacctggccgagtacttctgccagcagtaccacacctaccccctgaccttcggcggcggcaccaagctggagatcaagcggagcgaccccgccaccaccacccccgccccccggccccccacccccgcccccaccatcgccagccagcccctgagcctgcggcccgaggcctgccggcccgccgccggcggcgccgtgcacacccggggcctggacttcgcctgcgacaaggaccccaagttctgggtgctggtggtggtgggcggcgtgctggcctgctacagcctgctggtgaccgtggccttcatcatcttctgggtgcggagcaagcggagccggctgctgcacagcgactacatgaacatgaccccccggcggcccggccccacccggaagcactaccagccctacgcccccccccgggacttcgccgcctaccggagcctgcgggtgaagttcagccggagcgccgacgcccccgcctaccagcagggccagaaccagctgtacaacgagctgaacctgggccggcgggaggagtacgacgtgctggacaagcggcggggccgggaccccgagatgggcggcaagccccggcggaagaacccccaggagggcctgtacaacgagctgcagaaggacaagatggccgaggcctacagcgagatcggcatgaagggcgagcggcggcggggcaagggccacgacggcctgtaccagggcctgagcaccgccaccaaggacacctacgacgccctgcacatgcaggccctgcccccccggcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccactagtcgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 260-299 - skipped (40 entries)

SEQ ID NO: 300 - RNA, 100 nt, synthetic construct
  source 1..100: mol_type = other RNA
  misc_feature 1..4: note = Phosphorothioate linkage
  misc_feature 97..100: note = Phosphorothioate linkage
  modified_base 1..3: mod_base = OTHER; note = 2'-O-methyl nucleotide
  modified_base 29..40: mod_base = OTHER; note = 2'-O-methyl nucleotide
  modified_base 69..100: mod_base = OTHER; note = 2'-O-methyl nucleotide
ggccacggagcgagacatctgttttagagctagaaatagcaagttaaaataaggctagtccgttatcaacttgaaaaagtggcaccgagtcggtgctttt

SEQ ID NO: 301 - RNA, 20 nt, synthetic construct
  source 1..20: mol_type = other RNA
ggccacggagcgagacatct

SEQ ID NO: 302 - RNA, 100 nt, synthetic construct
  source 1..100: mol_type = other RNA
  misc_feature 1..4: note = Phosphorothioate linkage
  misc_feature 97..100: note = Phosphorothioate linkage
  modified_base 1..3: mod_base = OTHER; note = 2'-O-methyl nucleotide
  modified_base 29..40: mod_base = OTHER; note = 2'-O-methyl nucleotide
  modified_base 69..100: mod_base = OTHER; note = 2'-O-methyl nucleotide
cccccggacggttcaagcaagttttagagctagaaatagcaagttaaaataaggctagtccgttatcaacttgaaaaagtggcaccgagtcggtgctttt

SEQ ID NO: 303 - RNA, 20 nt, synthetic construct
  source 1..20: mol_type = other RNA
cccccggacggttcaagcaa

SEQ ID NO: 304 - RNA, 100 nt, synthetic construct
  source 1..100: mol_type = other RNA
  misc_feature 1..4: note = Phosphorothioate linkage
  misc_feature 97..100: note = Phosphorothioate linkage
  modified_base 1..3: mod_base = OTHER; note = 2'-O-methyl nucleotide
  modified_base 29..40: mod_base = OTHER; note = 2'-O-methyl nucleotide
  modified_base 69..100: mod_base = OTHER; note = 2'-O-methyl nucleotide
acagcgacgccgcgagccaggttttagagctagaaatagcaagttaaaataaggctagtccgttatcaacttgaaaaagtggcaccgagtcggtgctttt

SEQ ID NO: 305 - DNA, 20 bp, synthetic construct
  source 1..20: mol_type = other DNA
acagcgacgccgcgagccag

SEQ ID NO: 306-315 - skipped (10 entries)

SEQ ID NO: 316 - protein, 1379 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKV

SEQ ID NO: 317 - DNA, 4140 bp, synthetic construct
  source 1..4140: mol_type = other DNA
atggacaagaagtacagcatcggactggacatcggaacaaacagcgtcggatgggcagtcatcacagacgaatacaaggtcccgagcaagaagttcaaggtcctgggaaacacagacagacacagcatcaagaagaacctgatcggagcactgctgttcgacagcggagaaacagcagaagcaacaagactgaagagaacagcaagaagaagatacacaagaagaaagaacagaatctgctacctgcaggaaatcttcagcaacgaaatggcaaaggtcgacgacagcttcttccacagactggaagaaagcttcctggtcgaagaagacaagaagcacgaaagacacccgatcttcggaaacatcgtcgacgaagtcgcataccacgaaaagtacccgacaatctaccacctgagaaagaagctggtcgacagcacagacaaggcagacctgagactgatctacctggcactggcacacatgatcaagttcagaggacacttcctgatcgaaggagacctgaacccggacaacagcgacgtcgacaagctgttcatccagctggtccagacatacaaccagctgttcgaagaaaacccgatcaacgcaagcggagtcgacgcaaaggcaatcctgagcgcaagactgagcaagagcagaagactggaaaacctgatcgcacagctgccgggagaaaagaagaacggactgttcggaaacctgatcgcactgagcctgggactgacaccgaacttcaagagcaacttcgacctggcagaagacgcaaagctgcagctgagcaaggacacatacgacgacgacctggacaacctgctggcacagatcggagaccagtacgcagacctgttcctggcagcaaagaacctgagcgacgcaatcctgctgagcgacatcctgagagtcaacacagaaatcacaaaggcaccgctgagcgcaagcatgatcaagagatacgacgaacaccaccaggacctgacactgctgaaggcactggtcagacagcagctgccggaaaagtacaaggaaatcttcttcgaccagagcaagaacggatacgcaggatacatcgacggaggagcaagccaggaagaattctacaagttcatcaagccgatcctggaaaagatggacggaacagaagaactgctggtcaagctgaacagagaagacctgctgagaaagcagagaacattcgacaacggaagcatcccgcaccagatccacctgggagaactgcacgcaatcctgagaagacaggaagacttctacccgttcctgaaggacaacagagaaaagatcgaaaagatcctgacattcagaatcccgtactacgtcggaccgctggcaagaggaaacagcagattcgcatggatgacaagaaagagcgaagaaacaatcacaccgtggaacttcgaagaagtcgtcgacaagggagcaagcgcacagagcttcatcgaaagaatgacaaacttcgacaagaacctgccgaacgaaaaggtcctgccgaagcacagcctgctgtacgaatacttcacagtctacaacgaactgacaaaggtcaagtacgtcacagaaggaatgagaaagccggcattcctgagcggagaacagaagaaggcaatcgtcgacctgctgttcaagacaaacagaaaggtcacagtcaagcagctgaaggaagactacttcaagaagatcgaatgcttcgacagcgtcgaaatcagcggagtcgaagacagattcaacgcaagcctgggaacataccacgacctgctgaagatcatcaaggacaaggacttcctggacaacgaagaaaacgaagacatcctggaagacatcgtcctgacactgacactgttcgaagacagagaaatgatcgaagaaagactgaagacatacgcacacctgttcgacgacaaggtcatgaagcagctgaagagaagaagatacacaggatggggaagactgagcagaaagctgatcaacggaatcagagacaagcagagcggaaagacaatcctggacttcctgaagagcgacggattcgcaaacagaaacttcatgcagctgatccacgacgacagcctgacattcaaggaagacatccagaaggcacaggtcagcggacagggagacagcctgcacgaacacatcgcaaacctggcaggaagcccggcaatcaagaagggaatcctgcagacagtcaaggtcgtcgacgaactggtcaaggtcatgggaagacacaagccggaaaacatcgtcatcgaaatggcaagagaaaaccagacaacacagaagggacagaagaacagcagagaaagaatgaagagaatcgaagaaggaatcaaggaactgggaagccagatcctgaaggaacacccggtcgaaaacacacagctgcagaacgaaaagctgtacctgtactacctgcagaacggaagagacatgtacgtcgaccaggaactggacatcaacagactgagcgactacgacgtcgaccacatcgtcccgcagagcttcctgaaggacgacagcatcgacaacaaggtcctgacaagaagcgacaagaacagaggaaagagcgacaacgtcccgagcgaagaagtcgtcaagaagatgaagaactactggagacagctgctgaacgcaaagctgatcacacagagaaagttcgacaacctgacaaaggcagagagaggaggactgagcgaactggacaaggcaggattcatcaagagacagctggtcgaaacaagacagatcacaaagcacgtcgcacagatcctggacagcagaatgaacacaaagtacgacgaaaacgacaagctgatcagagaagtcaaggtcatcacactgaagagcaagctggtcagcgacttcagaaaggacttccagttctacaaggtcagagaaatcaacaactaccaccacgcacacgacgcatacctgaacgcagtcgtcggaacagcactgatcaagaagtacccgaagctggaaagcgaattcgtctacggagactacaaggtctacgacgtcagaaagatgatcgcaaagagcgaacaggaaatcggaaaggcaacagcaaagtacttcttctacagcaacatcatgaacttcttcaagacagaaatcacactggcaaacggagaaatcagaaagagaccgctgatcgaaacaaacggagaaacaggagaaatcgtctgggacaagggaagagacttcgcaacagtcagaaaggtcctgagcatgccgcaggtcaacatcgtcaagaagacagaagtccagacaggaggattcagcaaggaaagcatcctgccgaagagaaacagcgacaagctgatcgcaagaaagaaggactgggacccgaagaagtacggaggattcgacagcccgacagtcgcatacagcgtcctggtcgtcgcaaaggtcgaaaagggaaagagcaagaagctgaagagcgtcaaggaactgctgggaatcacaatcatggaaagaagcagcttcgaaaagaacccgatcgacttcctggaagcaaagggatacaaggaagtcaagaaggacctgatcatcaagctgccgaagtacagcctgttcgaactggaaaacggaagaaagagaatgctggcaagcgcaggagaactgcagaagggaaacgaactggcactgccgagcaagtacgtcaacttcctgtacctggcaagccactacgaaaagctgaagggaagcccggaagacaacgaacagaagcagctgttcgtcgaacagcacaagcactacctggacgaaatcatcgaacagatcagcgaattcagcaagagagtcatcctggcagacgcaaacctggacaaggtcctgagcgcatacaacaagcacagagacaagccgatcagagaacaggcagaaaacatcatccacctgttcacactgacaaacctgggagcaccggcagcattcaagtacttcgacacaacaatcgacagaaagagatacacaagcacaaaggaagtcctggacgcaacactgatccaccagagcatcacaggactgtacgaaacaagaatcgacctgagccagctgggaggagacggaggaggaagcccgaagaagaagagaaaggtctag

SEQ ID NO: 318 - DNA, 4140 bp, synthetic construct
  source 1..4140: mol_type = other DNA
atggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtga

SEQ ID NO: 319 - RNA, 4197 nt, synthetic construct
  source 1..4197: mol_type = other RNA
atggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtccgagtccgccacccccgagtccgtgtccggctggcggctgttcaagaagatctcctga

SEQ ID NO: 320-324 - skipped (5 entries)

SEQ ID NO: 325 - protein, 1379 aa, synthetic construct; identical to SEQ ID NO: 316

SEQ ID NO: 326 - protein, 1398 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKVSESATPESVSGWRLFKKIS

SEQ ID NO: 327-799 - skipped (473 entries)

SEQ ID NO: 800 - protein, 1379 aa, synthetic construct; identical to SEQ ID NO: 316

SEQ ID NO: 801 - DNA, 4140 bp, synthetic construct; identical to SEQ ID NO: 317
  source 1..4140: mol_type = other DNA

SEQ ID NO: 802 - DNA, 4140 bp, synthetic construct; identical to SEQ ID NO: 318
  source 1..4140: mol_type = other DNA

SEQ ID NO: 803 - RNA, 4197 nt, synthetic construct; identical to SEQ ID NO: 319
  source 1..4197: mol_type = other RNA

SEQ ID NO: 804-808 - skipped (5 entries)

SEQ ID NO: 809 - protein, 1379 aa, synthetic construct; identical to SEQ ID NO: 316

SEQ ID NO: 810 - protein, 1398 aa, synthetic construct; identical to SEQ ID NO: 326

SEQ ID NO: 811-816 - skipped (6 entries)

SEQ ID NO: 817 - RNA, 4140 nt, synthetic construct
  source 1..4140: mol_type = other RNA
atggacaagaagtacagcatcggactggacatcggaacaaacagcgtcggatgggcagtcatcacagacgaatacaaggtcccgagcaagaagttcaaggtcctgggaaacacagacagacacagcatcaagaagaacctgatcggagcactgctgttcgacagcggagaaacagcagaagcaacaagactgaagagaacagcaagaagaagatacacaagaagaaagaacagaatctgctacctgcaggaaatcttcagcaacgaaatggcaaaggtcgacgacagcttcttccacagactggaagaaagcttcctggtcgaagaagacaagaagcacgaaagacacccgatcttcggaaacatcgtcgacgaagtcgcataccacgaaaagtacccgacaatctaccacctgagaaagaagctggtcgacagcacagacaaggcagacctgagactgatctacctggcactggcacacatgatcaagttcagaggacacttcctgatcgaaggagacctgaacccggacaacagcgacgtcgacaagctgttcatccagctggtccagacatacaaccagctgttcgaagaaaacccgatcaacgcaagcggagtcgacgcaaaggcaatcctgagcgcaagactgagcaagagcagaagactggaaaacctgatcgcacagctgccgggagaaaagaagaacggactgttcggaaacctgatcgcactgagcctgggactgacaccgaacttcaagagcaacttcgacctggcagaagacgcaaagctgcagctgagcaaggacacatacgacgacgacctggacaacctgctggcacagatcggagaccagtacgcagacctgttcctggcagcaaagaacctgagcgacgcaatcctgctgagcgacatcctgagagtcaacacagaaatcacaaaggcaccgctgagcgcaagcatgatcaagagatacgacgaacaccaccaggacctgacactgctgaaggcactggtcagacagcagctgccggaaaagtacaaggaaatcttcttcgaccagagcaagaacggatacgcaggatacatcgacggaggagcaagccaggaagaattctacaagttcatcaagccgatcctggaaaagatggacggaacagaagaactgctggtcaagctgaacagagaagacctgctgagaaagcagagaacattcgacaacggaagcatcccgcaccagatccacctgggagaactgcacgcaatcctgagaagacaggaagacttctacccgttcctgaaggacaacagagaaaagatcgaaaagatcctgacattcagaatcccgtactacgtcggaccgctggcaagaggaaacagcagattcgcatggatgacaagaaagagcgaagaaacaatcacaccgtggaacttcgaagaagtcgtcgacaagggagcaagcgcacagagcttcatcgaaagaatgacaaacttcgacaagaacctgccgaacgaaaaggtcctgccgaagcacagcctgctgtacgaatacttcacagtctacaacgaactgacaaaggtcaagtacgtcacagaaggaatgagaaagccggcattcctgagcggagaacagaagaaggcaatcgtcgacctgctgttcaagacaaacagaaaggtcacagtcaagcagctgaaggaagactacttcaagaagatcgaatgcttcgacagcgtcgaaatcagcggagtcgaagacagattcaacgcaagcctgggaacataccacgacctgctgaagatcatcaaggacaaggacttcctggacaacgaagaaaacgaagacatcctggaagacatcgtcctgacactgacactgttcgaagacagagaaatgatcgaagaaagactgaagacatacgcacacctgttcgacgacaaggtcatgaagcagctgaagagaagaagatacacaggatggggaagactgagcagaaagctgatcaacggaatcagagacaagcagagcggaaagacaatcctggacttcctgaagagcgacggattcgcaaacagaaacttcatgcagctgatccacgacgacagcctgacattcaaggaagacatccagaaggcacaggtcagcggacagggagacagcctgcacgaacacatcgcaaacctggcaggaagcccggcaatcaagaagggaatcctgcagacagtcaaggtcgtcgacgaactggtcaaggtcatgggaagacacaagccggaaaacatcgtcatcgaaatggcaagagaaaaccagacaacacagaagggacagaagaacagcagagaaagaatgaagagaatcgaagaaggaatcaaggaactgggaagccagatcctgaaggaacacccggtcgaaaacacacagctgcagaacgaaaagctgtacctgtactacctgcagaacggaagagacatgtacgtcgaccaggaactggacatcaacagactgagcgactacgacgtcgaccacatcgtcccgcagagcttcctgaaggacgacagcatcgacaacaaggtcctgacaagaagcgacaagaacagaggaaagagcgacaacgtcccgagcgaagaagtcgtcaagaagatgaagaactactggagacagctgctgaacgcaaagctgatcacacagagaaagttcgacaacctgacaaaggcagagagaggaggactgagcgaactggacaaggcaggattcatcaagagacagctggtcgaaacaagacagatcacaaagcacgtcgcacagatcctggacagcagaatgaacacaaagtacgacgaaaacgacaagctgatcagagaagtcaaggtcatcacactgaagagcaagctggtcagcgacttcagaaaggacttccagttctacaaggtcagagaaatcaacaactaccaccacgcacacgacgcatacctgaacgcagtcgtcggaacagcactgatcaagaagtacccgaagctggaaagcgaattcgtctacggagactacaaggtctacgacgtcagaaagatgatcgcaaagagcgaacaggaaatcggaaaggcaacagcaaagtacttcttctacagcaacatcatgaacttcttcaagacagaaatcacactggcaaacggagaaatcagaaagagaccgctgatcgaaacaaacggagaaacaggagaaatcgtctgggacaagggaagagacttcgcaacagtcagaaaggtcctgagcatgccgcaggtcaacatcgtcaagaagacagaagtccagacaggaggattcagcaaggaaagcatcctgccgaagagaaacagcgacaagctgatcgcaagaaagaaggactgggacccgaagaagtacggaggattcgacagcccgacagtcgcatacagcgtcctggtcgtcgcaaaggtcgaaaagggaaagagcaagaagctgaagagcgtcaaggaactgctgggaatcacaatcatggaaagaagcagcttcgaaaagaacccgatcgacttcctggaagcaaagggatacaaggaagtcaagaaggacctgatcatcaagctgccgaagtacagcctgttcgaactggaaaacggaagaaagagaatgctggcaagcgcaggagaactgcagaagggaaacgaactggcactgccgagcaagtacgtcaacttcctgtacctggcaagccactacgaaaagctgaagggaagcccggaagacaacgaacagaagcagctgttcgtcgaacagcacaagcactacctggacgaaatcatcgaacagatcagcgaattcagcaagagagtcatcctggcagacgcaaacctggacaaggtcctgagcgcatacaacaagcacagagacaagccgatcagagaacaggcagaaaacatcatccacctgttcacactgacaaacctgggagcaccggcagcattcaagtacttcgacacaacaatcgacagaaagagatacacaagcacaaaggaagtcctggacgcaacactgatccaccagagcatcacaggactgtacgaaacaagaatcgacctgagccagctgggaggagacggaggaggaagcccgaagaagaagagaaaggtctag

SEQ ID NO: 818 - protein, 14 aa, synthetic construct
EGKSSGSGSESKVD

(19) United States
(12) Patent Application Publication
(10) Pub. No.: US 2025/0268940 A1
(43) Pub. Date: Aug. 28, 2025

(54) CHIMERIC ANTIGEN RECEPTOR COMPOSITIONS AND USES

(71) Applicant: Intellia Therapeutics, Inc., Cambridge, MA (US)

(72) Inventors: Birgit Schultes, Arlington, MA (US); Yong Zhang, Bedford, MA (US); Ishina Balwani, Malden, MA (US); Utsav Jetley, Roslindale, MA (US)

(21) Appl. No.: 18/857,966

(22) PCT Filed: Apr. 18, 2023
(86) PCT No.: PCT/US23/18946

§ 371 Date: Oct. 18, 2024

Related U.S. Application Data
(60) Provisional application No. 63/414,428, filed on Oct. 7, 2022.
(60) Provisional application No. 63/336,842, filed on Apr. 29, 2022.
(60) Provisional application No. 63/332,564, filed on Apr. 19, 2022.

(51) Int. Cl. A61K 35/17; A61K 40/11; A61K 40/31; A61K 40/42; A61P 35/00; C07K 14/705; C07K 14/725; C07K 16/28; C12N 5/0783; C12N 9/22; C12N 15/11

(52) U.S. Cl. CPC A61K 35/17; A61K 40/11; A61K 40/31; A61K 40/4215; A61P 35/00; C07K 14/7051; C07K 14/70517; C07K 14/70521; C07K 16/2878; C12N 5/0636; C12N 9/226; C12N 15/111; A61K 2239/13; A61K 2239/17; A61K 2239/21; A61K 2239/48; C07K 2317/565; C07K 2317/622; C12N 2310/20

(57) ABSTRACT

Compositions and methods for expressing a chimeric antigen receptor in a cell for use e.g., in adoptive cell transfer therapies.

### CROSS-REFERENCE TO RELATED APPLICATIONS

This application claims the benefit of U.S. Provisional Application No. 63/332,564, filed on Apr. 19, 2022, U.S. Provisional Application No. 63/336,842, filed on Apr. 29, 2022, and U.S. Provisional Application No. 63/414,428, filed on Oct. 7, 2022, the content of each of which is hereby incorporated by reference in its entirety.

### SUMMARY

In certain aspects, disclosed herein are chimeric antigen receptors (CARs) and engineered cells comprising these CARs. In certain embodiments, the CARs are specific for CD30. In certain embodiments, the engineered cells expressing the CARs are autologous therapeutic cells. In certain embodiments, the engineered cells expressing the CARs are allogeneic therapeutic cells that can be modified to provide advantageous properties, e.g., reduce or prevent graft versus host disease (GVHD) in a host receiving the allogeneic cells; or evade the host immune response. In certain embodiments, the engineered cells expressing the CARs have reduced expression of, e.g., HLA-A, HLA class II proteins, or T cell receptors.

In certain aspects, provided herein is chimeric antigen receptor (CAR) comprising: (a) a binder domain (e.g., a binder domain that binds to CD30); (b) a hinge domain (e.g., a hinge domain comprising a hinge sequence selected from an IgG1-CH2-CH3 hinge sequence, an IgG1-CH3 hinge sequence, a CD8a hinge sequence, and a CD28 hinge sequence); (c) a transmembrane domain (e.g., a transmembrane domain comprising a transmembrane sequence selected from a CD28 transmembrane sequence and a CD8a transmembrane sequence; (d) a costimulatory domain (e.g., a costimulatory domain comprising a costimulatory sequence selected from a CD28 costimulatory sequence and a 41BB costimulatory sequence); and (e) an activation domain (e.g., comprising a CD3z activation sequence). In certain embodiments, the CAR comprises: (a) a binder domain that binds CD30; (b) a CD8a hinge domain; (c) a CD28 transmembrane domain; (d) a costimulatory domain; and (e) an activation domain. In some embodiments, the CAR also comprises an N-terminal signal peptide. In some embodiments the signal peptide comprises a sequence of MDFQVQIFSFLLISASVIMSRMA SEQ ID NO: 1; (HRS3 Signal peptide). In some embodiments, the signal peptide comprises as sequence of MALPVTALLLPLALLLHAARP (SEQ ID NO: 2; 5F11 signal peptide).

In some embodiments, the CAR comprises a binder domain that binds to CD30. In certain embodiments, the binder domain comprises a CD30 antibody or antigen binding fragment thereof (e.g., an scFv). In some embodiments, the CARs provided herein comprise a sequence at least 80% (e.g., at least 85%, at least 90%, at least 91%, at least 92%, at least 93%, at least 94%, at least 95%, at least 96%, at least 97%, at least 98%, at least 99%, or 100%) identical to a sequence selected from the binder domain sequences listed in Table 1 or 1A.

In some embodiments, the CD30 antibody or antigen binding fragment thereof comprises a heavy chain variable region (VH) complementarity-determining region 1 (CDR1) sequence TFTTYT (SEQ ID NO: 3); a VH CDR2 sequence INPSSGCSD (SEQ ID NO: 4) or INPSSGYSD (SEQ ID NO: 5); a VH CDR3 sequence RADYGNYEYTWFAY (SEQ ID NO: 6); a light chain variable region (VL) CDR1 sequence ASQNVGTNVA (SEQ ID NO: 7); a VL CDR2 sequence SASYRYS (SEQ ID NO: 8); and a VL CDR3 sequence QQYHTYP (SEQ ID NO: 9). In certain embodiments, the VH amino acid sequence has at least 80% (e.g., at least 85%, at least 90%, at least 91%, at least 92%, at least 93%, at least 94%, at least 95%, at least 96%, at least 97%, at least 98%, at least 99%, or 100%) identity to sequence SEQ ID NO: 24 and the VL amino acid sequence has at least 80% (e.g., at least 85%, at least 90%, at least 91%, at least 92%, at least 93%, at least 94%, at least 95%, at least 96%, at least 97%, at least 98%, at least 99%, or 100%) identity to SEQ ID NO: 25. In some embodiments, the binder domain is HRS3.

In some embodiments, the CD30 antibody or antigen binding fragment thereof comprises a heavy chain variable region (VH) complementarity-determining region 1 (CDR1) sequence TFTTYT (SEQ ID NO: 3); a VH CDR2 sequence INPSSGCSD (SEQ ID NO: 4) or INPSSGYSD (SEQ ID NO: 5); a VH CDR3 sequence RADYGNYEYTWFAY (SEQ ID NO: 6); a light chain variable region (VL) CDR1 sequence ASQNVGTNVA (SEQ ID NO: 7); a VL CDR2 sequence SASYRYS (SEQ ID NO: 8); and a VL CDR3 sequence QQYHTYP (SEQ ID NO: 9). In certain embodiments, the VH amino acid sequence has at least 80% (e.g., at least 85%, at least 90%, at least 91%, at least 92%, at least 93%, at least 94%, at least 95%, at least 96%, at least 97%, at least 98%, at least 99%, or 100%) identity to sequence SEQ ID NO: 26 and the VL amino acid sequence has at least 80% (e.g., at least 85%, at least 90%, at least 91%, at least 92%, at least 93%, at least 94%, at least 95%, at least 96%, at least 97%, at least 98%, at least 99%, or 100%) identity to SEQ ID NO: 27. In some embodiments, the binder domain is HRS3.

In some embodiments, the CD30 antibody or antigen binding fragment thereof comprises a heavy chain variable region (VH) complementarity-determining region 1 (CDR1) sequence TFTTYT (SEQ ID NO: 3); a VH CDR2 sequence INPSSGCSD (SEQ ID NO: 4) or INPSSGYSD (SEQ ID NO: 5); a VH CDR3 sequence RADYGNYEYTWFAY (SEQ ID NO: 6); a light chain variable region (VL) CDR1 sequence ASQNVGTNVA (SEQ ID NO: 7); a VL CDR2 sequence SASYRYS (SEQ ID NO: 8); and a VL CDR3 sequence QQYHTYP (SEQ ID NO: 9). In certain embodiments, the VH amino acid sequence has at least 80% (e.g., at least 85%, at least 90%, at least 91%, at least 92%, at least 93%, at least 94%, at least 95%, at least 96%, at least 97%, at least 98%, at least 99%, or 100%) identity to sequence SEQ ID NO: 28 and the VL amino acid sequence has at least 80% (e.g., at least 85%, at least 90%, at least 91%, at least 92%, at least 93%, at least 94%, at least 95%, at least 96%, at least 97%, at least 98%, at least 99%, or 100%) identity to SEQ ID NO: 25. In some embodiments, the binder domain is HRS3.

In some embodiments, the CD30 antibody or antigen binding fragment thereof comprises a heavy chain variable region (VH) complementarity-determining region 1 (CDR1) sequence TFTTYT (SEQ ID NO: 3); a VH CDR2 sequence INPSSGCSD (SEQ ID NO: 4) or INPSSGYSD (SEQ ID NO: 5); a VH CDR3 sequence RADYGNYEYTWFAY (SEQ ID NO: 6); a light chain variable region (VL) CDR1 sequence ASQNVGTNVA (SEQ ID NO: 7); a VL CDR2 sequence SASYRYS (SEQ ID NO: 8); and a VL CDR3 sequence QQYHTYP (SEQ ID NO: 9). In certain embodiments, the VH amino acid sequence has at least 80% (e.g., at least 85%, at least 90%, at least 91%, at least 92%, at least 93%, at least 94%, at least 95%, at least 96%, at least 97%, at least 98%, at least 99%, or 100%) identity to sequence SEQ ID NO: 29 and the VL amino acid sequence has at least 80% (e.g., at least 85%, at least 90%, at least 91%, at least 92%, at least 93%, at least 94%, at least 95%, at least 96%, at least 97%, at least 98%, at least 99%, or 100%) identity to SEQ ID NO: 30. In some embodiments, the binder domain is HRS3.

In some embodiments, the CD30 antibody or antigen binding fragment thereof comprises a heavy chain variable region (VH) complementarity-determining region 1 (CDR1) sequence AYYWS (SEQ ID NO: 10); a VH CDR2 sequence DINHGGGTNYNPSLKS (SEQ ID NO: 11); a VH CDR3 sequence LTAY (SEQ ID NO: 12); a light chain variable region (VL) CDR1 sequence RASQGISSWLT (SEQ ID NO: 13); a VL CDR2 sequence AASSLQS (SEQ ID NO: 14); and a VL CDR3 sequence QQYDSYPIT (SEQ ID NO: 15). In certain embodiments, the VH amino acid sequence has at least 80% (e.g., at least 85%, at least 90%, at least 91%, at least 92%, at least 93%, at least 94%, at least 95%, at least 96%, at least 97%, at least 98%, at least 99%, or 100%) identity to sequence SEQ ID NO: 31 and the VL amino acid sequence has at least 80% (e.g., at least 85%, at least 90%, at least 91%, at least 92%, at least 93%, at least 94%, at least 95%, at least 96%, at least 97%, at least 98%, at least 99%, or 100%) identity to SEQ ID NO: 32. In certain embodiments, the binder domain is 5F11.

In some embodiments, the binder domain comprises the sequence of SEQ ID NO: 20, 21, or 22.

In some embodiments of the CARs provided herein, the hinge domain comprises a sequence having at least 80% (e.g., at least 85%, at least 90%, at least 91%, at least 92%, at least 93%, at least 94%, at least 95%, at least 96%, at least 97%, at least 98%, at least 99%, or 100%) identity to a sequence selected from the hinge domain sequences listed in Table 2.

In some embodiments of the CARs provided herein, the CAR comprises a linker between the hinge domain and the transmembrane domain. In some embodiments, the linker comprises a sequence of KPDK (SEQ ID NO: 16).

In some embodiments of the CARs provided herein, the transmembrane domain comprises a sequence having at least 80% (e.g., at least 85%, at least 90%, at least 91%, at least 92%, at least 93%, at least 94%, at least 95%, at least 96%, at least 97%, at least 98%, at least 99%, or 100%) identity to a sequence selected from the transmembrane domain sequence provided in Table 3.

In some embodiments of the CARs provided herein, the costimulatory domain is a CD28 costimulatory domain (e.g., a wild-type CD28 costimulatory domain; a CD28 costimulatory domain that comprises a mutation that eliminates lck binding). In some embodiments, the costimulatory domain comprises a sequence having at least 80% (e.g., at least 85%, at least 90%, at least 91%, at least 92%, at least 93%, at least 94%, at least 95%, at least 96%, at least 97%, at least 98%, at least 99%, or 100%) identity to a sequence selected from the costimulatory domain sequences provided in Table 4.

In certain embodiments of the CARs provided herein, the activation domain is a CD3z activation domain (e.g., a wild-type CD3z activation domain n, e.g., a wild-type CD3z activation domain comprising an N-terminal leucine or arginine, or a modified CD3z activation domain, e.g., SEQ ID NOs: 60-62). In some embodiments, the activation domain comprises a sequence having at least 80% (e.g., at least 85%, at least 90%, at least 91%, at least 92%, at least 93%, at least 94%, at least 95%, at least 96%, at least 97%, at least 98%, at least 99%, or 100%) identity to a sequence selected from the activation domain sequences listed in Table 5.

In some embodiments of the CARs provided herein, the hinge domain comprises a CD8a hinge sequence; the transmembrane domain comprises a CD28 transmembrane sequence; the costimulatory domain comprises a wild-type CD28 costimulatory sequence; and the activation domain comprises a wild-type CD3z activation sequence comprising an N-terminal leucine. In some embodiments, the CD8a hinge sequence comprises the sequence of SEQ ID NO: 50; the CD28 transmembrane sequence comprises the sequence of SEQ ID NO: 54; the CD28 costimulatory sequence comprises the sequence of SEQ ID NO: 57; and the CD3z activation sequence comprises the sequence of SEQ ID NO: 61.

In some embodiments of the CARs provided herein, the hinge domain comprises an IgG1-CH3 hinge sequence; the transmembrane domain comprises a CD28 transmembrane sequence; the costimulatory domain comprises a CD28 costimulatory sequence comprising a mutation that eliminates lck binding; and the activation domain comprises a wild-type CD3z activation sequence comprising an N-terminal leucine. In some embodiments, the IgG1-CH3 hinge sequence comprises the sequence of SEQ ID NO: 53; the CD28 transmembrane sequence comprises the sequence of SEQ ID NO: 54; the CD28 costimulatory sequence comprises the sequence of SEQ ID NO: 58; and the CD3z activation sequence comprises the sequence of SEQ ID NO: 61.

In some embodiments of the CARs provided herein, the hinge domain comprises an IgG1-CH2-CH3 hinge sequence; the transmembrane domain comprises a CD28 transmembrane sequence; the costimulatory domain comprises a wild-type CD28 costimulatory sequence; and the activation domain comprises a CD3z activation sequence comprising an additional leucine compared to a wild-type CD3z activation sequence comprising an N-terminal arginine. In some embodiments, the IgG1-CH2-CH3 hinge sequence comprises the sequence of SEQ ID NO: 52; the CD28 transmembrane sequence comprises the sequence of SEQ ID NO: 54; the CD28 costimulatory sequence comprises the sequence of SEQ ID NO: 57; and the CD3z activation sequence comprises the sequence of SEQ ID NO: 61.

In some embodiments of the CARs provided herein, the hinge domain comprises a CD28 hinge sequence; the transmembrane domain comprises a CD28 transmembrane sequence; the costimulatory domain comprises a CD28 costimulatory sequence comprising a mutation that eliminates lck binding; and the activation domain comprises a wild-type CD3z activation sequence having an N-terminal arginine. In some embodiments, the CD28 hinge sequence comprises the sequence of SEQ ID NO: 51; the CD28 transmembrane sequence comprises the sequence of SEQ ID NO: 54; the CD28 costimulatory sequence comprises the sequence of SEQ ID NO: 58; and the CD3z activation sequence comprises the sequence of SEQ ID NO: 60.

In some embodiments of the CARs provided herein, the hinge domain comprises a CD8a hinge sequence; the transmembrane domain comprises a CD28 transmembrane sequence; the costimulatory domain comprises a wild-type CD28 costimulatory sequence; and the activation domain comprises a wild-type CD3z activation sequence having an N-terminal arginine. In some embodiments, the CD8a hinge sequence comprises the sequence of SEQ ID NO: 50; the CD28 transmembrane sequence comprises the sequence of SEQ ID NO: 54; the CD28 costimulatory sequence comprises the sequence of SEQ ID NO: 57; and the CD3z activation sequence comprises the sequence of SEQ ID NO: 60.

In certain embodiments of the CARs provided herein, the hinge domain comprises a CD8a hinge sequence; the transmembrane domain comprises a CD8a transmembrane sequence; the costimulatory domain comprises a 41BB costimulatory sequence; and the activation domain comprises a modified CD3z activation sequence. In certain embodiments, the CD8a hinge sequence comprises the sequence of SEQ ID NO: 50; the CD8a transmembrane sequence comprises the sequence of SEQ ID NO: 55; the 41BB costimulatory sequence comprises the sequence of SEQ ID NO: 59; and the CD3z activation sequence comprises the sequence of SEQ ID NO: 62.

In some embodiments of the CARs provided herein, the hinge domain comprises a CD8a hinge sequence; the transmembrane domain comprises a CD28 transmembrane sequence; the costimulatory domain comprises a wild-type CD28 costimulatory sequence; and the activation domain comprises a modified CD3z activation sequence. In some embodiments, the CD8a hinge sequence comprises the sequence of SEQ ID NO: 50; the CD28 transmembrane sequence comprises the sequence of SEQ ID NO: 54; the CD28 costimulatory sequence comprises the sequence of SEQ ID NO: 57; and the CD3z activation sequence comprises the sequence of SEQ ID NO: 62.

In certain embodiments, the CAR provided herein comprises a sequence having at least 80% (e.g., at least 85%, at least 90%, at least 91%, at least 92%, at least 93%, at least 94%, at least 95%, at least 96%, at least 97%, at least 98%, at least 99%, or 100%) identity to a sequence selected from the CAR sequences provided in Table 6.

In some embodiments, the CAR comprises a sequence selected from the CAR sequences SEQ ID NO: 65, SEQ ID NO: 66, SEQ ID NO: 67, SEQ ID NO: 68, SEQ ID NO: 69, SEQ ID NO: 71, SEQ ID NO: 72, SEQ ID NO: 73, SEQ ID NO: 74

In some aspects, provided herein is an engineered cell comprising a CAR provided herein. In some embodiments, the engineered cell is derived from a T cell or a NK cell.

In some embodiments, the engineered cell is derived from a T cell. In certain embodiments, the engineered cell has reduced expression of a T cell receptor (TCR) on its surface relative to the T cell from which it was derived. In some embodiments, the engineered cell comprises a nucleic acid sequence encoding the CAR located in an endogenous TRAC locus of the engineered cell. In some embodiments, the nucleic acid sequence encoding the CAR disrupts the coding sequence of a TCR in the TRAC locus. In certain embodiments, the engineered cell does not express a TCR.

In some embodiments, the engineered cell comprises at least one genetic modification in a MHC class II gene. In some embodiments, the engineered cell does not express the MHC class II gene. In some embodiments, the MHC class II gene is a HLA-DM gene, a HLA-DO gene, a HLA-DP gene, a HLA-DQ gene, or a HLA-DR gene.

In certain embodiments, the engineered cell comprises at least one genetic modification in a CIITA gene. In certain embodiments, the engineered cell does not express a functional CIITA protein.

In some embodiments, the engineered cell comprises at least one modification in a MHC class I gene. In some embodiments, the MHC class I gene is a HLA-A gene, a HLA-B gene, or a HLA-C gene. In some embodiments, the cell does not express the HLA-A gene. In some embodiments, the engineered cells modified to reduce or eliminate HLA-A expression are homozygous for HLA-B and HLA-C. In certain embodiments, the HLA-B allele and the HLA-C allele of the engineered cell are HLA matched to a subject who is to be administered the engineered cell.

In certain aspects, provided herein is population of cells comprising an engineered cell provided herein.

In certain aspects, provided herein is a pharmaceutical composition comprising an engineered cell or population of cells provided herein.

In some aspects, provided herein is a method of treating a disease or disorder in a subject, the method comprising administering to the subject an engineered cell, population of cells, or pharmaceutical composition provided herein. In some embodiments the engineered cell is HLA matched to the subject (e.g., matched to the subject at one or more MHC class I gene, such as the HLA-B or HLA-C genes). In certain embodiments, the disease or disorder is a cancer, an infectious disease, or an autoimmune disease.

In some embodiments of the methods provided herein, the disease or disorder is a cancer. IN some embodiments, the cancer is a hematologic cancer (e.g., a CD30-expressing hematologic cancer). In certain embodiments, the CD30-expressing hematologic cancer is relapsed or refractory classical Hodgkin's Lymphoma.

In certain aspects, provided herein is method of preventing or reducing graft versus host disease in a subject receiving an allogenic cell treatment, the method comprising administering to the subject the engineered cell, population of cells, or pharmaceutical composition provided herein.

In some aspects, provided herein is an engineered cell, population of cells, or pharmaceutical composition disclosed herein, for use in an adoptive cell transfer (ACT) therapy.

In certain aspects, provided herein is a nucleic encoding the CAR provided herein. In some aspects, provided herein is a vector comprising a nucleic acid encoding a CAR provided herein. In certain aspects, provided herein is a cell comprising a nucleic acid or vector provided herein.

In certain aspects, provided herein is a method of making a CAR expressing engineered cell (e.g., an engineered T cell or NK cell), the method comprising delivering a nucleic acid or vector provided herein to a recipient cell. In some embodiments, the method further comprises delivering to the recipient cell a gRNA targeting a locus for inserting into the locus the nucleic acid sequence encoding the CAR. In some embodiments, the method further comprises delivering to the recipient cell a nuclease or a nucleic acid encoding a nuclease (e.g., a Cas9 nuclease). In some embodiments, the method further comprises delivering to the recipient cell a gRNA that targets the HLA-A gene. In some embodiments, the method further comprises delivering to the cell a gRNA that targets the CIITA gene. In some embodiments, the method further comprises delivering to the recipient cell a gRNA that targets the TRAC or TRBC locus. In some embodiments, the method further comprises delivering to the cell a gRNA that targets the B2M gene. In certain embodiments, provided herein is an engineered cell generated according to a method provided herein.

Further embodiments are provided throughout and including in the claims and Figures.

### BRIEF DESCRIPTION OF THE DRAWINGS

FIGS. 1A and 1B show T cell killing as the percent of HH tumor cell lysis with different CD30 constructs. FIG. 1A shows an exemplary plate form Donor 1, and FIG. 1B shows an exemplary plate from Donor 2.

FIGS. 2A and 2B show concentrations of cytokines released from cells after treatment. FIG. 2A shows the concentration of IFNγ released from cells post-treatment, and FIG. 2B shows the concentration of IL-2.

FIG. 3 shows the total green integrated object intensity (GCU×μm²/image) for CD30 constructs following tumor rechallenge over the course of 18 days.

FIG. 4 is a graph showing that CD30 CAR-T constructs demonstrate varied HH-Luc2 tumor control as measured by in vivo. Mice were injected at a dose of 5×10⁶ T cells.

FIG. 5 is a graph showing that cells expressing the CD30 CAR encoded by Constructs 3884 and 3887 were observed to have the maximum HH-Luc2 tumor control. Mice were injected at a dose of 5×10⁶ T cells.

FIGS. 6A and 6B show total flux (photons/second) from luciferase expressing tumor cells present at the various time points after injection. FIG. 6A shows the total flux observed after T cell administration with two different doses. FIG. 6B shows the total flux observed after rechallenge.

FIGS. 7A and 7B illustrate the total green integrated object intensity (GCU×μm²/image) for CD30 constructs that vary in the binder domain, following tumor rechallenge (at 400,000 cells/well (FIG. 7A) or 800,000 cells/well (FIG. 7B)) over the course of 16 days.

FIGS. 8A and 8B show in vivo tumor control using CARs that vary in the binder domain. FIG. 8A shows total flux (photons/second) from luciferase expressing tumor cells present at the various time points after injection, as observed after T cell administration or controls; tumor rechallenge at Day 34 is indicated by the dotted vertical line. FIG. 8B illustrates T cell proliferation at the indicated days post T cell injection.

FIG. 9 illustrates T cell killing as the percent of HH tumor cell lysis with different anti-CD30 CAR-T cells expressing anti-CD30 CARs that vary in the binder domain.

FIGS. 10A and 10B show concentrations of cytokines released from engineered T cells after co-culture with HH tumor cells. FIG. 10A shows the concentration of IFNγ released from engineered T cells post-treatment, and FIG. 10B shows the concentration of IL-2 released from engineered T cells post-treatment.

FIGS. 11A and 11B show the total flux (p/s) from luciferase-expressing tumor cells at various timepoints following treatment with either triple knockout engineered or single knockout engineered CAR T Cells. FIG. 11A shows the total flux observed after treating with constructs 3884 and 3875, single knockout engineered by Preparation Method 1. FIG. 11B shows the total flux observed following treatment with triple knockout engineered T cells engineered by Preparation Method 1 or by Preparation Method 2.

FIGS. 12A, 12B and 12C show various conditions of the CAR T Cells following editing. FIG. 12A shows fold expansion. FIG. 12B shows phenotype and percent editing. FIG. 12C shows rates of the CD30-CAR-T cells edited across 4 donors.

FIGS. 13A and 13B show the normalized proliferation of partially matched and mis-matched host T cells against different edited CD30 CAR-T cells for two T cell donors. FIG. 13A shows the proliferation for Donor 1, and FIG. 13B shows the proliferation for Donor 2.

### DETAILED DESCRIPTION

### A. General

The present disclosure provides chimeric antigen receptors (CARs) (e.g., CARs specific for CD30) to engineer human cells that are useful, for example, for adoptive cell transfer (ACT) therapies. In certain aspects, provided herein is a chimeric antigen receptor (CAR) comprising: (a) a binder domain (e.g., a binder domain that binds to CD30); (b) a hinge domain (e.g., a hinge domain comprising a hinge sequence selected from an IgG1-CH2-CH3 hinge sequence, an IgG1-CH3 hinge sequence, a CD8a hinge sequence, and a CD28 hinge sequence); (c) a transmembrane domain (e.g., a transmembrane domain comprising a transmembrane sequence selected from a CD28 transmembrane sequence and a CD8a transmembrane sequence); (d) a costimulatory domain (e.g., a costimulatory domain comprising a costimulatory sequence selected from a CD28 costimulatory sequence and a 41BB costimulatory sequence); and (e) an activation domain (e.g., comprising a CD3z activation sequence). In certain embodiments, the CAR comprises: (a) a binder domain that binds CD30; (b) a CD8a hinge domain; (c) a CD28 transmembrane domain; (d) a costimulatory domain; and (e) an activation domain.

As described herein, the CARs of the present disclosure can be used in autologous as well as allogeneic therapeutic cells. Methods of genetically modifying a cell to engineer a therapeutic cell having desirable properties, including e.g., reduced immunogenicity in a host immune system (e.g., to reduce/prevent GVHD), reducing susceptibility of an allogeneic cell to rejection by the host immune system, and increased genetic compatibility with greater subjects for transplant, are known in the art. For example, a gene encoding a MHC class I or class II can be modified to reduce the expression of MHC class I or class II protein on the surface of the cell expressing a CAR of the present disclosure. See, e.g., PCT/US2021/062946, PCT/US2021/064933, and PCT/US2021/064930, the contents of which are hereby incorporated in their entireties. In some embodiments, the disclosure provides engineered cells that express a CAR with reduced or eliminated surface expression of MHC class II as a result of a genetic modification in the CIITA gene. In some embodiments, the disclosure provides engineered cells that express a CAR, wherein the engineered cells have reduced or eliminated surface expression of MHC class I as a result of a genetic modification in the HLA gene (e.g., HLA-A). In some embodiments, the disclosure provides compositions and methods for engineering a cell that expresses a CAR of the present disclosure, wherein the cells have reduced or eliminated expression of MHC class I or II protein and compositions and methods to further reduce the cell's susceptibility to immune rejection. In some embodiments, the present disclosure further provides compositions and methods to reduce or eliminate surface expression of MHC class I protein in the cell by genetically modifying B2M (β-2-microgloblin). The B2M protein forms a heterodimer with MHC class I molecules and is required for MHC class I protein expression on the cell surface. In some embodiments that include a B2M genetic modification, the disclosure further provides expression of an NK cell inhibitor molecule by the cell to reduce or eliminate the lytic activity of NK cells.

The disclosure also provides methods and compositions for genetically modifying the engineered cell expressing a CAR to reduce expression of the endogenous T cell receptor (TCR) on the surface of the cell. Reduction or elimination of TCR expression in the engineered cell reduces or eliminates graft versus host disease (GVHD) in a subject that receives an engineered cell disclosed herein. In some embodiments, the disclosure provides engineered cells that express a CAR with reduced or eliminated surface expression of the endogenous TCR as a result of a genetic modification in the TCR gene.

### B. Definitions

Unless stated otherwise, the following terms and phrases as used herein are intended to have the following meanings:

The term “about” or “approximately” means an acceptable error for a particular value as determined by one of ordinary skill in the art, which depends in part on how the value is measured or determined, or a degree of variation that does not substantially affect the properties of the described subject matter, or within the tolerances accepted in the art, e.g., within 10%, 5%, 2%, or 1%. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

The term “or combinations thereof” as used herein refers to all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, CBBA, CABA, and so forth. The skilled artisan will understand that typically there is no limit on the number of items or terms in any combination, unless otherwise apparent from the context.

An “allogeneic” cell, as used herein, refers to a cell originating from a donor subject of the same species as a recipient subject, wherein the donor subject and recipient subject have genetic dissimilarity, e.g., genes at one or more loci that are not identical. Thus, e.g., a cell is allogeneic with respect to the subject to be administered the cell. As used herein, a cell that is removed or isolated from a donor, that will not be re-introduced into the original donor, is considered an allogeneic cell.

An “autologous” cell, as used herein, refers to a cell derived from the same subject to whom the material will later be re-introduced. Thus, e.g., a cell is considered autologous if it is removed from a subject and it will then be re-introduced into the same subject.

“β2M” or “B2M,” as used herein, refers to nucleic acid sequence or protein sequence of “β-2 microglobulin”; the human gene has accession number NC_000015 (range 44711492 . . . 44718877), reference GRCh38.p13. The B2M protein is associated with MHC class I molecules as a heterodimer on the surface of nucleated cells and is required for MHC class I protein expression.

“CIITA” or “CIITA” or “C2TA,” as used herein, refers to the nucleic acid sequence or protein sequence of “class II major histocompatibility complex transactivator;” the human gene has accession number NC_000016.10 (range 10866208 . . . 10941562), reference GRCh38.p13. The CIITA protein in the nucleus acts as a positive regulator of MHC class II gene transcription and is required for MHC class II protein expression.

As used herein, “MHC” or “MHC molecule(s)” or “MHC protein” or “MHC complex(es),” refers to a major histocompatibility complex molecule (or plural), and includes e.g., MHC class I and MHC class II molecules. In humans, MHC molecules are referred to as “human leukocyte antigen” complexes or “HLA molecules” or “HLA protein.” The use of terms “MHC” and “HLA” are not meant to be limiting; as used herein, the term “MHC” may be used to refer to human MHC molecules, i.e., HLA molecules. Therefore, the terms “MHC” and “HLA” are used interchangeably herein.

The term “HLA-A,” as used herein in the context of HLA-A protein, refers to the MHC class I protein molecule, which is a heterodimer consisting of a heavy chain (encoded by the HLA-A gene) and a light chain (i.e., beta-2 microglobulin). The term “HLA-A” or “HLA-A gene,” as used herein in the context of nucleic acids refers to the gene encoding the heavy chain of the HLA-A protein molecule. The HLA-A gene is also referred to as “HLA class I histocompatibility, A alpha chain;” the human gene has accession number NC_000006.12 (29942532 . . . 29945870). The HLA-A gene is known to have thousands of different genotypic versions of the HLA-A gene across the population (and an individual may receive two different alleles of the HLA-A gene). A public database for HLA-A alleles, including sequence information, may be accessed at IPD-IMGT/HLA: www.ebi.ac.uk/ipd/imgt/hla/. All alleles of HLA-A are encompassed by the terms “HLA-A” and “HLA-A gene.”

“HLA-B” as used herein in the context of nucleic acids refers to the gene encoding the heavy chain of the HLA-B protein molecule. The HLA-B is also referred to as “HLA class I histocompatibility, B alpha chain;” the human gene has accession number NC_000006.12 (31353875 . . . 31357179).

“HLA-C” as used herein in the context of nucleic acids refers to the gene encoding the heavy chain of the HLA-C protein molecule. The HLA-C is also referred to as “HLA class I histocompatibility, C alpha chain;” the human gene has accession number NC_000006.12 (31268749 . . . 31272092).

As used herein, an HLA “allele” can refer to a named HLA-A, HLA-B, or HLA-C gene wherein the first four digits (or the first two sets of digits separated by a colon, e.g., HLA-A*02:101:01:02N where the first two sets of digits are bolded and in italics) of the name following “HLA-A,” HLA-B,” or “HLA-C” are specified. As known in the art, the first four digits (or first two sets of digits separated by a colon) specify the protein of the allele. For example, HLA-A*02:01 and HLA-A*01:02 are distinct HLA-A alleles. Further genotypes of each allele exist, such as, e.g., HLA-A*02:01:02:01. Further genotypes of a given allele are considered to be identical alleles, e.g., HLA-A*02:01:02:01 and HLA-A*02:01 are identical alleles. Thus, HLA alleles are homozygous when the alleles are identical (i.e., when the alleles have the same first four digits or same first two sets of digits separated by a colon).

As used herein, the term “homozygous” refers to having two identical alleles of a particular gene.

“Matching” or “matched” refers to shared alleles between the donor and the recipient, e.g., identical alleles.

As used herein, the term “within the genomic coordinates” includes the boundaries of the genomic coordinate range given. For example, if chr6:29942854-chr6:29942913 is given, the coordinates chr6:29942854-chr6:29942913 are encompassed. Throughout this application, the referenced genomic coordinates are based on genomic annotations in the GRCh38 (also referred to as hg38) assembly of the human genome from the Genome Reference Consortium, available at the National Center for Biotechnology Information website. Tools and methods for converting genomic coordinates between one assembly and another are known in the art and can be used to convert the genomic coordinates provided herein to the corresponding coordinates in another assembly of the human genome, including conversion to an earlier assembly generated by the same institution or using the same algorithm (e.g., from GRCh38 to GRCh37), and conversion of an assembly generated by a different institution or algorithm (e.g., from GRCh38 to NCBI33, generated by the International Human Genome Sequencing Consortium). Available methods and tools known in the art include, but are not limited to, NCBI Genome Remapping Service, available at the National Center for Biotechnology Information website, UCSC LiftOver, available at the UCSC Genome Brower website, and Assembly Converter, available at the Ensembl.org website.

“Polynucleotide” and “nucleic acid” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion see The Biochemistry of the Nucleic Acids 5-36, Adams et al., ed., 11^{th} ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). RNA and DNA have different sugar moieties and can differ by the presence of uracil or analogs thereof in RNA and thymine or analogs thereof in DNA.

“Guide RNA,” “gRNA,” and simply “guide” are used herein interchangeably to refer to the guide nucleic acid that directs an RNA-guided DNA binding agent to a target DNA and can be a single guide RNA or the combination of a crRNA and a trRNA (also known as tracrRNA). Exemplary gRNAs include Class II Cas nuclease guide RNAs, in modified or unmodified forms. The crRNA and trRNA may be associated as a single RNA molecule (single guide RNA, sgRNA) or in two separate RNA strands (dual guide RNA, dgRNA). “Guide RNA” or “gRNA” refers to each type. The trRNA may be a naturally occurring sequence, or a trRNA sequence with modifications or variations compared to naturally-occurring sequences.

As used herein, a “guide sequence” refers to a sequence within a guide RNA that is complementary to a target sequence and functions to direct a guide RNA to a target sequence for binding or modification (e.g., cleavage) by an RNA-guided DNA binding agent. A “guide sequence” may also be referred to as a “targeting sequence,” or a “spacer sequence.” A guide sequence can be 20 base pairs in length, e.g., in the case of Streptococcus pyogenes (i.e., Spy Cas9 (SpCas9)) and related Cas9 homologs/orthologs. Shorter or longer sequences can also be used as guides, e.g., 15-, 16-, 17-, 18-, 19-, 21-, 22-, 23-, 24-, or 25-nucleotides in length. In some embodiments, the target sequence is in a gene or on a chromosome, for example, and is complementary to the guide sequence. In some embodiments, the degree of complementarity or identity between a guide sequence and its corresponding target sequence may be about 75%, 80%, 85%, 90%, 95%, 96%, 97%, 98%, 99%, or 100%. In some embodiments, the guide sequence and the target region may be 100% complementary or identical. In other embodiments, the guide sequence and the target region may contain at least one mismatch. For example, the guide sequence and the target sequence may contain 1, 2, 3, or 4 mismatches, where the total length of the target sequence is at least 17, 18, 19, 20 or more base pairs. In some embodiments, the guide sequence and the target region may contain 1-4 mismatches where the guide sequence comprises at least 17, 18, 19, 20 or more nucleotides. In some embodiments, the guide sequence and the target region may contain 1, 2, 3, or 4 mismatches where the guide sequence comprises 20 nucleotides.

Target sequences for RNA-guided DNA binding agents include both the positive and negative strands of genomic DNA (i.e., the sequence given and the sequence's reverse compliment), as a nucleic acid substrate for an RNA-guided DNA binding agent is a double stranded nucleic acid. Accordingly, where a guide sequence is said to be “complementary to a target sequence”, it is to be understood that the guide sequence may direct a guide RNA to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

As used herein, an “RNA-guided DNA binding agent” means a polypeptide or complex of polypeptides having RNA and DNA binding activity, or a DNA-binding subunit of such a complex, wherein the DNA binding activity is sequence-specific and depends on the sequence of the RNA. Exemplary RNA-guided DNA binding agents include Cas cleavases/nickases and inactivated forms thereof (“dCas DNA binding agents”). “Cas nuclease”, also called “Cas protein” as used herein, encompasses Cas cleavases, Cas nickases, and dCas DNA binding agents. Cas cleavases/nickases and dCas DNA binding agents include a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. As used herein, a “Class 2 Cas nuclease” is a single-chain polypeptide with RNA-guided DNA binding activity. Class 2 Cas nucleases include Class 2 Cas cleavases/nickases (e.g., H840A, D10A, or N863A variants), which further have RNA-guided DNA cleavases or nickase activity, and Class 2 dCas DNA binding agents, in which cleavase/nickase activity is inactivated. Class 2 Cas nucleases include, for example, Cas9, Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9(1.0) (e.g., K810A, K1003A, R1060A variants), and eSPCas9(1.1) (e.g., K848A, K1003A, R1060A variants) proteins and modifications thereof. Cpf1 protein, Zetsche et al., Cell, 163: 1-13 (2015), is homologous to Cas9, and contains a RuvC-like nuclease domain. Cpf1 sequences of Zetsche are incorporated by reference in their entirety. See, e.g., Zetsche, Tables S1 and S3. See, e.g., Makarova et al., Nat Rev Microbiol, 13(11): 722-36 (2015); Shmakov et al., Molecular Cell, 60:385-397 (2015).

As used herein, the term “fusion protein” refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

As used herein, “open reading frame” or “ORF” of a gene refers to a sequence consisting of a series of codons that specify the amino acid sequence of the protein that the gene codes for. The ORF begins with a start codon (e.g., ATG in DNA or AUG in RNA) and ends with a stop codon, e.g., TAA, TAG or TGA in DNA or UAA, UAG, or UGA in RNA.

As used herein, “ribonucleoprotein” (RNP) or “RNP complex” refers to a guide RNA together with an RNA-guided DNA binding agent, such as a Cas nuclease, e.g., a Cas cleavase, Cas nickase, or dCas DNA binding agent (e.g., Cas9). In some embodiments, the guide RNA guides the RNA-guided DNA binding agent such as Cas9 to a target sequence, and the guide RNA hybridizes with and the agent binds to the target sequence; in cases where the agent is a cleavase or nickase, binding can be followed by cleaving or nicking.

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server is generally appropriate.

“mRNA” is used herein to refer to a polynucleotide and comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof.

As used herein, “indels” refer to insertion/deletion mutations consisting of a number of nucleotides that are either inserted or deleted, e.g. at the site of double-stranded breaks (DSBs), in a target nucleic acid.

As used herein, “reduced or eliminated” expression of a protein on a cell refers to a partial or complete loss of expression of the protein relative to an unmodified cell. In some embodiments, the surface expression of a protein on a cell is measured by flow cytometry and has “reduced or eliminated” surface expression relative to an unmodified cell as evidenced by a reduction in fluorescence signal upon staining with the same antibody against the protein. A cell that has “reduced or eliminated” surface expression of a protein by flow cytometry relative to an unmodified cell may be referred to as “negative” for expression of that protein as evidenced by a fluorescence signal similar to a cell stained with an isotype control antibody. The “reduction or elimination” of protein expression can be measured by other known techniques in the field with appropriate controls known to those skilled in the art.

As used herein, “knockdown” refers to a decrease in expression of a particular gene product (e.g., protein, mRNA, or both), e.g., as compared to expression of an unedited target sequence. Knockdown of a protein can be measured by detecting total cellular amount of the protein from a sample, such as a tissue, fluid, or cell population of interest. It can also be measured by measuring a surrogate, marker, or activity for the protein. Methods for measuring knockdown of mRNA are known and include analyzing mRNA isolated from a sample of interest. In some embodiments, “knockdown” may refer to some loss of expression of a particular gene product, for example a decrease in the amount of mRNA transcribed or a decrease in the amount of protein expressed by a cell or population of cells (including in vivo populations such as those found in tissues).

As used herein, “knockout” refers to a loss of expression from a particular gene or of a particular protein in a cell. Knockout can result in a decrease in expression below the level of detection of the assay. Knockout can be measured either by detecting total cellular amount of a protein in a cell, a tissue or a population of cells. In some embodiments, the methods disclosed herein “knockout” TRBC1, TRBC2 or TRAC in one or more cells (e.g., in a population of cells. In some embodiments, a knockout is the complete loss of expression of a protein component of the T-cell receptor (e.g. TRBC1, TRBC2 or TRAC) in a cell, rather than the formation of a mutant T-cell receptor protein.

As used herein, “TRBC1” and “TRBC2” refer to two homologous genes encoding the T-cell receptor β-chain, which are the gene products of the TRBC1 or TRBC2 genes.

“TRBC” is used herein to refer to TRBC1 and TRBC2.

The human wild-type TRBC1 sequence is available at NCBI Gene ID: 28639; Ensembl: ENSG00000211751. T-cell receptor Beta Constant, V_segment Translation Product, BV05S1J2.2, TCRBC1, and TCRB are gene synonyms for TRBC1.

The human wild-type TRBC2 sequence is available at NCBI Gene ID: 28638; Ensembl: ENSG00000211772. T-cell receptor Beta Constant, V_segment Translation Product, and TCRBC2 are gene synonyms for TRBC2.

The human wild-type TRAC sequence is available at NCBI Gene ID: 28755; Ensembl: ENSG00000277734. T-cell receptor Alpha Constant, TCRA, IMD7, TRCA and TRA are gene synonyms for TRAC.

As used herein, a “target sequence” or “genomic target sequence” refers to a sequence of nucleic acid in a target gene that has complementarity to the guide sequence of the gRNA. The interaction of the target sequence and the guide sequence directs an RNA-guided DNA binding agent to bind, and potentially nick or cleave (depending on the activity of the agent), within the target sequence.

As used herein, “CD30” refers to a protein expressed by B and T cells and is involved in the activation of NF-kappaB. The human wild-type CD30 is encoded by the CD30 gene, which is available at NCBI Gene ID: 943; Ensemble: ENSG00000120949. TNFRSF8 is a synonym for the CD30 gene, among others readily known in the art.

As used herein, “treatment” refers to any administration or application of a therapeutic for disease or disorder in a subject, and includes inhibiting the disease, arresting its development, relieving one or more symptoms of the disease, curing the disease, or preventing one or more symptoms of the disease, including recurrence of the symptom.

Reference will now be made in detail to certain embodiments disclosed herein, examples of which are illustrated in the accompanying drawings. While the compositions and methods provided herein are described in conjunction with the illustrated embodiments, it will be understood that they are not intended to limit the invention to those embodiments. On the contrary, the invention is intended to cover all alternatives, modifications, and equivalents, which may be included within the invention as defined by the appended claims and included embodiments.

Before describing the present teachings in detail, it is to be understood that the disclosure is not limited to specific compositions or process steps, as such may vary. It should be noted that, as used in this specification and the appended claims, the singular form “a,” “an,” and “the” include plural references unless the context clearly dictates otherwise. Thus, for example, reference to “a conjugate” includes a plurality of conjugates and reference to “a cell” includes a plurality of cells and the like.

Numeric ranges are inclusive of the numbers defining the range. Measured and measurable values are understood to be approximate, taking into account significant digits and the error associated with the measurement. Also, the use of “comprise,” “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims). The term “or” is used in an inclusive sense, i.e., equivalent to “and/or,” unless the context clearly indicates otherwise.

The section headings used herein are for organizational purposes only and are not to be construed as limiting the desired subject matter in any way. In the event that any material incorporated by reference contradicts any term defined in this specification or any other express content of this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### C. Chimeric Antigen Receptors (CARs)

In various embodiments, genetically engineered receptors that redirect cytotoxicity of immune effector cells toward cells (e.g., CD30-expressing cells) are provided. These genetically engineered receptors are referred to herein as chimeric antigen receptors (CARs). CARs are molecules that combine specificity, e.g., antibody-based, for a desired target (e.g., CD30) with a T cell receptor-activating intracellular domain to generate a chimeric protein that exhibits a specific cellular immune activity. As used herein, the term, “chimeric,” describes being composed of parts of different proteins or DNAs from different origins.

In some embodiments, CARs provided herein comprise an extracellular domain, which is also referred to as a binder domain that binds to a target such as CD30, a transmembrane domain, and an intracellular signaling domain. In certain embodiments the binder domain is derived from an antibody fragment (e.g., an scFv, VHH, nanobody). Engagement of the binder domain of the CAR with its target (e.g., CD30) on the surface of a target cell results in clustering of the CAR and delivers an activation stimulus to the CAR-containing cell. The main characteristic of CARs is their ability to redirect immune effector cell specificity, thereby triggering proliferation, cytokine production, phagocytosis or production of molecules that can mediate cell death of the target antigen expressing cell in a major histocompatibility (MHC) independent manner, exploiting the cell specific targeting abilities of monoclonal antibodies, soluble ligands, or cell-specific co-receptors.

Accordingly, the present disclosure provides a chimeric antigen receptor (CAR), nucleic acids encoding CARs and cells comprising such CAR proteins and nucleic acids. In some embodiments, the nucleic acid encodes a CAR polypeptide that is expressed on the surface of the cell (i.e., a cell-surface bound protein). In some embodiments, the CAR is a targeting receptor. A “targeting receptor” is a receptor present on the surface of a cell, e.g., a T cell, to permit binding of the cell to a target site, e.g., a specific cell or tissue in an organism.

In some embodiments, the binder domain of the CARs provided is operably linked through at least a transmembrane domain to an internal signaling domain capable of activating a T cell upon binding of the binder domain. In certain embodiment, the CARs provided herein are composed of four regions: a binder domain (an antigen recognition domain), a hinge domain (an extracellular hinge region), a transmembrane domain, and an intracellular T cell signaling domain. The intracellular T cell signaling domain can include a costimulatory domain and an activation domain. In some embodiments, the CAR comprises at least one linker that links two of the domains referenced above together. In some embodiments, the CAR comprises a spacer.

In some embodiments, the CAR also comprises an N-terminal signal peptide. In some embodiments the signal peptide comprises a sequence of MDFQVQIFSFLLISASVIMSRMA (SEQ ID NO: 1; HRS3 Signal peptide). In some embodiments, the signal peptide comprises as sequence of MALPVTALLLPLALLLHAARP (SEQ ID NO: 2; 5F11 signal peptide).

### 1. Binder Domain

In particular embodiments, the CARs provided herein comprise a binder domain that comprises an antibody or antigen binding fragment thereof (e.g., an anti-CD30 antibody or antigen fragment thereof) that specifically binds to a target antigen (e.g., CD30) expressed on a target cell. As used herein, the terms, “binder domain,” “binding domain,” “extracellular domain,” “extracellular binding domain,” “antigen-specific binding domain,” and “extracellular antigen specific binding domain,” are used interchangeably and provide a CAR with the ability to specifically bind to the target antigen of interest, e.g., CD30.

In some embodiments, the binder domain of a CAR described herein can be any peptide that binds to an antigen. For example, the binding domain can be an Fab fragment (Fab), F(ab)2 fragment, diabody, triabody, tetrabody, single-chain variable region fragment (scFv), or a disulfide-stabilized variable region fragment (dsF), and the like. In some embodiments, the binding domain specifically binds to an antigen.

The terms “specific binding affinity” or “specifically binds” or “specifically bound” or “specific binding” or “specifically targets” or “binds to” as used herein, describe a binder domain (or a CAR comprising the same) engagement to a target at greater binding affinity than background binding. A binder domain (or a CAR comprising a binder domain) “specifically binds” to a target if it binds to the target with an affinity or Kₐ (i.e., an equilibrium association constant of a particular binding interaction with units of 1/M) of, for example, greater than or equal to about 10⁵ M⁻¹. In certain embodiments, a binder domain (or a CAR containing a binder domain) binds to a target with a Kₐ greater than or equal to about 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 103 M⁻¹. “High affinity” binder domains (or CARs containing such binder domains) refers to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater. In some embodiments, the affinity of specific binding is about 2 times greater than background binding, about 5 times greater than background binding, about 10 times greater than background binding, about 20 times greater than background binding, about 50 times greater than background binding, about 100 times greater than background binding, or about 1000 times greater than background binding or more.

In some embodiments, the CAR comprises a binder domain that binds to CD30. In certain embodiments, the binder domain comprises a CD30 antibody or antigen binding fragment thereof (e.g., an scFv).

In certain embodiments, the binder domain is a HRS3 binder domain or a variant thereof. Exemplary HRS3 binder domains and variant HRS3 binder domains can be found in U.S. Pat. No. 10,808,035 and US Pat. App. Pub. No. US 2016/0200824, each of which are incorporated by reference in their entirety. “HRS3” as used herein refers to the HRS3 binder domains disclosed herein or known in the art.

In certain embodiments, the binder domain is a 5F11 binder domain or a variant thereof. Exemplary 5F11 binder domains and variant 5F11 binder domains can be found in U.S. Pat. No. 8,088,377 and PCT App. Pub. No. WO 2017/066122, each of which are incorporated by reference in their entirety. “5F11” as used herein refers to the 5F11 binder domains disclosed herein or known in the art.

In some embodiments the binders of the CARs provided herein comprise a sequence at least 80% (e.g., at least 85%, at least 90%, at least 91%, at least 92%, at least 93%, at least 94%, at least 95%, at least 96%, at least 97%, at least 98%, at least 99%, or 100%) identical to a sequence selected from Table 1 or 1A. In some embodiments, the binder domain comprises an amino acid sequence that has no more than 10, 9, 8, 7, 6, 5, 4, 3, 2, or 1 amino acid substitutions (e.g., conservative amino acid substitutions) compared to a sequence listed in Table 1 or 1A.

TABLE 1
Exemplary Binder Domain Sequences

| | | SEQ ID |
|---|---|---|
| Component | Sequence | NO. |
| HRS3 | QVQLQQSGAELARPGASVKMSCKASGYTFTTYTIHWVRRRPGHDLEWIG | 20 |
| Binder | YINPSSGCSDYNQNFKGKTTLTADKSSNTAYMQLNSLTSEDSAVYYCARR | |
| 3884 | ADYGNYEYTWFAYWGQGTTVTVSSSGGGSGGGGSGGGGSVIELTQSPKF | |
| | MSTSVGDRVNVTYKASQNVGTNVAWFQQKPGQSPKVLIYSASYRYSGVP | |
| | DRFTGSGSGTDFTLTISNVQSEDLAEYFCQQYHTYPLTFGGGTKLEIKR | |
| HRS3 | QVQLQQSGAELARPGASVKMSCKASGYTFTTYTIHWVRQRPGHDLEWIG | 21 |
| Binder | YINPSSGYSDYNQNFKGKTTLTADKSSNTAYMQLNSLTSEDSAVYYCARR | |
| 4621 | ADYGNYEYTWFAYWGQGTTVTVSSGGGGSGGGGSGGGGSDIELTQSPKF | |
| | MSTSVGDRVNVTYKASQNVGTNVAWFQQKPGQSPKVLIYSASYRYSGVP | |
| | DRFTGSGSGTDFTLTISNVQSEDLAEYFCQQYHTYPLTFGGGTKLEIKR | |
| HRS3 | QVQLQQSGAELARPGASVKMSCKASGYTFTTYTIHWVRRRPGHDLEWIG | 22 |
| Binder | YINPSSGYSDYNQNFKGKTTLTADKSSNTAYMQLNSLTSEDSAVYYCARR | |
| 4622 | ADYGNYEYTWFAYWGQGTTVTVSSSGGGSGGGGSGGGGSVIELTQSPKF | |
| | MSTSVGDRVNVTYKASQNVGTNVAWFQQKPGQSPKVLIYSASYRYSGVP | |
| | DRFTGSGSGTDFTLTISNVQSEDLAEYFCQQYHTYPLTFGGGTKLEIKR | |
| 5F11 | DIQMTQSPTSLSASVGDRVTITCRASQGISSWLTWYQQKPEKAPKSLIYAA | 23 |
| Binder | SSLQSGVPSRFSGSGSGTDFTLTISSLQPEDFATYYCQQYDSYPITFGQGTR | |
| | LEIKGSTSGSGKPGSGEGSTKGQVQLQQWGAGLLKPSETLSLTCAVYGGS | |
| | FSAYYWSWIRQPPGKGLEWIGDINHGGGTNYNPSLKSRVTISVDTSKNQF | |
| | SLKLNSVTAADTAVYYCASLTAYWGQGSLVTVSS | |

TABLE 1A
Exemplary Binder VH and VL Sequences

| VH | VL |
|---|---|
| QVQLQQSGAELARPGASVKMSCKASGY | VIELTQSPKFMSTSVGDRVNVTYKASQNVGTNV |
| TFTTYTIHWVRRRPGHDLEWIGYINPSSG | AWFQQKPGQSPKVLIYSASYRYSGVPDRFTGSGS |
| CSDYNQNFKGKTTLTADKSSNTAYMQL | GTDFTLTISNVQSEDLAEYFCQQYHTYPLTFGGG |
| NSLTSEDSAVYYCARRADYGNYEYTWF | TKLEIKRSDP (SEQ ID NO: 25) |
| AYWGQGTTVTVSS (SEQ ID NO: 24) | |
| QVQLQQSGAELARPGASVKMSCKASGY | DIELTQSPKFMSTSVGDRVNVTYKASQNVGTNV |
| TFTTYTIHWVRQRPGHDLEWIGYINPSSG | AWFQQKPGQSPKVLIYSASYRYSGVPDRFTGSGS |
| YSDYNQNFKGKTTLTADKSSNTAYMQL | GTDFTLTISNVQSEDLAEYFCQQYHTYPLTFGGG |
| NSLTSEDSAVYYCARRADYGNYEYTWF | TKLEIKRSDP (SEQ ID NO: 27) |
| AYWGQGTTVTVSS (SEQ ID NO: 26) | |
| QVQLQQSGAELARPGASVKMSCKASGY | VIELTQSPKFMSTSVGDRVNVTYKASQNVGTNV |
| TFTTYTIHWVRRRPGHDLEWIGYINPSSG | AWFQQKPGQSPKVLIYSASYRYSGVPDRFTGSGS |
| YSDYNQNFKGKTTLTADKSSNTAYMQL | GTDFTLTISNVQSEDLAEYFCQQYHTYPLTFGGG |
| NSLTSEDSAVYYCARRADYGNYEYTWF | TKLEIKRSDP (SEQ ID NO: 25) |
| AYWGQGTTVTVSS (SEQ ID NO: 28) | |
| EVKLQQSGAELARPGASVKMSCKASGY | DIELTQSPKFMSTSVGDRVNVTCKASQNVGTNV |
| TFTTYTIHWVRQRPGHDLEWIGYINPSSG | AWFQQKPGQSPKVLIYSASYRYSGVPDRFTGSGS |
| YSDYNQNFKGKTTLTADKSSNTAYMQL | GTDFTLTISNVQSEDLAEYFCQQYHTYPLTFGGG |
| NSLTSEDSAVYYCARRADYGNYEYTWF | TKLEIKRSDP (SEQ ID NO: 30) |
| AYWGQGTTVTVSS (SEQ ID NO: 29) | |
| QVQLQQWGAGLLKPSETLSLTCAVYGG | DIQMTQSPTSLSASVGDRVTITCRASQGISSWLT |
| SFSAYYWSWIRQPPGKGLEWIGDINHGG | WYQQKPEKAPKSLIYAASSLQSGVPSRFSGSGSG |
| GTNYNPSLKSRVTISVDTSKNQFSLKLNS | TDFTLTISSLQPEDFATYYCQQYDSYPITFGQGTR |
| VTAADTAVYYCASLTAYWGQGSLVTVS | LEIK (SEQ ID NO: 32) |
| S (SEQ ID NO: 31) | |

In some embodiments, the CD30 antibody or antigen binding fragment thereof comprises a heavy chain variable region (VH) complementarity-determining region 1 (CDR1) sequence TFTTYT (SEQ ID NO: 3); a VH CDR2 sequence INPSSGCSD (SEQ ID NO: 4) or INPSSGYSD (SEQ ID NO: 5); a VH CDR3 sequence RADYGNYEYTWFAY (SEQ ID NO: 6); a light chain variable region (VL) CDR1 sequence ASQNVGTNVA (SEQ ID NO: 7); a VL CDR2 sequence SASYRYS (SEQ ID NO: 8); and a VL CDR3 sequence QQYHTYP (SEQ ID NO: 9). In certain embodiments, the VH amino acid sequence has at least 80% (e.g., at least 85%, at least 90%, at least 91%, at least 92%, at least 93%, at least 94%, at least 95%, at least 96%, at least 97%, at least 98%, at least 99%, or 100%) identity to sequence SEQ ID NO: 24, SEQ ID NO: 26, SEQ ID NO: 28, or SEQ ID NO: 29 and the VL amino acid sequence has at least 80% (e.g., at least 85%, at least 90%, at least 91%, at least 92%, at least 93%, at least 94%, at least 95%, at least 96%, at least 97%, at least 98%, at least 99%, or 100%) identity to SEQ ID NO: 25, SEQ ID NO: 27, or SEQ ID NO: 30. In some embodiments, the binder domain is HRS3 (see also U.S. Pat. No. 10,808,035 and US Pat. App. Pub. No. US 2016/0200824, the contents of each are incorporated by reference in their entirety; Protein Eng Des Sel. 2004 December; 17(12):847-60; Protein Eng Des Sel. 2015; 28(4):93-106; and DE19640733, incorporated by reference in its entirety).

In some embodiments, the CD30 antibody or antigen binding fragment thereof comprises a heavy chain variable region (VH) complementarity-determining region 1 (CDR1) sequence AYYWS (SEQ ID NO: 10); a VH CDR2 sequence DINHGGGTNYNPSLKS (SEQ ID NO: 11); a VH CDR3 sequence LTAY (SEQ ID NO: 12); a light chain variable region (VL) CDR1 sequence RASQGISSWLT (SEQ ID NO: 13); a VL CDR2 sequence AASSLQS (SEQ ID NO: 14); and a VL CDR3 sequence QQYDSYPIT (SEQ ID NO: 15). In certain embodiments, the VH amino acid sequence has at least 80% (e.g., at least 85%, at least 90%, at least 91%, at least 92%, at least 93%, at least 94%, at least 95%, at least 96%, at least 97%, at least 98%, at least 99%, or 100%) identity to sequence SEQ ID NO: 31 and the VL amino acid sequence has at least 80% (e.g., at least 85%, at least 90%, at least 91%, at least 92%, at least 93%, at least 94%, at least 95%, at least 96%, at least 97%, at least 98%, at least 99%, or 100%) identity to SEQ ID NO: 32. In certain embodiments, the binder domain is 5F11 (see also, U.S. Pat. No. 8,088,377 and PCT App. Pub. No. WO 2017/066122, the contents of each incorporated by reference in their entirety).

### 2. Linker

In certain embodiments, the CARs provided herein comprise linkers between the various domains, e.g., between VH and VL regions (a “variable region linking sequence”) of the binder domain (see, e.g., U.S. Pat. No. 11,279,769, incorporated by reference in its entirety). Linkers may add spacing that facilitates proper conformation of the CAR. In some embodiments, the linker is a variable region linking sequence. An extracellular binder domain comprising a variable region linking sequence retains specific binding affinity to the same target molecule as an antibody that comprises the same light and heavy chain variable regions. In particular embodiments, a linker separates one or more heavy or light chain variable domains, hinge domains, transmembrane domains, co-stimulatory domains, or activation domains. In some embodiments, a CAR can comprise one, two, three, four, or five or more linkers. In some embodiments, the length of a linker is about 1 to about 25 amino acids, about 5 to about 20 amino acids, or about 10 to about 20 amino acids, or any intervening length of amino acids. In some embodiments, the linker is 1, 2, 3, 4, 5, 6, 7, 8, 9, 10, 11, 12, 13, 14, 15, 16, 17, 18, 19, 20, 21, 22, 23, 24, 25, or more amino acids long.

In some embodiments, a linker comprises glycine polymers (G)n; glycine-serine polymers (G1-5S1-5)n, where n is an integer of at least one, two, three, four, or five; glycine-alanine polymers; alanine-serine polymers; and other flexible linkers known in the art. Glycine and glycine-serine polymers are relatively unstructured.

In some embodiments, a linker comprises, but are not limited to, the following amino acid sequences: GGG; DGGGS (SEQ ID NO: 40); TGEKP (SEQ ID NO: 41) (see, e.g., Liu et al., PNAS 5525-5530 (1997)); GGRR (SEQ ID NO: 42) (Pomerantz et al. 1995, supra); (GGGGS)n wherein n=1, 2, 3, 4 or 5 (SEQ ID NO: 43) (Kim et al., PNAS 93, 1156-1160 (1996.); EGKSSGSGSESKVD (SEQ ID NO: 818) (Chaudhary et al., 1990, Proc. Natl. Acad. Sci. U.S.A. 87:1066-1070); KESGSVSSEQLAQFRSLD (SEQ ID NO: 44) (Bird et al., 1988, Science 242:423-426), GGRRGGGS (SEQ ID NO: 45); LRQRDGERP (SEQ ID NO: 46); LRQKDGGGSERP (SEQ ID NO: 47); LRQKD(GGGS)2 ERP (SEQ ID NO: 48). In some embodiments, a flexible linker can be rationally designed using a computer program to model both DNA-binding sites and the peptides themselves (Desjarlais & Berg, PNAS 90:2256-2260 (1993), PNAS 91:11099-11103 (1994) or by phage display methods. In one embodiment, the linker comprises the amino acid sequence GSTSGSGKPGSGEGSTKG(SEQ ID NO: 49) (Cooper et al., Blood, 101(4): 1637-1644 (2003)).

In some embodiments, the linker comprises a sequence of KPDK (SEQ ID NO: 16).

In some embodiments, the CARs provided herein comprise any of the linkers provided herein between the hinge domain and the transmembrane domain. In some embodiments, the linker between the hinge domain and the transmembrane domain comprises a sequence of KPDK (SEQ ID NO: 16).

### 3. Hinge Domain

The binder domain of the CAR is generally followed by one or more “hinge domains,” which play a role in positioning the antigen binding domain away from the effector cell surface to enable proper cell/cell contact, antigen binding and activation. A CAR generally comprises one or more hinge domains between the binding domain and the transmembrane domain (TM). The hinge domain may be derived either from a natural, synthetic, semi-synthetic, or recombinant source. The hinge domain can include the amino acid sequence of a naturally occurring immunoglobulin hinge region or an altered immunoglobulin hinge region. In some embodiments, the hinge domain comprises a portion of an immunoglobulin, including, but not limited to, one or more heavy chain constant regions, e.g., CH2 and CH3. In some embodiments, the hinge domain comprises the CH2 and CH3 domains of IgG1, IgG4, or IgD.

In some embodiments, the hinge domain comprises a portion of other transmembrane proteins, e.g. CD8a or CD28.

In some embodiments, the CAR comprises a binder domain that is adjacent to a hinge domain. The term “hinge domain” or “hinge region” as used herein refers to a polypeptide that links the transmembrane domain of a CAR to the extracellular binding domain. In some embodiments, the term “hinge domain” or “hinge region” can be used interchangeably with “spacer domain”. The hinge region can provide extra mobility and accessibility to the extracellular binding domain.

In some embodiments, the hinge domain is an IgG1-CH2-CH3 domain known in the art including, e.g., SEQ ID NO: 52. In some embodiments, the hinge domain is an IgG1-CH3 hinge domain known in the art including, e.g., SEQ ID NO: 53. In some embodiments, the hinge domain is a CD8a hinge domain known in the art including, e.g., SEQ ID NO: 50. In some embodiments, the hinge domain is a CD28 hinge domain known in the art including, e.g., SEQ ID NO: 51. The term “CD8a hinge domain” as used herein encompasses known CD8a hinge domain sequences, including wild-type and the specific sequence disclosed herein. The term “CD28 hinge domain” as used herein encompasses known CD28 hinge domain sequences, including wild-type and the specific sequence disclosed herein. The term “IgG1-CH2-CH3 hinge domain” as used herein encompasses known IgG1-CH2-CH3 hinge domain sequences, including wild-type and the specific sequence disclosed herein. The term “IgG1-CH3 hinge domain” as used herein encompasses known IgG1-CH3 hinge domain sequences, including wild-type and the specific sequence disclosed herein.

Table 2 provides a list of exemplary hinge domain sequences. In some embodiments, the hinge domain comprises an amino acid sequence that is at least 70%, 75%, 80%, 85%, 90%, 95%, 97%, 99%, 99.5% identical to a sequence listed in Table 2. In some embodiments, the hinge domain comprises an amino acid sequence that has no more than 10, 9, 8, 7, 6, 5, 4, 3, 2, or 1 amino acid substitutions (e.g., conservative amino acid substitutions) compared to a sequence listed in Table 2.

TABLE 2
Exemplary Hinge Domain Sequences

| | | SEQ ID |
|---|---|---|
| Component | Sequence | NO. |
| CD8α | TTTPAPRPPTPAPTIASQPLSLRPEACRPAAGGAVHTRGLDFACD | 50 |
| Hinge | | |
| CD28 | IEVMYPPPYLDNEKSNGTIIHVKGKHLCPSPLFPGPSKP | 51 |
| Hinge | | |
| IgG1-CH2- | PCPAPPVAGPSVFLFPPKPKDTLMIARTPEVTCVVVDVSHEDPEVKFNWY | 52 |
| CH3 Hinge | VDGVEVHNAKTKPREEQYNSTYRVVSVLTVLHQDWLNGKEYKCKVSNK | |
| | ALPAPIEKTISKAKGQPREPQVYTLPPSRDELTKNQVSLTCLVKGFYPSDIA | |
| | VEWESNGQPENNYKTTPPVLDSDGSFFLYSKLTVDKSRWQQGNVFSCSV | |
| | MHEALHNHYTQKSLSLSPGK | |
| IgG1-CH3 | GQPREPQVYTLPPSRDELTKNQVSLTCLVKGFYPSDIAVEWESNGQPENN | 53 |
| Hinge | YKTTPPVLDSDGSFFLYSKLTVDKSRWQQGNVFSCSVMHEALHNHYTQK | |
| | SLSLSPGK | |

### 4. Transmembrane Domain

The “transmembrane domain” is the portion of the CAR that fuses the extracellular binding portion and intracellular signaling domain and anchors the CAR to the plasma membrane of the immune effector cell. The TM domain may be derived either from a natural, synthetic, semi-synthetic, or recombinant source.

In certain embodiments, the CAR provided herein comprises a transmembrane domain that is linked at its N-terminus to a binding domain via the hinge, and to a costimulatory domain on its C-terminus. In some embodiments of the CARs provided herein, the CAR comprises a linker between the hinge domain and the transmembrane domain. In some embodiments, the linker comprises a sequence of KPDK (SEQ ID NO: 16).

In some embodiments, the TM domain may be derived from (i.e., comprise at least the transmembrane region(s)) of the alpha or beta chain of the T-cell receptor, CDδ, CD3ε, CD3γ, CD3ζ, CD4, CD5, CD8α, CD9, CD 16, CD22, CD27, CD28, CD32, CD37, CD45, CD64, CD80, CD86, CD 134, CD137, CD152, CD154, and PD1. In some embodiments, the transmembrane domain is a CD28 transmembrane domain (e.g., SEQ ID NO: 54). In some embodiments, the transmembrane domain is a CD8a transmembrane domain (e.g., SEQ ID NO: 55).

Table 3 provides a list of exemplary transmembrane domain sequences. In some embodiments, the transmembrane domain comprises an amino acid sequence that is at least 70%, 75%, 80%, 85%, 90%, 95%, 97%, 99%, 99.5% identical to a sequence listed in Table 3 (e.g., SEQ ID NO: 54). In some embodiments, the transmembrane domain comprises an amino acid sequence that has no more than 10, 9, 8, 7, 6, 5, 4, 3, 2, or 1 amino acid substitutions (e.g., conservative amino acid substitutions) compared to a sequence listed in Table 3 (e.g., SEQ ID NO: 54 or SEQ ID NO: 55).

TABLE 3
Exemplary Transmembrane Domain Sequences

| | | SEQ ID |
|---|---|---|
| Component | Sequence | NO. |
| CD28 | FWVLVVVGGVLACYSLLVTVAFIIFWV | 54 |
| Transmembrane | | |
| Domain | | |
| CD8a | IYIWAPLAGTCGVLLLSLVIT | 55 |
| Transmembrane | | |
| Domain | | |

### 5. Intracellular Signaling Domain

In particular embodiments, CARs provided herein comprise an intracellular signaling domain. An “intracellular signaling domain,” refers to the part of a CAR that participates in transducing the message of effective target binding into the interior of the immune effector cell to elicit effector cell function, e.g., activation, cytokine production, proliferation and cytotoxic activity, including the release of cytotoxic factors to the CAR-bound target cell, or other cellular responses elicited with antigen binding to the extracellular CAR domain.

The term “effector function” refers to a specialized function of an immune effector cell. Effector function of the T cell, for example, may be cytolytic activity or helper activity or activity including the secretion of a cytokine. Thus, the term “intracellular signaling domain” refers to the portion of a protein which transduces the effector function signal and that directs the cell to perform a specialized function. While usually the entire intracellular signaling domain can be employed, in many cases it is not necessary to use the entire domain. To the extent that a truncated portion of an intracellular signaling domain is used, such truncated portion may be used in place of the entire domain as long as it transduces the effector function signal. The term intracellular signaling domain is meant to include any truncated portion of the intracellular signaling domain sufficient to transduce effector function signal.

Typically, signals generated through the TCR alone are insufficient for full activation of the T cell and that a secondary or co-stimulatory signal is also required. Thus, T cell activation can be said to be mediated by two distinct classes of intracellular signaling domains: primary signaling domains that initiate antigen-dependent primary activation through the activation domains of the TCR (e.g., a TCR/CD3 complex) and co-stimulatory signaling domains that act in an antigen-independent manner to provide a secondary or co-stimulatory signal. In certain embodiments, the CARs comprises an intracellular signaling domain that comprises both a “costimulatory domain” and an “activation domain.”

### 6. Costimulatory Domain

In particular embodiments, CARs provided herein comprise one or more costimulatory domains to enhance the efficacy and expansion of T cells expressing CAR receptors. As used herein, the term, “costimulatory domain”, refers to an intracellular signaling domain of a costimulatory molecule. Costimulatory molecules are cell surface molecules other than antigen receptors or Fc receptors that provide a second signal required for efficient activation and function of T lymphocytes upon binding to antigen. Illustrative examples of such co-stimulatory molecules include TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, NKD2C, SLP76, TRIM, and ZAP70.

In some embodiments, the CAR comprises a costimulatory domain. In some embodiments, a CAR comprises one or more costimulatory domains selected from the group consisting of CD28 and 41BB. In some embodiments, the CAR comprises a CD28 costimulatory domain (e.g., SEQ ID NO: 57), sometimes referred to herein as wild-type CD28 costimulatory domain. In some embodiments, the CD28 costimulatory domain comprises point mutation that eliminates lck binding (SEQ ID NO: 58). In some embodiments, the CAR comprises a 41BB costimulatory domain (SEQ ID NO: 59). “CD28 costimulatory domain” as used herein encompasses either wild-type or a variant form, unless indicated otherwise by context.

Table 4 provides a list of exemplary costimulatory domain sequences. In some embodiments, the costimulatory domain comprises an amino acid sequence that is at least 70%, 75%, 80%, 85%, 90%, 95%, 97%, 99%, 99.5% identical to a sequence listed in Table 4. In some embodiments, the costimulatory domain comprises an amino acid sequence that has no more than 10, 9, 8, 7, 6, 5, 4, 3, 2, or 1 amino acid substitutions (e.g., conservative amino acid substitutions) compared to a sequence listed in Table 4.

TABLE 4
Exemplary Costimulatory Domain Sequences

| | | SEQ |
|---|---|---|
| | | ID |
| Component | Sequence | NO. |
| CD28 | RSKRSRLLHSDYMNMTPRRPGPTRKHYQPYAPP | 57 |
| Costimulatory | RDFAAYRS | |
| Domain | | |
| CD28* | RSKRSRLLHSDYMNMTPRRPGPTRKHYQAYAAA | 58 |
| Costimulatory | RDFAAYRS | |
| Domain | | |
| 41BB | KRGRKKLLYIFKQPFMRPVQTTQEEDGCSCRFP | 59 |
| Costimulatory | EEEEGGCEL | |
| Domain | | |

### 7. Activation Domain

Activation domains regulate primary activation of the TCR complex either in a stimulatory way, or in an inhibitory way. An activation domain that acts in a stimulatory manner may contain signaling motifs which are known as immunoreceptor tyrosine-based activation motifs or ITAMs.

Illustrative examples of ITAM containing activation domains that are suitable for use in particular embodiments include those derived from FcRγ, FcRβ, CD3γ, CD3δ, CD3ε, CD3ζ (or CD3z), CD22, CD79a, CD79b, and CD66d. In certain embodiments, the CAR provided herein comprises a CD3z activation domain.

Accordingly, in some embodiments, the CAR comprises an activation domain. In some embodiments, the CAR comprises a wild type CD3z activation domain having an N-terminal arginine (e.g., SEQ ID NO: 60). In some embodiments, the CD3z activation domain comprises is a wild type activation domain comprising an N-terminal leucine residue (e.g., SEQ ID NO: 61). In some embodiments, the CD3z activation domain comprises the sequence SEQ ID NO: 62.

Table 5 provides a nonlimiting list of exemplary activation domain sequences. In some embodiments, the activation domain comprises an amino acid sequence that is at least 70%, 75%, 80%, 85%, 90%, 95%, 97%, 99%, 99.5% identical to a sequence listed in Table 5. In some embodiments, the activation domain comprises an amino acid sequence that has no more than 10, 9, 8, 7, 6, 5, 4, 3, 2, or 1 amino acid substitutions (e.g., conservative amino acid substitutions) compared to a sequence listed in Table 5.

TABLE 5
Exemplary Activation Domain Sequences

| | | SEQ ID |
|---|---|---|
| Component | Sequence | NO. |
| CD3ζ | RVKFSRSADAPAYQQGQNQLYNELNLGRREEYDVLDKRRGRDPEMGG | 60 |
| Activation | KPRRKNPQEGLYNELQKDKMAEAYSEIGMKGERRRGKGHDGLYQGLS | |
| Domain | TATKDTYDALHMQALPPR | |
| CD3ζ* | LRVKFSRSADAPAYQQGQNQLYNELNLGRREEYDVLDKRRGRDPEMG | 61 |
| Activation | GKPRRKNPQEGLYNELQKDKMAEAYSEIGMKGERRRGKGHDGLYQGL | |
| Domain | STATKDTYDALHMQALPPR | |
| CD3ζ** | RVKFSRSADAPAYKQGQNQLYNELNLGRREEYDVLDKRRGRDPEMGG | 62 |
| Activation | KPRRKNPQEGLYNELQKDKMAEAYSEIGMKGERRRGKGHDGLYQGLS | |
| Domain | TATKDTYDALHMQALPPR. | |

### 8. Exemplary CARs

The CARs disclosed herein comprise various combinations of the domains described herein. In certain embodiments, the CAR comprises: (a) a binder domain (e.g., a binder domain that binds to CD30, such as HRS3 or 5F11); (b) a hinge domain (e.g., a hinge domain comprising a hinge sequence selected from an IgG1-CH2-CH3 hinge sequence, an IgG1-CH3 hinge sequence, a CD8a hinge sequence, and a CD28 hinge sequence); (c) a transmembrane domain (e.g., a transmembrane domain comprising a transmembrane sequence selected from a CD28 transmembrane sequence and a CD8a transmembrane sequence); (d) a costimulatory domain (e.g., a costimulatory domain comprising a costimulatory sequence selected from a CD28 costimulatory sequence and a 41BB costimulatory sequence); and (e) an activation domain (e.g., comprising a CD3z activation sequence). In certain embodiments, the CAR comprises: (a) a binder domain that binds CD30; (b) a CD8a hinge domain; (c) a CD28 transmembrane domain; (d) a costimulatory domain; and (e) an activation domain. In some embodiments, the CAR also comprises an N-terminal signal peptide. In some embodiments the signal peptide comprises a sequence of MDFQVQIFSFLLISASVIMSRMA (SEQ ID NO: 1; HRS3 Signal peptide). In some embodiments, the signal peptide comprises as sequence of MALPVTALLLPLALLLHAARP (SEQ ID NO: 2; 511 signal peptide).

Table 6 provides a list of exemplary CAR sequences. In some embodiments, the CAR comprises an amino acid sequence that is at least 70%, 75%, 80%, 85%, 90%, 95%, 96%, 97%, 98%, 99%, 99.5% identical to a sequence listed in Table 6. In some embodiments, the CAR comprises an amino acid sequence that has no more than 10, 9, 8, 7, 6, 5, 4, 3, 2, or 1 amino acid substitutions (e.g., conservative amino acid substitutions) compared to a sequence listed in Table 6. The Additional Sequences Table 17 provides a list of the nucleic acid sequences encoding the CARs disclosed herein.

TABLE 6
Exemplary CAR Sequences

| | | SEQ |
|---|---|---|
| Con- | | ID |
| struct | Sequence | NO |
| 3881 | QVQLQQSGAELARPGASVKMSCKASGYTFTTYTIHWVRRRPGHDLEWIGYI | 63 |
| | NPSSGCSDYNQNFKGKTTLTADKSSNTAYMQLNSLTSEDSAVYYCARRADY | |
| | GNYEYTWFAYWGQGTTVTVSSSGGGSGGGGSGGGGSVIELTQSPKFMSTSV | |
| | GDRVNVTYKASQNVGTNVAWFQQKPGQSPKVLIYSASYRYSGVPDRFTGSG | |
| | SGTDFTLTISNVQSEDLAEYFCQQYHTYPLTFGGGTKLEIKRSDPAEPKSPDKT | |
| | HTCPPCPAPPVAGPSVFLFPPKPKDTLMIARTPEVTCVVVDVSHEDPEVKFNW | |
| | YVDGVEVHNAKTKPREEQYNSTYRVVSVLTVLHQDWLNGKEYKCKVSNKA | |
| | LPAPIEKTISKAKGQPREPQVYTLPPSRDELTKNQVSLTCLVKGFYPSDIAVEW | |
| | ESNGQPENNYKTTPPVLDSDGSFFLYSKLTVDKSRWQQGNVFSCSVMHEAL | |
| | HNHYTQKSLSLSPGKKDPKFWVLVVVGGVLACYSLLVTVAFIIFWVRSKRSR | |
| | LLHSDYMNMTPRRPGPTRKHYQAYAAARDFAAYRSLRVKFSRSADAPAYQ | |
| | QGQNQLYNELNLGRREEYDVLDKRRGRDPEMGGKPRRKNPQEGLYNELQK | |
| | DKMAEAYSEIGMKGERRRGKGHDGLYQGLSTATKDTYDALHMQALPPR | |
| 3882 | QVQLQQSGAELARPGASVKMSCKASGYTFTTYTIHWVRRRPGHDLEWIGYI | 64 |
| | NPSSGCSDYNQNFKGKTTLTADKSSNTAYMQLNSLTSEDSAVYYCARRADY | |
| | GNYEYTWFAYWGQGTTVTVSSSGGGSGGGGSGGGGSVIELTQSPKFMSTSV | |
| | GDRVNVTYKASQNVGTNVAWFQQKPGQSPKVLIYSASYRYSGVPDRFTGSG | |
| | SGTDFTLTISNVQSEDLAEYFCQQYHTYPLTFGGGTKLEIKRSDPAEPKSPDKT | |
| | HTCPGQPREPQVYTLPPSRDELTKNQVSLTCLVKGFYPSDIAVEWESNGQPEN | |
| | NYKTTPPVLDSDGSFFLYSKLTVDKSRWQQGNVFSCSVMHEALHNHYTQKS | |
| | LSLSPGKKDPKFWVLVVVGGVLACYSLLVTVAFIIFWVRSKRSRLLHSDYMN | |
| | MTPRRPGPTRKHYQAYAAARDFAAYRSLRVKFSRSADAPAYQQGQNQLYN | |
| | ELNLGRREEYDVLDKRRGRDPEMGGKPRRKNPQEGLYNELQKDKMAEAYS | |
| | EIGMKGERRRGKGHDGLYQGLSTATKDTYDALHMQALPPR | |
| 3883 | QVQLQQSGAELARPGASVKMSCKASGYTFTTYTIHWVRRRPGHDLEWIGYI | 65 |
| | NPSSGCSDYNQNFKGKTTLTADKSSNTAYMQLNSLTSEDSAVYYCARRADY | |
| | GNYEYTWFAYWGQGTTVTVSSSGGGSGGGGSGGGGSVIELTQSPKFMSTSV | |
| | GDRVNVTYKASQNVGTNVAWFQQKPGQSPKVLIYSASYRYSGVPDRFTGSG | |
| | SGTDFTLTISNVQSEDLAEYFCQQYHTYPLTFGGGTKLEIKRSDPAEPKSPDKT | |
| | HTCPPCPAPPVAGPSVFLFPPKPKDTLMIARTPEVTCVVVDVSHEDPEVKFNW | |
| | YVDGVEVHNAKTKPREEQYNSTYRVVSVLTVLHQDWLNGKEYKCKVSNKA | |
| | LPAPIEKTISKAKGQPREPQVYTLPPSRDELTKNQVSLTCLVKGFYPSDIAVEW | |
| | ESNGQPENNYKTTPPVLDSDGSFFLYSKLTVDKSRWQQGNVFSCSVMHEAL | |
| | HNHYTQKSLSLSPGKKDPKFWVLVVVGGVLACYSLLVTVAFIIFWVRSKRSR | |
| | LLHSDYMNMTPRRPGPTRKHYQPYAPPRDFAAYRSLRVKFSRSADAPAYQQ | |
| | GQNQLYNELNLGRREEYDVLDKRRGRDPEMGGKPRRKNPQEGLYNELQKD | |
| | KMAEAYSEIGMKGERRRGKGHDGLYQGLSTATKDTYDALHMQALPPR* | |
| 3884 | QVQLQQSGAELARPGASVKMSCKASGYTFTTYTIHWVRRRPGHDLEWIGYI | 66 |
| | NPSSGCSDYNQNFKGKTTLTADKSSNTAYMQLNSLTSEDSAVYYCARRADY | |
| | GNYEYTWFAYWGQGTTVTVSSSGGGSGGGGSGGGGSVIELTQSPKFMSTSV | |
| | GDRVNVTYKASQNVGTNVAWFQQKPGQSPKVLIYSASYRYSGVPDRFTGSG | |
| | SGTDFTLTISNVQSEDLAEYFCQQYHTYPLTFGGGTKLEIKRSDPATTTPAPRP | |
| | PTPAPTIASQPLSLRPEACRPAAGGAVHTRGLDFACDKDPKFWVLVVVGGVL | |
| | ACYSLLVTVAFIIFWVRSKRSRLLHSDYMNMTPRRPGPTRKHYQPYAPPRDF | |
| | AAYRSLRVKFSRSADAPAYQQGQNQLYNELNLGRREEYDVLDKRRGRDPE | |
| | MGGKPRRKNPQEGLYNELQKDKMAEAYSEIGMKGERRRGKGHDGLYQGLS | |
| | TATKDTYDALHMQALPPR | |
| 4620 | QVQLQQSGAELARPGASVKMSCKASGYTFTTYTIHWVRRRPGHDLEWIGYI | 67 |
| | NPSSGCSDYNQNFKGKTTLTADKSSNTAYMQLNSLTSEDSAVYYCARRADY | |
| | GNYEYTWFAYWGQGTTVTVSSSGGGSGGGGSGGGGSVIELTQSPKFMSTSV | |
| | GDRVNVTYKASQNVGTNVAWFQQKPGQSPKVLIYSASYRYSGVPDRFTGSG | |
| | SGTDFTLTISNVQSEDLAEYFCQQYHTYPLTFGGGTKLEIKRSDPATTTPAPRP | |
| | PTPAPTIASQPLSLRPEACRPAAGGAVHTRGLDFACDFWVLVVVGGVLACYS | |
| | LLVTVAFIIFWVRSKRSRLLHSDYMNMTPRRPGPTRKHYQPYAPPRDFAAYR | |
| | SLRVKFSRSADAPAYQQGQNQLYNELNLGRREEYDVLDKRRGRDPEMGGKP | |
| | RRKNPQEGLYNELQKDKMAEAYSEIGMKGERRRGKGHDGLYQGLSTATKD | |
| | TYDALHMQALPPR | |
| 4621 | QVQLQQSGAELARPGASVKMSCKASGYTFTTYTIHWVRQRPGHDLEWIGYI | 68 |
| | NPSSGYSDYNQNFKGKTTLTADKSSNTAYMQLNSLTSEDSAVYYCARRADY | |
| | GNYEYTWFAYWGQGTTVTVSSGGGGSGGGGSGGGGSDIELTQSPKFMSTSV | |
| | GDRVNVTYKASQNVGTNVAWFQQKPGQSPKVLIYSASYRYSGVPDRFTGSG | |
| | SGTDFTLTISNVQSEDLAEYFCQQYHTYPLTFGGGTKLEIKRSDPATTTPAPRP | |
| | PTPAPTIASQPLSLRPEACRPAAGGAVHTRGLDFACDKDPKFWVLVVVGGVL | |
| | ACYSLLVTVAFIIFWVRSKRSRLLHSDYMNMTPRRPGPTRKHYQPYAPPRDF | |
| | AAYRSLRVKFSRSADAPAYQQGQNQLYNELNLGRREEYDVLDKRRGRDPE | |
| | MGGKPRRKNPQEGLYNELQKDKMAEAYSEIGMKGERRRGKGHDGLYQGLS | |
| | TATKDTYDALHMQALPPR | |
| 4622 | QVQLQQSGAELARPGASVKMSCKASGYTFTTYTIHWVRRRPGHDLEWIGYI | 69 |
| | NPSSGYSDYNQNFKGKTTLTADKSSNTAYMQLNSLTSEDSAVYYCARRADY | |
| | GNYEYTWFAYWGQGTTVTVSSSGGGSGGGGSGGGGSVIELTQSPKFMSTSV | |
| | GDRVNVTYKASQNVGTNVAWFQQKPGQSPKVLIYSASYRYSGVPDRFTGSG | |
| | SGTDFTLTISNVQSEDLAEYFCQQYHTYPLTFGGGTKLEIKRSDPATTTPAPRP | |
| | PTPAPTIASQPLSLRPEACRPAAGGAVHTRGLDFACDKDPKFWVLVVVGGVL | |
| | ACYSLLVTVAFIIFWVRSKRSRLLHSDYMNMTPRRPGPTRKHYQPYAPPRDF | |
| | AAYRSLRVKFSRSADAPAYQQGQNQLYNELNLGRREEYDVLDKRRGRDPE | |
| | MGGKPRRKNPQEGLYNELQKDKMAEAYSEIGMKGERRRGKGHDGLYQGLS | |
| | TATKDTYDALHMQALPPR | |
| 3885 | DIQMTQSPTSLSASVGDRVTITCRASQGISSWLTWYQQKPEKAPKSLIYAASS | 70 |
| | LQSGVPSRFSGSGSGTDFTLTISSLQPEDFATYYCQQYDSYPITFGQGTRLEIK | |
| | GSTSGSGKPGSGEGSTKGQVQLQQWGAGLLKPSETLSLTCAVYGGSFSAYY | |
| | WSWIRQPPGKGLEWIGDINHGGGTNYNPSLKSRVTISVDTSKNQFSLKLNSVT | |
| | AADTAVYYCASLTAYWGQGSLVTVSSIEVMYPPPYLDNEKSNGTIIHVKGKH | |
| | LCPSPLFPGPSKPFWVLVVVGGVLACYSLLVTVAFIIFWVRSKRSRLLHSDYM | |
| | NMTPRRPGPTRKHYQPYAPPRDFAAYRSRVKFSRSADAPAYQQGQNQLYNE | |
| | LNLGRREEYDVLDKRRGRDPEMGGKPRRKNPQEGLYNELQKDKMAEAYSEI | |
| | GMKGERRRGKGHDGLYQGLSTATKDTYDALHMQALPPR | |
| 3886 | DIQMTQSPTSLSASVGDRVTITCRASQGISSWLTWYQQKPEKAPKSLIYAASS | 71 |
| | LQSGVPSRFSGSGSGTDFTLTISSLQPEDFATYYCQQYDSYPITFGQGTRLEIK | |
| | GSTSGSGKPGSGEGSTKGQVQLQQWGAGLLKPSETLSLTCAVYGGSFSAYY | |
| | WSWIRQPPGKGLEWIGDINHGGGTNYNPSLKSRVTISVDTSKNQFSLKLNSVT | |
| | AADTAVYYCASLTAYWGQGSLVTVSSIEVMYPPPYLDNEKSNGTIIHVKGKH | |
| | LCPSPLFPGPSKPFWVLVVVGGVLACYSLLVTVAFIIFWVRSKRSRLLHSDYM | |
| | NMTPRRPGPTRKHYQAYAAARDFAAYRSRVKFSRSADAPAYQQGQNQLYN | |
| | ELNLGRREEYDVLDKRRGRDPEMGGKPRRKNPQEGLYNELQKDKMAEAYS | |
| | EIGMKGERRRGKGHDGLYQGLSTATKDTYDALHMQALPPR | |
| 3887 | DIQMTQSPTSLSASVGDRVTITCRASQGISSWLTWYQQKPEKAPKSLIYAASS | 72 |
| | LQSGVPSRFSGSGSGTDFTLTISSLQPEDFATYYCQQYDSYPITFGQGTRLEIK | |
| | GSTSGSGKPGSGEGSTKGQVQLQQWGAGLLKPSETLSLTCAVYGGSFSAYY | |
| | WSWIRQPPGKGLEWIGDINHGGGTNYNPSLKSRVTISVDTSKNQFSLKLNSVT | |
| | AADTAVYYCASLTAYWGQGSLVTVSSTTTPAPRPPTPAPTIASQPLSLRPEAC | |
| | RPAAGGAVHTRGLDFACDFWVLVVVGGVLACYSLLVTVAFIIFWVRSKRSR | |
| | LLHSDYMNMTPRRPGPTRKHYQPYAPPRDFAAYRSRVKFSRSADAPAYQQG | |
| | QNQLYNELNLGRREEYDVLDKRRGRDPEMGGKPRRKNPQEGLYNELQKDK | |
| | MAEAYSEIGMKGERRRGKGHDGLYQGLSTATKDTYDALHMQALPPR* | |
| 3765 | MALPVTALLLPLALLLHAARPDIQMTQSPTSLSASVGDRVTITCRASQGISSW | 73 |
| | LTWYQQKPEKAPKSLIYAASSLQSGVPSRFSGSGSGTDFTLTISSLQPEDFATY | |
| | YCQQYDSYPITFGQGTRLEIKGSTSGSGKPGSGEGSTKGQVQLQQWGAGLLK | |
| | PSETLSLTCAVYGGSFSAYYWSWIRQPPGKGLEWIGDINHGGGTNYNPSLKS | |
| | RVTISVDTSKNQFSLKLNSVTAADTAVYYCASLTAYWGQGSLVTVSSTTTPA | |
| | PRPPTPAPTIASQPLSLRPEACRPAAGGAVHTRGLDFACDIYIWAPLAGTCGV | |
| | LLLSLVITLYCKRGRKKLLYIFKQPFMRPVQTTQEEDGCSCRFPEEEEGGCEL | |
| | RVKFSRSADAPAYKQGQNQLYNELNLGRREEYDVLDKRRGRDPEMGGKPR | |
| | RKNPQEGLYNELQKDKMAEAYSEIGMKGERRRGKGHDGLYQGLSTATKDT | |
| | YDALHMQALPPR | |
| 3764 | MALPVTALLLPLALLLHAARPDIQMTQSPTSLSASVGDRVTITCRASQGISSW | 74 |
| | LTWYQQKPEKAPKSLIYAASSLQSGVPSRFSGSGSGTDFTLTISSLQPEDFATY | |
| | YCQQYDSYPITFGQGTRLEIKGSTSGSGKPGSGEGSTKGQVQLQQWGAGLLK | |
| | PSETLSLTCAVYGGSFSAYYWSWIRQPPGKGLEWIGDINHGGGTNYNPSLKS | |
| | RVTISVDTSKNQFSLKLNSVTAADTAVYYCASLTAYWGQGSLVTVSSTTTPA | |
| | PRPPTPAPTIASQPLSLRPEACRPAAGGAVHTRGLDFACDFWVLVVVGGVLA | |
| | CYSLLVTVAFIIFWVRSKRSRLLHSDYMNMTPRRPGPTRKHYQPYAPPRDFA | |
| | AYRSRVKFSRSADAPAYKQGQNQLYNELNLGRREEYDVLDKRRGRDPEMG | |
| | GKPRRKNPQEGLYNELQKDKMAEAYSEIGMKGERRRGKGHDGLYQGLSTA | |
| | TKDTYDALHMQALPPR | |

In some embodiments of the CARs provided herein, the hinge domain comprises a CD8a hinge sequence; the transmembrane domain comprises a CD28 transmembrane sequence; the costimulatory domain comprises a wild-type CD28 costimulatory sequence; and the activation domain comprises a wild-type CD3z activation sequence comprising an N-terminal leucine. In some embodiments, the CD8a hinge sequence comprises the sequence of SEQ ID NO: 50; the CD28 transmembrane sequence comprises the sequence of SEQ ID NO: 54; the CD28 costimulatory sequence comprises the sequence of SEQ ID NO: 57; and the CD3z activation sequence comprises the sequence of SEQ ID NO: 61.

In some embodiments of the CARs provided herein, the hinge domain comprises an IgG1-CH3 hinge sequence; the transmembrane domain comprises a CD28 transmembrane sequence; the costimulatory domain comprises a CD28 costimulatory sequence comprising a mutation that eliminates lck binding; and the activation domain comprises a wild-type CD3z activation sequence comprising an N-terminal leucine. In some embodiments, the IgG1-CH3 hinge sequence comprises the sequence of SEQ ID NO: 53; the CD28 transmembrane sequence comprises the sequence of SEQ ID NO: 54; the CD28 costimulatory sequence comprises the sequence of SEQ ID NO: 58; and the CD3z activation sequence comprises the sequence of SEQ ID NO: 62.

In some embodiments of the CARs provided herein, the hinge domain comprises an IgG1-CH2-CH3 hinge sequence; the transmembrane domain comprises a CD28 transmembrane sequence; the costimulatory domain comprises a wild-type CD28 costimulatory sequence; and the activation domain comprises a CD3z activation sequence comprising an additional leucine compared to a wild-type CD3z activation sequence having an N-terminal arginine. In some embodiments, the IgG1-CH2-CH3 hinge sequence comprises the sequence of SEQ ID NO: 52; the CD28 transmembrane sequence comprises the sequence of SEQ ID NO: 54; the CD28 costimulatory sequence comprises the sequence of SEQ ID NO: 57; and the CD3z activation sequence comprises the sequence of SEQ ID NO: 61.

In some embodiments of the CARs provided herein, the hinge domain comprises a CD28 hinge sequence; the transmembrane domain comprises a CD28 transmembrane sequence; the costimulatory domain comprises a CD28 costimulatory sequence comprising a mutation that eliminates lck binding; and the activation domain comprises a wild-type CD3z activation sequence having an N-terminal argenine. In some embodiments, the CD28 hinge sequence comprises the sequence of SEQ ID NO: 51; the CD28 transmembrane sequence comprises the sequence of SEQ ID NO: 54; the CD28 costimulatory sequence comprises the sequence of SEQ ID NO: 58; and the CD3z activation sequence comprises the sequence of SEQ ID NO: 60.

In some embodiments of the CARs provided herein, the hinge domain comprises a CD8a hinge sequence; the transmembrane domain comprises a CD28 transmembrane sequence; the costimulatory domain comprises a wild-type CD28 costimulatory sequence; and the activation domain comprises a wild-type CD3z activation sequence having an N-terminal arginine. In some embodiments, the CD8a hinge sequence comprises the sequence of SEQ ID NO: 50; the CD28 transmembrane sequence comprises the sequence of SEQ ID NO: 54; the CD28 costimulatory sequence comprises the sequence of SEQ ID NO: 57; and the CD3z activation sequence comprises the sequence of SEQ ID NO: 60.

In certain embodiments of the CARs provided herein, the hinge domain comprises a CD8a hinge sequence; the transmembrane domain comprises a CD8a transmembrane sequence; the costimulatory domain comprises a 41BB costimulatory sequence; and the activation domain comprises a modified CD3z activation sequence. In certain embodiments, the CD8a hinge sequence comprises the sequence of SEQ ID NO: 50; the CD8a transmembrane sequence comprises the sequence of SEQ ID NO: 55; the 41BB costimulatory sequence comprises the sequence of SEQ ID NO: 59; and the CD3z activation sequence comprises the sequence of SEQ ID NO: 62.

In some embodiments of the CARs provided herein, the hinge domain comprises a CD8a hinge sequence; the transmembrane domain comprises a CD28 transmembrane sequence; the costimulatory domain comprises a wild-type CD28 costimulatory sequence; and the activation domain comprises a modified CD3z activation sequence. In some embodiments, the CD8a hinge sequence comprises the sequence of SEQ ID NO: 50; the CD28 transmembrane sequence comprises the sequence of SEQ ID NO: 54; the CD28 costimulatory sequence comprises the sequence of SEQ ID NO: 57; and the CD3z activation sequence comprises the sequence of SEQ ID NO: 62.

### D. Engineered Human Cell Compositions

In some embodiments, the CAR of the present disclosure is expressed in an engineered cell (e.g., T cell) in which one or more endogenous gene has been modified to reduce or eliminate the expression of the one or more gene. In some embodiments, the CAR of the present disclosure is expressed in an engineered cell that has been modified to reduce or eliminate surface expression of MHC class I protein in the cell. For example, the engineered cell has reduced or eliminated surface expression of MHC class I protein in the cell, e.g., by genetically modifying B2M (β-2-microgloblin) or by genetically modifying the HLA-A gene. The B2M protein forms a heterodimer with MHC class I molecules and is required for MHC class I protein expression on the cell surface. In some embodiments comprising a B2M genetic modification, the disclosure further provides expression of an NK cell inhibitor molecule by the cell to reduce or eliminate the lytic activity of NK cells. In some embodiments, the CAR of the present disclosure is expressed in an engineered cell that has been modified to reduce or eliminate surface expression of MHC class II protein in the cell. In some embodiments, the CAR of the present disclosure is expressed in an engineered cell that has been modified to reduce or eliminate surface expression of the endogenous TCR.

### HLA-A Edit

In some embodiments, an engineered cell (e.g., T cell) comprises a modified HLA-A gene. In some embodiments, engineered human cells that have reduced or eliminated surface expression of HLA-A relative to an unmodified cell demonstrate persistence and are protective against mismatched T cell- and NK cell-mediated rejection.

Suitable methods for modifying a gene of interest is known in the art, as described herein. Modified and unmodified HLA-A gRNA sequences that can be used in a CRISPR/Cas9 gene editing system may be used in the context of the present disclosure. See, e.g., PCT/US2021/064930, incorporated by reference in its entirety. In some embodiments, the engineered human cell, which has reduced or eliminated surface expression of HLA-A relative to an unmodified cell, comprises a genetic modification in the HLA-A gene. In some embodiments, the engineered human cell, which has reduced or eliminated surface expression of HLA-A relative to an unmodified cell, comprises a genetic modification in the HLA-A gene, wherein the genetic modification comprises at least one nucleotide within the genomic coordinates chosen from: chr6:29942854-chr6:29942913 and chr6:29943518-chr6: 29943619. In some embodiments, the engineered cell is homozygous for HLA-B and homozygous for HLA-C.

In some embodiments, at least 65%, at least 70%, at least 80%, at least 90%, at least 92%, at least 93%, at least 94%, at least 95%, at least 96%, at least 97%, at least 98%, or at least 99% of the engineered cells of a population do not express a detectable level of HLA-A as measured by, e.g., flow cytometry.

In some embodiments, the modification to HLA-A comprises any one or more of an insertion, deletion, substitution or deamination of at least one nucleotide in a target sequence. In some embodiments, the modification to HLA-A comprises an insertion of 1, 2, 3, 4 or 5 or more nucleotides in a target sequence. In some embodiments, the modification to HLA-A comprises a deletion of 1, 2, 3, 4 or 5 or more nucleotides in a target sequence. In other embodiments, the modification to HLA-A comprises an insertion of 1, 2, 3, 4, 5, 6, 7, 8, 9, 10, 15, 20 or 25 or more nucleotides in a target sequence. In other embodiments, the modification to HLA-A comprises a deletion of 1, 2, 3, 4, 5, 6, 7, 8, 9, 10, 15, 20 or 25 or more nucleotides in a target sequence. In some embodiments, the modification to HLA-A comprises an indel, which is generally defined in the art as an insertion or deletion of less than 1000 base pairs (bp). In some embodiments, the modification to HLA-A comprises an indel which results in a frameshift mutation in a target sequence. In some embodiments, the modification to HLA-A comprises a substitution of 1, 2, 3, 4, 5, 6, 7, 8, 9, 10, 15, 20 or 25 or more nucleotides in a target sequence. In some embodiments, the modification to HLA-A comprises one or more of an insertion, deletion, or substitution of nucleotides resulting from the incorporation of a template nucleic acid. In some embodiments, the modification to HLA-A comprises an insertion of a donor nucleic acid in a target sequence. In some embodiments, the modification to HLA-A is not transient.

### CIITA Edit

In some embodiments, an engineered cell (e.g., T cell) comprises a modified CIITA gene. In some embodiments, engineered human cells that have reduced or eliminated expression of CIITA relative to an unmodified cell demonstrate persistence and are protective against CD4 and CD8-mediated rejection.

Suitable methods for modifying a gene of interest is known in the art, as described herein. Modified and unmodified CIITA gRNA sequences that can be used in a CRISPR/Cas9 gene editing system may be used in the context of the present disclosure. See, e.g., PCT/US2021/064933, incorporated by reference in its entirety. In some embodiments, the engineered human cell, which has reduced or eliminated expression of CTIIA relative to an unmodified cell, comprises a genetic modification in the CTIIA gene. In some embodiments, the engineered human cell, which has reduced or eliminated expression of CTIIA relative to an unmodified cell, comprises a genetic modification in the CTIIA gene, wherein the genetic modification comprises at least one nucleotide of an exon within the genomic coordinates chr16:10902662-chr16:10923285 or within the genomic coordinates chr16:10906542-chr16:10908121.

In some embodiments, at least 65%, at least 70%, at least 80%, at least 90%, at least 92%, at least 93%, at least 94%, at least 95%, at least 96%, at least 97%, at least 98%, or at least 99% of the engineered cells of a population do not express a detectable level of CTIIA as measured by, e.g., flow cytometry.

In some embodiments, the modification to CTIIA comprises any one or more of an insertion, deletion, substitution or deamination of at least one nucleotide in a target sequence. In some embodiments, the modification to CTIIA comprises an insertion of 1, 2, 3, 4 or 5 or more nucleotides in a target sequence. In some embodiments, the modification to CTIIA comprises a deletion of 1, 2, 3, 4 or 5 or more nucleotides in a target sequence. In other embodiments, the modification to CTIIA comprises an insertion of 1, 2, 3, 4, 5, 6, 7, 8, 9, 10, 15, 20 or 25 or more nucleotides in a target sequence. In other embodiments, the modification to CTIIA comprises a deletion of 1, 2, 3, 4, 5, 6, 7, 8, 9, 10, 15, 20 or 25 or more nucleotides in a target sequence. In some embodiments, the modification to CTIIA comprises an indel, which is generally defined in the art as an insertion or deletion of less than 1000 base pairs (bp). In some embodiments, the modification to CTIIA comprises an indel which results in a frameshift mutation in a target sequence. In some embodiments, the modification to CTIIA comprises a substitution of 1, 2, 3, 4, 5, 6, 7, 8, 9, 10, 15, 20 or 25 or more nucleotides in a target sequence. In some embodiments, the modification to CTIIA comprises one or more of an insertion, deletion, or substitution of nucleotides resulting from the incorporation of a template nucleic acid. In some embodiments, the modification to CTIIA comprises an insertion of a donor nucleic acid in a target sequence. In some embodiments, the modification to CTIIA is not transient.

### TRAC Edit

In some embodiments, an engineered cell (e.g., T cell) comprises a modified TRAC gene. In some embodiments, engineered human cells that have reduced or eliminated surface expression of TRAC relative to an unmodified cell exhibit various therapeutic benefits, e.g., reduced graft versus host disease (GVHD).

Suitable methods for modifying a gene of interest is known in the art, as described herein. Modified and unmodified TRAC gRNA sequences that can be used in a CRISPR/Cas9 gene editing system may be used in the context of the present disclosure. See, e.g., WO2019215500 or WO2020081613, each incorporated by reference in its entirety. In some embodiments, the engineered human cell, which has reduced or eliminated expression of TRAC relative to an unmodified cell, comprises a genetic modification in the TRAC gene. In some embodiments, the engineered human cell, which has reduced or eliminated expression of TRAC relative to an unmodified cell, comprises a genetic modification in the TRAC gene, wherein the genetic modification comprises at least one nucleotide within the genomic coordinates chosen from: Chr14: 22,547,506-Chr14:22,552,154.

In some embodiments, at least 65%, at least 70%, at least 80%, at least 90%, at least 92%, at least 93%, at least 94%, at least 95%, at least 96%, at least 97%, at least 98%, or at least 99% of the engineered cells of a population do not express a detectable level of TRAC as measured by, e.g., flow cytometry.

In some embodiments, the modification to TRAC comprises any one or more of an insertion, deletion, substitution or deamination of at least one nucleotide in a target sequence. In some embodiments, the modification to TRAC comprises an insertion of 1, 2, 3, 4 or 5 or more nucleotides in a target sequence. In some embodiments, the modification to TRAC comprises a deletion of 1, 2, 3, 4 or 5 or more nucleotides in a target sequence. In other embodiments, the modification to TRAC comprises an insertion of 1, 2, 3, 4, 5, 6, 7, 8, 9, 10, 15, 20 or 25 or more nucleotides in a target sequence. In other embodiments, the modification to TRAC comprises a deletion of 1, 2, 3, 4, 5, 6, 7, 8, 9, 10, 15, 20 or 25 or more nucleotides in a target sequence. In some embodiments, the modification to TRAC comprises an indel, which is generally defined in the art as an insertion or deletion of less than 1000 base pairs (bp). In some embodiments, the modification to TRAC comprises an indel which results in a frameshift mutation in a target sequence. In some embodiments, the modification to TRAC comprises a substitution of 1, 2, 3, 4, 5, 6, 7, 8, 9, 10, 15, 20 or 25 or more nucleotides in a target sequence. In some embodiments, the modification to TRAC comprises one or more of an insertion, deletion, or substitution of nucleotides resulting from the incorporation of a template nucleic acid. In some embodiments, the modification to TRAC comprises an insertion of a donor nucleic acid in a target sequence. In some embodiments, the modification to TRAC is not transient.

In some embodiments, the present disclosure provides methods for reducing expression of HLA-A, HLA class II protein, or TCR on the surface of a cell by genetically modifying the HLA-A, CIITA, or TCR gene comprising contacting the cell with a composition comprising an HLA-A, CIITA, or TCR guide RNA, the method further comprising contacting the cell with an exogenous nucleic acid.

In some embodiments, the present disclosure provides methods for reducing or eliminating expression of HLA-A, HLA class II protein, or TCR on the surface of a cell, comprising genetically modifying the cell with one or more compositions comprising a HLA-A, CIITA, or TCR guide RNA, an exogenous nucleic acid encoding a polypeptide (e.g., a targeting receptor), and an RNA-guided DNA binding agent or a nucleic acid encoding an RNA-guided DNA binding agent.

### E. Exemplary Gene Editing Systems

Various suitable gene editing systems may be used to make the engineered cells disclosed herein, including but not limited to the CRISPR/Cas system; zinc finger nuclease (ZFN) system; and the transcription activator-like effector nuclease (TALEN) system. Generally, the gene editing systems involve the use of engineered cleavage systems to induce a double strand break (DSB) or a nick (e.g., a single strand break, or SSB) in a target DNA sequence. Cleavage or nicking can occur through the use of specific nucleases such as engineered ZFN, TALENs, or using the CRISPR/Cas system with an engineered guide RNA to guide specific cleavage or nicking of a target DNA sequence. Further, targeted nucleases are being developed based on the Argonaute system (e.g., from T. thermophilus, known as ‘TtAgo’, see Swarts et al (2014) Nature 507(7491): 258-261), which also may have the potential for uses in gene editing and gene therapy.

In some embodiments, the gene editing system is a TALEN system. Transcription activator-like effector nucleases (TALEN) are restriction enzymes that can be engineered to cut specific sequences of DNA. They are made by fusing a TAL effector DNA-binding domain to a DNA cleavage domain (a nuclease which cuts DNA strands). Transcription activator-like effectors (TALEs) can be engineered to bind to a desired DNA sequence, to promote DNA cleavage at specific locations (see, e.g., Boch, 2011, Nature Biotech). The restriction enzymes can be introduced into cells, for use in gene editing or for gene editing in situ, a technique known as gene editing with engineered nucleases. Such methods and compositions for use therein are known in the art. See, e.g., WO2019147805, WO2014040370, WO2018073393, the contents of which are hereby incorporated in their entireties.

In some embodiments, the gene editing system is a zinc-finger system. Zinc-finger nucleases (ZFNs) are artificial restriction enzymes generated by fusing a zinc finger DNA-binding domain to a DNA-cleavage domain. Zinc finger domains can be engineered to target specific desired DNA sequences to enables zinc-finger nucleases to target unique sequences within complex genomes. The non-specific cleavage domain from the type IIs restriction endonuclease FokI is typically used as the cleavage domain in ZFNs. Cleavage is repaired by endogenous DNA repair machinery, allowing ZFN to precisely alter the genomes of higher organisms. Such methods and compositions for use therein are known in the art. See, e.g., WO2011091324, the contents of which are hereby incorporated in their entireties.

In some embodiments, the gene editing system is a CRISPR/Cas system, including e.g., a CRISPR guide RNA comprising a guide sequence and RNA-guided DNA binding agent, and described further herein.

Provided herein are guide sequences useful for modifying a target sequence, e.g., using a guide RNA comprising a disclosed guide sequence with an RNA-guided DNA binding agent (e.g., a CRISPR/Cas system). See, e.g., US20180112234, incorporated by reference in its entirety.

In some embodiments of the present disclosure, the nuclease system includes at least one nuclease. In some embodiments, the nuclease may comprise at least one DNA binding domain and at least one nuclease domain. In some embodiments, the nuclease domain may be heterologous to the DNA binding domain. In certain embodiments, the nuclease is a DNA endonuclease, and may cleave single or double-stranded DNA. In certain embodiments, the nuclease may cleave RNA.

In some embodiments, the nuclease may include a Cas protein (also called a “Cas nuclease”) from a CRISPR/Cas system. The Cas protein may comprise at least one domain that interacts with a guide RNA (gRNA). Additionally, the Cas protein may be directed to a target sequence by a guide RNA. The guide RNA interacts with the Cas protein as well as the target sequence such that, once directed to the target sequence, the Cas protein is capable of cleaving the target sequence. In certain embodiments, e.g., Cas9, the Cas protein is a single-protein effector, an RNA-guided nuclease. In some embodiments, the guide RNA provides the specificity for the targeted cleavage, and the Cas protein may be universal and paired with different guide RNAs to cleave different target sequences. The terms Cas protein and Cas nuclease are used interchangeably herein.

In some embodiments, the CRISPR/Cas system may comprise Type-I, Type-II, or Type-III system components. Updated classification schemes for CRISPR/Cas loci define Class 1 and Class 2 CRISPR/Cas systems, having Types I to V or VI. See, e.g., Makarova et al., Nat Rev Microbiol, 13(11): 722-36 (2015); Shmakov et al., Molecular Cell, 60:385-397 (2015). Class 2 CRISPR/Cas systems have single protein effectors. Cas proteins of Types II, V, and VI may be single-protein, RNA-guided endonucleases, herein called “Class 2 Cas nucleases.” Class 2 Cas nucleases include, for example, Cas9, Cpf1, C2c1, C2c2, and C2c3 proteins. Cpf1 protein, Zetsche et al., Cell, 163: 1-13 (2015), is homologous to Cas9, and contains a RuvC-like nuclease domain. Cpf1 sequences of Zetsche are incorporated by reference in their entirety. See, e.g., Zetsche, Tables 51 and S3.

In some embodiments, the Cas protein may be from a Type-II CRISPR/Cas system, i.e., a Cas9 protein from a CRISPR/Cas9 system. In some embodiments, the Cas protein may be from a Class 2 CRISPR/Cas system, i.e., a single-protein Cas nuclease such as a Cas9 protein or a Cpf1 protein. The Cas9 and Cpf1 family of proteins are enzymes with DNA endonuclease activity, and they can be directed to cleave a desired nucleic acid target by designing an appropriate guide RNA, as described further herein.

A Type-II CRISPR/Cas system component may be from a Type-IIA, Type-IIB, or Type-IIC system. Cas9 and its orthologs are encompassed. Non-limiting exemplary species that the Cas9 protein or other components may be from include Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gamma proteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, or Acaryochloris marina. In some embodiments, the Cas9 protein may be from Streptococcus pyogenes. In some embodiments, the Cas9 protein may be from Streptococcus thermophilus. In some embodiments, the Cas9 protein may be from Neisseria meningitidis. In some embodiments, the Cas9 protein may be from Staphylococcus aureus.

In some embodiments, a Cas protein may comprise more than one nuclease domain. For example, a Cas9 protein may comprise at least one RuvC-like nuclease domain (e.g. Cpf1) and at least one HNH-like nuclease domain (e.g. Cas9). In some embodiments, the Cas9 protein may be capable of introducing a DSB in the target sequence. In some embodiments, the Cas9 protein may be modified to contain only one functional nuclease domain. For example, the Cas9 protein may be modified such that one of the nuclease domains is mutated or fully or partially deleted to reduce its nucleic acid cleavage activity. In some embodiments, the Cas9 protein may be modified to contain no functional RuvC-like nuclease domain. In other embodiments, the Cas9 protein may be modified to contain no functional HNH-like nuclease domain. In some embodiments in which only one of the nuclease domains is functional, the Cas9 protein may be a nickase that is capable of introducing a single-stranded break (a “nick”) into the target sequence. In some embodiments, a conserved amino acid within a Cas9 protein nuclease domain is substituted to reduce or alter a nuclease activity. In some embodiments, the Cas protein nickase may comprise an amino acid substitution in the RuvC-like nuclease domain. Exemplary amino acid substitutions in the RuvC-like nuclease domain include D10A (based on the S. pyogenes Cas9 protein). In some embodiments, the nickase may comprise an amino acid substitution in the HNH-like nuclease domain. Exemplary amino acid substitutions in the HNH-like nuclease domain include E762A, H840A, N863A, H983A, and D986A (based on the S. pyogenes Cas9 protein). In some embodiments, the nuclease system described herein may comprise a nickase and a pair of guide RNAs that are complementary to the sense and antisense strands of the target sequence, respectively. The guide RNAs may direct the nickase to target and introduce a DSB by generating a nick on opposite strands of the target sequence (i.e., double nicking). Chimeric Cas9 proteins may also be used, where one domain or region of the protein is replaced by a portion of a different protein. For example, a Cas9 nuclease domain may be replaced with a domain from a different nuclease such as Fok1. A Cas9 protein may be a modified nuclease.

In alternative embodiments, the Cas protein may be from a Type-I CRISPR/Cas system. In some embodiments, the Cas protein may be a component of the Cascade complex of a Type-I CRISPR/Cas system. For example, the Cas protein may be a Cas3 protein. In some embodiments, the Cas protein may be from a Type-III CRISPR/Cas system. In some embodiments, the Cas protein may be from a Type-IV CRISPR/Cas system. In some embodiments, the Cas protein may be from a Type-V CRISPR/Cas system. In some embodiments, the Cas protein may be from a Type-VI CRISPR/Cas system. In some embodiments, the Cas protein may have an RNA cleavage activity.

In some embodiments of the present disclosure, a CRISPR/Cas nuclease system includes at least one guide RNA. In some embodiments, the guide RNA and the Cas protein may form a ribonucleoprotein (RNP), e.g., a CRISPR/Cas complex. The guide RNA may guide the Cas protein to a target sequence on a target nucleic acid molecule, where the guide RNA hybridizes with and the Cas protein cleaves the target sequence. In some embodiments, the CRISPR/Cas complex may be a Cpf1/guide RNA complex. In some embodiments, the CRISPR complex may be a Type-II CRISPR/Cas9 complex. In some embodiments, the Cas protein may be a Cas9 protein. In some embodiments, the CRISPR/Cas9 complex may be a Cas9/guide RNA complex.

A guide RNA for a CRISPR/Cas9 nuclease system comprises a CRISPR RNA (crRNA) and a tracr RNA (tracr). A guide RNA for a CRISPR/Cpf1 nuclease system comprises a crRNA. In some embodiments, the crRNA may comprise a targeting sequence that is complementary to and hybridizes with the target sequence on the target nucleic acid molecule. The crRNA may also comprise a flagpole that is complementary to and hybridizes with a portion of the tracrRNA. In some embodiments, the crRNA may parallel the structure of a naturally occurring crRNA transcribed from a CRISPR locus of a bacteria, where the targeting sequence acts as the spacer of the CRISPR/Cas9 system, and the flagpole corresponds to a portion of a repeat sequence flanking the spacers on the CRISPR locus.

The guide RNA may target any sequence of interest via the targeting sequence of the crRNA. In some embodiments, the degree of complementarity between the targeting sequence of the guide RNA and the target sequence on the target nucleic acid molecule may be about 60%, 65%, 70%, 75%, 80%, 85%, 90%, 95%, 97%, 98%, 99%, or 100%. In some embodiments, the targeting sequence of the guide RNA and the target sequence on the target nucleic acid molecule may be 100% complementary. In other embodiments, the targeting sequence of the guide RNA and the target sequence on the target nucleic acid molecule may contain at least one mismatch. For example, the targeting sequence of the guide RNA and the target sequence on the target nucleic acid molecule may contain 1, 2, 3, 4, 5, 6, 7, 8, 9, or 10 mismatches. In some embodiments, the targeting sequence of the guide RNA and the target sequence on the target nucleic acid molecule may contain 1-6 mismatches. In some embodiments, the targeting sequence of the guide RNA and the target sequence on the target nucleic acid molecule may contain 5 or 6 mismatches.

The length of the targeting sequence may depend on the CRISPR/Cas9 system and components used. For example, different Cas9 proteins from different bacterial species have varying optimal targeting sequence lengths. Accordingly, the targeting sequence may comprise 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length. In some embodiments, the targeting sequence may comprise 18-24 nucleotides in length. In some embodiments, the targeting sequence may comprise 19-21 nucleotides in length. In some embodiments, the targeting sequence may comprise 20 nucleotides in length.

The flagpole may comprise any sequence with sufficient complementarity with a tracr RNA to promote the formation of a functional CRISPR/Cas9 complex. In some embodiments, the flagpole may comprise all or a portion of the sequence (also called a “tag” or “handle”) of a naturally-occurring crRNA that is complementary to the tracr RNA in the same CRISPR/Cas9 system. In some embodiments, the flagpole may comprise all or a portion of a repeat sequence from a naturally-occurring CRISPR/Cas9 system. In some embodiments, the flagpole may comprise a truncated or modified tag or handle sequence. In some embodiments, the degree of complementarity between the tracr RNA and the portion of the flagpole that hybridizes with the tracr RNA along the length of the shorter of the two sequences may be about 40%, 50%, 60%, 70%, 80%, or higher, but lower than 100%. In some embodiments, the tracr RNA and the portion of the flagpole that hybridizes with the tracr RNA are not 100% complementary along the length of the shorter of the two sequences because of the presence of one or more bulge structures on the tracr or wobble base pairing between the tracr and the flagpole. The length of the flagpole may depend on the CRISPR/Cas9 system or the tracr RNA used. For example, the flagpole may comprise 10-50 nucleotides, or more than 50 nucleotides in length. In some embodiments, the flagpole may comprise 15-40 nucleotides in length. In other embodiments, the flagpole may comprise 20-30 nucleotides in length. In yet other embodiments, the flagpole may comprise 22 nucleotides in length. When a dual guide RNA is used, for example, the length of the flagpole may have no upper limit.

In some embodiments, the tracr RNA may comprise all or a portion of a wild-type tracr RNA sequence from a naturally-occurring CRISPR/Cas9 system. In some embodiments, the tracr RNA may comprise a truncated or modified variant of the wild-type tracr RNA. The length of the tracr RNA may depend on the CRISPR/Cas9 system used. In some embodiments, the tracr RNA may comprise 5, 6, 7, 8, 9, 10, 11, 12, 13, 14, 15, 16, 17, 18, 19, 20, 25, 30, 40, 50, 60, 70, 80, 90, 100, or more than 100 nucleotides in length. In certain embodiments, the tracr is at least 26 nucleotides in length. In additional embodiments, the tracr is at least 40 nucleotides in length. In some embodiments, the tracr RNA may comprise certain secondary structures, such as, e.g., one or more hairpins or stem-loop structures, or one or more bulge structures.

In some embodiments, the guide RNA may comprise two RNA molecules and is referred to herein as a “dual guide RNA” or “dgRNA”. In some embodiments, the dgRNA may comprise a first RNA molecule comprising a crRNA, and a second RNA molecule comprising a tracr RNA. The first and second RNA molecules may form a RNA duplex via the base pairing between the flagpole on the crRNA and the tracr RNA.

In additional embodiments, the guide RNA may comprise a single RNA molecule and is referred to herein as a “single guide RNA” or “sgRNA”. In some embodiments, the sgRNA may comprise a crRNA covalently linked to a tracr RNA. In some embodiments, the crRNA and the tracr RNA may be covalently linked via a linker. In some embodiments, the single-molecule guide RNA may comprise a stem-loop structure via the base pairing between the flagpole on the crRNA and the tracr RNA.

Certain embodiments of the invention also provide nucleic acids, e.g., vectors, encoding the guide RNA described herein. In some embodiments, the nucleic acid may be a DNA molecule. In other embodiments, the nucleic acid may be an RNA molecule. In some embodiments, the nucleic acid may comprise a nucleotide sequence encoding a crRNA. In some embodiments, the nucleotide sequence encoding the crRNA comprises a targeting sequence flanked by all or a portion of a repeat sequence from a naturally-occurring CRISPR/Cas system. In some embodiments, the nucleic acid may comprise a nucleotide sequence encoding a tracr RNA. In some embodiments, the crRNA and the tracr RNA may be encoded by two separate nucleic acids. In other embodiments, the crRNA and the tracr RNA may be encoded by a single nucleic acid. In some embodiments, the crRNA and the tracr RNA may be encoded by opposite strands of a single nucleic acid. In other embodiments, the crRNA and the tracr RNA may be encoded by the same strand of a single nucleic acid.

In certain embodiments, more than one guide RNA can be used with a CRISPR/Cas nuclease system. Each guide RNA may contain a different targeting sequence, such that the CRISPR/Cas system cleaves more than one target sequence. In some embodiments, one or more guide RNAs may have the same or differing properties such as activity or stability within the Cas9 RNP complex. Where more than one guide RNA is used, each guide RNA can be encoded on the same or on different vectors. The promoters used to drive expression of the more than one guide RNA may be the same or different.

F. Delivery of gRNA Compositions and Constructs

Lipid nanoparticles (LNP compositions) are a well-known means for delivery of nucleotide and protein cargo and may be used for delivery of guide RNAs, compositions, or pharmaceutical formulations disclosed herein. In some embodiments, the LNP compositions deliver nucleic acid, protein, or nucleic acid together with protein.

In some embodiments, provided herein is a method for delivering any one of the gRNAs disclosed herein to a cell, wherein the gRNA is formulated as an LNP. In some embodiments, the LNP comprises the gRNA and a Cas9 or an mRNA encoding the gRNA or the Cas9.

In some embodiments, provided herein is a composition comprising any one of the gRNAs disclosed and an LNP. In some embodiments, the composition further comprises a Cas9 or an mRNA encoding Cas9. In some embodiments, the composition further comprises a base editor, such as, but not limited to a cytidine or adenosine deaminase. In some embodiments, the composition comprises a first LNP comprising any one of the gRNAs disclosed herein and a second LNP comprising a Cas9 or mRNA encoding a Cas9.

In some embodiments, the LNP compositions comprise cationic or ionizable lipids. In some embodiments, the LNP compositions comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., lipids of WO/2017/173054 and references described therein. In some embodiments, the LNP compositions comprise molar ratios of a cationic lipid amine to RNA phosphate (N:P) of about 4.5, 5.0, 5.5, 6.0, or 6.5. In some embodiments, the term cationic and ionizable in the context of LNP lipids is interchangeable, e.g., wherein ionizable lipids are cationic depending on pH. In some embodiments, the LNP composition may also include, e.g., cholesterol, phospholipids, or stealth lipids.

Electroporation is a well-known means for delivery of cargo, and any electroporation methodology may be used for delivery of any one of the gRNAs disclosed herein. In some embodiments, electroporation may be used to deliver any one of the gRNAs disclosed herein and Cas9 or an mRNA encoding Cas9.

In some embodiments, provided herein is a method for delivering any one of the gRNAs disclosed herein to an ex vivo cell, wherein the gRNA is formulated as an LNP or not formulated as an LNP. In some embodiments, the LNP comprises the gRNA and a Cas9 or an mRNA encoding Cas9.

In some embodiments, the guide RNA compositions described herein, alone or encoded on one or more vectors, are formulated in or administered via a lipid nanoparticle; see e.g., WO/2017/173054 and WO 2019/067992, the contents of which are hereby incorporated by reference in their entirety.

In certain embodiments, provided herein are DNA or RNA vectors encoding any of the constructs or guide RNAs comprising any one or more of the guide sequences or construct sequences described herein. In some embodiments, in addition to constructs or guide RNA sequences, the vectors further comprise nucleic acids that do not encode constructs or guide RNAs. Nucleic acids that do not encode a construct or guide RNA include, but are not limited to, promoters, enhancers, regulatory sequences, and nucleic acids encoding an RNA-guided DNA nuclease, which can be a nuclease such as Cas9. In some embodiments, the vector comprises one or more nucleotide sequence(s) encoding a crRNA, a trRNA, or a crRNA and trRNA. In some embodiments, the vector comprises one or more nucleotide sequence(s) encoding a CAR construct described herein. In some embodiments, the vector comprises one or more nucleotide sequence(s) encoding a sgRNA and an mRNA encoding an RNA-guided DNA nuclease, which can be a Cas nuclease, such as Cas9 or Cpf1. In some embodiments, the vector comprises one or more nucleotide sequence(s) encoding a crRNA, a trRNA, and an mRNA encoding an RNA-guided DNA nuclease, which can be a Cas protein, such as, Cas9. In one embodiment, the Cas9 is from Streptococcus pyogenes (i.e., Spy Cas9). In some embodiments, the nucleotide sequence encoding the crRNA, trRNA, or crRNA and trRNA (which may be a sgRNA) comprises or consists of a guide sequence flanked by all or a portion of a repeat sequence from a naturally-occurring CRISPR/Cas system. The nucleic acid comprising or consisting of the crRNA, trRNA, or crRNA and trRNA may further comprise a vector sequence wherein the vector sequence comprises or consists of nucleic acids that are not naturally found together with the crRNA, trRNA, or crRNA and trRNA. In some embodiments, the constructs (e.g., CAR constructs) described herein may be delivered using vectors known in the art, e.g., lentiviral vectors, retroviral vectors or adeno-associated virus vectors (AAVs).

In some embodiments, “AAV” refers all serotypes, subtypes, and naturally-occurring AAV as well as recombinant AAV. “AAV” may be used to refer to the virus itself or a derivative thereof. The term “AAV” includes AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, nonprimate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. A “AAV vector” as used herein refers to an AAV vector comprising a heterologous sequence not of AAV origin (i.e., a nucleic acid sequence heterologous to AAV), typically comprising a sequence encoding a heterologous polypeptide of interest (e.g., AAT). The construct may comprise an AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, nonprimate AAV, and ovine AAV capside sequence. In general, the heterologous nucleic acid sequence (the transgene) is flanked by at least one, at least two, or at least three AAV inverted terminal repeat sequences (ITRs). An AAV vector may either be single-stranded (ssAAV) or self-complementary (scAAV).

In some embodiments, the lentivirus may be non-integrating. In some embodiments, the viral vector may be an adenovirus vector. In some embodiments, the adenovirus may be a high-cloning capacity or “gutless” adenovirus, where all coding viral regions apart from the 5′ and 3′ inverted terminal repeats (ITRs) and the packaging signal (T) are deleted from the virus to increase its packaging capacity.

### G. Therapeutic Methods and Uses

Any of the engineered cells and compositions described herein can be used in a method of treating a variety of diseases and disorders, e.g., cancer. In some embodiments, the genetically modified cell (engineered cell) or population of genetically modified cells (engineered cells) and compositions may be used in methods of treating a variety of diseases and disorders. In some embodiments, a method of treating any one of the diseases or disorders described herein is encompassed, comprising administering any one or more composition described herein.

In some embodiments, the methods comprise administering to a subject a composition comprising an engineered cell described herein as an adoptive cell transfer therapy. In some embodiments, the engineered cell is an allogeneic cell.

Immunotherapy is the treatment of disease by activating or suppressing the immune system. Immunotherapies designed to elicit or amplify an immune response are classified as activation immunotherapies. Cell-based immunotherapies have been demonstrated to be effective in the treatment of some cancers. Immune effector cells such as lymphocytes, macrophages, dendritic cells, natural killer cells, cytotoxic T lymphocytes (CTLs), T helper cells, B cells, or their progenitors such as hematopoietic stem cells (HSC) or induced pluripotent stem cells (iPSC) can be programmed to act in response to abnormal antigens expressed on the surface of tumor cells. Thus, cancer immunotherapy allows components of the immune system to destroy tumors or other cancerous cells. Cell-based immunotherapies have also been demonstrated to be effective in the treatment of autoimmune diseases or transplant rejection.

In some embodiments, the methods and compositions described herein may be used to treat diseases or disorders in need of delivery of a therapeutic agent. In some embodiments, provided herein is a method of providing an immunotherapy in a subject, the method including administering to the subject an effective amount of an engineered cell (or population of engineered cells) as described herein, for example, a cell of any of the aforementioned cell aspects and embodiments.

In some embodiments, provided herein is a method of preparing and using engineered cells (e.g., a population of engineered cells) that express a chimeric antigen receptor (e.g., a CD30 targeting CAR). The population of engineered cells may be used for immunotherapy.

In some embodiments, the methods comprise administering to a subject a composition comprising an engineered cell described herein, wherein the cell expresses a polypeptide (e.g., a chimeric antigen receptor, e.g., anti-CD30 CAR) useful for treatment of a disease or disorder in a subject. In some embodiments, the cell continuously expresses the polypeptide in vivo. In some embodiments, the cell continuously expresses the polypeptide following transplantation in vivo for at least 1, 2, 3, 4, 5, or 6 weeks. In some embodiments, the cell continuously expresses the polypeptide following transplantation in vivo for more than 6 weeks. In some embodiments, the engineered cells disclosed herein are used to treat CD30-expressing lymphomas, e.g., CD30-expressing hematologic cancers including relapsed or refractory classical Hodgkin's Lymphoma (cHL).

In some embodiments of the methods, the method includes administering a lymphodepleting agent or immunosuppressant prior to administering to the subject an effective amount of the engineered cell (or engineered cells) as described herein, for example, a cell of any of the aforementioned cell aspects and embodiments. In another aspect, provided herein is a method of preparing engineered cells (e.g., a population of engineered cells).

In some embodiments, the methods provide for administering the engineered cells to a subject, wherein the administration is an injection. In some embodiments, the methods provide for administering the engineered cells to a subject, wherein the administration is an intravenous administration, e.g., intravascular injection or infusion. In some embodiments, the methods provide for administering the engineered cells to a subject, wherein the administration is a single dose.

The precise amount of the compositions of the present invention to be administered can be determined by a physician with consideration of individual differences in age, weight, tumor size, extent of infection or metastasis, and condition of the patient (subject). The engineered cells may be administered at a single dose or multiple times at suitable dosages. The cells can be administered by using infusion techniques that are commonly known in immunotherapy (see, e.g., Rosenberg et al., New Eng. J. of Med. 319: 1676, 1988). A suitable dosage and treatment regime for a particular patient can readily be determined by one skilled in the art of medicine by monitoring the patient for signs of disease and adjusting the treatment accordingly. Strategies for CAR T cell dosing and scheduling have been discussed (Ertl et al, 2011, Cancer Res, 71:3175-81; Junghans, 2010, Journal of Translational Medicine, 8:55). Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

In some embodiments, the methods provide for reducing a sign or symptom associated of a subject's disease treated with a composition disclosed herein. In some embodiments, the subject has a response to treatment with a composition disclosed herein that lasts more than one week. In some embodiments, the subject has a response to treatment with a composition disclosed herein that lasts more than two weeks. In some embodiments, the subject has a response to treatment with a composition disclosed herein that lasts more than three weeks. In some embodiments, the subject has a response to treatment with a composition disclosed herein that lasts more than one month.

In some embodiments, the methods provide for administering the engineered cells to a subject, and wherein the subject has a response to the administered cell that comprises a reduction in a sign or symptom associated with the disease treated by the cell therapy. In some embodiments, the subject has a response that lasts more than one week. In some embodiments, the subject has a response that lasts more than one month. In some embodiments, the subject has a response that lasts for at least 1-6 weeks.

### EXEMPLARY EMBODIMENTS

The present disclosure provides, among other things, the following exemplary embodiments:

1. A chimeric antigen receptor (CAR) comprising:
(a) a binder domain;
(b) a hinge domain comprising a hinge sequence selected from a CD8a hinge sequence and a CD28 hinge sequence;
(c) a transmembrane domain comprising a transmembrane sequence selected from a CD28 transmembrane sequence and a CD8a transmembrane sequence;
(d) a costimulatory domain comprising a costimulatory sequence selected from a CD28 costimulatory sequence; and a 41BB costimulatory sequence; and
(e) an activation domain comprising a CD3z activation sequence.
   2. The CAR of embodiment 1, wherein the binder domain binds to CD30.
   3. A chimeric antigen receptor (CAR) comprising:
(a) a binder domain that binds CD30;
(b) a CD8a hinge domain;
(c) a CD28 transmembrane domain;
(d) a costimulatory domain; and
(e) an activation domain.
   4. The CAR of embodiment 2 or 3, wherein the binder domain comprises a CD30 antibody or antigen binding fragment thereof comprising a heavy chain variable region (VH) complementarity-determining region 1 (CDR1) sequence TFTTYT (SEQ ID NO: 3); a VH CDR2 sequence INPSSGCSD (SEQ ID NO: 4) or INPSSGYSD (SEQ ID NO: 5); a VH CDR3 sequence RADYGNYEYTWFAY (SEQ ID NO: 6); a light chain variable region (VL) CDR1 sequence ASQNVGTNVA (SEQ ID NO: 7); a VL CDR2 sequence SASYRYS (SEQ ID NO: 8); and a VL CDR3 sequence QQYHTYP (SEQ ID NO: 9).
   5. The CAR of embodiment 4, wherein the VH amino acid sequence has at least 80% identity to sequence SEQ ID NO: 24 and the VL amino acid sequence has at least 80% identity to SEQ ID NO: 25.
   6. The CAR of embodiment 4, wherein the VH amino acid sequence has at least 80% identity to sequence SEQ ID NO: 26 and the VL amino acid sequence has at least 80% identity to SEQ ID NO: 27.
   7. The CAR of embodiment 4, wherein the VH amino acid sequence has at least 80% identity to sequence SEQ ID NO: 28 and the VL amino acid sequence has at least 80% identity to SEQ ID NO: 25.
   8. The CAR of embodiment 4, wherein the VH amino acid sequence has at least 80% identity to sequence SEQ ID NO: 29 and the VL amino acid sequence has at least 80% identity to SEQ ID NO: 30.
   9. The CAR of embodiment 2 or 3, wherein the binder domain is HRS3.
   10. The CAR of embodiment 2 or 3, wherein the binder domain comprises a CD30 antibody or antigen binding fragment thereof comprising a heavy chain variable region (VH) complementarity-determining region 1 (CDR1) sequence AYYWS (SEQ ID NO: 10); a VH CDR2 sequence DINHGGGTNYNPSLKS (SEQ ID NO: 11); a VH CDR3 sequence LTAY (SEQ ID NO: 12); a light chain variable region (VL) CDR1 sequence RASQGISSWLT (SEQ ID NO: 13); a VL CDR2 sequence AASSLQS (SEQ ID NO: 14); and a VL CDR3 sequence QQYDSYPIT (SEQ ID NO: 15).
   11. The CAR of embodiment 10, wherein the VH amino acid sequence has at least 80% identity to sequence SEQ ID NO: 31 and the VL amino acid sequence has at least 80% identity to SEQ ID NO: 32.
   12 The CAR of embodiment 2 or 3, wherein the binder domain is 5F11.
   13. The CAR of embodiment 2 or 3, wherein the binder domain comprises a sequence selected from the binder domain sequences listed in Table 1 or 1A.
   14. The CAR of any one of embodiments 1, 2 or 4-13, wherein the hinge domain comprises a sequence selected from the hinge domain sequences listed in Table 2.
   15. The CAR of any one of embodiments 1-14, further comprising a linker between the hinge domain and the transmembrane domain.
   16. The CAR of embodiment 15, wherein the linker comprises a sequence of KPDK (SEQ ID NO: 16).
   17. The CAR of any one of embodiments 1-16, wherein the transmembrane domain comprises the sequence of SEQ ID NO: 54.
   18. The CAR of any one of embodiments 1-17, wherein the costimulatory domain is a CD28 costimulatory domain.
   19. The CAR of embodiment 18, wherein the CD28 costimulatory domain is a wild-type CD28 costimulatory domain.
   20. The CAR of embodiment 18, wherein the CD28 costimulatory domain comprises the sequence of SEQ ID NO: 58.
   21. The CAR of any one of embodiments 1-20, wherein the costimulatory domain comprises a sequence selected from the costimulatory domain sequences listed in Table 4.
   22. The CAR of any one of embodiments 1-21, wherein the activation domain is a wild-type CD3z activation domain having an N-terminal argenine or an N terminal leucine.
   23. The CAR of any one of embodiments 1-21, wherein the CD3z activation domain comprises a sequence selected from the activation domain sequences listed in Table 5.
   24. The CAR of any one of embodiments 1-23, wherein: the hinge domain comprises a CD8a hinge sequence; the transmembrane domain comprises a CD28 transmembrane sequence; the costimulatory domain comprises a wild-type CD28 costimulatory sequence; and the activation domain comprises a wild-type CD3z activation sequence comprising an N-terminal leucine.
   25. The CAR of embodiment 24, wherein the CD8a hinge sequence comprises the sequence of SEQ ID NO: 50; the CD28 transmembrane sequence comprises the sequence of SEQ ID NO: 54; the CD28 costimulatory sequence comprises the sequence of SEQ ID NO: 57; and the CD3z activation sequence comprises the sequence of SEQ ID NO: 61.
   26. The CAR of embodiment 25, wherein the binder domain comprises the sequence of SEQ ID NO: 20.
   27. The CAR of embodiment 25, wherein the binder domain comprises the sequence of SEQ ID NO: 21.
   28. The CAR of embodiment 25, wherein the binder domain comprises the sequence of any one of SEQ ID NOs: 22.
   29. The CAR of any one of embodiments 1-13, wherein: the hinge domain comprises a CD8a hinge sequence; the transmembrane domain comprises a CD28 transmembrane sequence; the costimulatory domain comprises a wild-type CD28 costimulatory sequence; and the activation domain comprises a wild-type CD3z activation sequence having an N-terminal argenine.
   30. The CAR of embodiment 29, wherein the CD8a hinge sequence comprises the sequence of SEQ ID NO: 50; the CD28 transmembrane sequence comprises the sequence of SEQ ID NO: 54; the CD28 costimulatory sequence comprises the sequence of SEQ ID NO: 57; and the CD3z activation sequence comprises the sequence of SEQ ID NO: 60.
   31. The CAR of any one of embodiments 1-13, wherein: the hinge domain comprises a CD8a hinge sequence; the transmembrane domain comprises a CD28 transmembrane sequence; the costimulatory domain comprises a CD28 costimulatory sequence; and the activation domain comprises a modified CD3z activation sequence.
   32. The CAR of embodiment 31, wherein the CD8a hinge sequence comprises the sequence of SEQ ID NO: 50; the CD28 transmembrane sequence comprises the sequence of SEQ ID NO: 54; the CD28 costimulatory sequence comprises the sequence of SEQ ID NO: 57; and the CD3z activation sequence comprises the sequence of SEQ ID NO:62.
   33. The CAR of any one of embodiments 1-13, wherein: the hinge domain comprises a CD8a hinge sequence; the transmembrane domain comprises a CD8a transmembrane sequence; the costimulatory domain comprises a 41BB costimulatory sequence; and the activation domain comprises a modified CD3z activation sequence.
   34. The CAR of embodiment 33, wherein the CD8a hinge sequence comprises the sequence of SEQ ID NO: 50; the CD8a transmembrane sequence comprises the sequence of SEQ ID NO: 55; the 41BB costimulatory sequence comprises the sequence of SEQ ID NO: 59; and the CD3z activation sequence comprises the sequence of SEQ ID NO: 62.
   35. The CAR of any one of embodiments 30, 33, or 34, wherein the binder domain comprises the sequence of SEQ ID NO: 23.
   36. The CAR of any one of embodiments 1-35, wherein the binder domain is an scFv.
   37. The CAR of embodiment 1 comprising a sequence selected from the CAR sequences listed in Table 6.
   38. The CAR of embodiment 1 comprising a sequence selected from the CAR sequences SEQ ID NO: 65, SEQ ID NO: 66, SEQ ID NO: 67, SEQ ID NO: 68, SEQ ID NO: 69, SEQ ID NO: 71, SEQ ID NO: 72, SEQ ID NO: 73, SEQ ID NO: 74.
   39. An engineered cell comprising a CAR of any one of embodiments 1-38.
   40. The engineered cell of embodiment 39, wherein the engineered cell is derived from a T cell or a NK cell.
   41. The engineered cell of embodiment 40, wherein the engineered cell is derived from a T cell.
   42. The engineered cell of embodiment 41, wherein the engineered cell has reduced expression of a T cell receptor (TCR) on its surface relative to the T cell from which it was derived.
   43. The engineered cell of any one of embodiments 39-42, wherein the engineered cell expresses the CAR at an endogenous TRAC locus of the engineered cell.
   44. The engineered cell of embodiment 43, wherein the nucleic acid sequence encoding the CAR disrupts the coding sequence of a TCR in the TRAC locus.
   45. The engineered cell of any one of embodiments 39-44, wherein the engineered cell does not express a TCR.
   46. The engineered cell of any one of embodiments 39-45, wherein the engineered cell comprises at least one genetic modification in a MHC class II gene.
   47. The engineered cell of embodiment 36, wherein the engineered cell does not express the MHC class II gene.
   48. The engineered cell of embodiment 46 or 47, wherein the MHC class II gene is a HLA-DM gene, a HLA-DO gene, a HLA-DP gene, a HLA-DQ gene, or a HLA-DR gene.
   49. The engineered cell of any one of embodiments 39-48, wherein the engineered cell comprises at least one genetic modification in a CIITA gene.
   50. The engineered cell of embodiment 49, wherein the cell does not express a functional CIITA protein.
   51. The engineered cell of embodiment 49, wherein the cell is homozygous for HLA-B and homozygous for HLA-C.
   52. The engineered cell of any one of embodiments 41-50, wherein the engineered cell comprises at least one modification in a MHC class I gene.
   53. The engineered cell of embodiment 52, wherein the MHC class I gene is a HLA-A gene, a HLA-B gene, or a HLA-C gene, or a combination thereof.
   54. The engineered cell of embodiment 53, wherein the cell does not express the HLA-A gene.
   55. The engineered cell of embodiment 53 or embodiment 54, wherein the HLA-B gene and the HLA-C gene are matched to a subject who is to be administered the engineered cell.
   56. A population of cells comprising the engineered cell of any one of embodiments 39-55.
   57. A pharmaceutical composition comprising the engineered cell of any one of embodiments 39-55 or the population of cells of embodiment 56.
   58. A method of treating a disease or disorder in a subject, the method comprising administering to the subject the engineered cell, population of cells, or pharmaceutical composition of any one of embodiments 39-57 to the subject.
   59. The method of embodiment 58, wherein the engineered cell is homozygous for HLA-B and homozygous for HLA-C.
   60. The method of embodiment 58 or 59, wherein the disease or disorder is a cancer, an infectious disease, or an autoimmune disease.
   61. The method of embodiment 60, wherein the disease or disorder is a cancer.
   62. The method of embodiment 61, wherein the cancer is a hematologic cancer.
   63. The method of embodiment 62, wherein the hematologic cancer is a CD30-expressing hematologic cancer.
   64. The method of embodiment 63, wherein the CD30-expressing hematologic cancer is relapsed or refractory classical Hodgkin Lymphoma.
   65. A method of preventing or reducing graft versus host disease in a subject receiving an allogenic cell treatment, the method comprising administering to the subject the engineered cell, population of cells, or pharmaceutical composition of any one of embodiments 39-57.
   66. The engineered cell, population of cells, or pharmaceutical composition of any one of embodiments 39-57, for use in an adoptive cell transfer (ACT) therapy.
   67. A nucleic acid encoding the CAR of any one of embodiments 1-38.
   68. A vector comprising a nucleic acid encoding a CAR of any one of embodiments 1-38.
   69. A cell comprising the nucleic acid of embodiment 67 or the vector of embodiment 68.
   70. A method of making a CAR expressing engineered cell, the method comprising delivering a vector of embodiment 68 to a donor cell.
   71. The method of embodiment 70 further comprising delivering to the donor cell a gRNA targeting a locus for inserting into the locus the nucleic acid sequence encoding the CAR.
   72. The method of embodiment 70 or 71, further comprising delivering to the donor cell a nuclease or a nucleic acid encoding a nuclease.
   73. The method of embodiment 72, wherein the nuclease is a Cas9 nuclease.
   74. The method of any one of embodiment 70-73, further comprising delivering to the donor cell a gRNA that targets the HLA-A gene.
   75. The method of any one of embodiments 70-74 further comprising delivering to the cell a gRNA that targets the CIITA gene.
   76. The method of any one of embodiments 70-75, further comprising delivering to the donor cell a gRNA that targets the TRAC or TRBC locus.
   77. The method of any one of embodiments 69-76 further comprising delivering to the cell a gRNA that targets the B2M gene.
   78. A cell generated according to the method of any one of embodiments 69-77.

### EXAMPLES

The following examples are provided to illustrate certain disclosed embodiments and are not to be construed as limiting the scope of this disclosure in any way.

### Example 1: General Methods

### 1.1. T Cell Culture Media Preparation

T cell culture media compositions used below are described here. “X-VIVO Base Media” comprises X-VIVO™ 15 Media, 1% Penicillin-Streptomycin, 50 μM Beta-Mercaptoethanol, 10 mM non-essential amino acids (NAC). In addition to above mentioned components, other variable media components used were: 1. Serum (Fetal Bovine Serum (FBS)); and 2. Cytokines (IL-2, IL-7, IL-15).

### 1.2. Preparation of Lipid Nanoparticles

The lipid components were dissolved in 100% ethanol at various molar ratios. The RNA cargos (e.g., Cas9 mRNA and sgRNA) were dissolved in 25 mM citrate buffer, 100 mM NaCl, pH 5.0, resulting in a concentration of RNA cargo of approximately 0.45 mg/mL.

The lipid nucleic acid assemblies contained ionizable Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate), Lipid nanoparticles used 50% Lipid A, 38.5% cholesterol, 10% DSPC, and 1.5% PEG2k-DMG by molarity. The lipid nucleic acid assemblies were formulated with a lipid amine to RNA phosphate (N:P) molar ratio of about 6, and a ratio of gRNA to mRNA of 1:2 by weight.

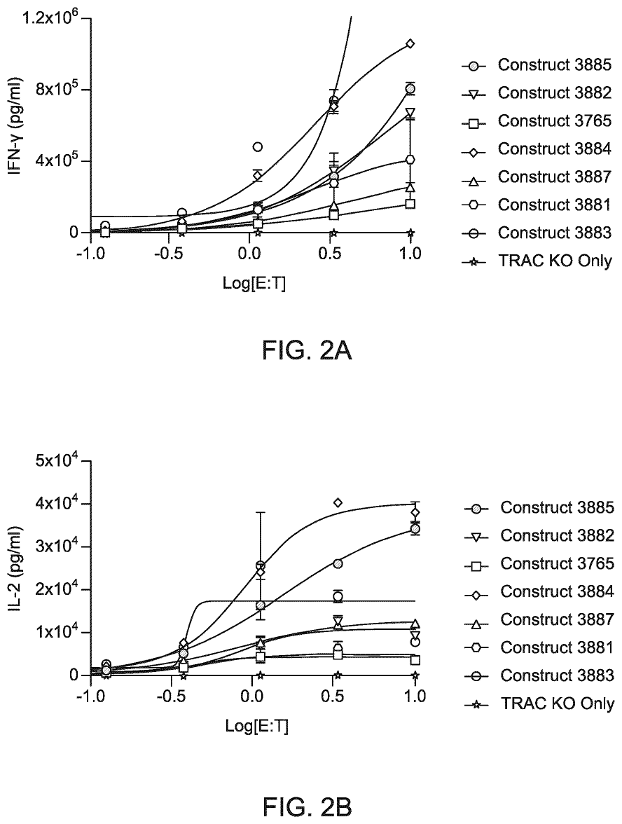

Lipid nanoparticles (LNP compositions) were prepared using a cross-flow technique utilizing impinging jet mixing of the lipid in ethanol with two volumes of RNA solutions and one volume of water. The lipids in ethanol were mixed through a mixing cross with the two volumes of RNA solution. A fourth stream of water was mixed with the outlet stream of the cross through an inline tee (See WO2016010840 FIG. 2.). The LNP compositions were held for 1 hour at room temperature (RT), and further diluted with water (approximately 1:1 v/v). LNP compositions were concentrated using tangential flow filtration on a flat sheet cartridge (Sartorius, 100 kD MWCO) and buffer exchanged using PD-10 desalting columns (GE) into 50 mM Tris, 45 mM NaCl, 5% (w/v) sucrose, pH 7.5 (TSS). Alternatively, the LNP's were optionally concentrated using 100 kDa Amicon spin filter and buffer exchanged using PD-10 desalting columns (GE) into TSS. The resulting mixture was then filtered using a 0.2 m sterile filter. The final LNP was stored at 4° C. or −80° C. until further use.

### 1.3. In Vitro Transcription (“IVT”) of mRNA

Capped and polyadenylated mRNA containing N1-methyl pseudo-U was generated by in vitro transcription using a linearized plasmid DNA template and T7 RNA polymerase. Plasmid DNA containing a T7 promoter, a sequence for transcription, and a polyadenylation sequence was linearized by incubating at 37° C. for 2 hours with XbaI with the following conditions: 200 ng/μL plasmid, 2 U/μL XbaI (NEB), and 1× reaction buffer. The XbaI was inactivated by heating the reaction at 65° C. for 20 min. The linearized plasmid was purified from enzyme and buffer salts. The IVT reaction to generate modified mRNA was performed by incubating 50 ng/μL linearized plasmid; 2-5 mM each of GTP, ATP, CTP, and N1-methyl pseudo-UTP (Trilink); 10-25 mM ARCA (Trilink); 5 U/μL T7 RNA polymerase (NEB); 1 U/μL Murine RNase inhibitor (NEB); 0.004 U/μL Inorganic E. coli pyrophosphatase (NEB); and 1× reaction buffer at 37° C. for 1.5-4 hours. TURBO DNase (ThermoFisher) was added to a final concentration of 0.01 U/μL, and the reaction was incubated for an additional 30 minutes to remove the DNA template. The mRNA was purified using a MegaClear Transcription Clean-up kit (ThermoFisher) or a RNeasy Maxi kit (Qiagen) per the manufacturers' protocols. Alternatively, the mRNA was purified through a precipitation protocol, which in some cases was followed by HPLC-based purification. Briefly, after the DNase digestion, mRNA is purified using LiCl precipitation, ammonium acetate precipitation and sodium acetate precipitation. For HPLC purified mRNA, after the LiCl precipitation and reconstitution, the mRNA was purified by RP-IP HPLC (see, e.g., Kariko, et al. Nucleic Acids Research, 2011, Vol. 39, No. 21 e142). The fractions chosen for pooling were combined and desalted by sodium acetate/ethanol precipitation as described above. In an alternative method, mRNA was purified with a LiCl precipitation method followed by further purification by tangential flow filtration. RNA concentrations were determined by measuring the light absorbance at 260 nm (Nanodrop), and transcripts were analyzed by capillary electrophoresis by Bioanlayzer (Agilent).

Streptococcus pyogenes (“Spy”) Cas9 mRNA was generated from plasmid DNA encoding an open reading frame having a nucleic acid sequence of one of SEQ ID NOs: 801-803 (see sequences in Table 17). When SEQ ID NOs: 801-803 are referred to below with respect to RNAs, it is understood that Ts should be replaced with Us (which were N1-methyl pseudouridines as described above). Messenger RNAs used in the Examples include a 5′ cap and a 3′ polyadenylation region, e.g., up to 100 nucleotides, and have a nucleic acid sequence of one of SEQ ID NOs: 801-803 in Table 17.

### Example 2: Anti-CD30 CAR-T Cell Tumor Killing Assay with HH Tumor Cells

T cells engineered with anti-CD30 CAR constructs that vary in the configuration of the binder, hinge, transmembrane, costimulatory, and activation domains were tested for their cytotoxicity against HH tumor cells.

### Example 2.1: Preparing T Cell Growth Media (TCGM)

Human AB Serum and Penicillin-Streptomycin were pre-thawed in 37° C. water bath. 24.0 mL of CTS supplement (Gibco, Cat. A1048402), 50.0 mL of Human AB Serum (GeminiBio, Cat. 100-512), 10.0 mL of GlutaMAX (Gibco, Cat. 35050-061), 10.0 ml of HEPES (Gibco, Cat. 15630-080), and 10.0 mL of Penicillin-Streptomycin (Gibco, Cat. 15070-063) were added to 1000 mL of CTS OpTmizer T cell expansion media (Gibco, Cat. A1048501). The media were then mixed and filtered through 0.2 m aPES filter (Thermo Scientific. Cat. 567-0020). The resulting filtered media is T cell growth media (TCGM) as in Example 1.1 to which IL-2, IL-7, and IL-15 were added before use as needed.

### Example 2.2: Thawing and Resting CAR-T and Control T Cells

Cryopreserved anti-CD30 CAR-T cells and the negative control were removed from liquid nitrogen tank and immediately thawed in a 37° C. water bath until a sliver of ice is remaining in the vial. T-cells were then transferred to a conical tube containing pre-warmed TCGM media and then washed by centrifugation at 500×G for 5 minutes followed by resuspending in fresh TCGM media. T-cells were then counted using Vi-Cell and resuspended to 1.5×10⁶ cells/ml in pre-warmed TCGM media and further supplemented with 100 U/mL of recombinant human interleukin-2 (Peprotech, Cat. 200-02), 5 ng/ml IL-7 (Peprotech, Cat. 200-07), 5 ng/ml IL-15 (Peprotech, Cat. 200-15). Cells were transferred to a T-75 flask and rested overnight in a 37° C. incubator.

### Example 2.3: Killing Assay Setup with CAR-T and Tumor Cells

HH GFP-Luciferase cells were counted using a Vi-cell and the desired number of cells were collected in a 15 mL conical tube. Tumor cells were then washed by centrifuging at 500 XG for 5 minutes, followed by resuspending in 1 mL pre-warmed TCGM media. The resuspended tumor cells were counted again by Vi-cell and then the concentration of tumor cells was adjusted to 2×10⁵ cells/mL. 0.1 mL of the tumor cell suspension was added to each well in a white 96-well plate (Thermo Scientific, Cat. 136101). All CAR-T cells and control T cells were counted using Vi-cell and desired number of cells were collected in a 15 mL conical tube. CAR-T cells and control T cells were then washed by centrifuging at 500 XG for 5 minutes followed by resuspending in 1 mL pre-warmed TCGM media. The resuspended T cells were counted again by Vi-cell. The concentration of T cells was adjusted to 2×10⁶ cells/mL, so that the starting T cell to tumor cell ratio is 10:1, and then serially diluted 3-fold 5 times. 100 μL of each serial diluted T cells were added to the tumor cell plate in each well according to the desired plate map. The plate was then transferred to a 37° C. incubator and incubated overnight.

### Example 2.4: Killing Assay Readout

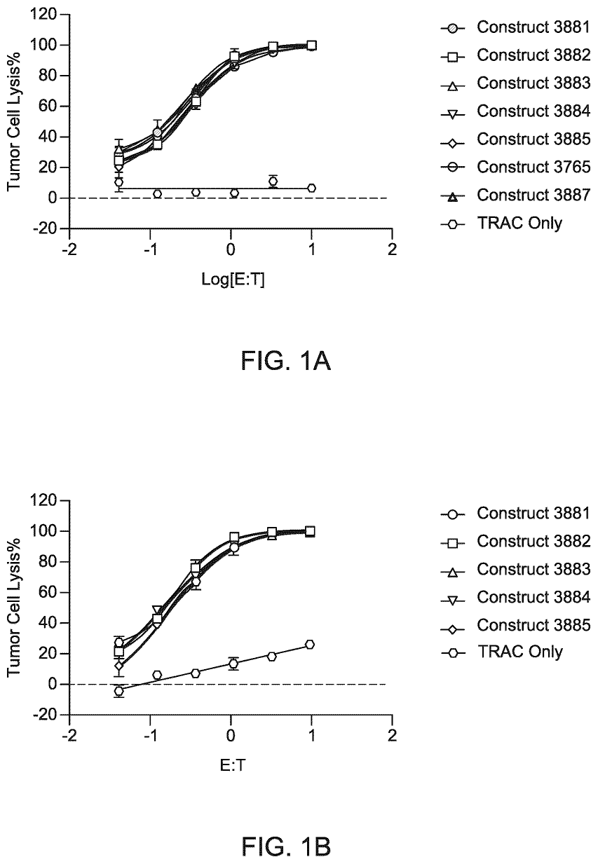

Bright-Glo™ Luciferase Assay System (Promega, Cat. E2620) was pre-thawed in dark at room temperature. The killing assay plate was taken out from the incubator, 50 μL of the supernatant in each well was carefully collected without disturbing the cells at the bottom of the well. 50 μL of Bright-Glo™ Luciferase Assay System was added to each well and the plate was shaken briefly on a shaker and then incubated in dark at room temperature for 5 minutes. The plate was then read for luminescence with a CLARIOstar plate reader. The percentage killing was calculated from the luminescence with the average of T cell to tumor cell ratio 0 as 0% killing. The results are shown in Tables 7 and 8 and FIGS. 1A and 1B.

TABLE 7
Mean HH Tumor Cell Killing, Donor 1

| | Construct | | | | | | | | | | | | | | | |
|---|---|---|---|---|---|---|---|---|---|---|---|---|---|---|---|---|
| | 3881 | | 3882 | | 3883 | | 3884 | | 3885 | | 3887 | | 3765 | | TRAC Only | |
| E:I | Mean | SD | Mean | SD | Mean | SD | Mean | SD | Mean | SD | Mean | SD | Mean | SD | Mean | SD |
| 10.00 | 99.93 | 0.01 | 99.92 | 0.05 | 99.95 | 0.03 | 99.93 | 0.02 | 99.81 | 0.04 | 99.66 | 0.14 | 99.41 | 0.12 | 6.50 | 0.37 |
| 3.33 | 99.32 | 0.21 | 99.25 | 0.74 | 99.02 | 0.07 | 98.90 | 0.10 | 97.77 | 3.43 | 98.31 | 0.23 | 95.45 | 1.20 | 11.10 | 3.97 |
| 1.11 | 92.95 | 1.94 | 92.71 | 2.16 | 91.33 | 3.46 | 88.27 | 3.80 | 92.83 | 1.76 | 93.26 | 4.55 | 86.21 | 2.24 | 3.38 | 1.08 |
| 0.37 | 65.54 | 3.64 | 63.12 | 1.68 | 68.55 | 2.17 | 61.13 | 2.92 | 67.59 | 3.75 | 71.60 | 2.78 | 65.27 | 1.75 | 3.69 | 1.07 |
| 0.12 | 43.08 | 0.02 | 35.26 | 3.44 | 44.66 | 2.43 | 37.27 | 1.24 | 40.32 | 2.52 | 43.43 | 7.85 | 34.57 | 1.26 | 2.84 | 0.89 |
| 0.04 | 28.33 | 3.81 | 24.77 | 3.12 | 32.22 | 6.18 | 23.45 | 2.57 | 20.60 | 7.26 | 29.66 | 4.24 | 24.36 | 0.63 | 10.48 | 6.48 |
| 0.00 | 99.93 | 0.01 | 99.92 | 0.05 | 99.95 | 0.03 | 99.91 | 0.02 | 99.81 | 0.04 | 99.66 | 0.14 | 99.41 | 0.12 | 6.50 | 0.37 |

| | | | | | | | | |
|---|---|---|---|---|---|---|---|---|
| EC50 | 0.3188 | 0.3515 | 0.3121 | 0.3500 | 0.2393 | 0.2684 | 0.3144 | ~186.4 |

TABLE 8
Mean HH Tumor Cell Killing, Donor 2

| | Construct | | | | | | | | | | | |
|---|---|---|---|---|---|---|---|---|---|---|---|---|
| | 3881 | | 3882 | | 3883 | | 3884 | | 3885 | | TRAC Only | |
| E:T | Mean | SD | Mean | SD | Mean | SD | Mean | SD | Mean | SD | Mean | SD |
| 10.00 | 99.87 | 0.04 | 99.95 | 0.01 | 99.85 | 0.01 | 99.95 | 0.01 | 99.92 | 0.01 | 25.78 | 2.02 |
| 3.33 | 99.09 | 0.20 | 99.68 | 0.18 | 97.87 | 0.93 | 99.09 | 0.40 | 97.70 | 1.58 | 17.80 | 2.19 |
| 1.11 | 89.91 | 5.24 | 96.31 | 0.71 | 89.55 | 1.50 | 91.05 | 0.95 | 90.63 | 0.01 | 13.24 | 3.92 |
| 0.37 | 66.94 | 4.99 | 76.02 | 5.18 | 71.25 | 2.33 | 70.03 | 1.00 | 66.59 | 2.54 | 6.71 | 1.95 |
| 0.12 | 40.86 | 2.37 | 43.89 | 4.37 | 45.02 | 2.63 | 48.41 | 2.16 | 38.92 | 2.77 | 5.64 | 2.04 |
| 0.04 | 27.47 | 4.09 | 22.10 | 2.66 | 22.81 | 1.75 | 21.21 | 4.81 | 11.76 | 6.79 | −5.03 | 3.68 |
| 0.00 | 99.87 | 0.04 | 99.95 | 0.01 | 99.85 | 0.01 | 99.95 | 0.01 | 99.92 | 0.01 | 25.78 | 2.02 |

| | | | | | | |
|---|---|---|---|---|---|---|
| EC50 | 0.3047 | 0.1966 | 0.1764 | 0.1265 | 0.1706 | ~67665 |

### 2.5. Engineered T Cell Cytokine Release

Engineered T cells prepared as described in Example 4.1 were co-cultured with HH tumor cells as described above in sections 2.2-2.4. Supernatants from the co-culture were collected as described in section 2.4 and assayed for the cytokine release profiles of each engineered T cell group as described below:

For each of the cytokines measured, biotinylated capture antibody from the U-PLEX Immuno-Oncology Group 1 (hu) Assays (MSD, Cat No. K151AEL-2) was added to the assigned linker according to the kit's protocol. The antibody-linker mixtures were vortexed and incubated at room temperature for 30 minutes, after which the free antibodies were quenched with the Stop Solution from the kit. The antibody-linker solutions for each cytokine to be measured were combined to form the coating mix according to the kit instructions. Post incubation, the plate was washed, sealed, and stored overnight.

The following day, calibrators containing standards for each of the cytokines (IL-2 and IFN-7) to be assayed were reconstituted as per the manufacturer's instructions and diluted to create a 4-fold standard curve. 50 μL of each standard was loaded in duplicates on the MSD plate. The supernatant from each co-culture sample was thawed on ice and was diluted 1:10 in Diluent 2 from the U-PLEX Immuno-Oncology Group 1 (hu) Assays kit (MSD, Cat No. K151AEL-2). 50 μL of diluted samples from each group were loaded onto the meso scale discovery (MSD) plate and incubated for 1 hour.

The plates were washed 3 times with 150 μL of wash buffer as per the kit instructions, and 50 μL of the detection antibody solution (dilution prepared according to kit instructions) was added to each well of the MSD plate. The plate was incubated for 1 hour.

After incubation, the plate was washed 3 times with 150 μL and read immediately on the MSD instrument. Cytokine release is shown in Tables 9-10 and FIGS. 2A-2B.

TABLE 9
Concentration of IFN- γ released following T cell treatment
IFN-γ

| | Construct | | | | | | | |
|---|---|---|---|---|---|---|---|---|
| | 3881 | | 3882 | | 3884 | | 3883 | |
| Log[peptide | Avg | Stdev | Avg | Stdev | Avg | Stdev | Avg | Stdev |
| (nM)] | IFN-γ | IFN-γ | IFN-γ | IFN-γ | IFN-γ | IFN-γ | IFN-γ | IFN-γ |
| 10.00 | 409616.2 | 224606.2 | 667264.2 | 25328.6 | 1057322.0 | 11985.5 | 1034028.5 | 38710.56 |
| 3.33 | 283235.2 | 162240.6 | 344567.5 | 53347.4 | 707531.1 | 36691.3 | 737031.6 | 58855.75 |
| 1.11 | 145761.3 | 28930.8 | 135739.2 | 40884.8 | 322363.2 | 32586.0 | 481081.5 | 0 |
| 0.37 | 31308.4 | 1804.9 | 28628.4 | 3067.4 | 62330.1 | 7321.5 | 114071.5 | 3554.626 |
| 0.12 | 12359.4 | 2608.1 | 7853.2 | 827.8 | 18455.8 | 1551.1 | 39036.045 | 3299.834 |
| 0.04 | 2986.1 | 119.2 | 1836.2 | 228.1 | 5304.1 | 976.6 | 12088.363 | 3735.174 |

| | Construct | | | | | | | |
|---|---|---|---|---|---|---|---|---|
| | 3885 | | 3765 | | 3887 | | TRAC KO Only | |
| Log[peptide | Avg | Stdev | Avg | Stdev | Avg | Stdev | Avg | Stdev |
| (nM)] | IFN-γ | IFN-γ | IFN-γ | IFN-γ | IFN-γ | IFN-γ | IFN-γ | IFN-γ |
| 10.00 | 806741.6 | 34549.9 | 162660.9 | 15673.6 | 258699.2 | 24943.8 | 146.6 | 26.9 |
| 3.33 | 320527.0 | 62517.6 | 96267.1 | 495.4 | 156520.7 | 6185.3 | 34.2 | 5.1 |
| 1.13 | 133111.8 | 5766.0 | 57250.4 | 2044.9 | 71529.8 | 8118.2 | 29.3 | 11.4 |
| 0.37 | 25450.4 | 345.5 | 20072.7 | 2013.4 | 23403.4 | 2032.8 | 12.6 | 1.2 |
| 0.12 | 9168.6 | 380.2 | 7967.9 | 1062.6 | 8470.6 | 1143.1 | 13.3 | 2.4 |
| 0.04 | 2086.9 | 288.0 | 2248.4 | 435.0 | 2819.3 | 144.6 | 12.7 | 4.7 |

TABLE 10
Concentration of IL-2 realease following T cell treatment
IL-2

| | Construct | | | | | | | |
|---|---|---|---|---|---|---|---|---|
| | 3881 | | 3882 | | 3884 | | 3883 | |
| Log[peptide | Avg | Stdev | Avg | Stdev | Avg | Stdev | Avg | Stdev |
| (nM)] | IL-2 | IL-2 | IL-2 | IL-2 | IL-2 | IL-2 | IL-2 | IL-2 |
| 10.00 | 3623.7 | 444.8 | 9072.0 | 580.3 | 37867.4 | 2374.8 | 7773.12 | 866.13 |
| 3.33 | 6361.4 | 1479.3 | 12400.6 | 1499.8 | 40117.6 | 451.5 | 18295.12 | 1475.05 |
| 1.11 | 4106.9 | 1211.1 | 6843.6 | 1723.8 | 23997.1 | 1830.2 | 25437.16 | 12380.76 |
| 0.37 | 1707.3 | 154.3 | 2246.3 | 385.7 | 7420.2 | 43.3 | 6461.87 | 190.74 |
| 0.12 | 642.7 | 12.3 | 731.0 | 119.2 | 2156.6 | 110.4 | 2445.28 | 57.09 |
| 0.04 | 175.2 | 28.8 | 200.7 | 3.9 | 678.6 | 3.8 | 999.23 | 254.54 |

| | Construct | | | | | | | |
|---|---|---|---|---|---|---|---|---|
| | 3885 | | 3765 | | 3887 | | TRAC KO Only | |
| Log[peptide | Avg | Stdev | Avg | Stdev | Avg | Stdev | Avg | Stdev |
| (nM)] | IL-2 | IL-2 | IL-2 | IL-2 | IL-2 | IL-2 | IL-2 | IL-2 |
| 10.00 | 34019.8 | 1244.3 | 3482.5 | 223.2 | 12077.0 | 769.9 | 12.8 | 2.0 |
| 3.33 | 25867.1 | 883.3 | 4823.6 | 220.1 | 11772.7 | 643.8 | 8.0 | 0.4 |
| 1.11 | 16295.5 | 1005.1 | 4156.5 | 15.0 | 7505.3 | 1448.9 | 5.0 | 3.8 |
| 0.37 | 5063.6 | 186.7 | 1814.0 | 161.7 | 3535.9 | 225.5 | 4.7 | 0.0 |
| 0.12 | 1626.6 | 133.1 | 578.6 | 49.6 | 1406.7 | 78.5 | 3.6 | 0.4 |
| 0.04 | 449.6 | 40.2 | 187.5 | 48.2 | 454.6 | 11.5 | 2.4 | 1.5 |

### Example 3: Rechallenging Anti-CD30 CAR-T Cells with HH Tumor Cells

### Example 3.1: Thawing and Resting CAR-T and Control T Cells

Cryopreserved T cells expressing anti-CD30 CAR constructs that vary in the configuration of the either HRS3 or 5F11 binder, hinge, transmembrane, costimulatory, and activation domains were thawed. T cells expressing the following anti-CD30 CAR constructs were tested: 3881, 3882, 3883, 3884, 3885, 3886, 3887, 3765, 3764, and T cells with edits in TRAC only. T-cells were transferred to a conical tube containing pre-warmed TCGM media composed of: CTS OpTmizer T-cell expansion SFM (Gibco, Cat. #A1048501), OpTmizer CTS T-cell expansion supplement (Gibco, Cat. #A1048402), GemCell human serum A (GeminiBio, Cat. #100-512), 1M HEPES (Gibco, Cat. #15630-080), GlutaMAX supplement (Gibco, Cat. #35050-061), and 5,000 U/mL Penicillin-Streptomycin (Gibco, Cat. #15070-063). Cells were then washed by centrifugation at 500×G for 5 minutes followed by resuspending in fresh TCGM media. T-cells were then counted using Vi-Cell and resuspended to 1.5×10⁶ cells/mL in pre-warmed TCGM media composed of OpTmizer TCGM containing 5% Human Ab serum, 1% Pen-Strep, 1× Glutamax and 1×HEPES and further supplemented with 100 U/mL of recombinant human interleukin-2 (Peprotech, Cat. 200-02), 5 ng/mL IL-7 (Peprotech, Cat. 200-07), 5 ng/mL IL-15 (Peprotech, Cat. 200-15). Cells were transferred to a T-75 flask and rested O/N in a 37° C. incubator.

### Example 3.2: Assay Setup with Effector and Target Cells

HH GFP-Luciferase cells were counted using a Vi-cell and desired number of cells were collected in a 15 mL conical tube. Tumor cells were then washed by centrifuging at 500 XG for 5 mins followed by resuspending in pre-warmed TCGM media at 1×10⁶ cells/mL. Using a sterile collage coated 24 well plate (Corning, Cat. 354408), 200 μl of tumor cells per well were added. All CAR-T cells and control T cells were removed from the incubator and counted using vi-cell and washed by centrifugation as described in previous steps. A desired number of T-cells were resuspended in TCGM media at 1×10⁶ cells/mL. 100 μl of T cells were added to its respective wells containing HH tumor cells such that the starting effector to target ratio was 1:2. 700 μl of TCGM media was added to bring up the total volume to 1 mL. The plate was transferred a live cell analysis instrument (Incuyte S3, Sartorius) at 37° C.; the images were captured every 4-6 hours via Incucyte 2019B Rev2 software using Phase and Green channels with 10× objective.

### Example 3.3: Rechallenge with Tumor Cells and Performing Flow Cytometry

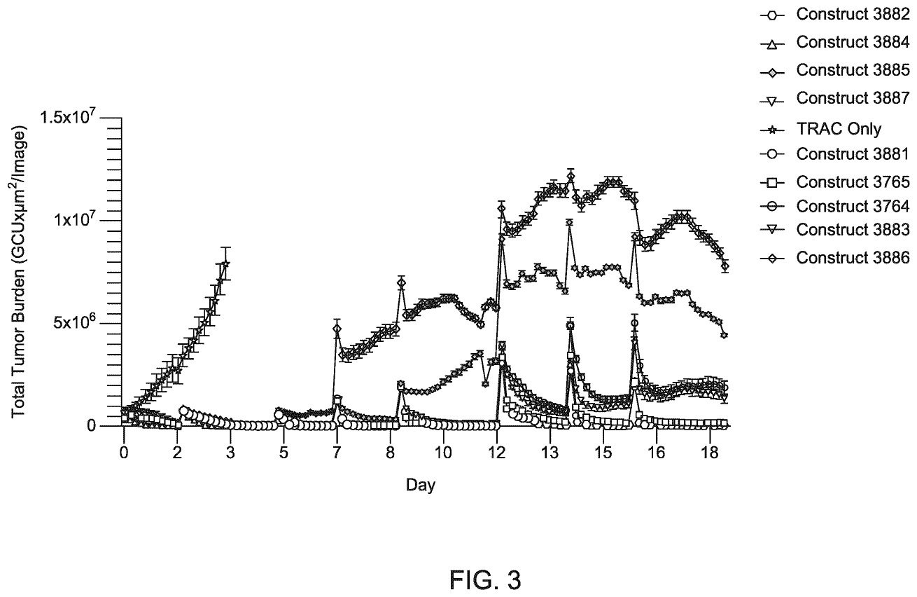

CAR-T Cells were rechallenged with HH cells on Day 2, 5, 7, 9, 12, 14 and 16. On days of rechallenge, a small number of cells were removed for flow cytometric analysis as described below and to measure cell counts using the Nexcelom Celleca cell counter. CAR-T cells were rechallenged with 400,000 cells/well of tumor cells on all the rechallenge days by first removing 500 μl of media and replacing with pre-warmed TCGM media containing HH cells. After adding HH cells, the plate was put back into the Incucyte for further analysis. FIG. 3 shows the total green integrated object intensity (GCU×μm²/image) for CD30 constructs.

For flow cytometry CAR-T cells were collected on Day 2, 7, 14, and 18 before adding new HH tumor cells and transferred to a 96-well V-bottom plate. Cells were briefly centrifuged followed by adding ViaKrome Live/Dead dye (Beckman Coulter, Cat. C36628) at 1:10,000 dilution. Recombinant—CD30 Fc was then added at 10 μg/ml followed by Anti-Human IgG Fc to detect CAR. Other T cell related activation, exhaustion and memory markers were added to the antibody cocktail as shown in Table 11 below. Bound fluorescent antibodies were detected using BD cytoflex LX flow cytometer.

TABLE 11
Antibody Panel for Tumor rechallenge with CD30 CAR constructs

| Marker | Fluorophore | Clone | Manufacturer | Cat# | Dilution |
|---|---|---|---|---|---|
| PD-1 | BV421 | EH12.2H7 | Biolegend | 329920 | 1:100 |
| Tim-3 | BV510 | F38-2E2 | Biolegend | 345030 | 1:100 |
| CD69 | BV650 | FN50 | Biolegend | 310933 | 1:100 |
| CD62L | BV785 | DREG-56 | Biolegend | 304830 | 1:100 |
| Target Cells (HH) | GFP | — | — | — | — |
| LAG-3 | PE | 11C3C65 | Biolegend | 369306 | 1:100 |
| CD8 | PerCP | SK1 | Biolegend | 980916 | 1:100 |
| CD45RO | PECy7 | UCHL1 | Biolegend | 304230 | 1:100 |
| Anti-CD30 | APC | M1310G05 | Biolegend | 410712 | 1:100 |
| CD25 | AF700 | BC96 | Biolegend | 302622 | 1:100 |
| CD4 | APCCy7 | RPA-T4 | Biolegend | 300518 | 1:100 |
| Live/Dead | ViaKrome808 | N/A | Beckman Coulter | C36628 | 1:10,000 |
| CountBright ™ Absolute | — | — | Thermo Fisher | C36950 | |
| Counting Beads | | | | | |
| Recombinant CD30-Fc | — | — | R&D Systems | 813- | 10 ug/ml |
| | | | | CD-100 | |

### Example 3.4: Resting T Cells Post Rechallenge and Final Killing with Tumor Cells

CAR-T cells that showed continuous tumor clearance across multiple rounds of rechallenge were then collected, washed, and rested in a 24 well G-Rex containing TCGM media supplemented with 100 U/mL of recombinant human interleukin-2 (Peprotech, Cat. 200-02), 5 ng/mL IL-7 (Peprotech, Cat. 200-07), and 5 ng/mL IL-15 (Peprotech, Cat. 200-15) for 7 days in a 37° C. incubator. Half of the media was changed every 2 days and supplemented with fresh cytokines.

On day 8 post rest, CAR-T cells were counted using Vi-cell and desired number of cells were taken out for a final killing assay with HH luciferase expressing tumor cells. 20,000 cells/well of tumor cells were added to a 96-well flat white-bottom plate. CAR-T cells were 3-fold serially diluted starting from 200,000 cells/well and co-cultured with tumor cells overnight. The next day, Bright Glo luciferase (Promega, Cat #E2610) substrate was added to each well and incubated at room temperature for 5 minutes before reading it in CLARIOstar plate reader.

### Example 4: In-Vivo Tumor Burden Assay

Female NOG mice were engrafted with 0.3×10⁶ HH-Luc2 tumor cells by intravenous injection into the tail vein. Four days later, mice were intravenously injected with 5×10⁶ engineered T cells expressing various constructs of CD30 CAR to assess efficacy in tumor growth suppression in vivo. The study groups include: T cells with tumor only (control); T cells with edits in TRAC only; T cells expressing construct 3881; T cells expressing construct 3882; T cells expressing construct 3883; T cells expressing construct 3884; and T cells expressing construct 3885.

### 4.1. Preparation of T Cells

T cells were isolated from peripheral blood of a healthy human donor. Briefly, a leukapheresis pack (HemaCare Technologies) was treated in ammonium chloride RBC lysis buffer (Stemcell Technologies; Cat. 07800) for 15 minutes to lyse red blood cells. Peripheral blood mononuclear cell (PBMC) count was determined post lysis and T cell isolation was performed using EasySep Human T cell isolation kit (Stemcell Technologies, Cat. 17951) according to manufacturer's protocol. Isolated CD3+ T cells were re-suspended in Cryostor CS10 media (Stemcell Technologies, Cat. 07930) and frozen down in liquid nitrogen until further use.

Frozen T cells were thawed at a cell concentration of 1.5×10⁶ cells/ml into T cell activation media (TCAM) composed of CTS Optimizer (Gibco A3705001), GlutaMAX (Gibco Cat. 35050061), 2.5% Human AB Serum (Gemini Cat. 100-512), HEPES (Gibco Cat. 15630080), Penicillin-Streptomycin (Cat. 15140122), 200 U/mL of recombinant human interleukin-2 (Peprotech, Cat. 200-02), 5 ng/mL IL-7 (Peprotech, Cat. 200-07), and 5 ng/mL IL-15 (Peprotech, Cat. 200-15). Cells were rested at 37° C. for 24 hours.

Twenty-four hours post thawing T cells were counted and resuspended at 1×10⁶ cells/ml in TCAM media and TransACT (Miltenyi Cat. 130-111-160) was added to a final concentration of 1/100 of the total volume. Cells were mixed and were incubated at 37° C. for 24 hours.

Forty-eight hours post activation all groups were transduced with EF1α-CAR AAV. AAV was removed from −80° C. and thawed on ice. Transduction media was generated from TCAM by adding ApoE3 (Peprotech, Cat. 350-02,) to a final concentration of 2.5 μg/mL, TRAC-targeted LNP to a final concentration of 2.5 μg/mL, and DNApki Compound 1 to a final concentration of 0.25 μM. Cells were collected, centrifuged at 500×G for 5 minutes and resuspended at 0.5e6 cells/mL in transduction media. Appropriate AAVs for CD30 constructs were added to cells at an MOI of 3e5 GC/cell. Cells were mixed and were incubated at 37° C. After 24 hours, cells were transferred to GREX plates (Wilson Wolf Cat. 80660M) and expanded for 7 days with regular changes in media and cytokines. After expansion, CAR insertion rates were quantified using flow cytometry, and cells were cryopreserved in Cryostor CS10 freezing media (StemCell Cat. 07930).

### 4.2. CD30 CAR-T Constructs Demonstrate Varied HH-Luc2 Tumor Control

For the in vivo study, HH-Luc2 tumor cells were harvested from culture, washed twice with HBSS (Gibco, Cat. No. 14025-092) and resuspended at 0.3×10⁶ cells for injection in 150 μl HBSS. Fifty-four female NOG mice (Taconic) were dosed by tail vein injection with HH-Luc2 tumor cells. Four days later, engineered T cells expressing CAR constructs 3881, 3882, 3883, 3884, or 3885 were thawed, washed with HBSS (Gibco, Cat. No. 14025-092) and resuspended at 5×10⁶ cells/mL for injection in 150 μL HBSS. Six HH-Luc2 engrafted mice per T cell group were dosed by tail vein injection.

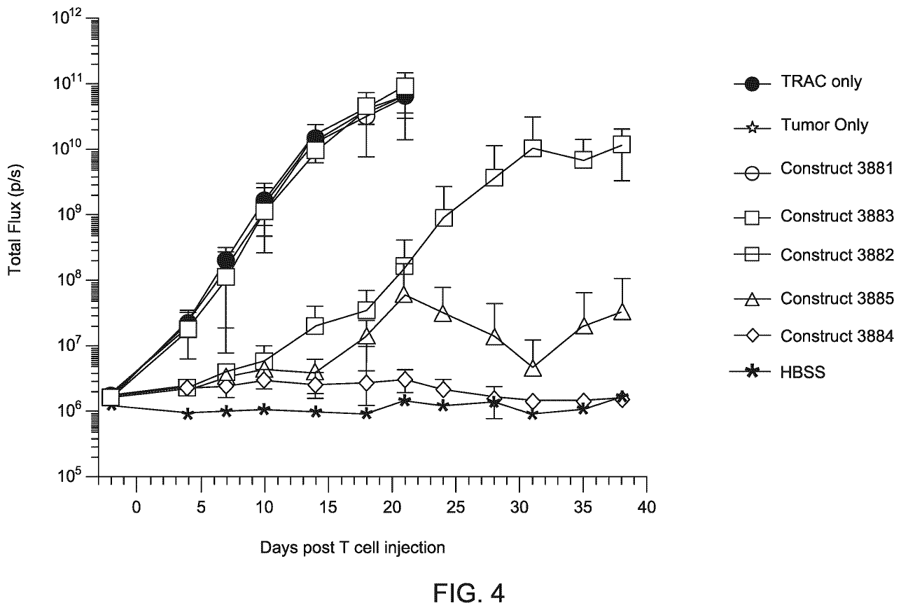

IVIS imaging of live mice was performed to identify luciferase-positive tumor cells by IVIS spectrum to study the tumor burden. IVIS imaging was done at day −2, 4, 7, 10, 14, 18, 21, 24, 28, 31, 35 and 38 post T cell injection. Mice were prepared for imaging with an injection of D-luciferin i.p. at 10 μL/g body weight per the manufacturer's recommendation, about 100 μL per animal. Animals were anesthetized and then placed in the IVIS imaging unit. The visualization was performed with the exposure time set to auto, field of view D, medium binning, and F/stop set to 1. Table 12 and FIG. 4 show total flux (photons/second) from luciferase expressing tumor cells present at the various time points after injection. Tumor control is depicted graphically in FIG. 4. These mice were sacrificed at day 34 to track T cells via flow cytometry.

TABLE 12
Total Flux (photons/s) from luciferase expressing tumor cells
in treated mice at intervals after T cell injection.

| | Construct | | | | | |
|---|---|---|---|---|---|---|
| Days | TRAC only | | | 3885 | | |
| post T- | Average | SD | | Average | SD | |
| cell | Total | Total | | Total | Total | |
| injection | Flux (p/s) | Flux (p/s) | N | Flux (p/s) | Flux (p/s) | N |
| −2 | 1.77E+06 | 3.32E+05 | 6 | 1.68E+06 | 4.46E+05 | 6 |
| 4 | 2.13E+07 | 5.88E+06 | 6 | 2.14E+06 | 3.59E+05 | 6 |
| 7 | 2.10E+08 | 8.96E+07 | 6 | 3.33E+06 | 5.28E+05 | 6 |
| 10 | 1.75E+09 | 1.14E+09 | 6 | 4.33E+06 | 1.83E+06 | 6 |
| 14 | 1.29E+10 | 6.08E+09 | 6 | 3.92E+06 | 1.91E+06 | 6 |
| 18 | 4.37E+10 | 1.51E+10 | 6 | 1.38E+07 | 9.07E+06 | 6 |
| 21 | 6.24E+10 | 4.30E+10 | 6 | 5.91E+07 | 9.88E+07 | 6 |
| 24 | | | | 3.06E+07 | 4.36E+07 | 6 |
| 28 | | | | 1.38E+07 | 2.71E+07 | 6 |
| 31 | | | | 4.60E+06 | 7.16E+06 | 6 |
| 35 | | | | 2.00E+07 | 4.09E+07 | 6 |
| 38 | | | | 3.28E+07 | 7.04E+07 | 6 |

| | Construct | | | | | |
|---|---|---|---|---|---|---|
| Days | 3882 | | | 3883 | | |
| post T- | Average | SD | | Average | SD | |
| cell | Total | Total | | Total | Total | |
| injection | Flux (p/s) | Flux (p/s) | N | Flux (p/s) | Flux (p/s) | N |
| −2 | 1.67E+06 | 356648.3 | 6 | 1.65E+06 | 343659.6 | 6 |
| 4 | 2.31E+06 | 308637.1 | 6 | 1.70E+07 | 9886931 | 6 |
| 7 | 3.93E+06 | 1060029 | 6 | 1.09E+08 | 81951408 | 6 |
| 10 | 5.94E+06 | 3155602 | 6 | 1.15E+09 | 4.05E+08 | 6 |
| 14 | 1.97E+07 | 18916602 | 6 | 9.36E+09 | 2.74E+09 | 6 |
| 18 | 3.40E+07 | 33149725 | 6 | 4.62E+10 | 1.97E+10 | 6 |
| 21 | 1.63E+08 | 2.34E+08 | 6 | 9.42E+10 | 5.33E+10 | 6 |
| 24 | 9.05E+08 | 1.63E+09 | 6 | | | |
| 28 | 3.73E+09 | 7.01E+09 | 6 | | | |
| 31 | 1.05E+10 | 1.93E+10 | 6 | | | |
| 35 | 6.99E+09 | 5.92E+09 | 6 | | | |
| 38 | 1.20E+10 | 6.04E+09 | 6 | | | |

| | Construct | | | | | |
|---|---|---|---|---|---|---|
| Days | 3884 | | | 3881 | | |
| post T- | Average | SD | | Average | SD | |
| cell | Total | Total | | Total | Total | |
| injection | Flux (p/s) | Flux (p/s) | N | Flux (p/s) | Flux (p/s) | N |
| −2 | 1.79E+06 | 226639.2 | 6 | 1.70E+06 | 173149.7 | 6 |
| 4 | 2.20E+06 | 460776 | 6 | 2.24E+07 | 7946701 | 6 |
| 7 | 2.35E+06 | 685077 | 6 | 2.11E+08 | 38466161 | 6 |
| 10 | 2.91E+06 | 654639.3 | 6 | 1.42E+09 | 1.05E+09 | 6 |
| 14 | 2.47E+06 | 794315.4 | 6 | 1.51E+10 | 5.34E+09 | 6 |
| 18 | 2.65E+06 | 1341649 | 6 | 3.20E+10 | 2.22E+10 | 6 |
| 21 | 3.05E+06 | 1120027 | 6 | 7.10E+10 | 3.5E+10 | 6 |
| 24 | 2.13E+06 | 693763.3 | 6 | | | |
| 28 | 1.55E+06 | 708141.3 | 6 | | | |
| 31 | 1.40E+06 | 205870.5 | 6 | | | |
| 35 | 1.44E+06 | 351468.1 | 6 | | | |
| 38 | 1.45E+06 | 165858 | 6 | | | |

| | Construct | | |
|---|---|---|---|
| | Tumor only- HH | | |
| Days post T- | Average Total | SD Total | |
| cell injection | Flux (p/s) | Flux (p/s) | N |
| −2 | 1.64E+06 | 104258.5 | 5 |
| 4 | 2.54E+07 | 7571561 | 5 |
| 7 | 1.46E+08 | 1.23E+08 | 5 |
| 10 | 1.43E+09 | 5.22E+08 | 5 |
| 14 | 1.65E+10 | 5.73E+09 | 5 |
| 18 | 4.36E+10 | 1.64E+10 | 5 |
| 21 | 6.14E+10 | 4.04E+10 | 5 |
| 24 | | | |
| 28 | | | |
| 31 | | | |
| 35 | | | |
| 38 | | | |

### Example 5: In-Vivo Tumor Burden Assay #2

Female NOG mice were engrafted with 0.3×10⁶ HH-Luc2 tumor cells followed by the injection of 5×10⁶ (5 M) engineered T cells engineered using various constructs of CD30 CAR 4 days later to assess efficacy in tumor growth suppression in vivo. Groups of T cells studied include: a control group of T cells with tumor only; T cells with edits in TRAC only (TCR KO); T cells engineered with construct 3881; T cells engineered with construct 3882; T cells engineered with construct 3884; T cells engineered with construct 3885; and T cells engineered with construct 3887. For T cells engineered with construct 3881 and 3884, T cells were injected intravenously (IV) or intraperitoneally (IP); other T cell constructs (3882, 3885, 3887) were dosed IV only.

### 5.1. Preparation of T Cells

T cells were isolated from peripheral blood of a healthy human donor with the following MHC I phenotype: HLA-A*02:01:01G, 03:01:01G, HLA-B*07:02:01G, HLA-C*07:02:01G. Briefly, a leukapheresis pack (HemaCare Technologies) was treated in ammonium chloride RBC lysis buffer (Stemcell Technologies; Cat. 07800) for 15 minutes to lyse red blood cells. Peripheral blood mononuclear cell (PBMC) count was determined post lysis and T cell isolation was performed using EasySep Human T cell isolation kit (Stemcell Technologies, Cat. 17951) according to manufacturer's protocol. Isolated CD3+ T cells were resuspended in Cryostor CS10 media (Stemcell Technologies, Cat. 07930) and frozen down in liquid nitrogen until further use.

Frozen T cells were thawed at a cell concentration of 1.5×10⁶ cells/mL into T cell activation media (TCAM) composed of CTS Optimizer (Gibco A3705001), GlutaMAX (Gibco Cat. 35050061), 2.5% Human AB Serum (Gemini Cat. 100-512), HEPES (Gibco Cat. 15630080), Penicillin-Streptomycin (Gibco Cat. 15140122), 200 U/mL of recombinant human interleukin-2 (Peprotech, Cat. 200-02), 5 ng/mL IL-7 (Peprotech, Cat. 200-07), and 5 ng/mL IL-15 (Peprotech, Cat. 200-15). Cells were rested at 37° C. for 24 hours.

Twenty-four hours post thawing T cells were counted and resuspended at 1×10⁶ cells/mL in TCAM media and TransACT (Miltenyi Cat. 130-111-160) was added to a final concentration of 1/100 of the total volume. Cells were mixed and were incubated at 37° C. for 24 hours.

Forty-eight hours post activation all groups were transduced with EF1α-CAR AAV. AAV was removed from −80° C. and thawed on ice. Transduction media was generated from TCAM by adding ApoE3 (Peprotech, Cat. 350-02,) to a final concentration of 2.5 μg/mL, TRAC-targeted LNP to a final concentration of 2.5 μg/mL, and DNApki Compound 1 to a final concentration of 0.25 μM. Cells were collected, centrifuged at 500 mXG for 5 minutes and resuspended at 0.5e6 cells/mL in transduction media. Appropriate AAVs for CD30 constructs were added to cells at an MOI of 3e5 G/cell. Cells were mixed and were incubated at 37° C. After 24 hours, Cells were transferred to GREX plates (Wilson Wolf Cat. 80660M) and expanded for 7 days with regular changes of media and cytokines. After expansion, CAR insertion rates were quantified using flow cytometry, and cells were cryopreserved in Cryostor CS10 freezing media (StemCell Cat. 07930).

### 5.2. Construct 3884 and Construct 3887 CD30 CAR-T Cells Observed to Show Maximum HH-Luc2 Tumor Control

For the in vivo study, HH-Luc2 tumor cells were harvested from culture, washed twice with HBSS (Gibco, Cat. No. 14025-092) and resuspended at 0.3×10⁶ cells for injection in 150 μl HBSS. Eighty female NOG mice (Taconic) were dosed by tail vein injection with HH-Luc2 tumor cells. Four days later, T cells expressing CAR constructs 3881, 3882, 3884, 3885, or 3887 were thawed, washed with HBSS (Gibco, Cat. No. 14025-092) and resuspended at 5×10⁶ cells/mL for injection in 150 μL HBSS. Five HH-Luc2 engrafted mice per T cell group were dosed by either tail vein injection or intraperitoneal injection.

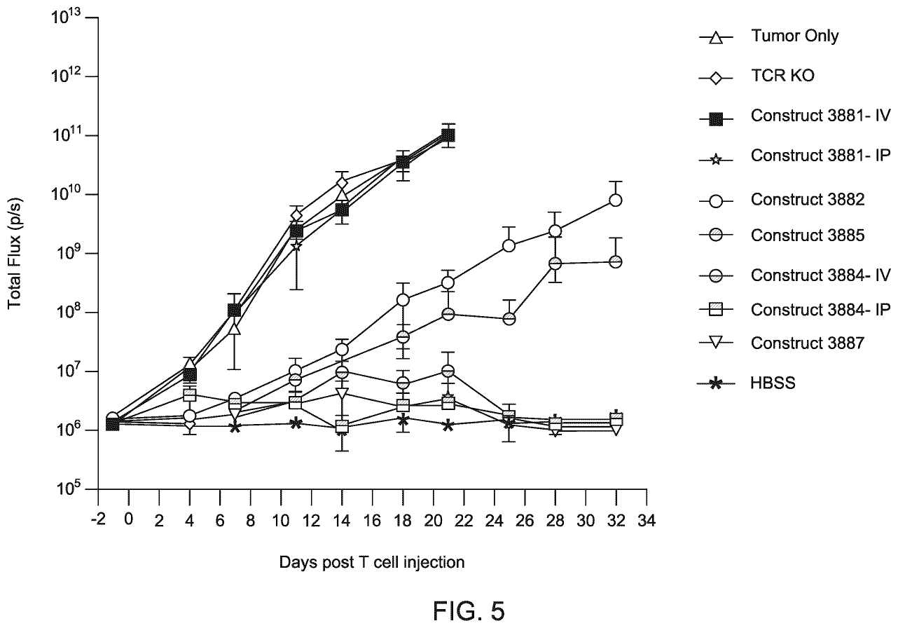

IVIS imaging of live mice was performed to identify luciferase-positive tumor cells by IVIS spectrum to study the tumor burden. IVIS imaging was done at day −1, 4, 7, 11, 14, 18, 21, 25, 28, and 32 post T cell injection. Mice were prepared for imaging with an injection of D-luciferin i.p. at 10 μL/g body weight per the manufacturer's recommendation, about 100 μL per animal. Animals were anesthetized and then placed in the IVIS imaging unit. The visualization was performed with the exposure time set to auto, field of view D, medium binning, and F/stop set to 1. Table 13 and FIG. 5 shows total flux (photons/second) from luciferase expressing tumor cells present at the various time points after injection. Tumor control is depicted graphically in FIG. 5, indicated intravenous or intraperitoneal delivery, as appropriate. Mice treated with constructs 3884 IV or IP and 3887 IV showed complete tumor regression. Mice treated with constructs 3882 and 3885 IV showed partial tumor control. These mice were sacrificed at day 32 to track T cells via flow cytometry. The majority of T cells were observed in blood from constructs 3882, 3884, and 3887. Higher T cell numbers were observed in constructs 3884 and 3887 compared to construct 3882. Solid tumor mass was observed in one mouse from construct 3882.

TABLE 13
Total Flux (photons/s) from luciferase expressing tumor cells
in treated mice at intervals after T cell injection.

| | Construct | | | | | |
|---|---|---|---|---|---|---|
| Days | Tumor only | | | TCR KO | | |
| post T- | Average | SD | | Average | SD | |
| cell | Total | Total | | Total | Total | |
| injection | Flux (p/s) | Flux (p/s) | N | Flux (p/s) | Flux (p/s) | N |
| −1 | 1.39E+06 | 6.66E+04 | 5 | 1.62E+06 | 3.26E+05 | 5 |
| 4 | 1.12E+07 | 2.28E+06 | 5 | 1.28E+07 | 2.89E+06 | 5 |
| 7 | 5.57E+07 | 5.25E+07 | 5 | 1.10E+08 | 8.81E+07 | 5 |
| 11 | 2.64E+09 | 9.03E+08 | 5 | 4.35E+09 | 1.76E+09 | 5 |
| 14 | 9.75E+09 | 3.57E+09 | 5 | 1.63E+10 | 6.38E+09 | 5 |
| 18 | 3.61E+10 | 1.05E+10 | 5 | 3.82E+10 | 1.85E+10 | 5 |
| 21 | | | | | | |
| 25 | | | | | | |
| 28 | | | | | | |
| 32 | | | | | | |
| | Construct | | | | | |
| Days | 3887 | | | 3881 | | |
| post T- | Average | SD | | Average | SD | |
| cell | Total | Total | | Total | Total | |
| injection | Flux (p/s) | Flux (p/s) | N | Flux (p/s) | Flux (p/s) | N |
| −1 | 1.48E+06 | 1.12E+05 | 5 | 1.27E+06 | 7.29E+04 | 5 |
| 4 | 1.55E+06 | 2.56E+05 | 5 | 9.06E+06 | 2.11E+06 | 5 |
| 7 | 1.77E+06 | 1.53E+05 | 5 | 1.04E+08 | 1.44E+07 | 5 |
| 11 | 3.00E+06 | 6.27E+05 | 5 | 2.40E+09 | 6.06E+08 | 5 |
| 14 | 4.21E+06 | 2.40E+06 | 5 | 5.22E+09 | 1.74E+09 | 5 |
| 18 | 2.64E+06 | 1.44E+06 | 5 | 3.38E+10 | 5.60E+09 | 5 |
| 21 | 3.52E+06 | 4.57E+06 | 5 | 1.04E+11 | 3.77E+10 | 4 |
| 25 | 1.32E+06 | 1.29E+05 | 5 | | | |
| 28 | 1.01E+06 | 9.16E+04 | 5 | | | |
| 32 | 1.05E+06 | 5.44E+04 | 5 | | | |
| | Construct | | | | | |
| Days | 3882 | | | 3884 | | |
| post T- | Average | SD | | Average | SD | |
| cell | Total | Total | | Total | Total | |
| injection | Flux (p/s) | Flux (p/s) | N | Flux (p/s) | Flux (p/s) | N |
| −1 | 1.81E+06 | 1.78E+05 | 5 | 1.41E+06 | 1.75E+05 | 5 |
| 4 | 1.77E+06 | 2.24E+05 | 5 | 1.33E+06 | 1.43E+05 | 5 |
| 7 | 3.55E+06 | 4.78E+05 | 5 | 2.10E+06 | 2.53E+05 | 5 |
| 11 | 1.05E+07 | 5.33E+06 | 5 | 3.19E+06 | 9.56E+05 | 5 |
| 14 | 2.49E+07 | 9.33E+06 | 5 | 9.87E+06 | 4.82E+06 | 5 |
| 18 | 1.69E+08 | 1.29E+08 | 5 | 6.53E+06 | 3.86E+06 | 5 |
| 21 | 3.27E+08 | 1.86E+08 | 5 | 1.05E+07 | 1.11E+07 | 5 |
| 25 | 1.35E+09 | 1.45E+09 | 5 | 1.64E+06 | 4.40E+05 | 5 |
| 28 | 2.48E+09 | 1.94E+09 | 5 | 1.15E+06 | 2.62E+05 | 5 |
| 32 | 8.14E+09 | 7.81E+09 | 5 | 1.25E+06 | 1.26E+05 | 5 |
| | Construct | | | | | |
| Days | 3885 | | | 3881- IP | | |
| post T- | Average | SD | | Average | SD | |
| cell | Total | Total | | Total | Total | |
| injection | Flux (p/s) | Flux (p/s) | N | Flux (p/s) | Flux (p/s) | N |
| −1 | 1.40E+06 | 7.62E+04 | 3 | 1.21E+06 | 9.31E+04 | 5 |
| 4 | 1.36E+06 | 9.10E+04 | 3 | 1.20E+07 | 4.82E+06 | 5 |
| 7 | 1.94E+06 | 2.17E+05 | 3 | 9.45E+07 | 4.10E+07 | 5 |
| 11 | 6.91E+06 | 2.61E+06 | 3 | 1.37E+09 | 1.00E+09 | 5 |
| 14 | 3.97E+05 | 4.10E+05 | 3 | 6.00E+09 | 2.47E+09 | 5 |
| 18 | 3.97E+07 | 1.82E+07 | 3 | 4.11E+10 | 8.96E+09 | 5 |
| 21 | 9.60E+07 | 1.01E+08 | 3 | 8.53E+10 | 1.18E+10 | 5 |
| 25 | 8.04E+07 | 6.93E+07 | 3 | | | |
| 28 | 6.92E+08 | 9.10E+08 | 3 | | | |
| 32 | 7.53E+08 | 9.09E+08 | 3 | | | |
| | Construct | | | | | |
| Days | 3884- IP | | | HBSS | | |
| post T- | Average | SD | | Average | SD | |
| cell | Total | Total | | Total | Total | |
| injection | Flux (p/s) | Flux (p/s) | N | Flux (p/s) | Flux (p/s) | N |
| −1 | 1.50E+06 | 3.77E+05 | 4 | 1.24E+06 | 7.87E+04 | 5 |
| 4 | 3.95E+06 | 1.53E+06 | 4 | 1.20E+06 | 1.19E+05 | 5 |
| 7 | 2.94E+06 | 9.73E+05 | 4 | 1.25E+06 | 2.06E+05 | 5 |
| 11 | 2.69E+06 | 3.66E+05 | 4 | 1.36E+06 | 5.90E+04 | 5 |
| 14 | 1.16E+06 | 6.06E+05 | 4 | 1.16E+06 | 1.23E+05 | 5 |
| 18 | 2.65E+06 | 2.72E+06 | 4 | 1.69E+06 | 6.98E+04 | 5 |
| 21 | 2.89E+06 | 2.84E+06 | 4 | 1.27E+06 | 4.73E+04 | 5 |
| 25 | 1.77E+06 | 9.25E+05 | 4 | 1.57E+06 | 1.14E+05 | 5 |
| 28 | 1.28E+06 | 1.73E+05 | 4 | 1.50E+06 | 8.48E+04 | 5 |
| 32 | 1.52E+06 | 1.01E+05 | 4 | 1.65E+06 | 1.53E+05 | 5 |

### Example 6: In-Vivo Tumor Burden Assay of Constructs 3884 and 3887

Female NOG mice were engrafted with 0.3×10⁶ HH-Luc2 tumor cells followed by the injection of 5×10⁶ (5 M), or 0.5×10⁶ (0.5 M) engineered T cells engineered using two different constructs of CD30 CAR 4 days later in order to assess efficacy of tumor growth suppression in vivo. Groups of T cells studied included: a control group of T cells with tumor only; T cells engineered with construct 3884 (0.5 M or 5 M); and T cells engineered with construct 3887 (0.5 M or 5 M). T cells were prepared as in Example 5.1.

### 6.1. CD30 CAR-T Cells Expressing Constructs 3884 or 3887 Demonstrate Significant Tumor Control in HH-Luc2 Model

For the in vivo study, HH-Luc2 tumor cells were harvested from culture, washed twice with HBSS (Gibco, Cat. No. 14025-092) and resuspended at 0.3×10⁶ cells for injection in 150 μl HBSS. Fifty female NOG mice (Taconic) were dosed by tail vein injection with HH-Luc2 tumor cells. Four days later, engineered T cells expressing either construct 3884 or construct 3887 were thawed, washed with HBSS (Gibco, Cat. No. 14025-092) and resuspended at 5×10⁶, or 0.5×10⁶ for injection in 150 μL HBSS. Five HH-Luc2 engrafted mice per T cell group were dosed by tail vein injection.

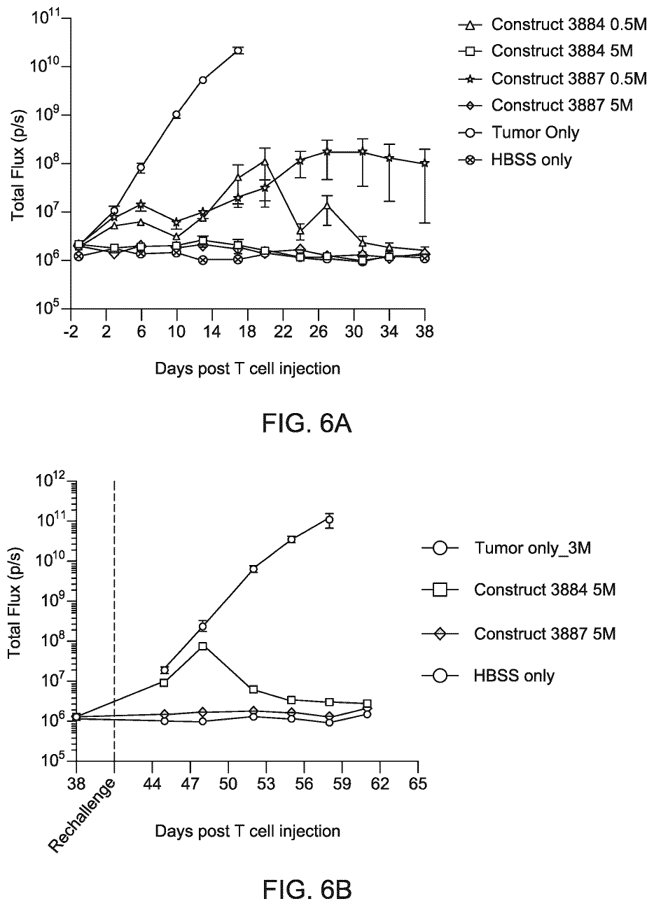

IVIS imaging of live mice was performed to identify luciferase-positive tumor cells by IVIS spectrum to study the tumor burden. IVIS imaging was done at day −1, 3, 6, 10, 13, 17, 20, 24, 27, 31, 34, 38, 41, 45, 48, 52, 55, 58 and 61 post T cell injection. Mice were prepared for imaging with an injection of D-luciferin i.p. at 10 μL/g body weight per the manufacturer's recommendation, about 100 μL per animal. Animals were anesthetized and then placed in the IVIS imaging unit. The visualization was performed with the exposure time set to auto, field of view D, medium binning, and F/stop set to 1. Tables 14 and 15 and FIGS. 6A and 6B show total flux (photons/second) from luciferase expressing tumor cells present at the various time points after injection. The tumor only group was euthanized by Day 17.

TABLE 14
Total Flux (photons/s) from luciferase expressing tumor
cells in treated mice at intervals after T cell injection

| | Construct | | | | | |
|---|---|---|---|---|---|---|
| Days post | 3884 0.5M | | 3884 5M | | Tumor only | |
| T-cell | Average | St Dev | Average | St Dev | Average | St Dev |
| injection | Flux (p/s) | Flux (p/s) | Flux (p/s) | Flux (p/s) | Flux (p/s) | Flux (p/s) |
| −1 | 1965600 | 408812.72 | 2201000 | 306583.7569 | 2071000 | 497165.566 |
| 3 | 5292600 | 1190928.646 | 1847000 | 143355.5022 | 10802600 | 5075041.304 |
| 6 | 6303000 | 2043622.959 | 1943200 | 377115.0488 | 84100000 | 39953838.86 |
| 10 | 3153600 | 789224.3281 | 2043400 | 334724.1252 | 1042000000 | 336672440.2 |
| 13 | 7750600 | 2174711.438 | 2566400 | 1366093.35 | 5304000000 | 1386174159 |
| 17 | 53523000 | 81305552.24 | 2078200 | 1366870.206 | 21832000000 | 7456367480 |
| 20 | 111229800 | 196232715.8 | 1626600 | 221317.5095 | | |
| 24 | 4196800 | 3085019.378 | 1176800 | 113642.2457 | | |
| 27 | 505651800 | 983758261.4 | 1234800 | 53823.41498 | | |
| 31 | 2332000 | 1424420.408 | 999160 | 83676.27143 | | |
| 34 | 1929000 | 708961.5645 | 1194200 | 72424.85761 | | |
| 38 | 1626250 | 505508.3456 | 1289200 | 102583.4295 | | |
| 41 | 1298750 | 266219.2471 | 1268400 | 68768.01582 | | |
| 45 | | | 9397800 | 4784396.739 | 20056000 | 3675990.207 |
| 48 | | | 77902200 | 44619508.15 | 239440000 | 52782027.24 |
| 52 | | | 6143800 | 4340960.097 | 6813600000 | 1060161044 |
| 55 | | | 3415600 | 1899893.008 | 36600000000 | 6283279399 |
| 58 | | | 2974960 | 1413111.293 | 1.15436E+11 | 37359073115 |
| 61 | | | 2772400 | 862029.8371 | | |

| | Construct | | | | | |
|---|---|---|---|---|---|---|
| Days post | 3887 0.5M | | 3887 5M | | HBSS | |
| T-cell | Average | St Dev | Average | St Dev | Average | St Dev |
| injection | Flux (p/s) | Flux (p/s) | Flux (p/s) | Flux (p/s) | Flux (p/s) | Flux (p/s) |
| −1 | 2079600 | 235496.5817 | 1939200 | 247453.7533 | 1249000 | 139124.4048 |
| 3 | 7741800 | 1064817.43 | 1442000 | 94269.8255 | 1708800 | 135456.8566 |
| 6 | 14072800 | 7076252.635 | 1972800 | 410068.9698 | 1386800 | 109023.6672 |
| 10 | 6075400 | 3089205.827 | 1863200 | 481268.9061 | 1481000 | 207852.8326 |
| 13 | 9842400 | 5131570.777 | 2124200 | 832494.6606 | 1042200 | 82001.73169 |
| 17 | 20081800 | 10336742.67 | 1762600 | 573436.5178 | 1057000 | 82999.39759 |
| 20 | 32342200 | 30072529.51 | 1494400 | 232016.0339 | 1428000 | 133012.7813 |
| 24 | 117110200 | 128754464.9 | 1690800 | 166926.8103 | 1180800 | 86469.41656 |
| 27 | 179597400 | 264186833.4 | 1215600 | 60641.89971 | 1091040 | 102023.5581 |
| 31 | 180313600 | 291492656.8 | 1294600 | 219501.7995 | 973020 | 57740.46761 |
| 34 | 133945000 | 233577486 | 1156800 | 85854.29518 | 1247000 | 88620.53938 |
| 38 | 103948000 | 195746106.3 | 1352400 | 41610.57558 | 1121200 | 60865.09673 |
| 41 | 24629000 | 39059103.48 | 1080840 | 63542.91778 | 1374400 | 145732.0829 |
| 45 | | | 1463400 | 213146.5224 | 1042250 | 24973.7362 |
| 48 | | | 1714800 | 165583.0909 | 1014125 | 47085.52724 |
| 52 | | | 1766000 | 58449.97861 | 1298250 | 59048.17948 |
| 55 | | | 1632200 | 195037.8425 | 1169000 | 102379.1971 |
| 58 | | | 1332800 | 88833.32708 | 958750 | 94461.381 |
| 61 | | | 2197800 | 179426.1965 | 1535000 | 80333.67911 |

At 5×10⁶ cell dose, both construct 3884 and construct 3887, showed complete tumor control. But at 0.5×10⁶ cell dose, partial HH-Luc2 tumor control was observed with construct 3887 and complete tumor control was observed with construct 3884. 3882 showed similar tumor control as “tumor only” control at 0.5×10⁶ cell dose, and 3885 did not demonstrate appreciable tumor control at 0.5×10⁶ cell dose (data not shown).

At day 38, the 5×10⁶ cell dose groups were bled to study T cell counts. T cells were not detected in mice treated with construct 3884; 100 cells/μL CD4+ T cells were detected with an outlier of 800 cells/μL in mice treated with construct 3887. Fewer than 75 cells/μL CD8+ T cells were detected in the mice treated with construct 3887. At day 41, the 5×10⁶ cell dose groups were rechallenged with 3×10⁶ HH-luc2 tumor cells. The rechallenge study observations are depicted in FIG. 6B. All mice were euthanized on Day 61.

TABLE 15
Total Flux (photons/s) from luciferase expressing tumor cells in treated
mice at intervals after T cell injection following re-challenge

| | Construct | | | | | |
|---|---|---|---|---|---|---|
| Days | 3884 5M | | | 3887 5M | | |
| post T- | Average | St Dev | | Average | St Dev | |
| cell | Flux | Flux | | Flux | Flux | |
| injection | (p/s) | (p/s) | N | (p/s) | (p/s) | N |
| 38 | 1290000.0 | 114673.4 | 5 | 1352000.0 | 46583.3 | 5 |
| 45 | 9402000.0 | 5350889.6 | 5 | 1464000.0 | 237865.5 | 5 |
| 48 | 77906000.0 | 49888798.1 | 5 | 1716000.0 | 186895.7 | 5 |
| 52 | 6152000.0 | 4868970.1 | 5 | 1766000.0 | 63482.3 | 5 |
| 55 | 3416000.0 | 2124354.0 | 5 | 1634000.0 | 219271.5 | 5 |
| 58 | 2975200.0 | 1580030.1 | 5 | 1332000.0 | 101833.2 | 5 |
| 61 | 2772000.0 | 962792.8 | 5 | 2198000.0 | 199925.0 | 5 |

| | Construct | | | | | |
|---|---|---|---|---|---|---|
| Days | HBSS Only | | | Tumor Only 3M | | |
| post T- | Average | St Dev | | Average | St Dev | |
| cell | Flux | Flux | | Flux | Flux | |
| injection | (p/s) | (p/s) | N | (p/s) | (p/s) | N |
| 38 | 1120000.0 | 67823.3 | 5 | | | |
| 45 | 1042500.0 | 29860.8 | 4 | 20080000 | 4103900.6 | 5 |
| 48 | 1014250.0 | 55667.9 | 4 | 2.4E+08 | 59061832 | 5 |
| 52 | 1297500.0 | 69940.5 | 4 | 6.81E+09 | 1.186E+09 | 5 |
| 55 | 1167500.0 | 118708.3 | 4 | 3.66E+10 | 7.005E+09 | 5 |
| 58 | 958250.0 | 108555.3 | 4 | 1.15E+11 | 4.195E+10 | 5 |
| 61 | 1537500.0 | 93229.1 | 4 | | | |

### Example 7: Rechallenging Construct 3884 Binder Variants with HH Tumor Cells

### Example 7.1: Thawing & Resting CAR-T and Control T-Cells

Cryopreserved T cells expressing anti-CD30 CAR constructs that vary in the HRS3 binder domain were thawed as in Example 3.1. T cells expressing the following anti-CD30 CAR constructs were tested: 3884, 4620, 4621, 4622, and T cells with edits in TRAC only. T cells were prepared as in Example 5.1.

### Example 7.2: Assay Setup with Effector and Target Cells

HH GFP-Luciferase cells were counted using a Vi-cell and desired number of cells were collected in a 15 mL conical tube. Tumor cells were then washed by centrifuging at 500XG for 5 mins followed by resuspending in pre-warmed TCGM media at 1×10⁶ cells/mL. Using a sterile collage coated 24 well plate (Corning, Cat. 354408), 100 μL of tumor cells (resuspended at either 8×10⁶ cells/mL or 4×10⁶ cells/mL) per well were added. All CAR-T cells and control T cells were removed from the incubator and counted using vi-cell and washed by centrifugation as described in previous steps. A desired number of T-cells were resuspended in TCGM media at 1×10⁶ cells/mL. 100 μL of T cells were added to respective wells containing HH tumor cells such that the starting effector to target cell ratio is 1:4. 800 μL of TCGM media was added to bring up the total volume to 1 mL. The plate was transferred to a live cell analysis instrument (Incucyte S3, Sartorius) at 37° C.; the images were captured every 4-6 hours via Incucyte 2019B Rev2 software using Phase and Green channels with 10× objective.

### Example 7.3: Rechallenge with Tumor Cells

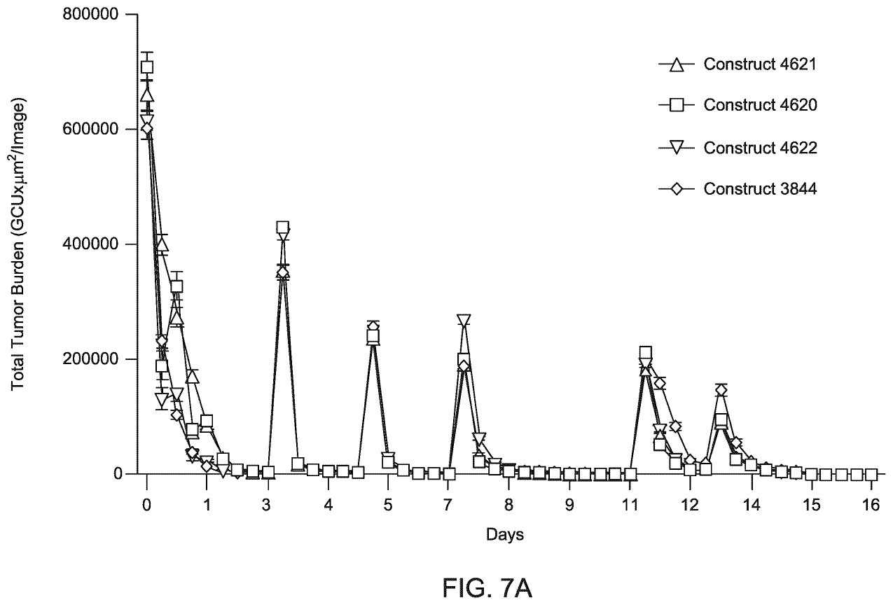

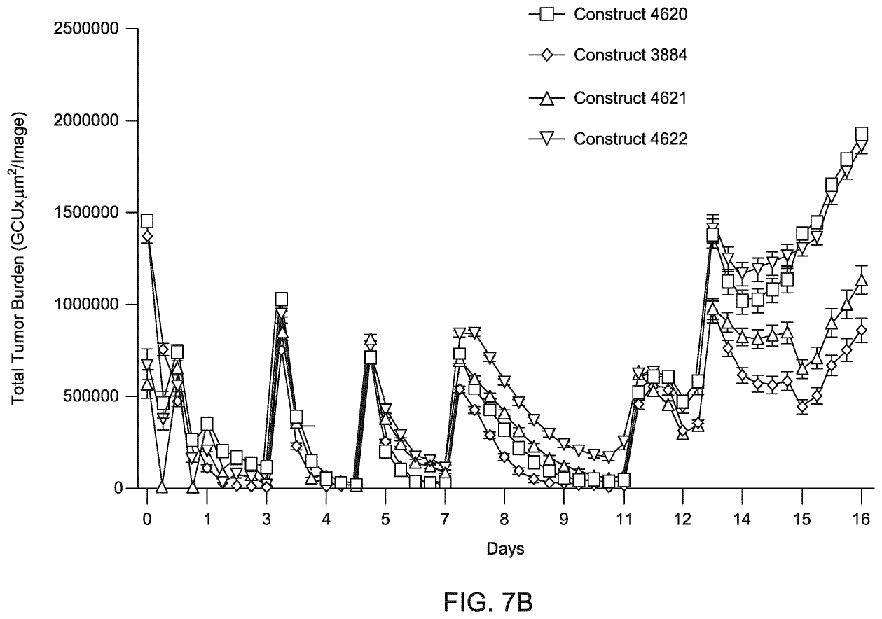

CAR-T Cells were rechallenged with HH cells on Day 2, 4, 7, 11, 13. CAR-T cells were rechallenged with either 400,000 cells/well or 800,000 cells/well of HH tumor cells on all the rechallenge days by first removing 500 μL of media and replacing with pre-warmed TCGM media containing HH cells. After adding HH cells, plate was returned to the Incucyte for further analysis. Results are illustrated in FIGS. 7A and 7B.

### Example 8: In-vivo Tumor Killing with Construct 3884 Binder Variants

Female NOG mice were engrafted with 0.3×10⁶ HH-Luc2 tumor cells and followed 5 days later by administering via injection 5×10⁶ engineered T cells comprising different constructs of CD30 CAR in order to assess efficacy of tumor growth suppression in vivo. The engineered T cells comprised construct 3884, 4620, 4621, or 4622. T cells were prepared as in Example 5.1. As a positive control, a group of mice were engrafted with tumor cells but were not administered engineered T cells. As a negative control, a group of mice were administered vehicle control.

For the in vivo study, HH-Luc2 tumor cells were harvested from culture, washed twice with HBSS (Gibco, Cat. No. 14025-092) and resuspended at 0.3×10⁶ cells for injection in 150 μl HBSS. Thirty-five female NOG mice (Taconic) were dosed by tail vein injection with HH-Luc2 tumor cells. Five days later, engineered T cells expressing CAR encoded by constructs 3884, 4620, 4621, or 4622 were thawed, washed with HBSS (Gibco, Cat. No. 14025-092), and resuspended at 5×10⁶ for injection in 150 μL HBSS. Five HH-Luc2 engrafted mice per T cell group were dosed by tail vein injection.

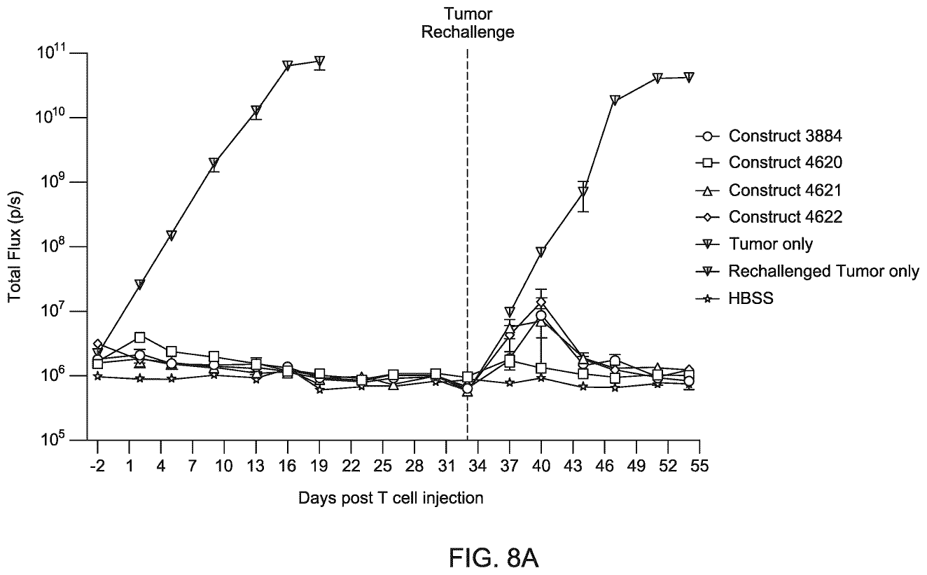

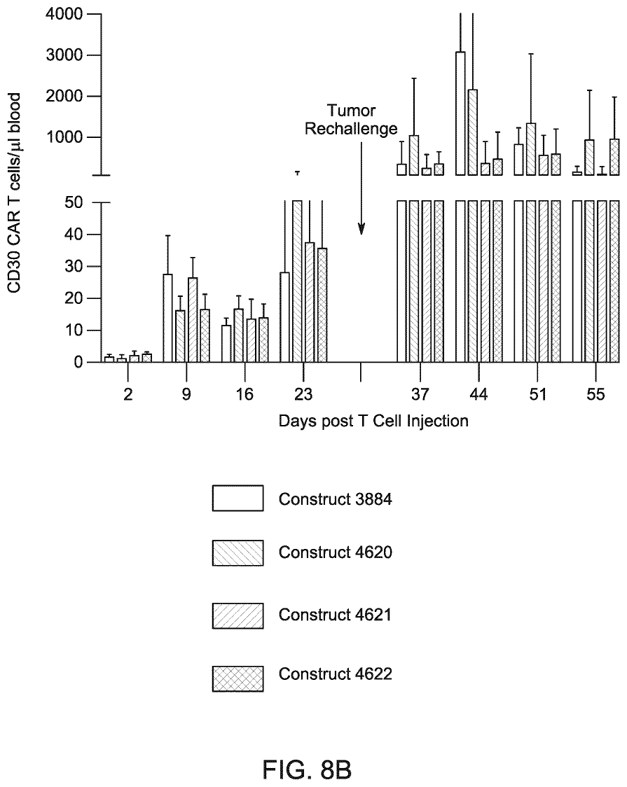

IVIS imaging of live mice was performed to identify luciferase-positive tumor cells by IVIS spectrum to study the tumor burden. IVIS imaging was done at day −2, 2, 5, 9, 13, 16, 19, 23, 26, 30, 33, 37, 40, 44, 47, 51, and 54 post T cell injection. Mice were prepared for imaging with an injection of D-luciferin i.p. at 10 μL/g body weight per the manufacturer's recommendation, about 150 μL per animal. Animals were anesthetized and then placed in the IVIS imaging unit. The visualization was performed with the exposure time set to auto, field of view D, medium binning, and F/stop set to 1. Table 16 and FIG. 8A show total flux (photons/second) from luciferase expressing tumor cells present at the various time points after injection. The tumor only group was euthanized by Day 19. At a 5×10⁶ cell dose, all constructs showed robust tumor control.

On day 2, 9, 16, 23, 30, 37, 44 and 51, all groups were bled to study T cell counts. Highest CD30 CAR-T cell proliferation was observed on days 37 and 44 via ddPCR, and it was highest in mice treated with constructs 3884 and 4620 as in Table 16 and FIG. 8B.

TABLE 16
CD30 CAR-T cells/μl blood from T cell expansion for construct 3884 binder variants

| | Day 2 | | | Day 9 | | | Day 16 | | | Day 37 | | |
|---|---|---|---|---|---|---|---|---|---|---|---|---|
| Construct | Mean | SD | N | Mean | SD | N | Mean | SD | N | Mean | SD | N |
| 3884 | 1.69 | 0.60 | 3 | 27.28 | 11.97 | 3 | 11.42 | 2.22 | 3 | 363.75 | 539.27 | 3 |
| 4620 | 1.15 | 1.16 | 3 | 15.97 | 4.62 | 3 | 16.55 | 3.95 | 3 | 1060.69 | 1382.24 | 3 |
| 4621 | 2.07 | 1.39 | 3 | 26.13 | 6.47 | 3 | 13.48 | 6.12 | 3 | 263.60 | 338.21 | 3 |
| 4622 | 2.52 | 0.58 | 2 | 16.30 | 4.86 | 3 | 13.70 | 4.46 | 3 | 352.22 | 317.79 | 3 |

| Construct | Day 37 | | |
|---|---|---|---|
| 3884 | Mean | SD | N |
| 4620 | 363.75 | 539.27 | 3 |
| 4621 | 1060.69 | 1382.24 | 3 |
| 4622 | 263.60 | 338.21 | 3 |

| | 352.22 | | | 317.79 | | | 3 | | |
|---|---|---|---|---|---|---|---|---|---|
| 4622 | Mean | SD | N | Mean | SD | N | Mean | SD | N |
| 3884 | 3073.43 | 2833.00 | 3 | 831.97 | 415.59 | 2 | 166.33 | 134.85 | 3 |
| 4620 | 2161.96 | 3297.18 | 3 | 1338.91 | 1681.41 | 3 | 955.19 | 1195.44 | 4 |
| 4621 | 375.00 | 528.06 | 3 | 584.10 | 477.95 | 3 | 119.52 | 165.09 | 5 |
| 4622 | 483.31 | 654.60 | 3 | 605.77 | 592.78 | 3 | 966.84 | 1009.10 | 5 |

At day 34, all the cell groups were rechallenged with 3×10⁶ HH-luc2 tumor cells. The rechallenge study observations are depicted in FIG. 8A following day 34. All mice were euthanized on Day 55. An initial tumor burden increase was observed with constructs 3884, 4621, and 4622 but after day 43, all constructs showed robust tumor control.

TABLE 17
Total Flux (photons/s) from luciferase expressing tumor cells
in treated mice at intervals after T cell injection.

| | Construct | | | | | |
|---|---|---|---|---|---|---|
| Days | 3884 | | 4620 | | Tumor only | |
| post T- | Average | St Dev | Average | St Dev | Average | St Dev |
| cell | Flux | Flux | Flux | Flux | Flux | Flux |
| injection | (p/s) | (p/s) | (p/s) | (p/s) | (p/s) | (p/s) |
| −2 | 1820000 | 118321.6 | 1616000 | 496014.1 | 2234000 | 417648.2 |
| 2 | 2138800 | 1093973 | 4012000 | 1730367 | 25540000 | 6809772 |
| 5 | 1566000 | 689623.1 | 2430000 | 227925.4 | 1.48E+08 | 34780742 |
| 9 | 1472000 | 265838.3 | 2020000 | 637024.3 | 1.92E+09 | 9.46E+08 |
| 13 | 1494600 | 994761.2 | 1543400 | 876283.5 | 1.25E+10 | 6.14E+09 |
| 16 | 1368000 | 376855.4 | 1242000 | 170792.3 | 6.25E+10 | 1.69E+10 |
| 19 | 915200 | 86799.77 | 1082800 | 211861.3 | 7.26E+10 | 4.27E+10 |
| 23 | 859200 | 78170.97 | 879200 | 109989.5 | | |
| 26 | 1013800 | 100200.8 | 1064200 | 137325.2 | | |
| 30 | 991000 | 41237.12 | 1082200 | 122258.7 | | |
| 33 Rechallenge | 671200 | 148622.7 | 992800 | 73683.11 | | |
| 37 | 1876000 | 1334440 | 1788000 | 765780.6 | 9822000 | 3731744.9 |
| 40 | 8766750 | 15092458 | 1348000 | 351098.3 | 83020000 | 23967311 |
| 44 | 1548500 | 1009836 | 1094600 | 205257.9 | 6.99E+08 | 768554961 |
| 47 | 1793000 | 734749.6 | 978200 | 161394.5 | 1.83E+10 | 7.663E+09 |
| 51 | 968666.7 | 419268.8 | 1060400 | 175249.3 | 4.02E+10 | 1.492E+10 |
| 54 | 867000 | 404656.6 | 1046000 | 167103.3 | 4.14E+10 | 2.157E+10 |

| | Construct | | | | | |
|---|---|---|---|---|---|---|
| Days | 4621 | | 4622 | | HBSS | |
| post T- | Average | St Dev | Average | St Dev | Average | St Dev |
| cell | Flux | Flux | Flux | Flux | Flux | Flux |
| injection | (p/s) | (p/s) | (p/s) | (p/s) | (p/s) | (p/s) |
| −2 | 1640000 | 146287.4 | 3160000 | 510294.03 | 1032400 | 115214.1 |
| 2 | 1822600 | 800389.8 | 1804000 | 567785.17 | 954400 | 106048.6 |
| 5 | 1578000 | 267712.5 | 1646000 | 472895.34 | 955800 | 148034.5 |
| 9 | 1394000 | 74699.4 | 1372600 | 344744.54 | 1093600 | 131357.5 |
| 13 | 1100800 | 313140.1 | 1338000 | 248837.3 | 909200 | 53298.22 |
| 16 | 1157600 | 204545.3 | 1209400 | 233606.08 | 1306000 | 74363.97 |
| 19 | 909400 | 86410.65 | 1004400 | 75619.442 | 642400 | 87245.63 |
| 23 | 983200 | 152740.6 | 862800 | 96086.94 | 720000 | 80128.02 |
| 26 | 763800 | 81346.17 | 888800 | 86038.945 | 739400 | 101908.3 |
| 30 | 996200 | 65385.01 | 977000 | 107480.23 | 847200 | 98013.77 |
| 33 Rechallenge | 604800 | 54568.31 | 622600 | 50142.796 | 888400 | 64852.14 |
| 37 | 5728000 | 4281865 | 4322000 | 4290357.8 | 803800 | 81444.46 |
| 40 | 7493800 | 7951062 | 14260000 | 17955134 | 973800 | 32506.92 |
| 44 | 1960000 | 702566.7 | 1824000 | 1077325.4 | 708200 | 86068 |
| 47 | 1343400 | 603835.9 | 1235400 | 649615.12 | 686000 | 73528.91 |
| 51 | 1358000 | 327673.6 | 957200 | 355989.04 | 796600 | 53252.23 |
| 54 | 1283200 | 270427.8 | 1264000 | 279338.5 | 787600 | 113326.1 |

### Example 9: Anti-CD30 CAR-T Cell Tumor Killing Assay with Construct 3884 Binder Variants Against HH Tumor Cells

### Example 9.1: Killing Assay Readout

T cells were engineered with anti-CD30 CAR constructs 3884, 4620, 4621, and 4622 as described in Example 4.1 and were tested for their cytotoxicity against HH tumor cells. T cell growth media was prepared as in Example 2.1 and cells were thawed and rested as in Example 2.2. The killing assay was set up with CAR-T and tumor cells as described in Example 2.3.

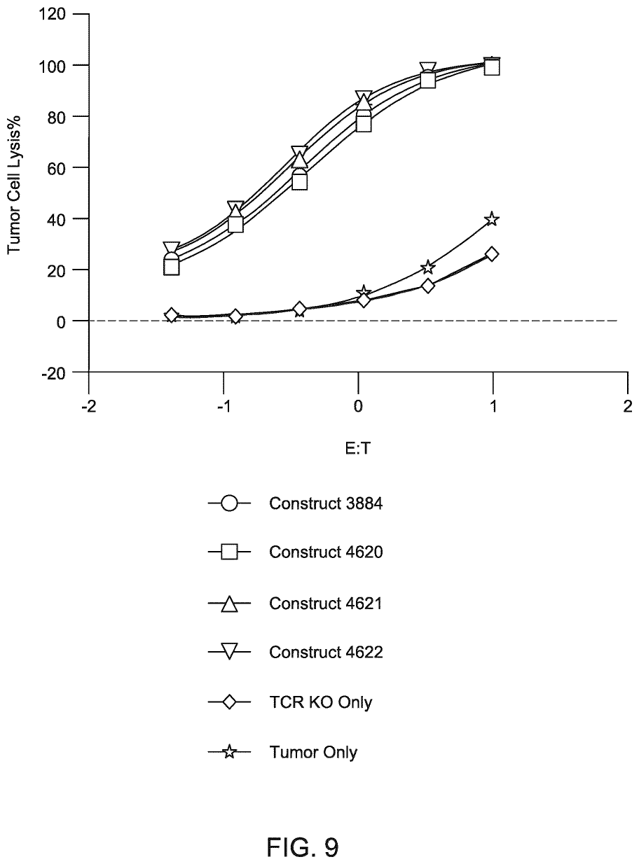

Bright-Glo™ Luciferase Assay System (Promega, Cat. E2620) was pre-thawed in dark at room temperature. The killing assay plate was taken out from the incubator, 50 μL of the supernatant in each well was carefully collected without disturbing the cells at the bottom of the well. 50 μL of Bright-Glo™ Luciferase Assay System was added to each well and the plate was shaken briefly on a shaker and then incubated in dark at room temperature for 5 minutes. The plate was then read for luminescence with a CLARIOstar plate reader. The percentage killing was calculated from the luminescence with the average of T cell to tumor cell ratio 0 as 0% killing. The results are shown in Table 18 and FIG. 9.

TABLE 18
Mean HH Tumor Cell Killing

| | Construct | | | | | | | | | | | |
|---|---|---|---|---|---|---|---|---|---|---|---|---|
| | 3884 | | 4620 | | 4621 | | 4622 | | TCR KO Only | | Tumor Only | |
| E:T | Mean | SD | Mean | SD | Mean | SD | Mean | SD | Mean | SD | Mean | SD |
| 10.00 | 99.44 | 0.31 | 99.07 | 0.42 | 99.71 | 0.11 | 99.70 | 0.11 | 26.28 | 0.72 | 39.54 | 0.18 |
| 3.33 | 95.18 | 0.64 | 93.84 | 0.62 | 97.48 | 0.33 | 98.19 | 0.40 | 13.96 | 1.38 | 21.00 | 0.87 |
| 1.11 | 80.39 | 3.81 | 76.82 | 2.00 | 84.95 | 0.51 | 87.19 | 1.72 | 8.11 | 1.42 | 10.93 | 1.85 |
| 0.37 | 57.22 | 3.42 | 54.22 | 0.78 | 62.97 | 1.46 | 65.37 | 0.60 | 4.74 | 0.70 | 3.84 | 0.29 |
| 0.12 | 39.15 | 0.93 | 37.41 | 0.99 | 42.63 | 0.33 | 43.82 | 1.53 | 1.96 | 2.02 | 1.44 | 0.30 |
| 0.04 | 23.92 | 1.14 | 21.01 | 2.10 | 26.25 | 3.69 | 27.16 | 2.92 | 1.92 | 0.78 | 1.94 | 0.48 |
| 0.00 | 99.44 | 0.31 | 99.07 | 0.42 | 99.71 | 0.11 | 99.70 | 0.11 | 26.28 | 0.72 | 39.54 | 0.18 |

| | | | | | | |
|---|---|---|---|---|---|---|
| EC50 | 0.3895 | 0.4112 | 0.3050 | 0.2814 | ~286234 | 12.95 |

### Example 9.2: Engineered T Cell Cytokine Release

Engineered T cells prepared as described in Example 4.1 were co-cultured with HH tumor cells as described above in sections 2.2-2.4. Supernatants from the co-culture were collected as described in section 2.4 and assayed for the cytokine release profiles of each engineered T cell group as described below:

For each of the cytokines measured, biotinylated capture antibody from the U-PLEX Immuno-Oncology Group 1 (hu) Assays (MSD, Cat No. K151AEL-2) was added to the assigned linker according to the kit's protocol. The antibody-linker mixtures were vortexed and incubated at room temperature for 30 minutes, after which the free antibodies were quenched with the Stop Solution from the kit. The antibody-linker solutions for each cytokine to be measured were combined to form the coating mix according to the kit instructions. Post incubation, the plate was washed, sealed, and stored overnight.

The following day, calibrators containing standards for each of the cytokines (IL-2 and IFN-7) to be assayed were reconstituted as per the manufacturer's instructions and diluted to create a 4-fold standard curve. 50 μL of each standard was loaded in duplicates on the MSD plate. The supernatant from each co-culture sample was thawed on ice and was diluted 1:10 in Diluent 2 from the U-PLEX Immuno-Oncology Group 1 (hu) Assays kit (MSD, Cat No. K151AEL-2). 50 μL of diluted samples from each group were loaded onto the meso scale discovery (MSD) plate and incubated for 1 hour.

The plates were washed 3 times with 150 μL of wash buffer as per the kit instructions, and 50 μL of the detection antibody solution (dilution prepared according to kit instructions) was added to each well of the MSD plate. The plate was incubated for 1 hour.

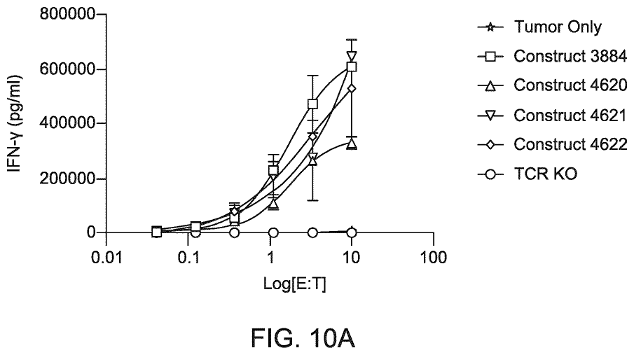

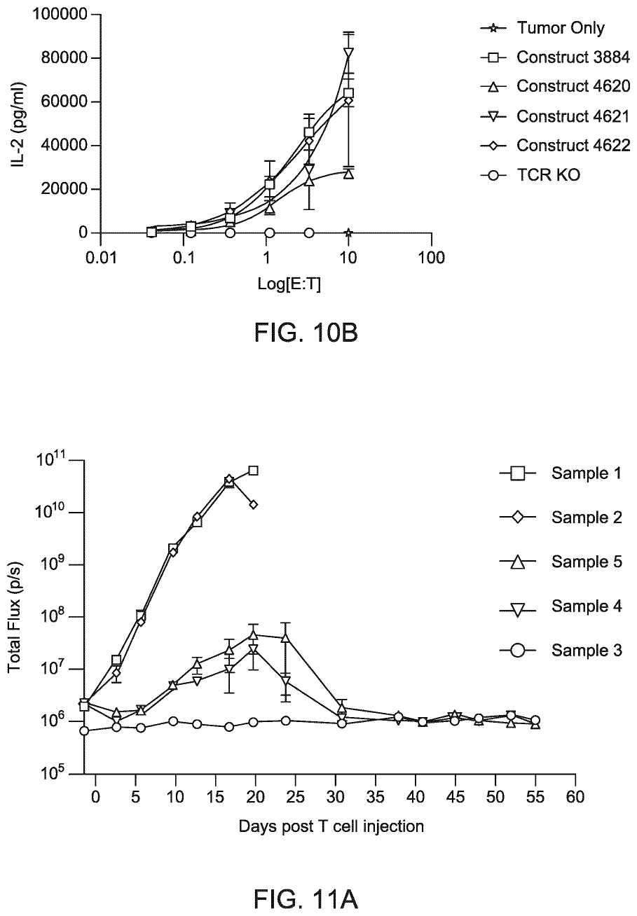

After incubation, the plate was washed 3 times with 150 μL and read immediately on the MSD instrument. Cytokine release is shown in Tables 19-20 and FIGS. 10A-10B.

TABLE 19
Concentration of IFN- γ released following T cell treatment
IFN-γ

| | Construct | | | | | | | | | | | |
|---|---|---|---|---|---|---|---|---|---|---|---|---|
| | 3884 | | 4620 | | 4621 | | 4622 | | TCR KO | | Tumor Only | |
| Log [peptide | Avg | Stdev | Avg | Stdev | Avg | Stdev | Avg | Stdev | Avg | Stdev | Avg | Stdev |
| (nM)] | IFN-γ | IFN-γ | IFN-γ | IFN-γ | IFN-γ | IFN-γ | IFN-γ | IFN-γ | IFN-γ | IFN-γ | IFN-γ | IFN-γ |
| 10.00 | 607662.12 | 3298.90 | 327999.17 | 21370.76 | 645765.48 | 6074.94 | 528808.91 | 178008.02 | 1110.87 | 1.41 | 6956.95 | 3209.48 |
| 3.33 | 471501.79 | 105519.57 | 265964.51 | 147001.55 | 275680.87 | 3270.42 | 353582.04 | 106629.04 | 163.92 | 71.11 | 1249.59 | 711.65 |
| 1.11 | 228976.46 | 3950.76 | 106391.05 | 22859.06 | 200629.08 | 61953.03 | 207223.88 | 79523.37 | 44.37 | 12.62 | 235.09 | 4.76 |
| 0.37 | 58465.40 | 3226.83 | 42632.99 | 3337.03 | 72142.40 | 28957.04 | 75075.34 | 33833.64 | 25.82 | 25.38 | 114.23 | 84.54 |
| 0.12 | 19662.71 | 2525.74 | 10531.78 | 6433.36 | 24800.01 | 12707.89 | 22155.77 | 10967.87 | 3.61 | 5.09 | 33.61 | 8.47 |
| 0.04 | 3723.62 | 172.31 | 2113.98 | 343.50 | 6839.23 | 678.00 | 6157.42 | 674.71 | 5.12 | 4.53 | 4.77 | 0.00 |

TABLE 20
Concentration of IL-2 release following T cell treatment
IL-2

| | Construct | | | | | | | | | | | |
|---|---|---|---|---|---|---|---|---|---|---|---|---|
| | 3884 | | 4620 | | 4621 | | 4622 | | TCR KO | | Tumor Only | |
| Log[peptide | Avg | Stdev | Avg | Stdev | Avg | Stdev | Avg | Stdev | Avg | Stdev | Avg | Stdev |
| (nM)] | IL-2 | IL-2 | IL-2 | IL-2 | IL-2 | IL-2 | IL-2 | IL-2 | IL-2 | IL-2 | IL-2 | IL-2 |
| 10.00 | 63773.88 | 6417.85 | 27289.54 | 2120.52 | 82158.26 | 9433.68 | 60392.52 | 30176.61 | | | 9.16 | 5.85 |
| 3.33 | 45835.73 | 8347.01 | 24463.37 | 13522.20 | 28898.12 | 861.03 | 41883.17 | 10412.98 | 1.09 | 0.00 | 4.87 | 4.68 |
| 1.11 | 22231.42 | 1716.81 | 11130.46 | 2710.29 | 21000.20 | 4799.57 | 23770.27 | 9116.22 | 5.93 | 0.00 | 4.56 | 1.61 |
| 0.37 | 6903.67 | 867.93 | 4958.13 | 526.59 | 8102.88 | 3131.05 | 9640.91 | 4163.08 | 0.96 | 0.85 | 2.94 | 2.62 |
| 0.12 | 2187.48 | 361.76 | 1373.58 | 740.47 | 2939.57 | 1536.43 | 3238.39 | 1524.69 | 6.61 | 0.00 | 2.15 | 0.16 |
| 0.04 | 478.36 | 65.32 | 334.65 | 98.58 | 798.58 | 64.72 | 962.87 | 49.84 | 2.14 | 1.16 | | |

### Example 10: In-Vivo CD30 Tumor Burden Assay Study

Female NOG mice were engrafted with 0.3×10⁶ HH-Luc2 tumor cells. Four days later, the mice were injected with 5×10⁶ engineered CD30 CAR T cells, which were generated using two different CD30 constructs, 3884 or 3875 having the same open reading frame encoding the CD30 CAR but having different vector backbones. Control of tumor growth in vivo was assessed.

### 10.1. Preparation of T Cells

### T Cell Preparation Method 1

T cells homozygous for HLA-B and HLA-C were isolated from peripheral blood of a healthy human donor with the following MHC I phenotype: HLA-A*02:01:01, 11:01:01, HLA-B*44:02:01, HLA-C*05:01:01 to generate cells having a triple knockout (at HLA-A, CIITA, and TRAC loci) and an insertion of either 3884 or 3875 at the disrupted TRAC locus (“triple knockout” cells). Briefly, a leukapheresis pack (Stemcell Technologies) was treated in ammonium chloride RBC lysis buffer (Stemcell Technologies, Cat. 07800) for 15 minutes to lyse red blood cells. Peripheral blood mononuclear cell (PBMC) count was determined post lysis and T cell isolation was performed using EasySep Human T cell isolation kit (Stemcell Technologies, Cat. 17951) according to manufacturer's protocol. Isolated CD3+ T cells were re-suspended in Cryostor CS10 media (Stemcell Technologies, Cat. 07930) and frozen down in liquid nitrogen until further use.

Frozen T cells were thawed at a cell concentration of 1.5×10⁶ cells/mL into T cell activation media (TCAM) composed of OpTmizer TCGM as described in Example 5.1 further supplemented with 100 U/mL of recombinant human interleukin-2 (Peprotech, Cat. 200-02), 5 ng/mL IL-7 (Peprotech, Cat. 200-07), 5 ng/mL IL-15 (Peprotech, Cat. 200-15). Cells were rested at 37° C. for 24 hours.

Twenty-four hours post thawing T cells were counted and resuspended at 2×10⁶ cells/mL in TCAM media; Transact was added at 1:50 dilution. LNPs containing mRNA encoding Cas9 (SEQ ID NO:802) and sgRNA targeting CIITA (G013675) (SEQ ID NO: 303) were formulated as described in Example 1. LNPs were diluted in Optmizer TCAM with 5 μg/mL recombinant human ApoE3 (Peprotech, Cat. 350-02). LNPs and T cells suspensions were mixed to yield final concentrations of 2.5 μg total RNA/mL of LNP in TCAM with 2.5% human AB serum and cytokines. All cells were incubated at 37° C. for 24 hours.

Forty-eight hours post activation all groups were transduced with AAV for construct 3884 or 3875. AAV was removed from −80° C. and thawed on ice. Cells were diluted to 0.5×10⁶ cells/mL in TCAM media and AAV was added to 1×10⁵ GC/cell. DNA-PK inhibitor Compound 1, ApoE3, and LNPs containing mRNA encoding cas9 (SEQ ID NO:802) and sgRNA targeting TRAC (G013006) (SEQ ID NO: 233)) were also added to final concentrations of 0.25 μM, 2.5 μg/mL, and 2.5 μg/mL, respectively. Cells were then incubated at 37° C. for 24 hours.

Seventy-two hours post activation, LNPs containing mRNA encoding cas9 (SEQ ID NO:802) and sgRNA targeting HLA-A (G018995) (SEQ ID NO: 305) were both added to a final concentration of 2.5 μg/mL. 24 hours after the final round of editing, cells were transferred to a GREX plate (Wilson Wolf 80240M) in TCAM media. On Day 10, cells were harvested, editing levels were confirmed via flow cytometry, and cells were cryopreserved in Cryostor CS10 media. Cells having an insertion of either construct 3884 or 3875 at the TRAC locus, but not disrupted at the CIITA or HLA-A loci (“single knockout”) were generated in the same manner omitting treated with the CIITA or HLA-A LNPs. The triple knockout and single knockout cells were also generated by Preparation Method 2 as described in Example 11.

### 10.2. Results

For the in vivo study, HH-Luc2 tumor cells were harvested from culture, washed twice with HBSS (Gibco, Cat. No. 14025-092) and resuspended at 0.3×10⁶ cells for injection in 150 μl HBSS. Forty-five female NOG mice (Taconic) were dosed by tail vein injection. Four days later, engineered T cells were thawed, washed with HBSS (Gibco, Cat. No. 14025-092) and resuspended at 5×10⁶ for injection in 150 μL HBSS. Five mice per T cell group were dosed by tail vein injection in HH-Luc2 engrafted mice.

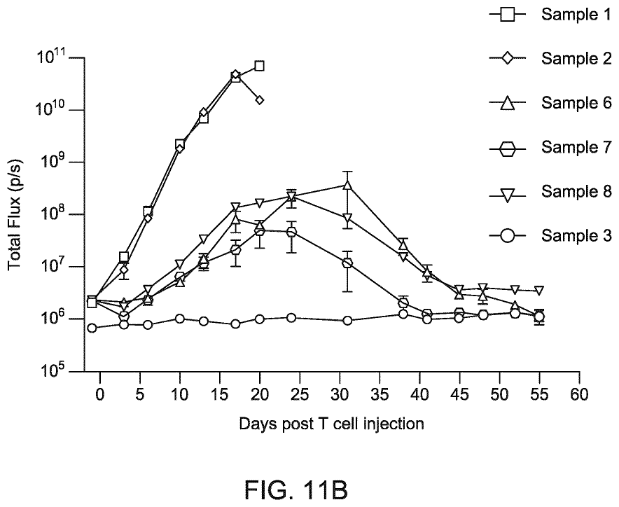

IVIS imaging of live mice was performed to identify luciferase-positive tumor cells by IVIS spectrum to study the tumor burden. IVIS imaging was done at day −1, 3, 6, 10, 13, 17, 20, 24, 31, 38, 41, 45, 48, 52, and 55 post T cell injection. Mice were prepared for imaging with an injection of D-luciferin i.p. at 10 μL/g body weight per the manufacturer's recommendation, about 150 μL per animal. Animals were anesthetized and then placed in the IVIS imaging unit. The visualization was performed with the exposure time set to auto, field of view D, medium binning, and F/stop set to 1. Table 21 and FIG. 11A show total flux (photons/second) from luciferase expressing tumor cells present at the various time points after injection. In vivo, tumor only group (Sample 1) was euthanized by Day 20. At 5×10⁶ cell dose, both constructs, construct 3884 and construct 3875, engineered with Preparation Method 1 showed partial tumor control by day 25 and complete tumor control by day 55. See Sample 4 (construct 3884, single knockout engineered by Preparation Method 1) and Sample 5 (construct 3875, single knockout engineered by Preparation Method 1) in FIG. 11A. FIG. 11B shows total flux (photons/second) from luciferase expressing tumor cells present at the various time points after injection, treated with triple knockout engineered T cells (Sample 6, triple knockout engineered by Preparation Method 1; Sample 7, triple knockout engineered by Preparation Method 2; and Sample 8, triple knockout engineered by Preparation Method 1). Sample 1 (tumor only), Sample 2 (TRAC knockout only), and Sample 3 (cells treated with HBSS only) are the same in both FIGS. 11A and 11B. Overall, efficient tumor regression is observed in both single knockout (FIG. 11A) and triple knockout (FIG. 11B) groups, for Preparation Methods 1 and 2.

TABLE 21
Total Flux (photons/s) from luciferase expressing tumor cells
in treated mice at intervals after T cell injection.

| | Sample 2 | | Sample 4 | | Sample 5 | |
|---|---|---|---|---|---|---|
| | Average | SEM | Average | SEM | Average | SEM |
| −1 | 2.36E+06 | 1.25E+05 | 2.25E+06 | 4.45E+05 | 2.16E+05 | 2.16E+05 |
| 3 | 8.91E+06 | 2.70E+06 | 1.06E+06 | 1.47E+05 | 2.31E+05 | 2.31E+05 |
| 6 | 8.46E+07 | 5.80E+06 | 1.75E+06 | 2.83E+05 | 2.88E+05 | 2.88E+05 |
| 10 | 1.80E+09 | 1.10E+08 | 4.97E+06 | 4.17E+05 | 7.27E+05 | 7.27E+05 |
| 13 | 8.99E+09 | 5.32E+08 | 6.22E+06 | 5.12E+05 | 4.20E+06 | 4.20E+06 |
| 17 | 4.74E+10 | 5.34E+09 | 1.04E+07 | 5.94E+06 | 1.37E+07 | 1.37E+07 |
| 20 | 1.52E+10 | 6.79E+08 | 2.55E+07 | 1.35E+07 | 2.51E+07 | 2.51E+07 |
| 24 | | | 6.14E+06 | 2.40E+06 | 3.50E+07 | 3.50E+07 |
| 31 | | | 1.22E+06 | 1.06E+05 | 7.55E+05 | 7.55E+05 |
| 38 | | | 1.09E+06 | 3.22E+04 | 1.32E+05 | 1.32E+05 |
| 41 | | | 1.03E+06 | 6.08E+04 | 7.99E+04 | 7.99E+04 |
| 45 | | | 1.19E+06 | 4.14E+04 | 6.84E+04 | 6.84E+04 |
| 48 | | | 1.06E+06 | 2.59E+04 | 6.00E+04 | 6.00E+04 |
| 52 | | | 1.37E+06 | 4.66E+04 | 5.21E+04 | 5.21E+04 |
| 55 | | | 9.34E+05 | 5.46E+04 | 6.39E+04 | 6.39E+04 |
| | Sample 6 | | Sample 7 | | Sample 8 | |
| | Average | SEM | Average | SEM | Average | SEM |
| −1 | 2.26E+05 | 2.26E+05 | 1.46E+05 | 1.46E+05 | 2.36E+06 | 2.99E+05 |
| 3 | 1.69E+05 | 1.69E+05 | 1.68E+05 | 1.68E+05 | 1.73E+06 | 1.77E+05 |
| 6 | 3.79E+05 | 3.79E+05 | 4.25E+05 | 4.25E+05 | 3.68E+06 | 5.35E+05 |
| 10 | 8.30E+05 | 8.30E+05 | 1.12E+06 | 1.12E+06 | 1.12E+07 | 2.38E+06 |
| 13 | 3.57E+06 | 3.57E+06 | 2.90E+06 | 2.90E+06 | 3.42E+07 | 1.18E+07 |
| 17 | 3.15E+07 | 3.15E+07 | 9.98E+06 | 9.98E+06 | 1.39E+08 | 5.63E+07 |
| 20 | 7.80E+06 | 7.80E+06 | 2.40E+07 | 2.40E+07 | 1.64E+08 | 4.12E+07 |
| 24 | 7.38E+07 | 7.38E+07 | 2.52E+07 | 2.52E+07 | 2.20E+08 | 8.31E+07 |
| 31 | 2.76E+08 | 2.76E+08 | 7.53E+06 | 7.53E+06 | 8.53E+07 | 4.44E+07 |
| 38 | 8.39E+06 | 8.39E+06 | 7.12E+05 | 7.12E+05 | 1.55E+07 | 8.30E+06 |
| 41 | 2.46E+06 | 2.46E+06 | 1.71E+05 | 1.71E+05 | 6.81E+06 | 1.52E+06 |
| 45 | 3.84E+05 | 3.84E+05 | 9.95E+04 | 9.95E+04 | 3.71E+06 | 1.09E+06 |
| 48 | 7.38E+05 | 7.38E+05 | 1.42E+05 | 1.42E+05 | 3.97E+06 | 1.44E+06 |
| 52 | 3.08E+05 | 3.08E+05 | 1.58E+05 | 1.58E+05 | 3.65E+06 | 1.21E+06 |
| 55 | 2.19E+05 | 2.19E+05 | 3.11E+05 | 3.11E+05 | 3.54E+06 | 1.11E+06 |

| | Sample 1 | | Sample 3 | |
|---|---|---|---|---|
| | Average | SEM | Average | SEM |
| −1 | 2.97E+05 | 2.97E+05 | 3.14E+04 | 3.14E+04 |
| 3 | 2.50E+06 | 2.50E+06 | 2.07E+04 | 2.07E+04 |
| 6 | 2.48E+07 | 2.48E+07 | 4.28E+04 | 4.28E+04 |
| 10 | 2.33E+08 | 2.33E+08 | 4.91E+04 | 4.91E+04 |
| 13 | 1.06E+09 | 1.06E+09 | 3.26E+04 | 3.26E+04 |
| 17 | 7.42E+09 | 7.42E+09 | 1.72E+04 | 1.72E+04 |
| 20 | 9.52E+09 | 9.52E+09 | 2.20E+04 | 2.20E+04 |
| 24 | | | 3.61E+04 | 3.61E+04 |
| 31 | | | 5.13E+04 | 5.13E+04 |
| 38 | | | 4.30E+04 | 4.30E+04 |
| 41 | | | 2.97E+04 | 2.97E+04 |
| 45 | | | 2.94E+04 | 2.94E+04 |
| 48 | | | 3.50E+04 | 3.50E+04 |
| 52 | | | 3.68E+04 | 3.68E+04 |
| 55 | | | 3.55E+04 | 3.55E+04 |

### Example 11: Multi-Editing CD30 CAR-T Cells with Sequential LNP Delivery

Anti-CD30 CAR-T cells were engineered with sequential editing to achieve high levels of HLA-A KO, CIITA KO, TCR KO, and anti-CD30 CAR insertion into the TRAC locus (triple knockout cells). Healthy donor cells were treated sequentially with three LNP compositions, each LNP co-formulated with mRNA encoding Cas9 and a sgRNA targeting either TRAC (G013006) (SEQ ID NO: 233), CIITA (G013675) (SEQ ID NO: 303), or HLA-A (G018995) (sgRNA comprising SEQ ID NO: 305, as shown in Table 22). LNP compositions were formulated with lipid A, cholesterol, DSPC, and PEG2k-DMG in a 50:38.5:10:1.5 molar ratio, respectively. The lipid nucleic acid assemblies were formulated with a lipid amine to RNA phosphate (N:P) molar ratio of about 6, and a ratio of gRNA to mRNA of 1:2 by weight. The CD30 targeting CAR sequence (CD30-CAR) (SEQ ID NO: 259) was integrated into the TRAC cut site by delivering a homology directed repair template delivered by AAV (e.g., AAV 3875 or AAV 3884 containing CD30 CAR).

### 11.1. T Cell Preparation

### T Cell Preparation Method 2

T cells were isolated from the leukapheresis products of three healthy HLA-A*02:01+ and/or HLA-A*03:01 donors (STEMCELL Technologies). T cells were isolated using EasySep Human T Cell Isolation kit (STEMCELL Technologies, Cat. 17951) following manufacturers protocol and cryopreserved using Cryostor CS10 (STEMCELL Technologies, Cat. 07930). The day before initiating T cell editing, cells were thawed and rested overnight in T cell activation media (TCAM): CTS OpTmizer (Thermofisher, Cat. A3705001) supplemented with appropriate growth factors (e.g., cytokines).

### 11.2. LNP Treatment and Expansion of T Cells

On day 1, CIITA-LNPs were prepared. Meanwhile, T cells were harvested, washed, and resuspended in TCAM with a 1:50 dilution of T Cell TransAct, human reagent (Miltenyi, Cat. 130-111-160). T cells and LNP media were mixed at a 1:1 ratio and T cells cultured in an Evolve 70/120 cell culture bag (OriGen Biomedical, Cat.EV120+F-M12) in a 37C, 5% CO2 incubator.

On day 3, T cells were mixed and counted. T cells were harvested and transferred to a second Evolve 70/120 cell culture bag (OriGen Biomedical, Cat.EV120+F-M12) with additional TCAM. TRAC-LNPs were added at final concentrations of 2.5 or 5 μg/mL. CD30 CAR-AAV was then added to at a MOI of 1×10⁵ genome copies/cell along with the DNA-PK inhibitor Compound 1 at a concentration of 0.25 μM.

On day 4, cells were again harvested, counted, and adjusted to a final density of 0.5×10\hat{ }6 cells/mL. HLA-A-LNPs were added to the cell suspension at a final concentration of 2.5 μg/mL in TCAM.

On day 5, T cells were transferred to a 2 L FlexSafe RM bags, with perfusion, DO, pH (Sartorius, Cat. DFP002L-SM) and brought to a final density of 0.5×10\hat{ }6 cells/mL in T cell expansion media (TCEM): CTS OpTmizer (Thermofisher, Cat. A3705001) supplemented with 5% CTS Immune Cell Serum Replacement (Thermofisher, Cat. A2596101) in appropriate buffer and growth factors (e.g., cytokines).

From days 5-11 cells were expanded using an automated rocking platform bioreactor (Biostat RM TX) with the appropriate agitation, platform angle, and gas flow rate, at a temperature of 37° C. Cell counts were collected daily, and culture volume adjusted to maintain a density of 0.5×10\hat{ }6 cells/mL up to a max volume of 1 L.

After expansion cells were harvested and counted with an NC200 Nucleocounter device (Chemometec) to determine cell viability and fold expansion. Edited T cells were assayed by flow cytometry to determine HLA-A*02:01 and/or HLA-A*03:01 knockout (HLA-A+), HLA-DR-DP-DQ knockdown (MHC II-) following knockdown of CIITA, CD30-CAR-T expression (CD3+ CD30Fc/anti-Fc PE+), the expression of residual endogenous TCRs (CD3+CD30 CAR-), T cell purity, T cell memory phenotype, and CD4/CD8 composition.

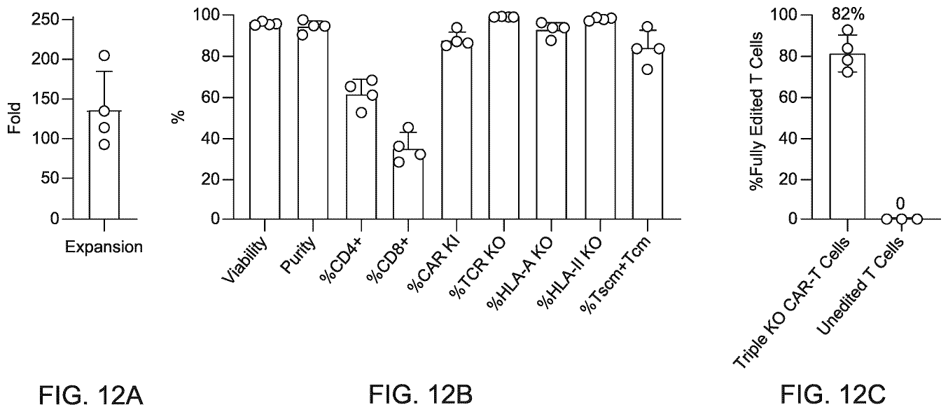

Fold expansion of total T cells from initiation to harvest is shown in Table 22 and FIG. 12A demonstrating high yield of CD30CAR-T cells with triple knockout generated from the sequential editing process. Editing rates (%) for CAR insertion (knock in; KI), endogenous TCR KO, HLA-A KO, CIITA KO (HLA-II KO) across 4 independent engineering runs are shown in Table 22 and FIG. 12B, along with T cell viability, T cell purity (100-% CD56+), % CD4+, % CD8+, and memory status Tscm (CD45RA+, CD62L+)+Tcm (CD45RA-, CD62L+). The total frequency of fully edited triple knockout CD30-CAR-T cells edited was defined by gating cells for CD30-CAR+, TCR-, HLA-A-, HLA-Class II- and is shown for CD8+ gated cells in Table 22 and FIG. 12C demonstrating a high rate of editing across all targets.

TABLE 22
Fold expansion, phenotype, and editing rates
of edited CD30-CAR-T cells across 4 donors

| | Donor 1 | Donor 2 | Donor 3 | Donor 4 | Mean | SD |
|---|---|---|---|---|---|---|
| Fold Expansion | 204.185 | 114 | 133.6 | 92.9 | 136.2 | 37.4 |
| Viability (%) | 97 | 96 | 95.4 | 95.9 | 96.1 | 0.5 |
| Purity (%) | 94.86 | 90.77 | 94.7 | 97.7 | 94.5 | 2.2 |
| % CD4+ | 52.6 | 65.4 | 68.3 | 61.4 | 61.9 | 5.3 |
| % CD8+ | 44.8 | 32.2 | 28.3 | 36.3 | 35.4 | 5.5 |
| % CAR KI | 93.6 | 87.4 | 85.2 | 86.6 | 88.2 | 2.9 |
| % TCR KO | 99.64 | 99.23 | 99.16 | 99.07 | 99.3 | 0.2 |
| % HLA-A KO | 94.2 | 96.4 | 88.4 | 94.1 | 93.3 | 2.6 |
| % HLA-II KO | 99.1 | 98.9 | 98.3 | 97.5 | 98.5 | 0.6 |
| % Tscm + Tcm | 94.7 | 84.1 | 73.7 | 84 | 84.1 | 6.6 |
| % Double KO- | 93.1 | 83.2 | 72.2 | 79.3 | 82.0 | 6.8 |
| CD30 CAR-T | | | | | | |

### Example 12: HvG (Host Vs Graft) Assay of CD30 CAR T Cells with Host Cells with Different HLA Matching Background

Anti-CD30 CAR-T cells (“HLA-A/CIITA KO CAR-T cell group”) were engineered with sequential editing to achieve high levels of HLA-A KO, CIITA KO, TCR KO, and anti-CD30 CAR insertion into the TRAC locus. B2M/CIITA KO anti-CD30 CAR-T cell control group were engineered similarly by substituting HLA-A sgRNA with B2M sgRNA (G000529) (SEQ ID NO: 301) (“B2M/CIITA KO CAR-T cell group”). Healthy donor cells were treated sequentially with three LNP compositions, each LNP co-formulated with mRNA encoding Cas9 and a sgRNA targeting either TRAC (G013006) (SEQ ID NO: 233), CIITA (G013675) (SEQ ID NO: 303), or HLA-A (G018995) (sgRNA comprising SEQ ID NO: 305). Similarly, healthy donor cells were treated sequentially with three LNP compositions, each LNP co-formulated with mRNA encoding Cas9 and a sgRNA targeting either TRAC (G013006) (SEQ ID NO: 233), CIITA (G013675) (SEQ ID NO: 303), or B2M (G000529) (SEQ ID NO: 301). LNP compositions were formulated with lipid A, cholesterol, DSPC, and PEG2k-DMG in a 50:38.5:10:1.5 molar ratio, respectively. The lipid nucleic acid assemblies were formulated with a lipid amine to RNA phosphate (N:P) molar ratio of about 6, and a ratio of gRNA to mRNA of 1:2 by weight. The CD30 targeting CAR sequence (CD30-CAR) (SEQ ID NO: 259) was integrated into the TRAC cut site by delivering a homology directed repair template delivered by AAV.

The degree of CD30 CAR T cell rejection by host PBMC due to HvG (Host vs Graft) was evaluated indirectly by measuring the proliferation of the host cells while co-cultured with irradiated donor T cells. Irradiated HLA-A/CIITA KO CAR-T cells, together with UED (Unedited) and B2M/CIITA KO CAR-T cells were co-cultured with CTV (Cell Tracer Dye Violet (Thermofisher, Cat. C34557)) labeled partially matched (match for HLA-B/C; mis-matched for HLA-A, HLA-DR/DP/DQ) and mismatched (mis-matched for all HLA) CD56 depleted host PBMC in a 96-well plate at 3:1 ratio for 6 days. Proliferation of the host cells were then quantified through CTV dilution using flow cytometry.

### 12.1. T Cell Preparation

T cells were isolated from the leukapheresis products of three healthy HLA-A*02:01+ and/or HLA-A*03:01 donors (STEMCELL Technologies). T cells were isolated using EasySep Human T Cell Isolation kit (STEMCELL Technologies, Cat. 17951) following manufacturer's protocol and cryopreserved using Cryostor CS10 (STEMCELL Technologies, Cat. 07930). The day before initiating T cell editing, cells were thawed and rested overnight in T cell activation media (TCAM): CTS OpTmizer (Thermofisher, Cat. A3705001) supplemented with appropriate growth factors (e.g., cytokines).

### 12.2. LNP Treatment and Expansion of T Cells

On day 1, CIITA-LNPs were prepared. Meanwhile, T cells were harvested, washed, and resuspended in TCAM with a 1:50 dilution of T Cell TransAct, human reagent (Miltenyi, Cat. 130-111-160). T cells and LNP-ApoE media were mixed at a 1:1 ratio and T cells cultured in an Evolve 70/120 cell culture bag (OriGen Biomedical, Cat.EV120+F-M12) in a 37° C., 5% CO2 incubator.

On day 3, T cells were mixed and counted. T cells were harvested and transferred to a second Evolve 70/120 cell culture bag (OriGen Biomedical, Cat.EV120+F-M12) with additional TCAM. TRAC-LNPs were added at final concentrations of 2.5 or 5 μg/mL. CD30 CAR-AAV was then added to at a MOI of 1×10\hat{ }⁵ genome copies/cell along with DNA-PK inhibitor Compound 1 at a concentration of 0.25 μM.

On day 4, cells were again harvested, counted, and adjusted to a final density of 0.5×10⁶ cells/mL. HLA-A-LNPs (for HLA-A/CIITA KO CAR-T cell group) or B2M-LNPs (for B2M/CIITA KO CAR-T cell group) were added to the cell suspension at a final concentration of 2.5 ug/mL in TCAM.

On day 5, T cells were transferred to a 2 L FlexSafe RM bags, with perfusion, DO, pH (Sartorius, Cat. DFP002L-SM) and brought to a final density of 0.5×10\hat{ }6 cells/mL in T cell expansion media (TCEM): CTS OpTmizer (Thermofisher, Cat. A3705001) supplemented with 5% CTS Immune Cell Serum Replacement (Thermofisher, Cat. A2596101) in appropriate buffer and growth factors (e.g., cytokines).

From days 5-11 cells were expanded using an automated rocking platform bioreactor (Biostat RM TX) with the appropriate agitation, platform angle, and gas flow rate, at a temperature of 37° C. Cell counts were collected daily, and culture volume adjusted to maintain a density of 0.5×10⁶ cells/mL up to a max volume of 1 L.

After expansion cells were harvested and counted with an NC200 Nucleocounter device (Chemometec) to determine cell viability and fold expansion. Edited T cells were assayed by flow cytometry to determine HLA-A*02:01 and/or HLA-A*03:01 knockout (HLA-A+), B2M knockout (B2M-), HLA-DR-DP-DQ knockdown (MHC II-) following knockdown CIITA, CD30-CAR-T expression (CD3+ CD30Fc/anti-Fc PE+), the expression of residual endogenous TCRs (CD3+ CD30 CAR-), T cell purity, T cell memory phenotype, and CD4/CD8 composition.

### 12.3. In Vitro HvG Assay of CD30 CAR T Cells

On day 0, all groups of donor T cells (UED, HLA-A/CIITA KO and B2M/CIITA KO) were thawed and resuspend in T cell growth media (TCGM): CTS OpTmizer (Thermofisher, Cat. A3705001) supplemented with 5% CTS Immune Cell Serum Replacement (Thermofisher, Cat. A2596101), 10% Human AB Serum (Gemini, 100-512), 1× GlutaMAX (Thermofisher, Cat. 35050061), 10 mM HEPES (Thermofisher, Cat. 15630080), 200 U/mL IL-2 (Peprotech, Cat. 200-02), IL-7 (Peprotech, Cat. 200-07), IL-15 (Peprotech, Cat. 200-15) at 1×10⁶ cells/mL. Host PBMC (HLA-B and -C matched host and HLA-B and -C mis-matched host) were thawed and resuspended in TCGM at 1×10⁶ cells/mL.

On day 1, CD56+ cells were depleted from host PBMC using CD56 MicroBeads (Miltenyi, Cat.130-050-401) following the manufacture protocol. CD56 depleted PBMC were then labeled with CTV (CellTracer Dye Violet (Thermofisher, Cat. C34557)), resuspended at 1×10⁶/mL in TCGM without cytokines. For donor T cells, all donor cells were irradiated at 4000 rad for 33 min, spun down and resuspended at a concentration of 1×10⁶/mL in TCGM without cytokines. To set up the co-culture assay at a Donor: Host ratio=3:1, 150,000 cells of donor T cells and 50,000 cells of CTV labeled CD56-depleted host PBMC were added together in a 96-well round bottom plate. Positive control of host PBMC proliferation was run by adding 1% T Cell TransAct, human reagent (Miltenyi, Cat. 130-111-160) to host PBMC only group.

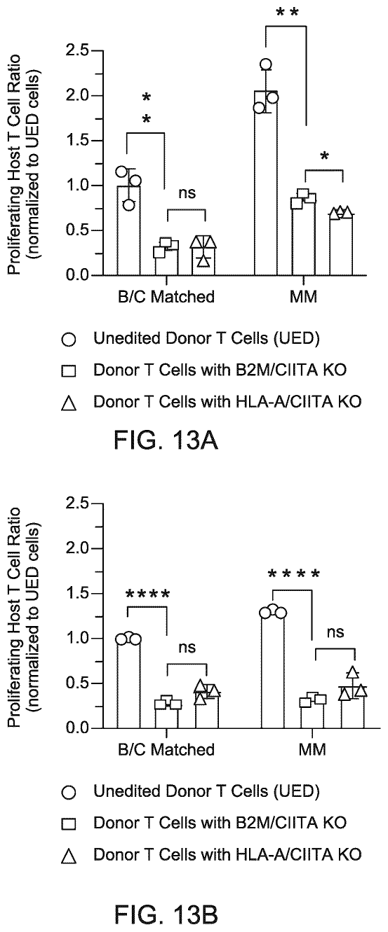

On day 6, co-cultured cells were stained in FACS buffer (PBS, 1% FBS, 2 mM EDTA) with an antibody cocktail targeting the following molecules: CD8 (Biolegend, Cat. 344704), CD3 (Biolegend, Cat. 317324) and 7-AAD Live/Dead dye (Biolegend, Cat. 420404). Cells were subsequently washed, analyzed on a Cytoflex LX instrument (Beckman Coulter) using the FlowJo software package. Proliferation of the host cells was then quantified through CTV dilution. Normalized proliferation rate was determined according to the formula: Normalized proliferation rate=(each group−host only group)/UED-PM group. Results are shown in FIGS. 13A and 13B and Table 23 and 24 for two T cell donors:

TABLE 23
Normalized proliferation of partially matched
and mis-matched host T cells against different
multi-edited CD30 CAR-T cells for Donor 1.

| | UED | | | B2M/CIITA KO | | | HLA-A/CIITA KO | | |
|---|---|---|---|---|---|---|---|---|---|
| PM | 0.79 | 1.06 | 1.16 | 0.33 | 0.36 | 0.26 | 0.4 | 0.17 | 0.4 |
| MM | 1.87 | 1.98 | 2.36 | 0.9 | 0.85 | 0.8 | 0.7 | 0.71 | 0.72 |

TABLE 24
Normalized proliferation of partially matched
and mis-matched host T cells against different
multi-edited CD30 CAR-T cells for Donor 2.

| | UED | | | B2M/CIITA KO | | | HLA-A/CIITA KO | | |
|---|---|---|---|---|---|---|---|---|---|
| PM | 0.99 | 0.99 | 1.02 | 0.31 | 0.25 | 0.26 | 0.43 | 0.48 | 0.33 |
| MM | 1.29 | 1.28 | 1.33 | 0.32 | 0.34 | 0.28 | 0.64 | 0.38 | 0.42 |

TABLE 25
Additional Sequences

| | SEQ | |
|---|---|---|
| | ID | |
| Description | NO | Sequence |
| G013006 guide | 231 | mC*mU*mC*UCAGCUGGUACACGGCAGUUUUAGAmGmCmU |
| RNA targeting | | mAmGmAmAmAmUmAmGmCAAGUUAAAAUAAGGCUAGUC |
| TRAC with | | CGUUAUCAmAmCmUmUmGmAmAmAmAmAmGmUmGmGmC |
| guide sequence | | mAmCmCmGmAmGmUmCmGmGmUmGmCmU*mU*mU*mU |
| SEQ ID NO: 233 | | |
| Exemplary guide | 233 | CUCUCAGCUGGUACACGGCA |
| sequence for | | |
| TRAC gene | | |
| G000529 guide | 300 | mG*mG*mC*CACGGAGCGAGACAUCUGUUUUAGAmGmCmU |
| RNA targeting | | mAmGmAmAmAmUmAmGmCAAGUUAAAAUAAGGCUAGUC |
| B2M with guide | | CGUUAUCAmAmCmUmUmGmAmAmAmAmAmGmUmGmGmC |
| sequence SEQ | | mAmCmCmGmAmGmUmCmGmGmUmGmCmU*mU*mU*mU |
| ID NO: 301 | | |
| Exemplary guide | 301 | GGCCACGGAGCGAGACAUCU |
| sequence for | | |
| B2M gene | | |
| G013675 guide | 302 | mC*mC*mC*CCGGACGGUUCAAGCAAGUUUUAGAmGmCmU |
| RNA targeting | | mAmGmAmAmAmUmAmGmCAAGUUAAAAUAAGGCUAGUC |
| CIITA with | | CGUUAUCAmAmCmUmUmGmAmAmAmAmAmGmUmGmGmC |
| guide sequence | | mAmCmCmGmAmGmUmCmGmGmUmGmCmU*mU*mU*mU |
| SEQ ID NO: 303 | | |
| Exemplary guide | 303 | CCCCCGGACGGUUCAAGCAA |
| sequence for | | |
| CIITA gene | | |
| G018995 guide | 304 | mA*mC*mA*GCGACGCCGCGAGCCAGGUUUUAGAmGmCmU |
| RNA targeting | | mAmGmAmAmAmUmAmGmCAAGUUAAAAUAAGGCUAGUC |
| HLA-A with | | CGUUAUCAmAmCmUmUmGmAmAmAmAmAmGmUmGmGmC |
| guide sequence | | mAmCmCmGmAmGmUmCmGmGmUmGmCmU*mU*mU*mU |
| SEQ ID NO: 305 | | |
| Exemplary guide | 305 | ACAGCGACGCCGCGAGCCAG |
| sequence for | | |
| HLA-A gene | | |
| Construct 3881 | 235 | ATGGACTTCCAGGTGCAGATCTTCAGCTTCCTGCTGATCAG |
| | | CGCCAGCGTGATCATGAGCCGGATGGCCCAGGTGCAGCTGC |
| | | AGCAGAGCGGCGCCGAGCTGGCCCGGCCCGGCGCCAGCGT |
| | | GAAGATGAGCTGCAAGGCCAGCGGCTACACCTTCACCACCT |
| | | ACACCATCCACTGGGTGCGGCGGCGGCCCGGCCACGACCTG |
| | | GAGTGGATCGGCTACATCAACCCCAGCAGCGGCTGCAGCG |
| | | ACTACAACCAGAACTTCAAGGGCAAGACCACCCTGACCGCC |
| | | GACAAGAGCAGCAACACCGCCTACATGCAGCTGAACAGCC |
| | | TGACCAGCGAGGACAGCGCCGTGTACTACTGCGCCCGGCGG |
| | | GCCGACTACGGCAACTACGAGTACACCTGGTTCGCCTACTG |
| | | GGGCCAGGGCACCACCGTGACCGTGAGCAGCAGCGGCGGC |
| | | GGCAGCGGCGGCGGCGGCAGCGGCGGCGGCGGCAGCGTGA |
| | | TCGAGCTGACCCAGAGCCCCAAGTTCATGAGCACCAGCGTG |
| | | GGCGACCGGGTGAACGTGACCTACAAGGCCAGCCAGAACG |
| | | TGGGCACCAACGTGGCCTGGTTCCAGCAGAAGCCCGGCCAG |
| | | AGCCCCAAGGTGCTGATCTACAGCGCCAGCTACCGGTACAG |
| | | CGGCGTGCCCGACCGGTTCACCGGCAGCGGCAGCGGCACC |
| | | GACTTCACCCTGACCATCAGCAACGTGCAGAGCGAGGACCT |
| | | GGCCGAGTACTTCTGCCAGCAGTACCACACCTACCCCCTGA |
| | | CCTTCGGCGGCGGCACCAAGCTGGAGATCAAGCGGAGCGA |
| | | CCCCGCCGAGCCCAAGAGCCCCGACAAGACCCACACCTGCC |
| | | CCCCCTGCCCCGCCCCCCCCGTGGCCGGCCCCAGCGTGTTC |
| | | CTGTTCCCCCCCAAGCCCAAGGACACCCTGATGATCGCCCG |
| | | GACCCCCGAGGTGACCTGCGTGGTGGTGGACGTGAGCCACG |
| | | AGGACCCCGAGGTGAAGTTCAACTGGTACGTGGACGGCGT |
| | | GGAGGTGCACAACGCCAAGACCAAGCCCCGGGAGGAGCAG |
| | | TACAACAGCACCTACCGGGTGGTGAGCGTGCTGACCGTGCT |
| | | GCACCAGGACTGGCTGAACGGCAAGGAGTACAAGTGCAAG |
| | | GTGAGCAACAAGGCCCTGCCCGCCCCCATCGAGAAGACCAT |
| | | CAGCAAGGCCAAGGGCCAGCCCCGGGAGCCCCAGGTGTAC |
| | | ACCCTGCCCCCCAGCCGGGACGAGCTGACCAAGAACCAGG |
| | | TGAGCCTGACCTGCCTGGTGAAGGGCTTCTACCCCAGCGAC |
| | | ATCGCCGTGGAGTGGGAGAGCAACGGCCAGCCCGAGAACA |
| | | ACTACAAGACCACCCCCCCCGTGCTGGACAGCGACGGCAGC |
| | | TTCTTCCTGTACAGCAAGCTGACCGTGGACAAGAGCCGGTG |
| | | GCAGCAGGGCAACGTGTTCAGCTGCAGCGTGATGCACGAG |
| | | GCCCTGCACAACCACTACACCCAGAAGAGCCTGAGCCTGAG |
| | | CCCCGGCAAGAAGGACCCCAAGTTCTGGGTGCTGGTGGTGG |
| | | TGGGCGGCGTGCTGGCCTGCTACAGCCTGCTGGTGACCGTG |
| | | GCCTTCATCATCTTCTGGGTGCGGAGCAAGCGGAGCCGGCT |
| | | GCTGCACAGCGACTACATGAACATGACCCCCCGGCGGCCCG |
| | | GCCCCACCCGGAAGCACTACCAGGCCTACGCCGCCGCCCGG |
| | | GACTTCGCCGCCTACCGGAGCCTGCGGGTGAAGTTCAGCCG |
| | | GAGCGCCGACGCCCCCGCCTACCAGCAGGGCCAGAACCAG |
| | | CTGTACAACGAGCTGAACCTGGGCCGGCGGGAGGAGTACG |
| | | ACGTGCTGGACAAGCGGCGGGGCCGGGACCCCGAGATGGG |
| | | CGGCAAGCCCCGGCGGAAGAACCCCCAGGAGGGCCTGTAC |
| | | AACGAGCTGCAGAAGGACAAGATGGCCGAGGCCTACAGCG |
| | | AGATCGGCATGAAGGGCGAGCGGCGGCGGGGCAAGGGCCA |
| | | CGACGGCCTGTACCAGGGCCTGAGCACCGCCACCAAGGAC |
| | | ACCTACGACGCCCTGCACATGCAGGCCCTGCCCCCCCGG |
| Construct 3882 | 237 | ATGGACTTCCAGGTGCAGATCTTCAGCTTCCTGCTGATCAG |
| | | CGCCAGCGTGATCATGAGCCGGATGGCCCAGGTGCAGCTGC |
| | | AGCAGAGCGGCGCCGAGCTGGCCCGGCCCGGCGCCAGCGT |
| | | GAAGATGAGCTGCAAGGCCAGCGGCTACACCTTCACCACCT |
| | | ACACCATCCACTGGGTGCGGCGGCGGCCCGGCCACGACCTG |
| | | GAGTGGATCGGCTACATCAACCCCAGCAGCGGCTGCAGCG |
| | | ACTACAACCAGAACTTCAAGGGCAAGACCACCCTGACCGCC |
| | | GACAAGAGCAGCAACACCGCCTACATGCAGCTGAACAGCC |
| | | TGACCAGCGAGGACAGCGCCGTGTACTACTGCGCCCGGCGG |
| | | GCCGACTACGGCAACTACGAGTACACCTGGTTCGCCTACTG |
| | | GGGCCAGGGCACCACCGTGACCGTGAGCAGCAGCGGCGGC |
| | | GGCAGCGGCGGCGGCGGCAGCGGCGGCGGCGGCAGCGTGA |
| | | TCGAGCTGACCCAGAGCCCCAAGTTCATGAGCACCAGCGTG |
| | | GGCGACCGGGTGAACGTGACCTACAAGGCCAGCCAGAACG |
| | | TGGGCACCAACGTGGCCTGGTTCCAGCAGAAGCCCGGCCAG |
| | | AGCCCCAAGGTGCTGATCTACAGCGCCAGCTACCGGTACAG |
| | | CGGCGTGCCCGACCGGTTCACCGGCAGCGGCAGCGGCACC |
| | | GACTTCACCCTGACCATCAGCAACGTGCAGAGCGAGGACCT |
| | | GGCCGAGTACTTCTGCCAGCAGTACCACACCTACCCCCTGA |
| | | CCTTCGGCGGCGGCACCAAGCTGGAGATCAAGCGGAGCGA |
| | | CCCCGCCGAGCCCAAGAGCCCCGACAAGACCCACACCTGCC |
| | | CCGGCCAGCCCCGGGAGCCCCAGGTGTACACCCTGCCCCCC |
| | | AGCCGGGACGAGCTGACCAAGAACCAGGTGAGCCTGACCT |
| | | GCCTGGTGAAGGGCTTCTACCCCAGCGACATCGCCGTGGAG |
| | | TGGGAGAGCAACGGCCAGCCCGAGAACAACTACAAGACCA |
| | | CCCCCCCCGTGCTGGACAGCGACGGCAGCTTCTTCCTGTAC |
| | | AGCAAGCTGACCGTGGACAAGAGCCGGTGGCAGCAGGGCA |
| | | ACGTGTTCAGCTGCAGCGTGATGCACGAGGCCCTGCACAAC |
| | | CACTACACCCAGAAGAGCCTGAGCCTGAGCCCCGGCAAGA |
| | | AGGACCCCAAGTTCTGGGTGCTGGTGGTGGTGGGCGGCGTG |
| | | CTGGCCTGCTACAGCCTGCTGGTGACCGTGGCCTTCATCATC |
| | | TTCTGGGTGCGGAGCAAGCGGAGCCGGCTGCTGCACAGCG |
| | | ACTACATGAACATGACCCCCCGGCGGCCCGGCCCCACCCGG |
| | | AAGCACTACCAGGCCTACGCCGCCGCCCGGGACTTCGCCGC |
| | | CTACCGGAGCCTGCGGGTGAAGTTCAGCCGGAGCGCCGAC |
| | | GCCCCCGCCTACCAGCAGGGCCAGAACCAGCTGTACAACG |
| | | AGCTGAACCTGGGCCGGCGGGAGGAGTACGACGTGCTGGA |
| | | CAAGCGGCGGGGCCGGGACCCCGAGATGGGCGGCAAGCCC |
| | | CGGCGGAAGAACCCCCAGGAGGGCCTGTACAACGAGCTGC |
| | | AGAAGGACAAGATGGCCGAGGCCTACAGCGAGATCGGCAT |
| | | GAAGGGCGAGCGGCGGCGGGGCAAGGGCCACGACGGCCTG |
| | | TACCAGGGCCTGAGCACCGCCACCAAGGACACCTACGACG |
| | | CCCTGCACATGCAGGCCCTGCCCCCCCGG |
| Construct 3883 | 239 | ATGGACTTCCAGGTGCAGATCTTCAGCTTCCTGCTGATCAG |
| | | CGCCAGCGTGATCATGAGCCGGATGGCCCAGGTGCAGCTGC |
| | | AGCAGAGCGGCGCCGAGCTGGCCCGGCCCGGCGCCAGCGT |
| | | GAAGATGAGCTGCAAGGCCAGCGGCTACACCTTCACCACCT |
| | | ACACCATCCACTGGGTGCGGCGGCGGCCCGGCCACGACCTG |
| | | GAGTGGATCGGCTACATCAACCCCAGCAGCGGCTGCAGCG |
| | | ACTACAACCAGAACTTCAAGGGCAAGACCACCCTGACCGCC |
| | | GACAAGAGCAGCAACACCGCCTACATGCAGCTGAACAGCC |
| | | TGACCAGCGAGGACAGCGCCGTGTACTACTGCGCCCGGCGG |
| | | GCCGACTACGGCAACTACGAGTACACCTGGTTCGCCTACTG |
| | | GGGCCAGGGCACCACCGTGACCGTGAGCAGCAGCGGCGGC |
| | | GGCAGCGGCGGCGGCGGCAGCGGCGGCGGCGGCAGCGTGA |
| | | TCGAGCTGACCCAGAGCCCCAAGTTCATGAGCACCAGCGTG |
| | | GGCGACCGGGTGAACGTGACCTACAAGGCCAGCCAGAACG |
| | | TGGGCACCAACGTGGCCTGGTTCCAGCAGAAGCCCGGCCAG |
| | | AGCCCCAAGGTGCTGATCTACAGCGCCAGCTACCGGTACAG |
| | | CGGCGTGCCCGACCGGTTCACCGGCAGCGGCAGCGGCACC |
| | | GACTTCACCCTGACCATCAGCAACGTGCAGAGCGAGGACCT |
| | | GGCCGAGTACTTCTGCCAGCAGTACCACACCTACCCCCTGA |
| | | CCTTCGGCGGCGGCACCAAGCTGGAGATCAAGCGGAGCGA |
| | | CCCCGCCGAGCCCAAGAGCCCCGACAAGACCCACACCTGCC |
| | | CCCCCTGCCCCGCCCCCCCCGTGGCCGGCCCCAGCGTGTTC |
| | | CTGTTCCCCCCCAAGCCCAAGGACACCCTGATGATCGCCCG |
| | | GACCCCCGAGGTGACCTGCGTGGTGGTGGACGTGAGCCACG |
| | | AGGACCCCGAGGTGAAGTTCAACTGGTACGTGGACGGCGT |
| | | GGAGGTGCACAACGCCAAGACCAAGCCCCGGGAGGAGCAG |
| | | TACAACAGCACCTACCGGGTGGTGAGCGTGCTGACCGTGCT |
| | | GCACCAGGACTGGCTGAACGGCAAGGAGTACAAGTGCAAG |
| | | GTGAGCAACAAGGCCCTGCCCGCCCCCATCGAGAAGACCAT |
| | | CAGCAAGGCCAAGGGCCAGCCCCGGGAGCCCCAGGTGTAC |
| | | ACCCTGCCCCCCAGCCGGGACGAGCTGACCAAGAACCAGG |
| | | TGAGCCTGACCTGCCTGGTGAAGGGCTTCTACCCCAGCGAC |
| | | ATCGCCGTGGAGTGGGAGAGCAACGGCCAGCCCGAGAACA |
| | | ACTACAAGACCACCCCCCCCGTGCTGGACAGCGACGGCAGC |
| | | TTCTTCCTGTACAGCAAGCTGACCGTGGACAAGAGCCGGTG |
| | | GCAGCAGGGCAACGTGTTCAGCTGCAGCGTGATGCACGAG |
| | | GCCCTGCACAACCACTACACCCAGAAGAGCCTGAGCCTGAG |
| | | CCCCGGCAAGAAGGACCCCAAGTTCTGGGTGCTGGTGGTGG |
| | | TGGGCGGCGTGCTGGCCTGCTACAGCCTGCTGGTGACCGTG |
| | | GCCTTCATCATCTTCTGGGTGCGGAGCAAGCGGAGCCGGCT |
| | | GCTGCACAGCGACTACATGAACATGACCCCCCGGCGGCCCG |
| | | GCCCCACCCGGAAGCACTACCAGCCCTACGCCCCCCCCCGG |
| | | GACTTCGCCGCCTACCGGAGCCTGCGGGTGAAGTTCAGCCG |
| | | GAGCGCCGACGCCCCCGCCTACCAGCAGGGCCAGAACCAG |
| | | CTGTACAACGAGCTGAACCTGGGCCGGCGGGAGGAGTACG |
| | | ACGTGCTGGACAAGCGGCGGGGCCGGGACCCCGAGATGGG |
| | | CGGCAAGCCCCGGCGGAAGAACCCCCAGGAGGGCCTGTAC |
| | | AACGAGCTGCAGAAGGACAAGATGGCCGAGGCCTACAGCG |
| | | AGATCGGCATGAAGGGCGAGCGGCGGCGGGGCAAGGGCCA |
| | | CGACGGCCTGTACCAGGGCCTGAGCACCGCCACCAAGGAC |
| | | ACCTACGACGCCCTGCACATGCAGGCCCTGCCCCCCCGGTA |
| | | A |
| Construct 3884 | 241 | ATGGACTTCCAGGTGCAGATCTTCAGCTTCCTGCTGATCAG |
| | | CGCCAGCGTGATCATGAGCCGGATGGCCCAGGTGCAGCTGC |
| | | AGCAGAGCGGCGCCGAGCTGGCCCGGCCCGGCGCCAGCGT |
| | | GAAGATGAGCTGCAAGGCCAGCGGCTACACCTTCACCACCT |
| | | ACACCATCCACTGGGTGCGGCGGCGGCCCGGCCACGACCTG |
| | | GAGTGGATCGGCTACATCAACCCCAGCAGCGGCTGCAGCG |
| | | ACTACAACCAGAACTTCAAGGGCAAGACCACCCTGACCGCC |
| | | GACAAGAGCAGCAACACCGCCTACATGCAGCTGAACAGCC |
| | | TGACCAGCGAGGACAGCGCCGTGTACTACTGCGCCCGGCGG |
| | | GCCGACTACGGCAACTACGAGTACACCTGGTTCGCCTACTG |
| | | GGGCCAGGGCACCACCGTGACCGTGAGCAGCAGCGGCGGC |
| | | GGCAGCGGCGGCGGCGGCAGCGGCGGCGGCGGCAGCGTGA |
| | | TCGAGCTGACCCAGAGCCCCAAGTTCATGAGCACCAGCGTG |
| | | GGCGACCGGGTGAACGTGACCTACAAGGCCAGCCAGAACG |
| | | TGGGCACCAACGTGGCCTGGTTCCAGCAGAAGCCCGGCCAG |
| | | AGCCCCAAGGTGCTGATCTACAGCGCCAGCTACCGGTACAG |
| | | CGGCGTGCCCGACCGGTTCACCGGCAGCGGCAGCGGCACC |
| | | GACTTCACCCTGACCATCAGCAACGTGCAGAGCGAGGACCT |
| | | GGCCGAGTACTTCTGCCAGCAGTACCACACCTACCCCCTGA |
| | | CCTTCGGCGGCGGCACCAAGCTGGAGATCAAGCGGAGCGA |
| | | CCCCGCCACCACCACCCCCGCCCCCCGGCCCCCCACCCCCG |
| | | CCCCCACCATCGCCAGCCAGCCCCTGAGCCTGCGGCCCGAG |
| | | GCCTGCCGGCCCGCCGCCGGCGGCGCCGTGCACACCCGGGG |
| | | CCTGGACTTCGCCTGCGACAAGGACCCCAAGTTCTGGGTGC |
| | | TGGTGGTGGTGGGCGGCGTGCTGGCCTGCTACAGCCTGCTG |
| | | GTGACCGTGGCCTTCATCATCTTCTGGGTGCGGAGCAAGCG |
| | | GAGCCGGCTGCTGCACAGCGACTACATGAACATGACCCCCC |
| | | GGCGGCCCGGCCCCACCCGGAAGCACTACCAGCCCTACGCC |
| | | CCCCCCCGGGACTTCGCCGCCTACCGGAGCCTGCGGGTGAA |
| | | GTTCAGCCGGAGCGCCGACGCCCCCGCCTACCAGCAGGGCC |
| | | AGAACCAGCTGTACAACGAGCTGAACCTGGGCCGGCGGGA |
| | | GGAGTACGACGTGCTGGACAAGCGGCGGGGCCGGGACCCC |
| | | GAGATGGGCGGCAAGCCCCGGCGGAAGAACCCCCAGGAGG |
| | | GCCTGTACAACGAGCTGCAGAAGGACAAGATGGCCGAGGC |
| | | CTACAGCGAGATCGGCATGAAGGGCGAGCGGCGGCGGGGC |
| | | AAGGGCCACGACGGCCTGTACCAGGGCCTGAGCACCGCCA |
| | | CCAAGGACACCTACGACGCCCTGCACATGCAGGCCCTGCCC |
| | | CCCCGG |
| Construct 3885 | 243 | ATGGCCCTGCCCGTGACCGCCCTGCTGCTGCCCCTGGCCCT |
| | | GCTGCTGCACGCCGCCCGGCCCGACATCCAGATGACCCAGA |
| | | GCCCCACCAGCCTGAGCGCCAGCGTGGGCGACCGGGTGAC |
| | | CATCACCTGCCGGGCCAGCCAGGGCATCAGCAGCTGGCTGA |
| | | CCTGGTACCAGCAGAAGCCCGAGAAGGCCCCCAAGAGCCT |
| | | GATCTACGCCGCCAGCAGCCTGCAGAGCGGCGTGCCCAGCC |
| | | GGTTCAGCGGCAGCGGCAGCGGCACCGACTTCACCCTGACC |
| | | ATCAGCAGCCTGCAGCCCGAGGACTTCGCCACCTACTACTG |
| | | CCAGCAGTACGACAGCTACCCCATCACCTTCGGCCAGGGCA |
| | | CCCGGCTGGAGATCAAGGGCAGCACCAGCGGCAGCGGCAA |
| | | GCCCGGCAGCGGCGAGGGCAGCACCAAGGGCCAGGTGCAG |
| | | CTGCAGCAGTGGGGCGCCGGCCTGCTGAAGCCCAGCGAGA |
| | | CCCTGAGCCTGACCTGCGCCGTGTACGGCGGCAGCTTCAGC |
| | | GCCTACTACTGGAGCTGGATCCGGCAGCCCCCCGGCAAGGG |
| | | CCTGGAGTGGATCGGCGACATCAACCACGGCGGCGGCACC |
| | | AACTACAACCCCAGCCTGAAGAGCCGGGTGACCATCAGCGT |
| | | GGACACCAGCAAGAACCAGTTCAGCCTGAAGCTGAACAGC |
| | | GTGACCGCCGCCGACACCGCCGTGTACTACTGCGCCAGCCT |
| | | GACCGCCTACTGGGGCCAGGGCAGCCTGGTGACCGTGAGC |
| | | AGCATCGAGGTGATGTACCCCCCCCCCTACCTGGACAACGA |
| | | GAAGAGCAACGGCACCATCATCCACGTGAAGGGCAAGCAC |
| | | CTGTGCCCCAGCCCCCTGTTCCCCGGCCCCAGCAAGCCCTTC |
| | | TGGGTGCTGGTGGTGGTGGGCGGCGTGCTGGCCTGCTACAG |
| | | CCTGCTGGTGACCGTGGCCTTCATCATCTTCTGGGTGCGGA |
| | | GCAAGCGGAGCCGGCTGCTGCACAGCGACTACATGAACAT |
| | | GACCCCCCGGCGGCCCGGCCCCACCCGGAAGCACTACCAGC |
| | | CCTACGCCCCCCCCCGGGACTTCGCCGCCTACCGGAGCCGG |
| | | GTGAAGTTCAGCCGGAGCGCCGACGCCCCCGCCTACCAGCA |
| | | GGGCCAGAACCAGCTGTACAACGAGCTGAACCTGGGCCGG |
| | | CGGGAGGAGTACGACGTGCTGGACAAGCGGCGGGGCCGGG |
| | | ACCCCGAGATGGGCGGCAAGCCCCGGCGGAAGAACCCCCA |
| | | GGAGGGCCTGTACAACGAGCTGCAGAAGGACAAGATGGCC |
| | | GAGGCCTACAGCGAGATCGGCATGAAGGGCGAGCGGCGGC |
| | | GGGGCAAGGGCCACGACGGCCTGTACCAGGGCCTGAGCAC |
| | | CGCCACCAAGGACACCTACGACGCCCTGCACATGCAGGCCC |
| | | TGCCCCCCCGG |
| Construct 3886 | 245 | ATGGCCCTGCCCGTGACCGCCCTGCTGCTGCCCCTGGCCCT |
| | | GCTGCTGCACGCCGCCCGGCCCGACATCCAGATGACCCAGA |
| | | GCCCCACCAGCCTGAGCGCCAGCGTGGGCGACCGGGTGAC |
| | | CATCACCTGCCGGGCCAGCCAGGGCATCAGCAGCTGGCTGA |
| | | CCTGGTACCAGCAGAAGCCCGAGAAGGCCCCCAAGAGCCT |
| | | GATCTACGCCGCCAGCAGCCTGCAGAGCGGCGTGCCCAGCC |
| | | GGTTCAGCGGCAGCGGCAGCGGCACCGACTTCACCCTGACC |
| | | ATCAGCAGCCTGCAGCCCGAGGACTTCGCCACCTACTACTG |
| | | CCAGCAGTACGACAGCTACCCCATCACCTTCGGCCAGGGCA |
| | | CCCGGCTGGAGATCAAGGGCAGCACCAGCGGCAGCGGCAA |
| | | GCCCGGCAGCGGCGAGGGCAGCACCAAGGGCCAGGTGCAG |
| | | CTGCAGCAGTGGGGCGCCGGCCTGCTGAAGCCCAGCGAGA |
| | | CCCTGAGCCTGACCTGCGCCGTGTACGGCGGCAGCTTCAGC |
| | | GCCTACTACTGGAGCTGGATCCGGCAGCCCCCCGGCAAGGG |
| | | CCTGGAGTGGATCGGCGACATCAACCACGGCGGCGGCACC |
| | | AACTACAACCCCAGCCTGAAGAGCCGGGTGACCATCAGCGT |
| | | GGACACCAGCAAGAACCAGTTCAGCCTGAAGCTGAACAGC |
| | | GTGACCGCCGCCGACACCGCCGTGTACTACTGCGCCAGCCT |
| | | GACCGCCTACTGGGGCCAGGGCAGCCTGGTGACCGTGAGC |
| | | AGCATCGAGGTGATGTACCCCCCCCCCTACCTGGACAACGA |
| | | GAAGAGCAACGGCACCATCATCCACGTGAAGGGCAAGCAC |
| | | CTGTGCCCCAGCCCCCTGTTCCCCGGCCCCAGCAAGCCCTTC |
| | | TGGGTGCTGGTGGTGGTGGGCGGCGTGCTGGCCTGCTACAG |
| | | CCTGCTGGTGACCGTGGCCTTCATCATCTTCTGGGTGCGGA |
| | | GCAAGCGGAGCCGGCTGCTGCACAGCGACTACATGAACAT |
| | | GACCCCCCGGCGGCCCGGCCCCACCCGGAAGCACTACCAG |
| | | GCCTACGCCGCCGCCCGGGACTTCGCCGCCTACCGGAGCCG |
| | | GGTGAAGTTCAGCCGGAGCGCCGACGCCCCCGCCTACCAGC |
| | | AGGGCCAGAACCAGCTGTACAACGAGCTGAACCTGGGCCG |
| | | GCGGGAGGAGTACGACGTGCTGGACAAGCGGCGGGGCCGG |
| | | GACCCCGAGATGGGCGGCAAGCCCCGGCGGAAGAACCCCC |
| | | AGGAGGGCCTGTACAACGAGCTGCAGAAGGACAAGATGGC |
| | | CGAGGCCTACAGCGAGATCGGCATGAAGGGCGAGCGGCGG |
| | | CGGGGCAAGGGCCACGACGGCCTGTACCAGGGCCTGAGCA |
| | | CCGCCACCAAGGACACCTACGACGCCCTGCACATGCAGGCC |
| | | CTGCCCCCCCGG |
| Construct 3887 | 247 | ATGGCCCTGCCCGTGACCGCCCTGCTGCTGCCCCTGGCCCT |
| | | GCTGCTGCACGCCGCCCGGCCCGACATCCAGATGACCCAGA |
| | | GCCCCACCAGCCTGAGCGCCAGCGTGGGCGACCGGGTGAC |
| | | CATCACCTGCCGGGCCAGCCAGGGCATCAGCAGCTGGCTGA |
| | | CCTGGTACCAGCAGAAGCCCGAGAAGGCCCCCAAGAGCCT |
| | | GATCTACGCCGCCAGCAGCCTGCAGAGCGGCGTGCCCAGCC |
| | | GGTTCAGCGGCAGCGGCAGCGGCACCGACTTCACCCTGACC |
| | | ATCAGCAGCCTGCAGCCCGAGGACTTCGCCACCTACTACTG |
| | | CCAGCAGTACGACAGCTACCCCATCACCTTCGGCCAGGGCA |
| | | CCCGGCTGGAGATCAAGGGCAGCACCAGCGGCAGCGGCAA |
| | | GCCCGGCAGCGGCGAGGGCAGCACCAAGGGCCAGGTGCAG |
| | | CTGCAGCAGTGGGGCGCCGGCCTGCTGAAGCCCAGCGAGA |
| | | CCCTGAGCCTGACCTGCGCCGTGTACGGCGGCAGCTTCAGC |
| | | GCCTACTACTGGAGCTGGATCCGGCAGCCCCCCGGCAAGGG |
| | | CCTGGAGTGGATCGGCGACATCAACCACGGCGGCGGCACC |
| | | AACTACAACCCCAGCCTGAAGAGCCGGGTGACCATCAGCGT |
| | | GGACACCAGCAAGAACCAGTTCAGCCTGAAGCTGAACAGC |
| | | GTGACCGCCGCCGACACCGCCGTGTACTACTGCGCCAGCCT |
| | | GACCGCCTACTGGGGCCAGGGCAGCCTGGTGACCGTGAGC |
| | | AGCACCACCACCCCCGCCCCCCGGCCCCCCACCCCCGCCCC |
| | | CACCATCGCCAGCCAGCCCCTGAGCCTGCGGCCCGAGGCCT |
| | | GCCGGCCCGCCGCCGGCGGCGCCGTGCACACCCGGGGCCTG |
| | | GACTTCGCCTGCGACTTCTGGGTGCTGGTGGTGGTGGGCGG |
| | | CGTGCTGGCCTGCTACAGCCTGCTGGTGACCGTGGCCTTCA |
| | | TCATCTTCTGGGTGCGGAGCAAGCGGAGCCGGCTGCTGCAC |
| | | AGCGACTACATGAACATGACCCCCCGGCGGCCCGGCCCCAC |
| | | CCGGAAGCACTACCAGCCCTACGCCCCCCCCCGGGACTTCG |
| | | CCGCCTACCGGAGCCGGGTGAAGTTCAGCCGGAGCGCCGA |
| | | CGCCCCCGCCTACCAGCAGGGCCAGAACCAGCTGTACAACG |
| | | AGCTGAACCTGGGCCGGCGGGAGGAGTACGACGTGCTGGA |
| | | CAAGCGGCGGGGCCGGGACCCCGAGATGGGCGGCAAGCCC |
| | | CGGCGGAAGAACCCCCAGGAGGGCCTGTACAACGAGCTGC |
| | | AGAAGGACAAGATGGCCGAGGCCTACAGCGAGATCGGCAT |
| | | GAAGGGCGAGCGGCGGCGGGGCAAGGGCCACGACGGCCTG |
| | | TACCAGGGCCTGAGCACCGCCACCAAGGACACCTACGACG |
| | | CCCTGCACATGCAGGCCCTGCCCCCCCGGTAA |
| Construct 3765 | 249 | ATGGCCTTACCAGTGACCGCCTTGCTCCTGCCGCTGGCCTTG |
| | | CTGCTCCACGCCGCCAGGCCGGATATTCAGATGACTCAATC |
| | | TCCAACCTCTCTCTCAGCAAGTGTTGGCGACAGAGTTACCA |
| | | TCACTTGCAGGGCAAGTCAGGGTATCAGCAGCTGGCTGACA |
| | | TGGTATCAACAGAAGCCAGAGAAAGCTCCTAAGAGTCTCAT |
| | | ATACGCTGCAAGCTCACTCCAGTCAGGAGTGCCCTCCAGGT |
| | | TCTCCGGGTCCGGATCAGGGACTGACTTTACCCTCACTATTA |
| | | GCAGCCTCCAGCCAGAGGACTTCGCTACATACTACTGTCAG |
| | | CAGTATGACAGTTACCCTATCACCTTTGGCCAGGGAACTCG |
| | | GCTGGAGATCAAGGGTTCTACTTCTGGTAGCGGGAAACCCG |
| | | GTAGCGGTGAAGGCAGCACTAAGGGACAGGTTCAGCTCCA |
| | | ACAGTGGGGCGCTGGGCTGCTGAAACCCAGCGAGACTCTG |
| | | AGTCTCACTTGCGCAGTGTACGGTGGTTCATTCAGCGCCTA |
| | | CTATTGGAGTTGGATAAGGCAGCCTCCAGGGAAAGGCCTGG |
| | | AATGGATCGGCGACATCAATCATGGCGGTGGAACCAACTAT |
| | | AACCCATCTCTGAAGAGTCGCGTGACAATTTCTGTTGATAC |
| | | CAGCAAGAATCAGTTTAGTCTGAAACTGAACTCCGTGACTG |
| | | CCGCAGATACTGCCGTTTACTACTGTGCCAGCCTGACCGCA |
| | | TACTGGGGCCAGGGTAGCCTCGTCACCGTGTCTAGTACCAC |
| | | TACCCCAGCACCGAGGCCACCCACCCCGGCTCCTACCATCG |
| | | CCTCCCAGCCTCTGTCCCTGCGTCCGGAGGCATGTAGACCC |
| | | GCAGCTGGTGGGGCCGTGCATACCCGGGGTCTTGACTTCGC |
| | | CTGCGATATCTACATTTGGGCCCCTCTGGCTGGTACTTGCGG |
| | | GGTCCTGCTGCTTTCACTCGTGATCACTCTTTACTGTAAGCG |
| | | CGGTCGGAAGAAGCTGCTGTACATCTTTAAGCAACCCTTCA |
| | | TGAGGCCTGTGCAGACTACTCAAGAGGAGGACGGCTGTTCA |
| | | TGCCGGTTCCCAGAGGAGGAGGAAGGCGGCTGCGAACTGC |
| | | GCGTGAAATTCAGCCGCAGCGCAGATGCTCCAGCCTACAAG |
| | | CAGGGGCAGAACCAGCTCTACAACGAACTCAATCTTGGTCG |
| | | GAGAGAGGAGTACGACGTGCTGGACAAGCGGAGAGGACGG |
| | | GACCCAGAAATGGGCGGGAAGCCGCGCAGAAAGAATCCCC |
| | | AAGAGGGCCTGTACAACGAGCTCCAAAAGGATAAGATGGC |
| | | AGAAGCCTATAGCGAGATTGGTATGAAAGGGGAACGCAGA |
| | | AGAGGCAAAGGCCACGACGGACTGTACCAGGGACTCAGCA |
| | | CCGCCACCAAGGACACCTATGACGCTCTTCACATGCAGGCC |
| | | CTGCCGCCTCGG |
| Construct 3764 | 251 | ATGGCCTTACCAGTGACCGCCTTGCTCCTGCCGCTGGCCTTG |
| | | CTGCTCCACGCCGCCAGGCCGGATATTCAGATGACTCAATC |
| | | TCCAACCTCTCTCTCAGCAAGTGTTGGCGACAGAGTTACCA |
| | | TCACTTGCAGGGCAAGTCAGGGTATCAGCAGCTGGCTGACA |
| | | TGGTATCAACAGAAGCCAGAGAAAGCTCCTAAGAGTCTCAT |
| | | ATACGCTGCAAGCTCACTCCAGTCAGGAGTGCCCTCCAGGT |
| | | TCTCCGGGTCCGGATCAGGGACTGACTTTACCCTCACTATTA |
| | | GCAGCCTCCAGCCAGAGGACTTCGCTACATACTACTGTCAG |
| | | CAGTATGACAGTTACCCTATCACCTTTGGCCAGGGAACTCG |
| | | GCTGGAGATCAAGGGTTCTACTTCTGGTAGCGGGAAACCCG |
| | | GTAGCGGTGAAGGCAGCACTAAGGGACAGGTTCAGCTCCA |
| | | ACAGTGGGGCGCTGGGCTGCTGAAACCCAGCGAGACTCTG |
| | | AGTCTCACTTGCGCAGTGTACGGTGGTTCATTCAGCGCCTA |
| | | CTATTGGAGTTGGATAAGGCAGCCTCCAGGGAAAGGCCTGG |
| | | AATGGATCGGCGACATCAATCATGGCGGTGGAACCAACTAT |
| | | AACCCATCTCTGAAGAGTCGCGTGACAATTTCTGTTGATAC |
| | | CAGCAAGAATCAGTTTAGTCTGAAACTGAACTCCGTGACTG |
| | | CCGCAGATACTGCCGTTTACTACTGTGCCAGCCTGACCGCA |
| | | TACTGGGGCCAGGGTAGCCTCGTCACCGTGTCTAGTACCAC |
| | | TACCCCAGCACCGAGGCCACCCACCCCGGCTCCTACCATCG |
| | | CCTCCCAGCCTCTGTCCCTGCGTCCGGAGGCATGTAGACCC |
| | | GCAGCTGGTGGGGCCGTGCATACCCGGGGTCTTGACTTCGC |
| | | CTGCGATTTCTGGGTGCTGGTCGTTGTGGGCGGCGTGCTGG |
| | | CCTGCTACAGCCTGCTGGTGACAGTGGCCTTCATCATCTTTT |
| | | GGGTGAGGAGCAAGCGGAGCAGACTGCTGCACAGCGACTA |
| | | CATGAACATGACCCCCCGGAGGCCTGGCCCCACCCGGAAGC |
| | | ACTACCAGCCCTACGCCCCTCCCAGGGATTTCGCCGCCTAC |
| | | CGGAGCCGCGTGAAATTCAGCCGCAGCGCAGATGCTCCAGC |
| | | CTACAAGCAGGGGCAGAACCAGCTCTACAACGAACTCAAT |
| | | CTTGGTCGGAGAGAGGAGTACGACGTGCTGGACAAGCGGA |
| | | GAGGACGGGACCCAGAAATGGGCGGGAAGCCGCGCAGAAA |
| | | GAATCCCCAAGAGGGCCTGTACAACGAGCTCCAAAAGGAT |
| | | AAGATGGCAGAAGCCTATAGCGAGATTGGTATGAAAGGGG |
| | | AACGCAGAAGAGGCAAAGGCCACGACGGACTGTACCAGGG |
| | | ACTCAGCACCGCCACCAAGGACACCTATGACGCTCTTCACA |
| | | TGCAGGCCCTGCCGCCTCGG |
| Construct 4620 | 253 | ATGGACTTCCAGGTGCAGATCTTCAGCTTCCTGCTGATCAG |
| | | CGCCAGCGTGATCATGAGCCGGATGGCCCAGGTGCAGCTGC |
| | | AGCAGAGCGGCGCCGAGCTGGCCCGGCCCGGCGCCAGCGT |
| | | GAAGATGAGCTGCAAGGCCAGCGGCTACACCTTCACCACCT |
| | | ACACCATCCACTGGGTGCGGCGGCGGCCCGGCCACGACCTG |
| | | GAGTGGATCGGCTACATCAACCCCAGCAGCGGCTGCAGCG |
| | | ACTACAACCAGAACTTCAAGGGCAAGACCACCCTGACCGCC |
| | | GACAAGAGCAGCAACACCGCCTACATGCAGCTGAACAGCC |
| | | TGACCAGCGAGGACAGCGCCGTGTACTACTGCGCCCGGCGG |
| | | GCCGACTACGGCAACTACGAGTACACCTGGTTCGCCTACTG |
| | | GGGCCAGGGCACCACCGTGACCGTGAGCAGCAGCGGCGGC |
| | | GGCAGCGGCGGCGGCGGCAGCGGCGGCGGCGGCAGCGTGA |
| | | TCGAGCTGACCCAGAGCCCCAAGTTCATGAGCACCAGCGTG |
| | | GGCGACCGGGTGAACGTGACCTACAAGGCCAGCCAGAACG |
| | | TGGGCACCAACGTGGCCTGGTTCCAGCAGAAGCCCGGCCAG |
| | | AGCCCCAAGGTGCTGATCTACAGCGCCAGCTACCGGTACAG |
| | | CGGCGTGCCCGACCGGTTCACCGGCAGCGGCAGCGGCACC |
| | | GACTTCACCCTGACCATCAGCAACGTGCAGAGCGAGGACCT |
| | | GGCCGAGTACTTCTGCCAGCAGTACCACACCTACCCCCTGA |
| | | CCTTCGGCGGCGGCACCAAGCTGGAGATCAAGCGGAGCGA |
| | | CCCCGCCACCACCACCCCCGCCCCCCGGCCCCCCACCCCCG |
| | | CCCCCACCATCGCCAGCCAGCCCCTGAGCCTGCGGCCCGAG |
| | | GCCTGCCGGCCCGCCGCCGGCGGCGCCGTGCACACCCGGGG |
| | | CCTGGACTTCGCCTGCGACTTCTGGGTGCTGGTGGTGGTGG |
| | | GCGGCGTGCTGGCCTGCTACAGCCTGCTGGTGACCGTGGCC |
| | | TTCATCATCTTCTGGGTGCGGAGCAAGCGGAGCCGGCTGCT |
| | | GCACAGCGACTACATGAACATGACCCCCCGGCGGCCCGGCC |
| | | CCACCCGGAAGCACTACCAGCCCTACGCCCCCCCCCGGGAC |
| | | TTCGCCGCCTACCGGAGCCTGCGGGTGAAGTTCAGCCGGAG |
| | | CGCCGACGCCCCCGCCTACCAGCAGGGCCAGAACCAGCTGT |
| | | ACAACGAGCTGAACCTGGGCCGGCGGGAGGAGTACGACGT |
| | | GCTGGACAAGCGGCGGGGCCGGGACCCCGAGATGGGCGGC |
| | | AAGCCCCGGCGGAAGAACCCCCAGGAGGGCCTGTACAACG |
| | | AGCTGCAGAAGGACAAGATGGCCGAGGCCTACAGCGAGAT |
| | | CGGCATGAAGGGCGAGCGGCGGCGGGGCAAGGGCCACGAC |
| | | GGCCTGTACCAGGGCCTGAGCACCGCCACCAAGGACACCTA |
| | | CGACGCCCTGCACATGCAGGCCCTGCCCCCCCGG |
| Construct 4621 | 255 | ATGGACTTCCAGGTGCAGATCTTCAGCTTCCTGCTGATCAG |
| | | CGCCAGCGTGATCATGAGCCGGATGGCCCAGGTGCAGCTGC |
| | | AGCAGAGCGGCGCCGAGCTGGCCCGGCCCGGCGCCAGCGT |
| | | GAAGATGAGCTGCAAGGCCAGCGGCTACACCTTCACCACCT |
| | | ACACCATCCACTGGGTGCGGCAGCGGCCCGGCCACGACCTG |
| | | GAGTGGATCGGCTACATCAACCCCAGCAGCGGCTACAGCG |
| | | ACTACAACCAGAACTTCAAGGGCAAGACCACCCTGACCGCC |
| | | GACAAGAGCAGCAACACCGCCTACATGCAGCTGAACAGCC |
| | | TGACCAGCGAGGACAGCGCCGTGTACTACTGCGCCCGGCGG |
| | | GCCGACTACGGCAACTACGAGTACACCTGGTTCGCCTACTG |
| | | GGGCCAGGGCACCACCGTGACCGTGAGCAGCGGCGGCGGC |
| | | GGCAGCGGCGGCGGCGGCAGCGGCGGCGGCGGCAGCGACA |
| | | TCGAGCTGACCCAGAGCCCCAAGTTCATGAGCACCAGCGTG |
| | | GGCGACCGGGTGAACGTGACCTACAAGGCCAGCCAGAACG |
| | | TGGGCACCAACGTGGCCTGGTTCCAGCAGAAGCCCGGCCAG |
| | | AGCCCCAAGGTGCTGATCTACAGCGCCAGCTACCGGTACAG |
| | | CGGCGTGCCCGACCGGTTCACCGGCAGCGGCAGCGGCACC |
| | | GACTTCACCCTGACCATCAGCAACGTGCAGAGCGAGGACCT |
| | | GGCCGAGTACTTCTGCCAGCAGTACCACACCTACCCCCTGA |
| | | CCTTCGGCGGCGGCACCAAGCTGGAGATCAAGCGGAGCGA |
| | | CCCCGCCACCACCACCCCCGCCCCCCGGCCCCCCACCCCCG |
| | | CCCCCACCATCGCCAGCCAGCCCCTGAGCCTGCGGCCCGAG |
| | | GCCTGCCGGCCCGCCGCCGGCGGCGCCGTGCACACCCGGGG |
| | | CCTGGACTTCGCCTGCGACAAGGACCCCAAGTTCTGGGTGC |
| | | TGGTGGTGGTGGGCGGCGTGCTGGCCTGCTACAGCCTGCTG |
| | | GTGACCGTGGCCTTCATCATCTTCTGGGTGCGGAGCAAGCG |
| | | GAGCCGGCTGCTGCACAGCGACTACATGAACATGACCCCCC |
| | | GGCGGCCCGGCCCCACCCGGAAGCACTACCAGCCCTACGCC |
| | | CCCCCCCGGGACTTCGCCGCCTACCGGAGCCTGCGGGTGAA |
| | | GTTCAGCCGGAGCGCCGACGCCCCCGCCTACCAGCAGGGCC |
| | | AGAACCAGCTGTACAACGAGCTGAACCTGGGCCGGCGGGA |
| | | GGAGTACGACGTGCTGGACAAGCGGCGGGGCCGGGACCCC |
| | | GAGATGGGCGGCAAGCCCCGGCGGAAGAACCCCCAGGAGG |
| | | GCCTGTACAACGAGCTGCAGAAGGACAAGATGGCCGAGGC |
| | | CTACAGCGAGATCGGCATGAAGGGCGAGCGGCGGCGGGGC |
| | | AAGGGCCACGACGGCCTGTACCAGGGCCTGAGCACCGCCA |
| | | CCAAGGACACCTACGACGCCCTGCACATGCAGGCCCTGCCC |
| | | CCCCGG |
| Construct 4622 | 257 | ATGGACTTCCAGGTGCAGATCTTCAGCTTCCTGCTGATCAG |
| | | CGCCAGCGTGATCATGAGCCGGATGGCCCAGGTGCAGCTGC |
| | | AGCAGAGCGGCGCCGAGCTGGCCCGGCCCGGCGCCAGCGT |
| | | GAAGATGAGCTGCAAGGCCAGCGGCTACACCTTCACCACCT |
| | | ACACCATCCACTGGGTGCGGCGGCGGCCCGGCCACGACCTG |
| | | GAGTGGATCGGCTACATCAACCCCAGCAGCGGCTACAGCG |
| | | ACTACAACCAGAACTTCAAGGGCAAGACCACCCTGACCGCC |
| | | GACAAGAGCAGCAACACCGCCTACATGCAGCTGAACAGCC |
| | | TGACCAGCGAGGACAGCGCCGTGTACTACTGCGCCCGGCGG |
| | | GCCGACTACGGCAACTACGAGTACACCTGGTTCGCCTACTG |
| | | GGGCCAGGGCACCACCGTGACCGTGAGCAGCAGCGGCGGC |
| | | GGCAGCGGCGGCGGCGGCAGCGGCGGCGGCGGCAGCGTGA |
| | | TCGAGCTGACCCAGAGCCCCAAGTTCATGAGCACCAGCGTG |
| | | GGCGACCGGGTGAACGTGACCTACAAGGCCAGCCAGAACG |
| | | TGGGCACCAACGTGGCCTGGTTCCAGCAGAAGCCCGGCCAG |
| | | AGCCCCAAGGTGCTGATCTACAGCGCCAGCTACCGGTACAG |
| | | CGGCGTGCCCGACCGGTTCACCGGCAGCGGCAGCGGCACC |
| | | GACTTCACCCTGACCATCAGCAACGTGCAGAGCGAGGACCT |
| | | GGCCGAGTACTTCTGCCAGCAGTACCACACCTACCCCCTGA |
| | | CCTTCGGCGGCGGCACCAAGCTGGAGATCAAGCGGAGCGA |
| | | CCCCGCCACCACCACCCCCGCCCCCCGGCCCCCCACCCCCG |
| | | CCCCCACCATCGCCAGCCAGCCCCTGAGCCTGCGGCCCGAG |
| | | GCCTGCCGGCCCGCCGCCGGCGGCGCCGTGCACACCCGGGG |
| | | CCTGGACTTCGCCTGCGACAAGGACCCCAAGTTCTGGGTGC |
| | | TGGTGGTGGTGGGCGGCGTGCTGGCCTGCTACAGCCTGCTG |
| | | GTGACCGTGGCCTTCATCATCTTCTGGGTGCGGAGCAAGCG |
| | | GAGCCGGCTGCTGCACAGCGACTACATGAACATGACCCCCC |
| | | GGCGGCCCGGCCCCACCCGGAAGCACTACCAGCCCTACGCC |
| | | CCCCCCCGGGACTTCGCCGCCTACCGGAGCCTGCGGGTGAA |
| | | GTTCAGCCGGAGCGCCGACGCCCCCGCCTACCAGCAGGGCC |
| | | AGAACCAGCTGTACAACGAGCTGAACCTGGGCCGGCGGGA |
| | | GGAGTACGACGTGCTGGACAAGCGGCGGGGCCGGGACCCC |
| | | GAGATGGGCGGCAAGCCCCGGCGGAAGAACCCCCAGGAGG |
| | | GCCTGTACAACGAGCTGCAGAAGGACAAGATGGCCGAGGC |
| | | CTACAGCGAGATCGGCATGAAGGGCGAGCGGCGGCGGGGC |
| | | AAGGGCCACGACGGCCTGTACCAGGGCCTGAGCACCGCCA |
| | | CCAAGGACACCTACGACGCCCTGCACATGCAGGCCCTGCCC |
| | | CCCCGG |
| Construct 3884 | 259 | TTGGCCACTCCCTCTCTGCGCGCTCGCTCGCTCACTGAGGCC |
| including the | | GGGCGACCAAAGGTCGCCCGACGCCCGGGCTTTGCCCGGGC |
| ITR sequences | | GGCCTCAGTGAGCGAGCGAGCGCGCAGAGAGGGAGTGGCC |
| and homology | | AACTCCATCACTAGGGGTTCCTagatcttgccaacataccataaacctcccattc |
| arms | | tgctaatgcccagcctaagttggggagaccactccagattccaagatgtacagtttgctttgctgggc |
| The underlined | | ctttttcccatgcctgcctttactctgccagagttatattgctggggttttgaagaagatcctattaaat |
| portion | | aaaagaataagcagtattattaagtagccctgcatttcaggtttccttgagtggcaggccaggcctggcc |
| indicates | | gtgaacgttcactgaaatcatggcctcttggccaagattgatagcttgtgcctgtccctgagtcccagt |
| the coding | | ccatcacgagcagctggtttctaagatgctatttcccgtataaagcatgagaccgtgacttgccagcc |
| sequence for | | ccacagagccccgcccttgtccatcactggcatctggactccagcctgggttggggcaaagaggg |
| the 3884 binder | | aaatgagatcatgtcctaaccctgatcctcttgtcccacagATATCCAGAACCCTGA |
| domain. Other | | CCCTGCGGCTCCGGTGCCCGTCAGTGGGCAGAGCGCACATC |
| binders use | | GCCCACAGTCCCCGAGAAGTTGGGGGGAGGGGTCGGCAAT |
| the same ITR | | TGAACCGGTGCCTAGAGAAGGTGGCGCGGGGTAAACTGGG |
| sequences | | AAAGTGATGTCGTGTACTGGCTCCGCCTTTTTCCCGAGGGT |
| | | GGGGGAGAACCGTATATAAGTGCAGTAGTCGCCGTGAACG |
| | | TTCTTTTTCGCAACGGGTTTGCCGCCAGAACACAGGTAAGT |
| | | GCCGTGTGTGGTTCCCGCGGGCCTGGCCTCTTTACGGGTTAT |
| | | GGCCCTTGCGTGCCTTGAATTACTTCCACGCCCCTGGCTGCA |
| | | GTACGTGATTCTTGATCCCGAGCTTCGGGTTGGAAGTGGGT |
| | | GGGAGAGTTCGAGGCCTTGCGCTTAAGGAGCCCCTTCGCCT |
| | | CGTGCTTGAGTTGAGGCCTGGCTTGGGCGCTGGGGCCGCCG |
| | | CGTGCGAATCTGGTGGCACCTTCGCGCCTGTCTCGCTGCTTT |
| | | CGATAAGTCTCTAGCCATTTAAAATTTTTGATGACCTGCTGC |
| | | GACGCTTTTTTTCTGGCAAGATAGTCTTGTAAATGCGGGCC |
| | | AAGATgTGCACACTGGTATTTCGGTTTTTGGGGCCGCGGGC |
| | | GGCGACGGGGCCCGTGCGTCCCAGCGCACATGTTCGGCGAG |
| | | GCGGGGCCTGCGAGCGCGGCCACCGAGAATCGGACGGGGG |
| | | TAGTCTCAAGCTGGCCGGCCTGCTCTGGTGCCTGGCCTCGC |
| | | GCCGCCGTGTATCGCCCCGCCCTGGGCGGCAAGGCTGGCCC |
| | | GGTCGGCACCAGTTGCGTGAGCGGAAAGATGGCCGCTTCCC |
| | | GGCCCTGCTGCAGGGAGCTCAAAATGGAGGACGCGGCGCT |
| | | CGGGAGAGCGGGCGGGTGAGTCACCCACACAAAGGAAAAG |
| | | GGCCTTTCCGTCCTCAGCCGTCGCTTCATGTGACTCCACGGA |
| | | GTACCGGGCGCCGTCCAGGCACCTCGATTAGTTCTCGAGCT |
| | | TTTGGAGTACGTCGTCTTTAGGTTGGGGGGAGGGGTTTTAT |
| | | GCGATGGAGTTTCCCCACACTGAGTGGGTGGAGACTGAAGT |
| | | TAGGCCAGCTTGGCACTTGATGTAATTCTCCTTGGAATTTGC |
| | | CCTTTTTGAGTTTGGATCTTGGTTCATTCTCAAGCCTCAGAC |
| | | AGTGGTTCAAAGTTTTTTTCTTCCATTTCAGGTGTCGTGAtGC |
| | | GGCCGCCACCATGGACTTCCAGGTGCAGATCTTCAGCTT |
| | | CCTGCTGATCAGCGCCAGCGTGATCATGAGCCGGATGG |
| | | CCCAGGTGCAGCTGCAGCAGAGCGGCGCCGAGCTGGCC |
| | | CGGCCCGGCGCCAGCGTGAAGATGAGCTGCAAGGCCAG |
| | | CGGCTACACCTTCACCACCTACACCATCCACTGGGTGCG |
| | | GCGGCGGCCCGGCCACGACCTGGAGTGGATCGGCTACA |
| | | TCAACCCCAGCAGCGGCTGCAGCGACTACAACCAGAAC |
| | | TTCAAGGGCAAGACCACCCTGACCGCCGACAAGAGCAG |
| | | CAACACCGCCTACATGCAGCTGAACAGCCTGACCAGCG |
| | | AGGACAGCGCCGTGTACTACTGCGCCCGGCGGGCCGAC |
| | | TACGGCAACTACGAGTACACCTGGTTCGCCTACTGGGG |
| | | CCAGGGCACCACCGTGACCGTGAGCAGCAGCGGCGGCG |
| | | GCAGCGGCGGCGGCGGCAGCGGCGGCGGCGGCAGCGT |
| | | GATCGAGCTGACCCAGAGCCCCAAGTTCATGAGCACCA |
| | | GCGTGGGCGACCGGGTGAACGTGACCTACAAGGCCAGC |
| | | CAGAACGTGGGCACCAACGTGGCCTGGTTCCAGCAGAA |
| | | GCCCGGCCAGAGCCCCAAGGTGCTGATCTACAGCGCCA |
| | | GCTACCGGTACAGCGGCGTGCCCGACCGGTTCACCGGC |
| | | AGCGGCAGCGGCACCGACTTCACCCTGACCATCAGCAA |
| | | CGTGCAGAGCGAGGACCTGGCCGAGTACTTCTGCCAGC |
| | | AGTACCACACCTACCCCCTGACCTTCGGCGGCGGCACC |
| | | AAGCTGGAGATCAAGCGGAGCGACCCCGCCACCACCAC |
| | | CCCCGCCCCCCGGCCCCCCACCCCCGCCCCCACCATCG |
| | | CCAGCCAGCCCCTGAGCCTGCGGCCCGAGGCCTGCCGG |
| | | CCCGCCGCCGGCGGCGCCGTGCACACCCGGGGCCTGGA |
| | | CTTCGCCTGCGACAAGGACCCCAAGTTCTGGGTGCTGG |
| | | TGGTGGTGGGCGGCGTGCTGGCCTGCTACAGCCTGCTG |
| | | GTGACCGTGGCCTTCATCATCTTCTGGGTGCGGAGCAA |
| | | GCGGAGCCGGCTGCTGCACAGCGACTACATGAACATGA |
| | | CCCCCCGGCGGCCCGGCCCCACCCGGAAGCACTACCAG |
| | | CCCTACGCCCCCCCCCGGGACTTCGCCGCCTACCGGAG |
| | | CCTGCGGGTGAAGTTCAGCCGGAGCGCCGACGCCCCCG |
| | | CCTACCAGCAGGGCCAGAACCAGCTGTACAACGAGCTG |
| | | AACCTGGGCCGGCGGGAGGAGTACGACGTGCTGGACAA |
| | | GCGGCGGGGCCGGGACCCCGAGATGGGCGGCAAGCCC |
| | | CGGCGGAAGAACCCCCAGGAGGGCCTGTACAACGAGCT |
| | | GCAGAAGGACAAGATGGCCGAGGCCTACAGCGAGATCG |
| | | GCATGAAGGGCGAGCGGCGGCGGGGCAAGGGCCACGA |
| | | CGGCCTGTACCAGGGCCTGAGCACCGCCACCAAGGACA |
| | | CCTACGACGCCCTGCACATGCAGGCCCTGCCCCCCCGG |
| | | CCTCGACTGTGCCTTCTAGTTGCCAGCCATCTGTTGTTTGCC |
| | | CCTCCCCCGTGCCTTCCTTGACCCTGGAAGGTGCCACTCCCA |
| | | CTGTCCTTTCCTAATAAAATGAGGAAATTGCATCGCATTGTC |
| | | TGAGTAGGTGTCATTCTATTCTGGGGGGTGGGGTGGGGCAG |
| | | GACAGCAAGGGGGAGGATTGGGAAGACAATAGCAGGCATG |
| | | CTGGGGATGCGGTGGGCTCTATGGCTTCTGAGGCGGAAAGA |
| | | ACCAGCTGGGGCTCTAGGGGGTATCCCCACTAGTCGTGTAC |
| | | CAGCTGAGAGACTCTAAATCCAGTGACAAGTCTGTCTGCCT |
| | | ATTCACCGATTTTGATTCTCAAACAAATGTGTCACAAAGTA |
| | | AGGATTCTGATGTGTATATCACAGACAAAACTGTGCTAGAC |
| | | ATGAGGTCTATGGACTTCAAGAGCAACAGTGCTGTGGCCTG |
| | | GAGCAACAAATCTGACTTTGCATGTGCAAACGCCTTCAACA |
| | | ACAGCATTATTCCAGAAGACACCTTCTTCCCCAGCCCAGgtaa |
| | | gggcagctttggtgccttcgcaggctgtttccttgcttcaggaatggccaggttctgcccagagctct |
| | | ggtcaatgatgtctaaaactcctctgattggtggtctcggccttatccattgccaccaaaaccctcttttt |
| | | actaagaaacagtgagccttgttctggcagtccagagaatgacacgggaaaaaagcagatgaaga |
| | | gaaggtggcaggagagggcacgtggcccagcctcagtctctAGATCTAGGAACCC |
| | | CTAGTGATGGAGTTGGCCACTCCCTCTCTGCGCGCTCGCTCG |
| | | CTCACTGAGGCCGCCCGGGCAAAGCCCGGGCGTCGGGCGA |
| | | CCTTTGGTCGCCCGGCCTCAGTGAGCGAGCGAGCGCGCAGA |
| | | GAGGGAGTGGCCAA |
| Recombinant | 800 | MDKKYSIGLDIGTNSVGWAVITDEYKVPSKKFKVLGNTDRHSI |
| Cas9-NLS amino | | KKNLIGALLFDSGETAEATRLKRTARRRYTRRKNRICYLQEIFS |
| acid sequence | | NEMAKVDDSFFHRLEESFLVEEDKKHERHPIFGNIVDEVAYHE |
| | | KYPTIYHLRKKLVDSTDKADLRLIYLALAHMIKFRGHFLIEGD |
| | | LNPDNSDVDKLFIQLVQTYNQLFEENPINASGVDAKAILSARLS |
| | | KSRRLENLIAQLPGEKKNGLFGNLIALSLGLTPNFKSNFDLAED |
| | | AKLQLSKDTYDDDLDNLLAQIGDQYADLFLAAKNLSDAILLS |
| | | DILRVNTEITKAPLSASMIKRYDEHHQDLTLLKALVRQQLPEK |
| | | YKEIFFDQSKNGYAGYIDGGASQEEFYKFIKPILEKMDGTEELL |
| | | VKLNREDLLRKQRTFDNGSIPHQIHLGELHAILRRQEDFYPFLK |
| | | DNREKIEKILTFRIPYYVGPLARGNSRFAWMTRKSEETITPWNF |
| | | EEVVDKGASAQSFIERMTNFDKNLPNEKVLPKHSLLYEYFTVY |
| | | NELTKVKYVTEGMRKPAFLSGEQKKAIVDLLFKTNRKVTVKQ |
| | | LKEDYFKKIECFDSVEISGVEDRFNASLGTYHDLLKIIKDKDFL |
| | | DNEENEDILEDIVLTLTLFEDREMIEERLKTYAHLFDDKVMKQ |
| | | LKRRRYTGWGRLSRKLINGIRDKQSGKTILDFLKSDGFANRNF |
| | | MQLIHDDSLTFKEDIQKAQVSGQGDSLHEHIANLAGSPAIKKGI |
| | | LQTVKVVDELVKVMGRHKPENIVIEMARENQTTQKGQKNSRE |
| | | RMKRIEEGIKELGSQILKEHPVENTQLQNEKLYLYYLQNGRDM |
| | | YVDQELDINRLSDYDVDHIVPQSFLKDDSIDNKVLTRSDKNRG |
| | | KSDNVPSEEVVKKMKNYWRQLLNAKLITQRKFDNLTKAERG |
| | | GLSELDKAGFIKRQLVETRQITKHVAQILDSRMNTKYDENDKL |
| | | IREVKVITLKSKLVSDFRKDFQFYKVREINNYHHAHDAYLNAV |
| | | VGTALIKKYPKLESEFVYGDYKVYDVRKMIAKSEQEIGKATA |
| | | KYFFYSNIMNFFKTEITLANGEIRKRPLIETNGETGEIVWDKGR |
| | | DFATVRKVLSMPQVNIVKKTEVQTGGFSKESILPKRNSDKLIA |
| | | RKKDWDPKKYGGFDSPTVAYSVLVVAKVEKGKSKKLKSVKE |
| | | LLGITIMERSSFEKNPIDFLEAKGYKEVKKDLIIKLPKYSLFELE |
| | | NGRKRMLASAGELQKGNELALPSKYVNFLYLASHYEKLKGSP |
| | | EDNEQKQLFVEQHKHYLDEIIEQISEFSKRVILADANLDKVLSA |
| | | YNKHRDKPIREQAENIIHLFTLTNLGAPAAFKYFDTTIDRKRYT |
| | | STKEVLDATLIHQSITGLYETRIDLSQLGGDGGGSPKKKRKV |
| ORF encoding | 801 | ATGGACAAGAAGTACAGCATCGGACTGGACATCGGAACAA |
| Sp. Cas9 | | ACAGCGTCGGATGGGCAGTCATCACAGACGAATACAAGGT |
| | | CCCGAGCAAGAAGTTCAAGGTCCTGGGAAACACAGACAGA |
| | | CACAGCATCAAGAAGAACCTGATCGGAGCACTGCTGTTCGA |
| | | CAGCGGAGAAACAGCAGAAGCAACAAGACTGAAGAGAAC |
| | | AGCAAGAAGAAGATACACAAGAAGAAAGAACAGAATCTGC |
| | | TACCTGCAGGAAATCTTCAGCAACGAAATGGCAAAGGTCG |
| | | ACGACAGCTTCTTCCACAGACTGGAAGAAAGCTTCCTGGTC |
| | | GAAGAAGACAAGAAGCACGAAAGACACCCGATCTTCGGAA |
| | | ACATCGTCGACGAAGTCGCATACCACGAAAAGTACCCGAC |
| | | AATCTACCACCTGAGAAAGAAGCTGGTCGACAGCACAGAC |
| | | AAGGCAGACCTGAGACTGATCTACCTGGCACTGGCACACAT |
| | | GATCAAGTTCAGAGGACACTTCCTGATCGAAGGAGACCTGA |
| | | ACCCGGACAACAGCGACGTCGACAAGCTGTTCATCCAGCTG |
| | | GTCCAGACATACAACCAGCTGTTCGAAGAAAACCCGATCAA |
| | | CGCAAGCGGAGTCGACGCAAAGGCAATCCTGAGCGCAAGA |
| | | CTGAGCAAGAGCAGAAGACTGGAAAACCTGATCGCACAGC |
| | | TGCCGGGAGAAAAGAAGAACGGACTGTTCGGAAACCTGAT |
| | | CGCACTGAGCCTGGGACTGACACCGAACTTCAAGAGCAACT |
| | | TCGACCTGGCAGAAGACGCAAAGCTGCAGCTGAGCAAGGA |
| | | CACATACGACGACGACCTGGACAACCTGCTGGCACAGATCG |
| | | GAGACCAGTACGCAGACCTGTTCCTGGCAGCAAAGAACCTG |
| | | AGCGACGCAATCCTGCTGAGCGACATCCTGAGAGTCAACAC |
| | | AGAAATCACAAAGGCACCGCTGAGCGCAAGCATGATCAAG |
| | | AGATACGACGAACACCACCAGGACCTGACACTGCTGAAGG |
| | | CACTGGTCAGACAGCAGCTGCCGGAAAAGTACAAGGAAAT |
| | | CTTCTTCGACCAGAGCAAGAACGGATACGCAGGATACATCG |
| | | ACGGAGGAGCAAGCCAGGAAGAATTCTACAAGTTCATCAA |
| | | GCCGATCCTGGAAAAGATGGACGGAACAGAAGAACTGCTG |
| | | GTCAAGCTGAACAGAGAAGACCTGCTGAGAAAGCAGAGAA |
| | | CATTCGACAACGGAAGCATCCCGCACCAGATCCACCTGGGA |
| | | GAACTGCACGCAATCCTGAGAAGACAGGAAGACTTCTACCC |
| | | GTTCCTGAAGGACAACAGAGAAAAGATCGAAAAGATCCTG |
| | | ACATTCAGAATCCCGTACTACGTCGGACCGCTGGCAAGAGG |
| | | AAACAGCAGATTCGCATGGATGACAAGAAAGAGCGAAGAA |
| | | ACAATCACACCGTGGAACTTCGAAGAAGTCGTCGACAAGG |
| | | GAGCAAGCGCACAGAGCTTCATCGAAAGAATGACAAACTT |
| | | CGACAAGAACCTGCCGAACGAAAAGGTCCTGCCGAAGCAC |
| | | AGCCTGCTGTACGAATACTTCACAGTCTACAACGAACTGAC |
| | | AAAGGTCAAGTACGTCACAGAAGGAATGAGAAAGCCGGCA |
| | | TTCCTGAGCGGAGAACAGAAGAAGGCAATCGTCGACCTGCT |
| | | GTTCAAGACAAACAGAAAGGTCACAGTCAAGCAGCTGAAG |
| | | GAAGACTACTTCAAGAAGATCGAATGCTTCGACAGCGTCGA |
| | | AATCAGCGGAGTCGAAGACAGATTCAACGCAAGCCTGGGA |
| | | ACATACCACGACCTGCTGAAGATCATCAAGGACAAGGACTT |
| | | CCTGGACAACGAAGAAAACGAAGACATCCTGGAAGACATC |
| | | GTCCTGACACTGACACTGTTCGAAGACAGAGAAATGATCGA |
| | | AGAAAGACTGAAGACATACGCACACCTGTTCGACGACAAG |
| | | GTCATGAAGCAGCTGAAGAGAAGAAGATACACAGGATGGG |
| | | GAAGACTGAGCAGAAAGCTGATCAACGGAATCAGAGACAA |
| | | GCAGAGCGGAAAGACAATCCTGGACTTCCTGAAGAGCGAC |
| | | GGATTCGCAAACAGAAACTTCATGCAGCTGATCCACGACGA |
| | | CAGCCTGACATTCAAGGAAGACATCCAGAAGGCACAGGTC |
| | | AGCGGACAGGGAGACAGCCTGCACGAACACATCGCAAACC |
| | | TGGCAGGAAGCCCGGCAATCAAGAAGGGAATCCTGCAGAC |
| | | AGTCAAGGTCGTCGACGAACTGGTCAAGGTCATGGGAAGA |
| | | CACAAGCCGGAAAACATCGTCATCGAAATGGCAAGAGAAA |
| | | ACCAGACAACACAGAAGGGACAGAAGAACAGCAGAGAAA |
| | | GAATGAAGAGAATCGAAGAAGGAATCAAGGAACTGGGAAG |
| | | CCAGATCCTGAAGGAACACCCGGTCGAAAACACACAGCTG |
| | | CAGAACGAAAAGCTGTACCTGTACTACCTGCAGAACGGAA |
| | | GAGACATGTACGTCGACCAGGAACTGGACATCAACAGACT |
| | | GAGCGACTACGACGTCGACCACATCGTCCCGCAGAGCTTCC |
| | | TGAAGGACGACAGCATCGACAACAAGGTCCTGACAAGAAG |
| | | CGACAAGAACAGAGGAAAGAGCGACAACGTCCCGAGCGAA |
| | | GAAGTCGTCAAGAAGATGAAGAACTACTGGAGACAGCTGC |
| | | TGAACGCAAAGCTGATCACACAGAGAAAGTTCGACAACCT |
| | | GACAAAGGCAGAGAGAGGAGGACTGAGCGAACTGGACAA |
| | | GGCAGGATTCATCAAGAGACAGCTGGTCGAAACAAGACAG |
| | | ATCACAAAGCACGTCGCACAGATCCTGGACAGCAGAATGA |
| | | ACACAAAGTACGACGAAAACGACAAGCTGATCAGAGAAGT |
| | | CAAGGTCATCACACTGAAGAGCAAGCTGGTCAGCGACTTCA |
| | | GAAAGGACTTCCAGTTCTACAAGGTCAGAGAAATCAACAA |
| | | CTACCACCACGCACACGACGCATACCTGAACGCAGTCGTCG |
| | | GAACAGCACTGATCAAGAAGTACCCGAAGCTGGAAAGCGA |
| | | ATTCGTCTACGGAGACTACAAGGTCTACGACGTCAGAAAGA |
| | | TGATCGCAAAGAGCGAACAGGAAATCGGAAAGGCAACAGC |
| | | AAAGTACTTCTTCTACAGCAACATCATGAACTTCTTCAAGA |
| | | CAGAAATCACACTGGCAAACGGAGAAATCAGAAAGAGACC |
| | | GCTGATCGAAACAAACGGAGAAACAGGAGAAATCGTCTGG |
| | | GACAAGGGAAGAGACTTCGCAACAGTCAGAAAGGTCCTGA |
| | | GCATGCCGCAGGTCAACATCGTCAAGAAGACAGAAGTCCA |
| | | GACAGGAGGATTCAGCAAGGAAAGCATCCTGCCGAAGAGA |
| | | AACAGCGACAAGCTGATCGCAAGAAAGAAGGACTGGGACC |
| | | CGAAGAAGTACGGAGGATTCGACAGCCCGACAGTCGCATA |
| | | CAGCGTCCTGGTCGTCGCAAAGGTCGAAAAGGGAAAGAGC |
| | | AAGAAGCTGAAGAGCGTCAAGGAACTGCTGGGAATCACAA |
| | | TCATGGAAAGAAGCAGCTTCGAAAAGAACCCGATCGACTTC |
| | | CTGGAAGCAAAGGGATACAAGGAAGTCAAGAAGGACCTGA |
| | | TCATCAAGCTGCCGAAGTACAGCCTGTTCGAACTGGAAAAC |
| | | GGAAGAAAGAGAATGCTGGCAAGCGCAGGAGAACTGCAGA |
| | | AGGGAAACGAACTGGCACTGCCGAGCAAGTACGTCAACTT |
| | | CCTGTACCTGGCAAGCCACTACGAAAAGCTGAAGGGAAGC |
| | | CCGGAAGACAACGAACAGAAGCAGCTGTTCGTCGAACAGC |
| | | ACAAGCACTACCTGGACGAAATCATCGAACAGATCAGCGA |
| | | ATTCAGCAAGAGAGTCATCCTGGCAGACGCAAACCTGGAC |
| | | AAGGTCCTGAGCGCATACAACAAGCACAGAGACAAGCCGA |
| | | TCAGAGAACAGGCAGAAAACATCATCCACCTGTTCACACTG |
| | | ACAAACCTGGGAGCACCGGCAGCATTCAAGTACTTCGACAC |
| | | AACAATCGACAGAAAGAGATACACAAGCACAAAGGAAGTC |
| | | CTGGACGCAACACTGATCCACCAGAGCATCACAGGACTGTA |
| | | CGAAACAAGAATCGACCTGAGCCAGCTGGGAGGAGACGGA |
| | | GGAGGAAGCCCGAAGAAGAAGAGAAAGGTCTAG |
| ORF encoding | 802 | ATGGACAAGAAGTACTCCATCGGCCTGGACATCGGCACCAA |
| Sp. Cas9 | | CTCCGTGGGCTGGGCCGTGATCACCGACGAGTACAAGGTGC |
| | | CCTCCAAGAAGTTCAAGGTGCTGGGCAACACCGACCGGCAC |
| | | TCCATCAAGAAGAACCTGATCGGCGCCCTGCTGTTCGACTC |
| | | CGGCGAGACCGCCGAGGCCACCCGGCTGAAGCGGACCGCC |
| | | CGGCGGCGGTACACCCGGCGGAAGAACCGGATCTGCTACCT |
| | | GCAGGAGATCTTCTCCAACGAGATGGCCAAGGTGGACGACT |
| | | CCTTCTTCCACCGGCTGGAGGAGTCCTTCCTGGTGGAGGAG |
| | | GACAAGAAGCACGAGCGGCACCCCATCTTCGGCAACATCGT |
| | | GGACGAGGTGGCCTACCACGAGAAGTACCCCACCATCTACC |
| | | ACCTGCGGAAGAAGCTGGTGGACTCCACCGACAAGGCCGA |
| | | CCTGCGGCTGATCTACCTGGCCCTGGCCCACATGATCAAGT |
| | | TCCGGGGCCACTTCCTGATCGAGGGCGACCTGAACCCCGAC |
| | | AACTCCGACGTGGACAAGCTGTTCATCCAGCTGGTGCAGAC |
| | | CTACAACCAGCTGTTCGAGGAGAACCCCATCAACGCCTCCG |
| | | GCGTGGACGCCAAGGCCATCCTGTCCGCCCGGCTGTCCAAG |
| | | TCCCGGCGGCTGGAGAACCTGATCGCCCAGCTGCCCGGCGA |
| | | GAAGAAGAACGGCCTGTTCGGCAACCTGATCGCCCTGTCCC |
| | | TGGGCCTGACCCCCAACTTCAAGTCCAACTTCGACCTGGCC |
| | | GAGGACGCCAAGCTGCAGCTGTCCAAGGACACCTACGACG |
| | | ACGACCTGGACAACCTGCTGGCCCAGATCGGCGACCAGTAC |
| | | GCCGACCTGTTCCTGGCCGCCAAGAACCTGTCCGACGCCAT |
| | | CCTGCTGTCCGACATCCTGCGGGTGAACACCGAGATCACCA |
| | | AGGCCCCCCTGTCCGCCTCCATGATCAAGCGGTACGACGAG |
| | | CACCACCAGGACCTGACCCTGCTGAAGGCCCTGGTGCGGCA |
| | | GCAGCTGCCCGAGAAGTACAAGGAGATCTTCTTCGACCAGT |
| | | CCAAGAACGGCTACGCCGGCTACATCGACGGCGGCGCCTCC |
| | | CAGGAGGAGTTCTACAAGTTCATCAAGCCCATCCTGGAGAA |
| | | GATGGACGGCACCGAGGAGCTGCTGGTGAAGCTGAACCGG |
| | | GAGGACCTGCTGCGGAAGCAGCGGACCTTCGACAACGGCT |
| | | CCATCCCCCACCAGATCCACCTGGGCGAGCTGCACGCCATC |
| | | CTGCGGCGGCAGGAGGACTTCTACCCCTTCCTGAAGGACAA |
| | | CCGGGAGAAGATCGAGAAGATCCTGACCTTCCGGATCCCCT |
| | | ACTACGTGGGCCCCCTGGCCCGGGGCAACTCCCGGTTCGCC |
| | | TGGATGACCCGGAAGTCCGAGGAGACCATCACCCCCTGGA |
| | | ACTTCGAGGAGGTGGTGGACAAGGGCGCCTCCGCCCAGTCC |
| | | TTCATCGAGCGGATGACCAACTTCGACAAGAACCTGCCCAA |
| | | CGAGAAGGTGCTGCCCAAGCACTCCCTGCTGTACGAGTACT |
| | | TCACCGTGTACAACGAGCTGACCAAGGTGAAGTACGTGACC |
| | | GAGGGCATGCGGAAGCCCGCCTTCCTGTCCGGCGAGCAGA |
| | | AGAAGGCCATCGTGGACCTGCTGTTCAAGACCAACCGGAA |
| | | GGTGACCGTGAAGCAGCTGAAGGAGGACTACTTCAAGAAG |
| | | ATCGAGTGCTTCGACTCCGTGGAGATCTCCGGCGTGGAGGA |
| | | CCGGTTCAACGCCTCCCTGGGCACCTACCACGACCTGCTGA |
| | | AGATCATCAAGGACAAGGACTTCCTGGACAACGAGGAGAA |
| | | CGAGGACATCCTGGAGGACATCGTGCTGACCCTGACCCTGT |
| | | TCGAGGACCGGGAGATGATCGAGGAGCGGCTGAAGACCTA |
| | | CGCCCACCTGTTCGACGACAAGGTGATGAAGCAGCTGAAGC |
| | | GGCGGCGGTACACCGGCTGGGGCCGGCTGTCCCGGAAGCT |
| | | GATCAACGGCATCCGGGACAAGCAGTCCGGCAAGACCATC |
| | | CTGGACTTCCTGAAGTCCGACGGCTTCGCCAACCGGAACTT |
| | | CATGCAGCTGATCCACGACGACTCCCTGACCTTCAAGGAGG |
| | | ACATCCAGAAGGCCCAGGTGTCCGGCCAGGGCGACTCCCTG |
| | | CACGAGCACATCGCCAACCTGGCCGGCTCCCCCGCCATCAA |
| | | GAAGGGCATCCTGCAGACCGTGAAGGTGGTGGACGAGCTG |
| | | GTGAAGGTGATGGGCCGGCACAAGCCCGAGAACATCGTGA |
| | | TCGAGATGGCCCGGGAGAACCAGACCACCCAGAAGGGCCA |
| | | GAAGAACTCCCGGGAGCGGATGAAGCGGATCGAGGAGGGC |
| | | ATCAAGGAGCTGGGCTCCCAGATCCTGAAGGAGCACCCCGT |
| | | GGAGAACACCCAGCTGCAGAACGAGAAGCTGTACCTGTAC |
| | | TACCTGCAGAACGGCCGGGACATGTACGTGGACCAGGAGC |
| | | TGGACATCAACCGGCTGTCCGACTACGACGTGGACCACATC |
| | | GTGCCCCAGTCCTTCCTGAAGGACGACTCCATCGACAACAA |
| | | GGTGCTGACCCGGTCCGACAAGAACCGGGGCAAGTCCGAC |
| | | AACGTGCCCTCCGAGGAGGTGGTGAAGAAGATGAAGAACT |
| | | ACTGGCGGCAGCTGCTGAACGCCAAGCTGATCACCCAGCGG |
| | | AAGTTCGACAACCTGACCAAGGCCGAGCGGGGCGGCCTGT |
| | | CCGAGCTGGACAAGGCCGGCTTCATCAAGCGGCAGCTGGTG |
| | | GAGACCCGGCAGATCACCAAGCACGTGGCCCAGATCCTGG |
| | | ACTCCCGGATGAACACCAAGTACGACGAGAACGACAAGCT |
| | | GATCCGGGAGGTGAAGGTGATCACCCTGAAGTCCAAGCTG |
| | | GTGTCCGACTTCCGGAAGGACTTCCAGTTCTACAAGGTGCG |
| | | GGAGATCAACAACTACCACCACGCCCACGACGCCTACCTGA |
| | | ACGCCGTGGTGGGCACCGCCCTGATCAAGAAGTACCCCAAG |
| | | CTGGAGTCCGAGTTCGTGTACGGCGACTACAAGGTGTACGA |
| | | CGTGCGGAAGATGATCGCCAAGTCCGAGCAGGAGATCGGC |
| | | AAGGCCACCGCCAAGTACTTCTTCTACTCCAACATCATGAA |
| | | CTTCTTCAAGACCGAGATCACCCTGGCCAACGGCGAGATCC |
| | | GGAAGCGGCCCCTGATCGAGACCAACGGCGAGACCGGCGA |
| | | GATCGTGTGGGACAAGGGCCGGGACTTCGCCACCGTGCGG |
| | | AAGGTGCTGTCCATGCCCCAGGTGAACATCGTGAAGAAGAC |
| | | CGAGGTGCAGACCGGCGGCTTCTCCAAGGAGTCCATCCTGC |
| | | CCAAGCGGAACTCCGACAAGCTGATCGCCCGGAAGAAGGA |
| | | CTGGGACCCCAAGAAGTACGGCGGCTTCGACTCCCCCACCG |
| | | TGGCCTACTCCGTGCTGGTGGTGGCCAAGGTGGAGAAGGGC |
| | | AAGTCCAAGAAGCTGAAGTCCGTGAAGGAGCTGCTGGGCA |
| | | TCACCATCATGGAGCGGTCCTCCTTCGAGAAGAACCCCATC |
| | | GACTTCCTGGAGGCCAAGGGCTACAAGGAGGTGAAGAAGG |
| | | ACCTGATCATCAAGCTGCCCAAGTACTCCCTGTTCGAGCTG |
| | | GAGAACGGCCGGAAGCGGATGCTGGCCTCCGCCGGCGAGC |
| | | TGCAGAAGGGCAACGAGCTGGCCCTGCCCTCCAAGTACGTG |
| | | AACTTCCTGTACCTGGCCTCCCACTACGAGAAGCTGAAGGG |
| | | CTCCCCCGAGGACAACGAGCAGAAGCAGCTGTTCGTGGAG |
| | | CAGCACAAGCACTACCTGGACGAGATCATCGAGCAGATCTC |
| | | CGAGTTCTCCAAGCGGGTGATCCTGGCCGACGCCAACCTGG |
| | | ACAAGGTGCTGTCCGCCTACAACAAGCACCGGGACAAGCC |
| | | CATCCGGGAGCAGGCCGAGAACATCATCCACCTGTTCACCC |
| | | TGACCAACCTGGGCGCCCCCGCCGCCTTCAAGTACTTCGAC |
| | | ACCACCATCGACCGGAAGCGGTACACCTCCACCAAGGAGG |
| | | TGCTGGACGCCACCCTGATCCACCAGTCCATCACCGGCCTG |
| | | TACGAGACCCGGATCGACCTGTCCCAGCTGGGCGGCGACGG |
| | | CGGCGGCTCCCCCAAGAAGAAGCGGAAGGTGTGA |
| Open reading | 803 | AUGGACAAGAAGUACUCCAUCGGCCUGGACAUCGGCACCA |
| frame for Cas9 | | ACUCCGUGGGCUGGGCCGUGAUCACCGACGAGUACAAGGU |
| with Hibit tag | | GCCCUCCAAGAAGUUCAAGGUGCUGGGCAACACCGACCGG |
| | | CACUCCAUCAAGAAGAACCUGAUCGGCGCCCUGCUGUUCG |
| | | ACUCCGGCGAGACCGCCGAGGCCACCCGGCUGAAGCGGAC |
| | | CGCCCGGCGGCGGUACACCCGGCGGAAGAACCGGAUCUGC |
| | | UACCUGCAGGAGAUCUUCUCCAACGAGAUGGCCAAGGUG |
| | | GACGACUCCUUCUUCCACCGGCUGGAGGAGUCCUUCCUGG |
| | | UGGAGGAGGACAAGAAGCACGAGCGGCACCCCAUCUUCGG |
| | | CAACAUCGUGGACGAGGUGGCCUACCACGAGAAGUACCCC |
| | | ACCAUCUACCACCUGCGGAAGAAGCUGGUGGACUCCACCG |
| | | ACAAGGCCGACCUGCGGCUGAUCUACCUGGCCCUGGCCCA |
| | | CAUGAUCAAGUUCCGGGGCCACUUCCUGAUCGAGGGCGAC |
| | | CUGAACCCCGACAACUCCGACGUGGACAAGCUGUUCAUCC |
| | | AGCUGGUGCAGACCUACAACCAGCUGUUCGAGGAGAACCC |
| | | CAUCAACGCCUCCGGCGUGGACGCCAAGGCCAUCCUGUCC |
| | | GCCCGGCUGUCCAAGUCCCGGCGGCUGGAGAACCUGAUCG |
| | | CCCAGCUGCCCGGCGAGAAGAAGAACGGCCUGUUCGGCAA |
| | | CCUGAUCGCCCUGUCCCUGGGCCUGACCCCCAACUUCAAG |
| | | UCCAACUUCGACCUGGCCGAGGACGCCAAGCUGCAGCUGU |
| | | CCAAGGACACCUACGACGACGACCUGGACAACCUGCUGGC |
| | | CCAGAUCGGCGACCAGUACGCCGACCUGUUCCUGGCCGCC |
| | | AAGAACCUGUCCGACGCCAUCCUGCUGUCCGACAUCCUGC |
| | | GGGUGAACACCGAGAUCACCAAGGCCCCCCUGUCCGCCUC |
| | | CAUGAUCAAGCGGUACGACGAGCACCACCAGGACCUGACC |
| | | CUGCUGAAGGCCCUGGUGCGGCAGCAGCUGCCCGAGAAGU |
| | | ACAAGGAGAUCUUCUUCGACCAGUCCAAGAACGGCUACGC |
| | | CGGCUACAUCGACGGCGGCGCCUCCCAGGAGGAGUUCUAC |
| | | AAGUUCAUCAAGCCCAUCCUGGAGAAGAUGGACGGCACCG |
| | | AGGAGCUGCUGGUGAAGCUGAACCGGGAGGACCUGCUGC |
| | | GGAAGCAGCGGACCUUCGACAACGGCUCCAUCCCCCACCA |
| | | GAUCCACCUGGGCGAGCUGCACGCCAUCCUGCGGCGGCAG |
| | | GAGGACUUCUACCCCUUCCUGAAGGACAACCGGGAGAAGA |
| | | UCGAGAAGAUCCUGACCUUCCGGAUCCCCUACUACGUGGG |
| | | CCCCCUGGCCCGGGGCAACUCCCGGUUCGCCUGGAUGACC |
| | | CGGAAGUCCGAGGAGACCAUCACCCCCUGGAACUUCGAGG |
| | | AGGUGGUGGACAAGGGCGCCUCCGCCCAGUCCUUCAUCGA |
| | | GCGGAUGACCAACUUCGACAAGAACCUGCCCAACGAGAAG |
| | | GUGCUGCCCAAGCACUCCCUGCUGUACGAGUACUUCACCG |
| | | UGUACAACGAGCUGACCAAGGUGAAGUACGUGACCGAGG |
| | | GCAUGCGGAAGCCCGCCUUCCUGUCCGGCGAGCAGAAGAA |
| | | GGCCAUCGUGGACCUGCUGUUCAAGACCAACCGGAAGGUG |
| | | ACCGUGAAGCAGCUGAAGGAGGACUACUUCAAGAAGAUC |
| | | GAGUGCUUCGACUCCGUGGAGAUCUCCGGCGUGGAGGACC |
| | | GGUUCAACGCCUCCCUGGGCACCUACCACGACCUGCUGAA |
| | | GAUCAUCAAGGACAAGGACUUCCUGGACAACGAGGAGAA |
| | | CGAGGACAUCCUGGAGGACAUCGUGCUGACCCUGACCCUG |
| | | UUCGAGGACCGGGAGAUGAUCGAGGAGCGGCUGAAGACC |
| | | UACGCCCACCUGUUCGACGACAAGGUGAUGAAGCAGCUGA |
| | | AGCGGCGGCGGUACACCGGCUGGGGCCGGCUGUCCCGGAA |
| | | GCUGAUCAACGGCAUCCGGGACAAGCAGUCCGGCAAGACC |
| | | AUCCUGGACUUCCUGAAGUCCGACGGCUUCGCCAACCGGA |
| | | ACUUCAUGCAGCUGAUCCACGACGACUCCCUGACCUUCAA |
| | | GGAGGACAUCCAGAAGGCCCAGGUGUCCGGCCAGGGCGAC |
| | | UCCCUGCACGAGCACAUCGCCAACCUGGCCGGCUCCCCCG |
| | | CCAUCAAGAAGGGCAUCCUGCAGACCGUGAAGGUGGUGG |
| | | ACGAGCUGGUGAAGGUGAUGGGCCGGCACAAGCCCGAGA |
| | | ACAUCGUGAUCGAGAUGGCCCGGGAGAACCAGACCACCCA |
| | | GAAGGGCCAGAAGAACUCCCGGGAGCGGAUGAAGCGGAU |
| | | CGAGGAGGGCAUCAAGGAGCUGGGCUCCCAGAUCCUGAA |
| | | GGAGCACCCCGUGGAGAACACCCAGCUGCAGAACGAGAAG |
| | | CUGUACCUGUACUACCUGCAGAACGGCCGGGACAUGUACG |
| | | UGGACCAGGAGCUGGACAUCAACCGGCUGUCCGACUACGA |
| | | CGUGGACCACAUCGUGCCCCAGUCCUUCCUGAAGGACGAC |
| | | UCCAUCGACAACAAGGUGCUGACCCGGUCCGACAAGAACC |
| | | GGGGCAAGUCCGACAACGUGCCCUCCGAGGAGGUGGUGA |
| | | AGAAGAUGAAGAACUACUGGCGGCAGCUGCUGAACGCCA |
| | | AGCUGAUCACCCAGCGGAAGUUCGACAACCUGACCAAGGC |
| | | CGAGCGGGGCGGCCUGUCCGAGCUGGACAAGGCCGGCUUC |
| | | AUCAAGCGGCAGCUGGUGGAGACCCGGCAGAUCACCAAGC |
| | | ACGUGGCCCAGAUCCUGGACUCCCGGAUGAACACCAAGUA |
| | | CGACGAGAACGACAAGCUGAUCCGGGAGGUGAAGGUGAU |
| | | CACCCUGAAGUCCAAGCUGGUGUCCGACUUCCGGAAGGAC |
| | | UUCCAGUUCUACAAGGUGCGGGAGAUCAACAACUACCACC |
| | | ACGCCCACGACGCCUACCUGAACGCCGUGGUGGGCACCGC |
| | | CCUGAUCAAGAAGUACCCCAAGCUGGAGUCCGAGUUCGUG |
| | | UACGGCGACUACAAGGUGUACGACGUGCGGAAGAUGAUC |
| | | GCCAAGUCCGAGCAGGAGAUCGGCAAGGCCACCGCCAAGU |
| | | ACUUCUUCUACUCCAACAUCAUGAACUUCUUCAAGACCGA |
| | | GAUCACCCUGGCCAACGGCGAGAUCCGGAAGCGGCCCCUG |
| | | AUCGAGACCAACGGCGAGACCGGCGAGAUCGUGUGGGAC |
| | | AAGGGCCGGGACUUCGCCACCGUGCGGAAGGUGCUGUCCA |
| | | UGCCCCAGGUGAACAUCGUGAAGAAGACCGAGGUGCAGA |
| | | CCGGCGGCUUCUCCAAGGAGUCCAUCCUGCCCAAGCGGAA |
| | | CUCCGACAAGCUGAUCGCCCGGAAGAAGGACUGGGACCCC |
| | | AAGAAGUACGGCGGCUUCGACUCCCCCACCGUGGCCUACU |
| | | CCGUGCUGGUGGUGGCCAAGGUGGAGAAGGGCAAGUCCA |
| | | AGAAGCUGAAGUCCGUGAAGGAGCUGCUGGGCAUCACCA |
| | | UCAUGGAGCGGUCCUCCUUCGAGAAGAACCCCAUCGACUU |
| | | CCUGGAGGCCAAGGGCUACAAGGAGGUGAAGAAGGACCU |
| | | GAUCAUCAAGCUGCCCAAGUACUCCCUGUUCGAGCUGGAG |
| | | AACGGCCGGAAGCGGAUGCUGGCCUCCGCCGGCGAGCUGC |
| | | AGAAGGGCAACGAGCUGGCCCUGCCCUCCAAGUACGUGAA |
| | | CUUCCUGUACCUGGCCUCCCACUACGAGAAGCUGAAGGGC |
| | | UCCCCCGAGGACAACGAGCAGAAGCAGCUGUUCGUGGAGC |
| | | AGCACAAGCACUACCUGGACGAGAUCAUCGAGCAGAUCUC |
| | | CGAGUUCUCCAAGCGGGUGAUCCUGGCCGACGCCAACCUG |
| | | GACAAGGUGCUGUCCGCCUACAACAAGCACCGGGACAAGC |
| | | CCAUCCGGGAGCAGGCCGAGAACAUCAUCCACCUGUUCAC |
| | | CCUGACCAACCUGGGCGCCCCCGCCGCCUUCAAGUACUUC |
| | | GACACCACCAUCGACCGGAAGCGGUACACCUCCACCAAGG |
| | | AGGUGCUGGACGCCACCCUGAUCCACCAGUCCAUCACCGG |
| | | CCUGUACGAGACCCGGAUCGACCUGUCCCAGCUGGGCGGC |
| | | GACGGCGGCGGCUCCCCCAAGAAGAAGCGGAAGGUGUCCG |
| | | AGUCCGCCACCCCCGAGUCCGUGUCCGGCUGGCGGCUGUU |
| | | CAAGAAGAUCUCCUGA |
| Amino acid | 809 | MDKKYSIGLDIGTNSVGWAVITDEYKVPSKKFKVLGNTDRHSI |
| sequence for | | KKNLIGALLFDSGETAEATRLKRTARRRYTRRKNRICYLQEIFS |
| Cas9 encoded by | | NEMAKVDDSFFHRLEESFLVEEDKKHERHPIFGNIVDEVAYHE |
| SEQ ID Nos. | | KYPTIYHLRKKLVDSTDKADLRLIYLALAHMIKFRGHFLIEGD |
| 801-802 | | LNPDNSDVDKLFIQLVQTYNQLFEENPINASGVDAKAILSARLS |
| | | KSRRLENLIAQLPGEKKNGLFGNLIALSLGLTPNFKSNFDLAED |
| | | AKLQLSKDTYDDDLDNLLAQIGDQYADLFLAAKNLSDAILLS |
| | | DILRVNTEITKAPLSASMIKRYDEHHQDLTLLKALVRQQLPEK |
| | | YKEIFFDQSKNGYAGYIDGGASQEEFYKFIKPILEKMDGTEELL |
| | | VKLNREDLLRKQRTFDNGSIPHQIHLGELHAILRRQEDFYPFLK |
| | | DNREKIEKILTFRIPYYVGPLARGNSRFAWMTRKSEETITPWNF |
| | | EEVVDKGASAQSFIERMTNFDKNLPNEKVLPKHSLLYEYFTVY |
| | | NELTKVKYVTEGMRKPAFLSGEQKKAIVDLLFKTNRKVTVKQ |
| | | LKEDYFKKIECFDSVEISGVEDRFNASLGTYHDLLKIIKDKDFL |
| | | DNEENEDILEDIVLTLTLFEDREMIEERLKTYAHLFDDKVMKQ |
| | | LKRRRYTGWGRLSRKLINGIRDKQSGKTILDFLKSDGFANRNF |
| | | MQLIHDDSLTFKEDIQKAQVSGQGDSLHEHIANLAGSPAIKKGI |
| | | LQTVKVVDELVKVMGRHKPENIVIEMARENQTTQKGQKNSRE |
| | | RMKRIEEGIKELGSQILKEHPVENTQLQNEKLYLYYLQNGRDM |
| | | YVDQELDINRLSDYDVDHIVPQSFLKDDSIDNKVLTRSDKNRG |
| | | KSDNVPSEEVVKKMKNYWRQLLNAKLITQRKFDNLTKAERG |
| | | GLSELDKAGFIKRQLVETRQITKHVAQILDSRMNTKYDENDKL |
| | | IREVKVITLKSKLVSDFRKDFQFYKVREINNYHHAHDAYLNAV |
| | | VGTALIKKYPKLESEFVYGDYKVYDVRKMIAKSEQEIGKATA |
| | | KYFFYSNIMNFFKTEITLANGEIRKRPLIETNGETGEIVWDKGR |
| | | DFATVRKVLSMPQVNIVKKTEVQTGGFSKESILPKRNSDKLIA |
| | | RKKDWDPKKYGGFDSPTVAYSVLVVAKVEKGKSKKLKSVKE |
| | | LLGITIMERSSFEKNPIDFLEAKGYKEVKKDLIIKLPKYSLFELE |
| | | NGRKRMLASAGELQKGNELALPSKYVNFLYLASHYEKLKGSP |
| | | EDNEQKQLFVEQHKHYLDEIIEQISEFSKRVILADANLDKVLSA |
| | | YNKHRDKPIREQAENIIHLFTLTNLGAPAAFKYFDTTIDRKRYT |
| | | STKEVLDATLIHQSITGLYETRIDLSQLGGDGGGSPKKKRKV |
| Amino acid | 810 | MDKKYSIGLDIGTNSVGWAVITDEYKVPSKKFKVLGNTDRHSI |
| sequence for | | KKNLIGALLFDSGETAEATRLKRTARRRYTRRKNRICYLQEIFS |
| Cas9 with Hibit | | NEMAKVDDSFFHRLEESFLVEEDKKHERHPIFGNIVDEVAYHE |
| tag | | KYPTIYHLRKKLVDSTDKADLRLIYLALAHMIKFRGHFLIEGD |
| | | LNPDNSDVDKLFIQLVQTYNQLFEENPINASGVDAKAILSARLS |
| | | KSRRLENLIAQLPGEKKNGLFGNLIALSLGLTPNFKSNFDLAED |
| | | AKLQLSKDTYDDDLDNLLAQIGDQYADLFLAAKNLSDAILLS |
| | | DILRVNTEITKAPLSASMIKRYDEHHQDLTLLKALVRQQLPEK |
| | | YKEIFFDQSKNGYAGYIDGGASQEEFYKFIKPILEKMDGTEELL |
| | | VKLNREDLLRKQRTFDNGSIPHQIHLGELHAILRRQEDFYPFLK |
| | | DNREKIEKILTFRIPYYVGPLARGNSRFAWMTRKSEETITPWNF |
| | | EEVVDKGASAQSFIERMTNFDKNLPNEKVLPKHSLLYEYFTVY |
| | | NELTKVKYVTEGMRKPAFLSGEQKKAIVDLLFKTNRKVTVKQ |
| | | LKEDYFKKIECFDSVEISGVEDRFNASLGTYHDLLKIIKDKDFL |
| | | DNEENEDILEDIVLTLTLFEDREMIEERLKTYAHLFDDKVMKQ |
| | | LKRRRYTGWGRLSRKLINGIRDKQSGKTILDFLKSDGFANRNF |
| | | MQLIHDDSLTFKEDIQKAQVSGQGDSLHEHIANLAGSPAIKKGI |
| | | LQTVKVVDELVKVMGRHKPENIVIEMARENQTTQKGQKNSRE |
| | | RMKRIEEGIKELGSQILKEHPVENTQLQNEKLYLYYLQNGRDM |
| | | YVDQELDINRLSDYDVDHIVPQSFLKDDSIDNKVLTRSDKNRG |
| | | KSDNVPSEEVVKKMKNYWRQLLNAKLITQRKFDNLTKAERG |
| | | GLSELDKAGFIKRQLVETRQITKHVAQILDSRMNTKYDENDKL |
| | | IREVKVITLKSKLVSDFRKDFQFYKVREINNYHHAHDAYLNAV |
| | | VGTALIKKYPKLESEFVYGDYKVYDVRKMIAKSEQEIGKATA |
| | | KYFFYSNIMNFFKTEITLANGEIRKRPLIETNGETGEIVWDKGR |
| | | DFATVRKVLSMPQVNIVKKTEVQTGGFSKESILPKRNSDKLIA |
| | | RKKDWDPKKYGGFDSPTVAYSVLVVAKVEKGKSKKLKSVKE |
| | | LLGITIMERSSFEKNPIDFLEAKGYKEVKKDLIIKLPKYSLFELE |
| | | NGRKRMLASAGELQKGNELALPSKYVNFLYLASHYEKLKGSP |
| | | EDNEQKQLFVEQHKHYLDEIIEQISEFSKRVILADANLDKVLSA |
| | | YNKHRDKPIREQAENIIHLFTLTNLGAPAAFKYFDTTIDRKRYT |
| | | STKEVLDATLIHQSITGLYETRIDLSQLGGDGGGSPKKKRKVSE |
| | | SATPESVSGWRLFKKIS |
| Open reading | 817 | AUGGACAAGAAGUACAGCAUCGGACUGGACAUCGGAACA |
| frame for Cas9 | | AACAGCGUCGGAUGGGCAGUCAUCACAGACGAAUACAAG |
| | | GUCCCGAGCAAGAAGUUCAAGGUCCUGGGAAACACAGAC |
| | | AGACACAGCAUCAAGAAGAACCUGAUCGGAGCACUGCUG |
| | | UUCGACAGCGGAGAAACAGCAGAAGCAACAAGACUGAAG |
| | | AGAACAGCAAGAAGAAGAUACACAAGAAGAAAGAACAGA |
| | | AUCUGCUACCUGCAGGAAAUCUUCAGCAACGAAAUGGCA |
| | | AAGGUCGACGACAGCUUCUUCCACAGACUGGAAGAAAGC |
| | | UUCCUGGUCGAAGAAGACAAGAAGCACGAAAGACACCCG |
| | | AUCUUCGGAAACAUCGUCGACGAAGUCGCAUACCACGAAA |
| | | AGUACCCGACAAUCUACCACCUGAGAAAGAAGCUGGUCGA |
| | | CAGCACAGACAAGGCAGACCUGAGACUGAUCUACCUGGCA |
| | | CUGGCACACAUGAUCAAGUUCAGAGGACACUUCCUGAUCG |
| | | AAGGAGACCUGAACCCGGACAACAGCGACGUCGACAAGCU |
| | | GUUCAUCCAGCUGGUCCAGACAUACAACCAGCUGUUCGAA |
| | | GAAAACCCGAUCAACGCAAGCGGAGUCGACGCAAAGGCAA |
| | | UCCUGAGCGCAAGACUGAGCAAGAGCAGAAGACUGGAAA |
| | | ACCUGAUCGCACAGCUGCCGGGAGAAAAGAAGAACGGAC |
| | | UGUUCGGAAACCUGAUCGCACUGAGCCUGGGACUGACACC |
| | | GAACUUCAAGAGCAACUUCGACCUGGCAGAAGACGCAAA |
| | | GCUGCAGCUGAGCAAGGACACAUACGACGACGACCUGGAC |
| | | AACCUGCUGGCACAGAUCGGAGACCAGUACGCAGACCUGU |
| | | UCCUGGCAGCAAAGAACCUGAGCGACGCAAUCCUGCUGAG |
| | | CGACAUCCUGAGAGUCAACACAGAAAUCACAAAGGCACCG |
| | | CUGAGCGCAAGCAUGAUCAAGAGAUACGACGAACACCACC |
| | | AGGACCUGACACUGCUGAAGGCACUGGUCAGACAGCAGCU |
| | | GCCGGAAAAGUACAAGGAAAUCUUCUUCGACCAGAGCAA |
| | | GAACGGAUACGCAGGAUACAUCGACGGAGGAGCAAGCCA |
| | | GGAAGAAUUCUACAAGUUCAUCAAGCCGAUCCUGGAAAA |
| | | GAUGGACGGAACAGAAGAACUGCUGGUCAAGCUGAACAG |
| | | AGAAGACCUGCUGAGAAAGCAGAGAACAUUCGACAACGG |
| | | AAGCAUCCCGCACCAGAUCCACCUGGGAGAACUGCACGCA |
| | | AUCCUGAGAAGACAGGAAGACUUCUACCCGUUCCUGAAG |
| | | GACAACAGAGAAAAGAUCGAAAAGAUCCUGACAUUCAGA |
| | | AUCCCGUACUACGUCGGACCGCUGGCAAGAGGAAACAGCA |
| | | GAUUCGCAUGGAUGACAAGAAAGAGCGAAGAAACAAUCA |
| | | CACCGUGGAACUUCGAAGAAGUCGUCGACAAGGGAGCAA |
| | | GCGCACAGAGCUUCAUCGAAAGAAUGACAAACUUCGACA |
| | | AGAACCUGCCGAACGAAAAGGUCCUGCCGAAGCACAGCCU |
| | | GCUGUACGAAUACUUCACAGUCUACAACGAACUGACAAA |
| | | GGUCAAGUACGUCACAGAAGGAAUGAGAAAGCCGGCAUU |
| | | CCUGAGCGGAGAACAGAAGAAGGCAAUCGUCGACCUGCU |
| | | GUUCAAGACAAACAGAAAGGUCACAGUCAAGCAGCUGAA |
| | | GGAAGACUACUUCAAGAAGAUCGAAUGCUUCGACAGCGU |
| | | CGAAAUCAGCGGAGUCGAAGACAGAUUCAACGCAAGCCU |
| | | GGGAACAUACCACGACCUGCUGAAGAUCAUCAAGGACAA |
| | | GGACUUCCUGGACAACGAAGAAAACGAAGACAUCCUGGA |
| | | AGACAUCGUCCUGACACUGACACUGUUCGAAGACAGAGA |
| | | AAUGAUCGAAGAAAGACUGAAGACAUACGCACACCUGUU |
| | | CGACGACAAGGUCAUGAAGCAGCUGAAGAGAAGAAGAUA |
| | | CACAGGAUGGGGAAGACUGAGCAGAAAGCUGAUCAACGG |
| | | AAUCAGAGACAAGCAGAGCGGAAAGACAAUCCUGGACUU |
| | | CCUGAAGAGCGACGGAUUCGCAAACAGAAACUUCAUGCA |
| | | GCUGAUCCACGACGACAGCCUGACAUUCAAGGAAGACAUC |
| | | CAGAAGGCACAGGUCAGCGGACAGGGAGACAGCCUGCACG |
| | | AACACAUCGCAAACCUGGCAGGAAGCCCGGCAAUCAAGAA |
| | | GGGAAUCCUGCAGACAGUCAAGGUCGUCGACGAACUGGU |
| | | CAAGGUCAUGGGAAGACACAAGCCGGAAAACAUCGUCAU |
| | | CGAAAUGGCAAGAGAAAACCAGACAACACAGAAGGGACA |
| | | GAAGAACAGCAGAGAAAGAAUGAAGAGAAUCGAAGAAGG |
| | | AAUCAAGGAACUGGGAAGCCAGAUCCUGAAGGAACACCC |
| | | GGUCGAAAACACACAGCUGCAGAACGAAAAGCUGUACCU |
| | | GUACUACCUGCAGAACGGAAGAGACAUGUACGUCGACCA |
| | | GGAACUGGACAUCAACAGACUGAGCGACUACGACGUCGAC |
| | | CACAUCGUCCCGCAGAGCUUCCUGAAGGACGACAGCAUCG |
| | | ACAACAAGGUCCUGACAAGAAGCGACAAGAACAGAGGAA |
| | | AGAGCGACAACGUCCCGAGCGAAGAAGUCGUCAAGAAGA |
| | | UGAAGAACUACUGGAGACAGCUGCUGAACGCAAAGCUGA |
| | | UCACACAGAGAAAGUUCGACAACCUGACAAAGGCAGAGA |
| | | GAGGAGGACUGAGCGAACUGGACAAGGCAGGAUUCAUCA |
| | | AGAGACAGCUGGUCGAAACAAGACAGAUCACAAAGCACG |
| | | UCGCACAGAUCCUGGACAGCAGAAUGAACACAAAGUACG |
| | | ACGAAAACGACAAGCUGAUCAGAGAAGUCAAGGUCAUCA |
| | | CACUGAAGAGCAAGCUG |
| | | GUCAGCGACUUCAGAAAGGACUUCCAGUUCUACAAGGUC |
| | | AGAGAAAUCAACAACUACCACCACGCACACGACGCAUACC |
| | | UGAACGCAGUCGUCGGAACAGCACUGAUCAAGAAGUACCC |
| | | GAAGCUGGAAAGCGAAUUCGUCUACGGAGACUACAAGGU |
| | | CUACGACGUCAGAAAGAUGAUCGCAAAGAGCGAACAGGA |
| | | AAUCGGAAAGGCAACAGCAAAGUACUUCUUCUACAGCAA |
| | | CAUCAUGAACUUCUUCAAGACAGAAAUCACACUGGCAAAC |
| | | GGAGAAAUCAGAAAGAGACCGCUGAUCGAAACAAACGGA |
| | | GAAACAGGAGAAAUCGUCUGGGACAAGGGAAGAGACUUC |
| | | GCAACAGUCAGAAAGGUCCUGAGCAUGCCGCAGGUCAACA |
| | | UCGUCAAGAAGACAGAAGUCCAGACAGGAGGAUUCAGCA |
| | | AGGAAAGCAUCCUGCCGAAGAGAAACAGCGACAAGCUGA |
| | | UCGCAAGAAAGAAGGACUGGGACCCGAAGAAGUACGGAG |
| | | GAUUCGACAGCCCGACAGUCGCAUACAGCGUCCUGGUCGU |
| | | CGCAAAGGUCGAAAAGGGAAAGAGCAAGAAGCUGAAGAG |
| | | CGUCAAGGAACUGCUGGGAAUCACAAUCAUGGAAAGAAG |
| | | CAGCUUCGAAAAGAACCCGAUCGACUUCCUGGAAGCAAAG |
| | | GGAUACAAGGAAGUCAAGAAGGACCUGAUCAUCAAGCUG |
| | | CCGAAGUACAGCCUGUUCGAACUGGAAAACGGAAGAAAG |
| | | AGAAUGCUGGCAAGCGCAGGAGAACUGCAGAAGGGAAAC |
| | | GAACUGGCACUGCCGAGCAAGUACGUCAACUUCCUGUACC |
| | | UGGCAAGCCACUACGAAAAGCUGAAGGGAAGCCCGGAAG |
| | | ACAACGAACAGAAGCAGCUGUUCGUCGAACAGCACAAGCA |
| | | CUACCUGGACGAAAUCAUCGAACAGAUCAGCGAAUUCAGC |
| | | AAGAGAGUCAUCCUGGCAGACGCAAACCUGGACAAGGUCC |
| | | UGAGCGCAUACAACAAGCACAGAGACAAGCCGAUCAGAG |
| | | AACAGGCAGAAAACAUCAUCCACCUGUUCACACUGACAAA |
| | | CCUGGGAGCACCGGCAGCAUUCAAGUACUUCGACACAACA |
| | | AUCGACAGAAAGAGAUACACAAGCACAAAGGAAGUCCUG |
| | | GACGCAACACUGAUCCACCAGAGCAUCACAGGACUGUACG |
| | | AAACAAGAAUCGACCUGAGCCAGCUGGGAGGAGACGGAG |
| | | GAGGAAGCCCGAAGAAGAAGAGAAAGGUCUAG |

Phosphorothioate (PS) linkage or bond refers to a bond where a sulfur is substituted for one nonbridging phosphate oxygen in a phosphodiester linkage, for example in the bonds between nucleotides bases. When phosphorothioates are used to generate oligonucleotides, the modified oligonucleotides may also be referred to as S-oligos.

A “*” may be used to depict a PS modification. In this application, the terms A*, C*, U*, or G* may be used to denote a nucleotide that is linked to the next (e.g., 3′) nucleotide with a PS bond.

The terms “mA,” “mC,” “mU,” or “mG” may be used to denote a nucleotide that has been modified with 2′-O-Methyl (2′-O-Me).

Modification of 2′-O-methyl can be depicted as follows:

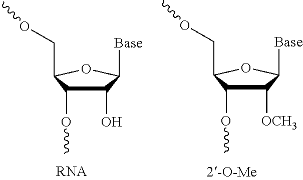

We claim:

1. An anti-CD30 chimeric antigen receptor (CAR) comprising:
(a) a binder domain that binds CD30;
(b) a hinge domain comprising a hinge sequence selected from a CD8a hinge sequence and a CD28 hinge sequence;
(c) a transmembrane domain comprising a transmembrane sequence selected from a CD28 transmembrane sequence and a CD8a transmembrane sequence;
(d) a costimulatory domain comprising a costimulatory sequence selected from a CD28 costimulatory sequence; and a 41BB costimulatory sequence; and
(e) an activation domain comprising a CD3z activation sequence.

2. An anti-CD30 chimeric antigen receptor (CAR) comprising:
(a) a binder domain that binds CD30;
(b) a CD8a hinge domain;
(c) a CD28 transmembrane domain;
(d) a costimulatory domain; and
(e) an activation domain.

3. The CAR of claim 1 or 2, wherein the binder domain comprises a CD30 antibody or antigen binding fragment thereof comprising:
(a) a heavy chain variable region (VH) complementarity-determining region 1 (CDR1) sequence TFTTYT (SEQ ID NO: 3);
(b) a VH CDR2 sequence INPSSGCSD (SEQ ID NO: 4) or INPSSGYSD (SEQ ID NO: 5);
(c) a VH CDR3 sequence RADYGNYEYTWFAY (SEQ ID NO: 6);
(d) a light chain variable region (VL) CDR1 sequence ASQNVGTNVA (SEQ ID NO: 7);
(e) a VL CDR2 sequence SASYRYS (SEQ ID NO: 8); and
(f) a VL CDR3 sequence QQYHTYP (SEQ ID NO: 9).

4. The CAR of claim 3, wherein the VH amino acid sequence has at least 80% identity to sequence SEQ ID NO: 24 and the VL amino acid sequence has at least 80% identity to SEQ ID NO: 25.

5. The CAR of claim 3, wherein the VH amino acid sequence has at least 80% identity to sequence SEQ ID NO: 26 and the VL amino acid sequence has at least 80% identity to SEQ ID NO: 27.

6. The CAR of claim 3, wherein the VH amino acid sequence has at least 80% identity to sequence SEQ ID NO: 28 and the VL amino acid sequence has at least 80% identity to SEQ ID NO: 25.

7. The CAR of claim 3, wherein the VH amino acid sequence has at least 80% identity to sequence SEQ ID NO: 29 and the VL amino acid sequence has at least 80% identity to SEQ ID NO: 30.

8. The CAR of claim 1 or 2, wherein the binder domain is HRS3.

9. The CAR of claim 1 or 2, wherein the binder domain comprises a CD30 antibody or antigen binding fragment thereof comprising a heavy chain variable region (VH) complementarity-determining region 1 (CDR1) sequence AYYWS (SEQ ID NO: 10); a VH CDR2 sequence DINHGGGTNYNPSLKS (SEQ ID NO: 11); a VH CDR3 sequence LTAY (SEQ ID NO: 12); a light chain variable region (VL) CDR1 sequence RASQGISSWLT (SEQ ID NO: 13); a VL CDR2 sequence AASSLQS (SEQ ID NO: 14); and a VL CDR3 sequence QQYDSYPIT (SEQ ID NO: 15).

10. The CAR of claim 9, wherein the VH amino acid sequence has at least 80% identity to sequence SEQ ID NO: 31 and the VL amino acid sequence has at least 80% identity to SEQ ID NO: 32.

11. The CAR of claim 1 or 2, wherein the binder domain is 5F11.

12. The CAR of claim 1 or 2, wherein the binder domain comprises a sequence selected from the binder domain sequences listed in Table 1 or 1A.

13. The CAR of any one of claims 1 or 3-12, wherein the hinge domain comprises a sequence selected from the hinge domain sequences listed in Table 2.

14. The CAR of any one of claims 1-13, further comprising a linker between the hinge domain and the transmembrane domain.

15. The CAR of claim 14, wherein the linker comprises a sequence of KPDK (SEQ ID NO: 16).

16. The CAR of any one of claims 1-15, wherein the transmembrane domain comprises the sequence of SEQ ID NO: 54.

17. The CAR of any one of claims 1-16, wherein the costimulatory domain is a CD28 costimulatory domain.

18. The CAR of claim 17, wherein the CD28 costimulatory domain is a wild-type CD28 costimulatory domain.

19. The CAR of claim 17, wherein the CD28 costimulatory domain comprises the sequence of SEQ ID NO: 58.

20. The CAR of any one of claims 1-19, wherein the costimulatory domain comprises a sequence selected from the costimulatory domain sequences listed in Table 4.

21. The CAR of any one of claims 1-20, wherein the activation domain is a wild-type CD3z activation domain having an N-terminal arginine or an N terminal leucine.

22. The CAR of any one of claims 1-20, wherein the CD3z activation domain comprises a sequence selected from the activation domain sequences listed in Table 5.

23. The CAR of any one of claims 1-22, wherein: the hinge domain comprises a CD8a hinge sequence; the transmembrane domain comprises a CD28 transmembrane sequence; the costimulatory domain comprises a wild-type CD28 costimulatory sequence; and the activation domain comprises a wild-type CD3z activation sequence comprising an N-terminal leucine.

24. The CAR of claim 23, wherein the CD8a hinge sequence comprises the sequence of SEQ ID NO: 50; the CD28 transmembrane sequence comprises the sequence of SEQ ID NO: 54; the CD28 costimulatory sequence comprises the sequence of SEQ ID NO: 57; and the CD3z activation sequence comprises the sequence of SEQ ID NO: 61.

25. The CAR of claim 24, wherein the binder domain comprises the sequence of SEQ ID NO: 20.

26. The CAR of claim 24, wherein the binder domain comprises the sequence of SEQ ID NO: 21.

27. The CAR of claim 24, wherein the binder domain comprises the sequence of SEQ ID NO: 22.

28. The CAR of any one of claims 1-12, wherein: the hinge domain comprises a CD8a hinge sequence; the transmembrane domain comprises a CD28 transmembrane sequence; the costimulatory domain comprises a wild-type CD28 costimulatory sequence; and the activation domain comprises a wild-type CD3z activation sequence having an N-terminal argenine.

29. The CAR of claim 28, wherein the CD8a hinge sequence comprises the sequence of SEQ ID NO: 50; the CD28 transmembrane sequence comprises the sequence of SEQ ID NO: 54; the CD28 costimulatory sequence comprises the sequence of SEQ ID NO: 57; and the CD3z activation sequence comprises the sequence of SEQ ID NO: 60.

30. The CAR of any one of claims 1-12, wherein: the hinge domain comprises a CD8a hinge sequence; the transmembrane domain comprises a CD28 transmembrane sequence; the costimulatory domain comprises a CD28 costimulatory sequence; and the activation domain comprises a modified CD3z activation sequence.

31. The CAR of claim 30, wherein the CD8a hinge sequence comprises the sequence of SEQ ID NO: 50; the CD28 transmembrane sequence comprises the sequence of SEQ ID NO: 54; the CD28 costimulatory sequence comprises the sequence of SEQ ID NO: 57; and the CD3z activation sequence comprises the sequence of SEQ ID NO:62.

32. The CAR of any one of claims 1-12, wherein: the hinge domain comprises a CD8a hinge sequence; the transmembrane domain comprises a CD8a transmembrane sequence; the costimulatory domain comprises a 41BB costimulatory sequence; and the activation domain comprises a modified CD3z activation sequence.

33. The CAR of claim 32, wherein the CD8a hinge sequence comprises the sequence of SEQ ID NO: 50; the CD8a transmembrane sequence comprises the sequence of SEQ ID NO: 55; the 41BB costimulatory sequence comprises the sequence of SEQ ID NO: 59; and the CD3z activation sequence comprises the sequence of SEQ ID NO: 62.

34. The CAR of any one of claims 29, 32, or 33, wherein the binder domain comprises the sequence of SEQ ID NO: 23.

35. The CAR of any one of claims 1-34, wherein the binder domain is an scFv.

36. The CAR of claim 1 comprising a sequence selected from the CAR sequences listed in Table 6.

37. The CAR of claim 1 comprising a sequence selected from the CAR sequences SEQ ID NO: 65, SEQ ID NO: 66, SEQ ID NO: 67, SEQ ID NO: 68, SEQ ID NO: 69, SEQ ID NO: 71, SEQ ID NO: 72, SEQ ID NO: 73, SEQ ID NO: 74.

38. An engineered cell comprising a CAR of any one of claims 1-37.

39. The engineered cell of claim 38, wherein the engineered cell is derived from a T cell or a NK cell.

40. The engineered cell of claim 39, wherein the engineered cell is derived from a T cell.

41. The engineered cell of claim 40, wherein the engineered cell has reduced expression of a T cell receptor (TCR) on its surface relative to the T cell from which it was derived.

42. The engineered cell of any one of claims 38-41, wherein the engineered cell expresses the CAR at an endogenous TRAC locus of the engineered cell.

43. The engineered cell of claim 42, wherein the nucleic acid sequence encoding the CAR disrupts the coding sequence of a TCR in the TRAC locus.

44. The engineered cell of any one of claims 38-43, wherein the engineered cell does not express a TCR.

45. The engineered cell of any one of claims 38-44, wherein the engineered cell comprises at least one genetic modification in a MHC class II gene.

46. The engineered cell of claim 45, wherein the engineered cell does not express the MHC class II gene.

47. The engineered cell of claim 45 or 46, wherein the MHC class II gene is a HLA-DM gene, a HLA-DO gene, a HLA-DP gene, a HLA-DQ gene, or a HLA-DR gene.

48. The engineered cell of any one of claims 38-47, wherein the engineered cell comprises at least one genetic modification in a CIITA gene.

49. The engineered cell of claim 48, wherein the cell does not express a functional CIITA protein.

50. The engineered cell of claim 48, wherein the cell is homozygous for HLA-B and homozygous for HLA-C.

51. The engineered cell of any one of claims 40-49, wherein the engineered cell comprises at least one modification in a MHC class I gene.

52. The engineered cell of claim 51, wherein the MHC class I gene is a HLA-A gene, a HLA-B gene, or a HLA-C gene, or a combination thereof.

53. The engineered cell of claim 52, wherein the cell does not express the HLA-A gene.

54. The engineered cell of claim 52 or claim 53, wherein the HLA-B gene and the HLA-C gene are matched to a subject who is to be administered the engineered cell.

55. A population of cells comprising the engineered cell of any one of claims 38-54.

56. A pharmaceutical composition comprising the engineered cell of any one of claims 38-54 or the population of cells of claim 55.

57. A method of treating a disease or disorder in a subject, the method comprising administering to the subject the engineered cell, population of cells, or pharmaceutical composition of any one of claims 38-56 to the subject.

58. The method of claim 57, wherein the engineered cell is homozygous for HLA-B and homozygous for HLA-C.

59. The method of claim 57 or 58, wherein the disease or disorder is a cancer, an infectious disease, or an autoimmune disease.

60. The method of claim 59, wherein the disease or disorder is a cancer.

61. The method of claim 60, wherein the cancer is a hematologic cancer.

62. The method of claim 61, wherein the hematologic cancer is a CD30-expressing hematologic cancer.

63. The method of claim 62, wherein the CD30-expressing hematologic cancer is relapsed or refractory classical Hodgkin Lymphoma.

64. A method of preventing or reducing graft versus host disease in a subject receiving an allogenic cell treatment, the method comprising administering to the subject the engineered cell, population of cells, or pharmaceutical composition of any one of claims 38-56.

65. The engineered cell, population of cells, or pharmaceutical composition of any one of claims 38-56, for use in an adoptive cell transfer (ACT) therapy.

66. A nucleic acid encoding the CAR of any one of claims 1-37.

67. A vector comprising a nucleic acid encoding a CAR of any one of claims 1-37.

68. A cell comprising the nucleic acid of claim 66 or the vector of claim 67.

69. A method of making a CAR expressing engineered cell, the method comprising delivering a vector of claim 67 to a donor cell.

70. The method of claim 69 further comprising delivering to the donor cell a gRNA targeting a locus for inserting into the locus the nucleic acid sequence encoding the CAR.

71. The method of claim 69 or 70, further comprising delivering to the donor cell a nuclease or a nucleic acid encoding a nuclease.

72. The method of claim 71, wherein the nuclease is a Cas9 nuclease.

73. The method of any one of claim 69-72, further comprising delivering to the donor cell a gRNA that targets the HLA-A gene.

74. The method of any one of claims 69-73 further comprising delivering to the cell a gRNA that targets the CIITA gene.

75. The method of any one of claims 69-74, further comprising delivering to the donor cell a gRNA that targets the TRAC or TRBC locus.

76. The method of any one of claims 68-75 further comprising delivering to the cell a gRNA that targets the B2M gene.

77. A cell generated according to the method of any one of claims 68-76.
